(19) United States
(12) Patent Application Publication
(10) Pub. No.: US 2008/0269193 A1
(43) Pub. Date: Oct. 30, 2008

(54) Tetrahydroindole and Tetrahydroindazole Derivatives

(76) Inventors: Kenneth He Huang, Chapel Hill, NC (US); Andy J. Ommen, Durham, NC (US); Thomas E. Barta, Carrboro, NC (US); Philip F. Hughes, Chapel Hill, NC (US); James Veal, Apex, NC (US); Wei Ma, Cary, NC (US); Emilie D. Smith, Apex, NC (US); Angela R. Woodward, Durham, NC (US); W. Stephen McCall, Raleigh, NC (US)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 12/102,128

(22) Filed: Apr. 14, 2008

Related U.S. Application Data
(60) Provisional application No. 60/912,083, filed on Apr. 16, 2007.
(60) Provisional application No. 61/017,469, filed on Dec. 28, 2007.
(60) Provisional application No. 61/019,732, filed on Jan. 8, 2008.

(51) Int. Cl. A61K 31/55; A61K 31/404; C07D 405/12; A61K 31/416; A61P 35/00; C07D 231/54; C07D 403/14

(52) U.S. Cl. 514/212.03; 548/465; 514/414; 514/406; 548/360.1; 540/524

(57) ABSTRACT

The invention provides indole and indazole compounds of Formula (I)

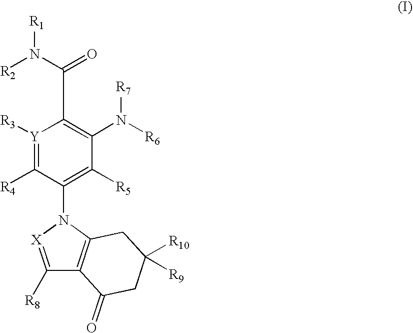

or pharmaceutically acceptable salts thereof which are useful for treating and/or preventing diseases and/or disorders ameliorated by the inhibition of Heat-Shock Protein 90. The invention further provides pharmaceutical compositions comprising compounds of Formula (I) and methods of preparing compounds of Formula (I).

### CROSS-REFERENCE TO RELATED APPLICATIONS

This application claims priority of U.S. Provisional Application No. 60/912,083, filed Apr. 16, 2007, U.S. Provisional Application No. 61/017,469, filed Dec. 28, 2007, and U.S. Provisional Application No. 61/019,732, filed Jan. 8, 2008.

### BACKGROUND OF THE INVENTION

1. Field of the Invention

The invention relates to tetrahydroinoles and tetrahydroindazoles, and pharmaceutical compositions thereof, useful in the treatment and/or prevention of diseases and/or conditions ameliorated by inhibition of heat-shock protein 90 (“HSP-90”) such as cancer, inflammation and inflammation-associated disorders, and conditions associated with angiogenesis. Compounds of the invention are also useful in the treatment and/or prevention of infectious diseases, in particular, fungal and viral infections. The invention also relates to methods of preparing compounds of the invention.

2. Description of the Related Art

Cancer is characterized by abnormal cellular proliferation. Cancer cells exhibit a number of properties that make them dangerous to the host, typically including an ability to invade other tissues and to induce capillary ingrowth, which assures that the proliferating cancer cells have an adequate supply of blood. A hallmark of cancerous cells is their abnormal response to control mechanisms that regulate cell division in normal cells and continue to divide until they ultimately kill the host.

Angiogenesis is a highly regulated process under normal conditions, however many diseases are driven by persistent unregulated angiogenesis. Unregulated angiogenesis may either cause a particular disease directly or exacerbate an existing pathological condition. For example, ocular neovascularization has not only been implicated as the most common cause of blindness, but also is believed the dominant cause of many eye diseases. Further, in certain existing conditions, for example arthritis, newly formed capillary blood vessels invade the joints and destroy cartilage, or in the case of diabetes, new capillaries formed in the retina invade the vitreous, bleed, and cause blindness. Growth and metastasis of solid tumors are also dependent on angiogenesis (Folkman, J., Cancer Res., 46, 467-473 1986; and Folkman, J., Journal of the National Cancer Institute, 82, 4-6 1989). It has been shown, for example, that tumors which enlarge to greater than 2 mm must obtain their own blood supply and do so by inducing the growth of new capillary blood vessels. Once these new blood vessels become embedded in the tumor, they provide a means for tumor cells to enter the circulation and metastasize to distant sites such as liver, lung or bone (Weidner, N., et al. The New England Journal of Medicine, 324(1), 1-8 1991). Under conditions of unregulated angiogenesis, therapeutic methods designed to control, repress, and/or inhibit angiogenesis could lead to the abrogation or mitigation of these conditions and diseases.

Inflammation is related to a variety of disorders such as pain, headaches, fever, arthritis, asthma, bronchitis, menstrual cramps, tendonitis, bursitis, psoriasis, eczema, burns, dermatitis, inflammatory bowel syndrome, Crohn's disease, gastritis, irritable bowel syndrome, ulcerative colitis, vascular diseases, Hodgkin's disease, sclerodoma, rheumatic fever, type I diabetes, myasthenia gravis, sarcoidosis, nephrotic syndrome, Behcet's syndrome, polymyositis, hypersensitivity, conjunctivitis, gingivitis, post-injury swelling, myocardial ischemia, and the like.

Heat-shock protein 90 (HSP-90) is a cellular chaperone protein required for the activation of several eukaryotic protein kinases, including the cyclin-dependent kinase CDK4. Geldanamycin, an inhibitor of the protein-refolding activity of HSP-90, has been shown to have antiproliferative and antitumor activities.

HSP-90 also guides the intracellular disposition and proteolytic turnover of many key regulators of cell growth and survival. Its function is subverted during oncogenesis to make malignant transformation possible and to facilitate rapid somatic evolution, and to allow mutant proteins to retain or even gain function. Inhibition of HSP-90 slows these processes and thus has therapeutic use (Whitesell L, Lindquist, S. L., Nature Rev. Cancer, 2005, 10, 761-72).

Ansamycin antibiotics, e.g., herbimycin A (HA), geldanamycin (GM), and 17-allylaminogeldanamycin (17-AAG) are thought to exert their anticancerous effects by tight binding of the N-terminus pocket of HSP-90, thereby destabilizing substrates that normally interact with HSP-90 (Stebbins, C. et al. Cell 1997, 89, 239-250). This pocket is highly conserved and has weak homology to the ATP-binding site of DNA gyrase (Stebbins, C. et al., supra; Grenert, J. P. et al. J. Biol. Chem. 1997, 272, 23843-50).

In vitro and in vivo studies have demonstrated that occupancy of this N-terminal pocket by ansamycins and other HSP-90 inhibitors alters HSP-90 function and inhibits protein folding. At high concentrations, ansamycins and other HSP-90 inhibitors have been shown to prevent binding of protein substrates to HSP-90 (Scheibel, T. H. et al. Proc. Natl. Acad. Sci. USA 1999, 96, 1297-302; Schulte, T. W. et al. J. Biol. Chem. 1995, 270, 24585-8; Whitesell, L. et al. Proc. Natl. Acad. Sci. USA 1994, 91, 8324-8328). Ansamycins have also been demonstrated to inhibit the ATP-dependent release of chaperone-associated protein substrates (Schneider, C. L. et al. Proc. Natl. Acad. Sci., USA 1996, 93, 14536-41; Sepp-Lorenzinoet et al. J. Biol. Chem. 1995, 270, 16580-16587). In either event, the substrates are degraded by a ubiquitin-dependent process in the proteasome (Schneider, C. L., supra; Sepp-Lorenzino, L., et al. J. Biol. Chem. 1995, 270, 16580-16587; Whitesell, L. et al. Proc. Natl. Acad. Sci. USA 1994, 91, 8324-8328). HSP-90 substrate destabilization occurs in tumor and non-transformed cells alike and has been shown to be especially effective on a subset of signaling regulators, e.g., Raf (Schulte, T. W. et al., Biochem. Biophys. Res. Commun. 1997, 239, 655-9 Schulte, T. W., et al., J. Biol. Chem. 1995, 270, 24585-8), nuclear steroid receptors (Segnitz, B.; U. Gehring J. Biol. Chem. 1997, 272, 18694-18701; Smith, D. F. et al. Mol. Cell. Biol. 1995, 15, 6804-12), v-Src (Whitesell, L. et al. Proc. Natl. Acad. Sci. USA 1994, 91, 8324-8328) and certain transmembrane tyrosine kinases (Sepp-Lorenzino, L. et al. J. Biol. Chem. 1995, 270, 16580-16587) such as EGF receptor (EGFR) and HER2/Neu (Hartmann, F., et al. Int. J. Cancer 1997, 70, 221-9; Miller, P. et al. Cancer Res. 1994, 54, 2724-2730; Mimnaugh, E. G., et al. J. Biol. Clzem. 1996, 271, 22796-801; Schnur, R. et al. J. Med. Chenu. 1995, 38, 3806-3812), CDK4, and mutant p53 (Erlichman et al. Proc. AACR 2001, 42, abstract 4474). The ansamycin-induced loss of these proteins leads to the selective disruption of certain regulatory pathways and results in growth arrest at specific phases of the cell cycle (Muise-Heimericks, R. C. et al. J. Biol. Chem. 1998, 273, 29864-72), and apoptosis, and/or differentiation of cells so treated (Vasilevskaya, A. et al. Cancer Res., 1999, 59, 3935-40). Therefore, compounds of the invention, as inhibitors of HSP-90, are useful for the treatment and/or prevention of many types of cancers and proliferative disorders, and are also useful in combination therapies with radiation treatments or other chemotherapy agents.

Inhibition of HSP-90 is also known to result in up regulation of the expression of the chaperone HSP-70. HSP-70 up regulation is considered to be of therapeutic benefit for treatment of a wide range of neurodegenerative diseases including, but not limited to: Alzheimer's disease; Parkinson's disease; Dementia with Lewy bodies; Amyotropic lateral scleriosis (ALS); Polyglutamine disease; Huntington's disease; Spinal and bulbar muscular atrophy (SBMA); and Spinocerebellar ataxias (SCA1-3,7). Therefore, compounds of the invention are useful for treatment of such neurodegenerative diseases (Muchowski, P. J., Wacker J. L., Nat. Rev. Neurosci. 2005, 6, 11-22; Shen H. Y., et al. J. Biol. Chem. 2005, 280, 39962-9).

Inhibition of HSP-90 also has anti-fungal activity, both as a stand alone therapy and in combination with standard anti-fungal therapies such as the azole class of drugs. Therefore, the compounds of the invention are useful for treatment of fungal infections including, but not limited to, systemic fungal infections (Cowen, L. E., Lindquist, S., Science 2005, 309, 2185-9; and Cell. 1997 Apr. 18; 89(2):239-50).

HSP-90 has also been shown to be important to viral transcription and replication, in particular for such processes in HIV-1 and Hepatitis C virus. See J Biol. Chem. 2000 Jan. 7; 275(1):279-87; J. Virol. 2004 December; 78(23):13122-31; and Biochem Biophys Res Commun. 2007 Feb. 23; 353(4):882-8. Epub 2006 Dec. 22. Inhibitors of HSP-90 have been shown to attenuate infection in animal models of polio infection. See Genes Dev. 2007 (21) 195-205.

Inhibitors of HSP-90 have been shown to attenuate inflammation via lowering the level of a number of client proteins associated inflammation process. See FASEB J. 2007 Jul.; 21(9):2113-23.

Inhibition of HSP-90 is also expected to result in antimalarial activity; thus, inhibitors of this protein are useful as antimalarial drugs.

Inhibition of HSP-90 of Plasmodium falciparum, a protozoan parasite that causes malaria in humans, produces antimalarial activity. Therefore, compounds of the invention, as inhibitors of this protein, are useful as antimalarial drugs (Rajinder Kumar, Alla Musiyenko, Sailen Barik Malaria Journal 2003 2:30)

Therefore, there is a continuing need in the art for new methods of treating cancer, inflammation and inflammation-associated disorders, neurodegenerative diseases, fungal infections, malaria, and conditions or diseases related to uncontrolled angiogenesis.

### SUMMARY OF THE INVENTION

In one aspect, the invention provides compounds of Formula (I):

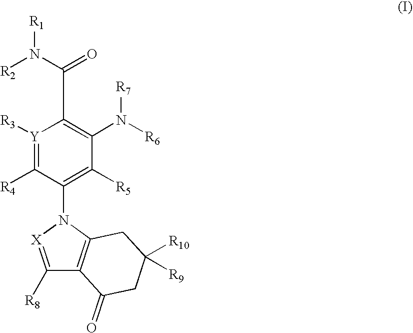

or a pharmaceutically acceptable salt thereof, wherein

R₁ and R₂ are independently —H or C₁-C₈ alkyl;

R₃ is absent or —H, —F, or —OCH₃;

R₄ and R₅ are independently —H, —F, or —OCH₃;

R₆ is (C₂-C₁₄) alkenyl, (C₁-C₁₄) alkyl, (C₂-C₁₄) alkynyl, aryl, aryl(C₁-C₈)alkyl, (C₃-C₈)cycloalkenyl, (C₃-C₈)cycloalkenyl(C₁-C₈)alkyl, (C₃-C₈)cycloalkyl, (C₃-C₈)cycloalkyl(C₁-C₈)alkyl, heteroaryl, heteroaryl(C₁-C₈)alkyl, heteroaryl(C₁-C₈)alkylthio(C₁-C₈)alkyl, heteroarylthio(C₁-C₈)alkyl, heterocyclyl, heterocycle (C₁-C₈)alkyl, or hydroxy(C₁-C₈)alkyl, wherein the R₆ group is optionally substituted with 1, 2, 3, 4, or 5 groups that are (C₂-C₈)alkenyl, (C₁-C₈)alkoxy, (C₁-C₈)alkoxy(C₁-C₈)alkyl, (C₁-C₈)alkoxycarbonyl, (C₁-C₈)alkyl, (C₁-C₈)alkylcarbonyl, (C₁-C₈)alkylcarbonyloxy, (C₁-C₈)alkylsulfinyl, (C₁-C₈)alkylsulfonyl, (C₁-C₈)alkylthio, (C₂-C₈)alkynyl, carboxy, carboxy(C₁-C₈)alkyl, cyano, cyano(C₁-C₈)alkyl, (C₃-C₈)cycloalkyl, (C₃-C₈)cycloalkyl(C₁-C₈)alkyl, formyl, halo(C₁-C₈)alkoxy, halo(C₁-C₈)alkyl, halogen, hydroxy, hydroxy(C₁-C₈)alkoxy, hydroxy(C₁-C₈)alkoxy(C₁-C₈)alkoxy, hydroxy(C₁-C₈)alkyl, mercapto, nitro, —NR₁₁R₁₂, (NR₁₁R₁₂) (C₁-C₈)alkyl, (NR₁₁R₁₂)carbonyl, oxo, HOCH₂CH(NH₂)C(O)O—, NH₂(CH₂)ₘC(O)O—, CH₃NH(CH₂)ₘC(O)O—, (CH₃)₂N(CH₂)ₘC(O)O—, NH₂(CH₂)ₜC(O)NH(CH₂)ₘC(O)O—, R₁₂CH(NH₂)C(O)O—, NH₂ (CH₂)ₘC(R₁₂)₂ (CH₂)ₘC(O)O—, NH₂CH₂CH₂C(O)O—, R₁₁ON═CH(CH₂)ₙO—, or HONHC(O) (CH₂)ₙO—; or

R₅ and R₆ taken together with the nitrogen atom to which they are attached form a heteroaryl, wherein the heteroaryl is imidazolyl, oxazolyl, pyrazolyl, pyridazinyl, pyridinyl, pyrazinyl, pyrrolyl, or thiazolyl, wherein the heteroaryl is optionally substituted with (C₁-C₈)alkoxy, (C₁-C₈)alkoxycarbonyl, (C₁-C₈)alkyl, (C₁-C₈)alkylcarbonyl, (C₁-C₈)alkylcarbonyloxy, aryl, aryl(C₁-C₈)alkyl, cyano, cyano(C₁-C₈)alkyl, (C₃-C₈) cycloalkyl, (C₃-C₈) cycloalkyl (C₁-C₈) alkyl, halo (C₁-C₈) alkoxy, halo (C₁-C₈) alkyl, halogen, heteroaryl, heteroaryl (C₁-C₈) alkyl, heterocyclyl, heterocycle (C₁-C₈)alkyl, hydroxy, hydroxy(C₁-C₈)alkyl, mercapto, nitro, —NR₁₁R₁₂, (NR₁₁R₁₂) (C₁-C₈)alkyl, or (NR₁₁R₁₂)carbonyl;

R₇ is —H or (C₁-C₈) alkyl;

R₈ is (C₂-C₈) alkenyl, (C₁-C₈) alkyl, (C₂-C₈) alkynyl, (C₃-C₈) cycloalkenyl, (C₃-C₈) cycloalkenyl (C₁-C₈) alkyl, (C₃-C₈) cycloalkyl, (C₃-C₈) cycloalkyl (C₁-C₈) alkyl, aryl, aryl (C₁-C₈) alkyl, halo (C₁-C₈) alkyl, heteroaryl, heteroaryl (C₁-C₈)alkyl, heterocyclyl, heterocycle (C₁-C₈)alkyl, or hydroxy (C₁-C₈) alkyl;

R₉ and R₁₀ are independently —H or (C₁-C₈)alkyl; or

R₉ and R₁₀ taken together with the carbon atom to which they are attached form (C₃-C₈)cycloalkyl;

R₁₁ and R₁₂ are independently —H, (C₂-C₈) alkenyl, (C₁-C₈)alkyl, (C₁-C₈)alkylcarbonyl, (C₂-C₈)alkynyl, aryl, aryl(C₁-C₈) alkyl, (C₃-C₈) cycloalkyl, (C₃-C₈) cycloalkyl(C₁-C₈) alkyl, formyl, heteroaryl, heteroaryl(C₁-C₈)alkyl, heterocyclyl, or heterocycle(C₁-C₈) alkyl; or

two R₁₂ groups together with the carbon to which they are attached form a (C₃-C₈)cycloalkyl group;

n is 1, 2, 3, 4, 5, or 6;

each m is independently 1, 2, 3, or 4;

each t is independently 1, 2, 3, or 4;

X is N or CR₁₃;

Y is C or N; and

R₁₃ is —H or (C₁-C₈) alkyl.

Further, the invention encompasses compounds of pharmaceutical compositions containing compounds of Formula I and methods employing such compounds or compositions in the treatment of diseases and/or conditions related to HSP-90 inhibition and/or cell proliferation, such as cancer, inflammation, arthritis, angiogenesis, infection, or the like.

In another aspect, the invention provides a pharmaceutical composition comprising a therapeutically effective amount of a compound of Formula (I), or a pharmaceutically acceptable salt thereof, and at least one pharmaceutically acceptable carrier, solvent, adjuvant or diluent.

In another aspect, the invention provides a method of treating inflammation comprising administering a therapeutically effective amount of a compound of Formula (I), or a pharmaceutically acceptable salt thereof, to a patient in need of such treatment.

In another aspect, the invention provides a method of treating arthritis comprising administering a therapeutically effective amount of a compound of Formula (I), or a pharmaceutically acceptable salt thereof, to a patient in need of such treatment.

In another aspect, the invention provides a method of treating angiogenesis comprising administering a therapeutically effective amount of a compound of Formula (I), or a pharmaceutically acceptable salt thereof, to a patient in need of such treatment.

In another aspect, the invention provides a method of treating neurodegenerative disease comprising administering a therapeutically effective amount of a compound of Formula (I), or a pharmaceutically acceptable salt thereof, to a patient in need of such treatment.

In another aspect, the invention provides a method of treating fungal infections comprising administering a therapeutically effective amount of a compound of Formula (I), or a pharmaceutically acceptable salt thereof, to a patient in need of such treatment.

In another aspect, the invention provides a method of treating malaria comprising administering a therapeutically effective amount of a compound of Formula (I), or a pharmaceutically acceptable salt thereof, to a patient in need of such treatment.

In another aspect, the invention provides a method of treating a disease or disorder related to cell proliferation comprising administering a therapeutically effective amount of a compound of Formula (I), or a pharmaceutically acceptable salt thereof, to a patient in need of such treatment.

In another aspect, the invention provides a method of inhibiting cell proliferation comprising administering a therapeutically effective amount of a compound of Formula (I), or a pharmaceutically acceptable salt thereof, to a patient in need of such treatment.

In another aspect, the invention provides a method of treating a disease or disorder related to Heat-shock protein 90 comprising administering a therapeutically effective amount of a compound of Formula (I), or a pharmaceutically acceptable salt thereof, to a patient in need of such treatment.

In another aspect, the invention provides a method of inhibiting Heat-shock protein 90 comprising administering a therapeutically effective amount of a compound of Formula (I), or a pharmaceutically acceptable salt thereof, to a patient in need of such treatment.

In another aspect, the invention provides a method of treating cancer comprising administering a therapeutically effective amount of a compound of Formula (I), or a pharmaceutically acceptable salt thereof, to a patient in need of such treatment.

In another aspect, the invention provides a method of treating breast cancer comprising administering a therapeutically effective amount of a compound of Formula (I), or a pharmaceutically acceptable salt thereof, to a patient in need of such treatment.

In another aspect, the invention provides a method of treating uterine cancer comprising administering a therapeutically effective amount of a compound of Formula (I), or a pharmaceutically acceptable salt thereof, to a patient in need of such treatment.

The invention also provides methods of treating and/or preventing viral infections in patients in need of such treatment comprising administration of a compound or salt of formula I.

In another aspect, the invention provides a method of treating Plasmodium falciparum comprising administering a therapeutically effective amount of a compound of Formula (I) or a pharmaceutically acceptable salt thereof to a patient in need of such treatment.

It is to be understood that both the foregoing general description and the following detailed description are merely exemplary of the invention, and are intended to provide an overview or framework for understanding the nature and character of the invention as it is claimed.

### DETAILED DESCRIPTION OF THE INVENTION

In another aspect, the invention provides compounds of Formula (I) wherein R₁ and R₂ are —H; R₃, R₄, and R₅ are independently —H, —F, or —OCH₃; and R₆ is (C₁-C₁₄)alkyl, (C₃-C₈)cycloalkenyl, (C₃-C₈)cycloalkyl, (C₃-C₈)cycloalkyl(C₁-C₈)alkyl, heteroaryl, heteroaryl(C₁-C₈)alkylthio(C₁-C₈)alkyl, heteroarylthio(C₁-C₈)alkyl, heterocyclyl, or hydroxy(C₁-C₈)alkyl, wherein the R₆ group is optionally substituted with 1, 2, 3, or 4 groups that are (C₂-C₈)alkenyl, (C₁-C₈) alkoxy, (C₁-C₈) alkoxy (C₁-C₈) alkyl, (C₁-C₈) alkoxycarbonyl, (C₁-C₈) alkyl, (C₁-C₈) alkylcarbonyl, (C₂-C₈) alkynyl, (C₃-C₈) cycloalkyl, (C₃-C₈) cycloalkyl (C₁-C₈) alkyl, hydroxy, hydroxy (C₁-C₈) alkoxy, hydroxy (C₁-C₈) alkoxy (C₁-C₈) alkoxy, hydroxy(C₁-C₈) alkyl, —NR₁₁R₁₂, (NR₁₁R₁₂) (C₁-C₈) alkyl, oxo, HOCH₂CH(NH₂)ₘC(O)O—, NH₂ (CH₂)ₘC(O)O—, CH₃NH(CH₂)ₘC(O)O—, (CH₃)₂N(CH₂)ₘC(O)O—, NH₂ (CH₂)ₜC(O)NH(CH₂)ₘC(O)O—, R₁₂CH(NH₂)C(O)O—, NH₂ (CH₂)ₘC(R₁₂)₂ (CH₂)ₘC(O)O—, NH₂CH₂CH₂C(O)O—, R₁₁ON═CH(CH₂)ₙO—, or HONHC(O) (CH₂) O—; or R₅ and R₆ taken together with the nitrogen atom to which they are attached form a heteroaryl, wherein the heteroaryl is imidazolyl optionally substituted with (C₁-C₈) alkyl or aryl; R₇ is —H; R₈ is (C₁-C₈) alkyl, (C₃-C₈) cycloalkyl (C₁-C₈) alkyl, aryl, aryl (C₁-C₈) alkyl, halo (C₁-C₈) alkyl, heteroaryl (C₁-C₈) alkyl, or hydroxy (C₁-C₈) alkyl; R₉ and R₁₀ are independently (C₁-C₈) alkyl; R₁₁ and R₁₂ are independently —H, (C₁-C₈) alkyl, (C₂-C₈) alkynyl, (C₃-C₈) cycloalkyl, (C₃-C₈)cycloalkyl (C₁-C₈) alkyl, or two R₁₂ groups together with the carbon to which they are attached form a (C₃-C₈)cycloalkyl group; n is 1; each m is independently 1 or 2; each t is independently 1 or 2 4; X is N or CR₁₃; Y is C; and R₁₃ is —H or (C₁-C₈) alkyl.

Compounds of the invention where at least one of R₃, R₄, and R₅ is —F have improved oral bioavailability as compared to compounds where R₃, R₄, and R₅ are —H. For example, a compound of the invention, Example 50, is orally bioavailable in rat (F>40%, 10 mg/kg over 24 hours) whereas the corresponding non-fluorinated analog is not orally bioavailable (F<5%).

Further, compounds of the invention, where at least one of R₃, R₄, and R₅ is —F, and in particular where R₃ is —F, are less potent inhibitors of the hERG channel. The hERG (human ether-a-go-go) gene encodes the pore forming subunit of a voltage-gated K+ channel critical for cardiac re-polarization. Inhibition of the hERG channel is known to be a significant risk factor against cardiac safety. For example, a compound of the invention, Example 57, has an IC₅₀ value 3 times higher (or less potent) in an hERG assay (Essen IonWorks, Ann Arbor, Mich. 48108) than that of its parent non-fluorinated analog.

Further still, compounds of the invention, where at least one of R₃, R₄, and R₅ is —F, and in particular where R₃ is —F, have greater potencies against broader numbers of cancer cell lines, particularly those cell lines that have already developed resistance to other anti-cancer agents or existing treatments. For example, a compound of the invention, Example 48, is 3-10 times more potent against adriamycin-resistant human breast cancer NCl/ADR-RES cell line, and 4-6 times more potent against a multi-drug resistant MES-SA/Dx5 (MDR+) cell line, than its non-fluorinated parent analog.

Compounds of the invention with one or more unexpected properties related to bioavailability, reduced cardiac toxicity, and/or increased activity against cancer cells include, but are not limited to, Examples 3, 6, 10, 17, 19, 20, 25, 28, 32, 39-43, 45, and 48-100. These physical and biological effects can act synergistically, making compounds of the invention, where at least one of R₃, R₄, and R₅ is —F, preferred agents for possible anti cancer applications.

Accordingly, in another aspect, the invention provides compounds of Formula (I) wherein R₁ and R₂ are —H; R₃ is —F; R₄ and R₅ are —H; R₆ is (C₁-C₁₄)alkyl, (C₃-C₈)cycloalkenyl, (C₃-C₈)cycloalkyl, (C₃-C₈)cycloalkyl(C₁-C₈)alkyl, heteroaryl(C₁-C₈)alkylthio(C₁-C₈)alkyl, or heterocyclyl, wherein the R₆ group is optionally substituted with 1 or 2 groups that are (C₁-C₈)alkoxy, (C₁-C₈)alkoxycarbonyl, (C₁-C₈)alkylcarbonyl, hydroxy, oxo, R₁₂CH(NH₂)C(O)O—, NH₂CH₂C(O)O—, NH₂CH₂CH₂C(O)O— or —NR₁₁R₁₂; R₇ is —H; R₈ is (C₁-C₈) alkyl, (C₃-C₈) cycloalkyl (C₁-C₈) alkyl, or halo (C₁-C₈) alkyl; R₉ and R₁₀ are independently (C₁-C₈)alkyl; R₁₁ and R₁₂ are independently —H or (C₁-C₈)alkyl; X is N; and Y is C.

In another aspect, the invention provides compounds of Formula (I) wherein R₁ and R₂ are —H; R₃ is —F; R₄ and R₅ are —H; R₆ is (C₁-C₁₄)alkyl substituted with 1 or 2 groups that are (C₁-C₈)alkoxy, (C₁-C₈)alkoxycarbonyl, or hydroxy; R₇ is —H; R₈ is (C₁-C₈)alkyl, (C₃-C₈)cycloalkyl(C₁-C₈)alkyl, or halo(C₁-C₈)alkyl; R₉ and R₁₀ are independently (C₁-C₈)alkyl; X is N; and Y is C.

In another aspect, the invention provides compounds of Formula (I) wherein R₁ and R₂ are —H; R₃ is —F; R₄ and R₅ are —H; R₆ is (C₁-C₁₄)alkyl substituted with 1 or 2 groups that are (C₁-C₈)alkoxy, (C₁-C₈)alkoxycarbonyl, or hydroxy; R₇ is —H; R₈ is methyl, ethyl, cyclopropyl, or trifluoromethyl; R₉ and R₁₀ are independently (C₁-C₈)alkyl; X is N; and Y is C.

In another aspect, the invention provides compounds of Formula (I) wherein R₁ and R₂ are —H; R₃ is —F; R₄ and R₅ are —H; R₆ is 2-hydroxyethyl, 1,3-dihydroxypropyl, 2,2-dimethoxyethyl, or 4-hydroxy-1-methoxy-1-oxobutan-2-yl; R₇ is —H; R₈ is methyl, ethyl, cyclopropylmethyl, or trifluoromethyl; R₉ and R₁₀ are methyl; X is N; and Y is C.

In another aspect, the invention provides compounds of Formula (I) wherein R₁ and R₂ are —H; R₃ is —F; R₄ and R₅ are —H; R₆ is (C₃-C₈) cycloalkenyl; R₇ is —H; R is (C₁-C₈) alkyl, (C₃-C₈) cycloalkyl (C₁-C₈) alkyl, or halo (C₁-C₈) alkyl; R₉ and R₁₀ are independently (C₁-C₈)alkyl; X is N; and Y is C.

In another aspect, the invention provides compounds of Formula (I) wherein R₁ and R₂ are —H; R₃ is —F; R₄ and R₅ are —H; R₆ is (C₃-C₈)cycloalkenyl; R₇ is —H; R is methyl, ethyl, cyclopropylmethyl, or trifluoromethyl; R₉ and R₁₀ are methyl; X is N; and Y is C.

In another aspect, the invention provides compounds of Formula (I) wherein R₁ and R₂ are —H; R₃ is —F; R₄ and R₅ are —H; R₆ is cyclopentenyl; R₇ is —H; R₈ is methyl, ethyl, cyclopropylmethyl, or trifluoromethyl; R₉ and R₁₀ are methyl; X is N; and Y is C.

In another aspect, the invention provides compounds of Formula (I) wherein R₁ and R₂ are —H; R₃ is —F; R₄ and R₅ are —H; R₆ is (C₃-C₈)cycloalkyl optionally substituted with one group that is (C₁-C₈)alkoxy, hydroxy, R₁₂CH(NH₂)C(O)O—, NH₂CH₂C(O)O—, NH₂CH₂CH₂C(O)O— or —NR₁₁R₁₂; R₇ is —H; R₈ is (C₁-C₈)alkyl, (C₃-C₈)cycloalkyl(C₁-C₈)alkyl, or halo(C₁-C₈)alkyl; R₉ and R₁₀ are independently (C₁-C₈)alkyl; R₁₁ and R₁₂ are independently —H or (C₁-C₈)alkyl; X is N; and Y is C.

In another aspect, the invention provides compounds of Formula (I) wherein R₁ and R₂ are —H; R₃ is —F; R₄ and R₅ are —H; R₆ is cyclobutyl, cyclopentyl, cyclohexyl, or cycloheptyl, wherein each is optionally substituted with one group that is (C₁-C₈)alkoxy, hydroxy, R₁₂CH(NH₂)C(O)O—, NH₂CH₂C(O)O—, NH₂CH₂CH₂C(O)O— or —NR₁₁R₁₂; R₇ is —H; R₈ is methyl, ethyl, cyclopropylmethyl, or trifluoromethyl; R₉ and R₁₀ are methyl;

R₁₁ and R₁₂ are independently —H or (C₁-C₈)alkyl; X is N; and Y is C.

In another aspect, the invention provides compounds of Formula (I) wherein R₁ and R₂ are —H; R₃ is —F; R₄ and R₅ are —H; R₆ is cyclobutyl, cyclopentyl, cycloheptyl, 2-hydroxycyclopentyl, 2-(2-aminoacetoxy)cyclopentyl, 2-(2-aminoacetoxy)cyclohexyl, 4-(2-aminoacetoxy)cyclohexyl, 2-aminocyclohexyl, 2-hydroxycyclohexyl, 4-hydroxycyclohexyl, 2-neopentylaminocyclohexyl, 4-neopentylaminocyclohexyl, or 4-methoxycyclohexyl; R₇ is —H; R₈ is methyl, ethyl, cyclopropylmethyl, or trifluoromethyl; R₉ and R₁₀ are methyl; X is N; and Y is C.

In another aspect, the invention provides compounds of Formula (I) wherein R₁ and R₂ are —H; R₃ is —F; R₄ and R₅ are —H; R₆ is cyclobutyl, cyclopentyl, cycloheptyl, trans-2-hydroxycyclopentyl, trans-2-(2-aminoacetoxy)cyclohexyl, trans-4-(2-aminoacetoxy)cyclohexyl, trans-2-aminocyclohexyl, trans-2-hydroxycyclohexyl, trans-4-hydroxycyclohexyl, trans-2-neopentylaminocyclohexyl, trans-4-neopentylaminocyclohexyl, or trans-4-methoxycyclohexyl; R₇ is —H; R₈ is methyl, ethyl, cyclopropylmethyl, or trifluoromethyl; R₉ and R₁₀ are methyl; X is N; and Y is C.

In another aspect, the invention provides compounds of Formula (I) wherein R₁ and R₂ are —H; R₃ is —F; R₄ and R₅ are —H; R₆ is (C₃-C₈)cycloalkyl(C₁-C₈)alkyl; R₇ is —H; R₈ is (C₁-C₈)alkyl, (C₃-C₈)cycloalkyl(C₁-C₈)alkyl, or halo(C₁-C₈)alkyl; R₉ and R₁₀ are independently (C₁-C₈)alkyl; X is N; and Y is C.

In another aspect, the invention provides compounds of Formula (I) wherein R₁ and R₂ are —H; R₃ is —F; R₄ and R₅ are —H; R₆ is cyclopropylmethyl; R₇ is —H; R₈ is methyl, ethyl, cyclopropylmethyl, or trifluormethyl; R₉ and R₁₀ are methyl; X is N; and Y is C.

In another aspect, the invention provides compounds of Formula (I) wherein R₁ and R₂ are —H; R₃ is —F; R₄ and R₅ are —H; R₆ is heteroaryl(C₁-C₈)alkylthio(C₁-C₈)alkyl; R₇ is —H; R₈ is (C₁-C₈)alkyl, (C₃-C₈)cycloalkyl(C₁-C₈)alkyl, or halo(C₁-C₈)alkyl; R₉ and R₁₀ are independently (C₁-C₈)alkyl; X is N; and Y is C.

In another aspect, the invention provides compounds of Formula (I) wherein R₁ and R₂ are —H; R₃ is —F; R₄ and R₅ are —H; R₆ is ethyl 2-((1H-imidazolyl)methylthio)ethyl; R₇ is —H; R₈ is methyl, ethyl, cyclopropylmethyl, or trifluoromethyl; R₉ and R₁₀ are methyl; X is N; and Y is C.

In another aspect, the invention provides compounds of Formula (I) wherein R₁ and R₂ are —H; R₃ is —F; R₄ and R₅ are —H; R₆ is heterocyclyl optionally substituted with 1 group that is (C₁-C₈)alkylcarbonyl or oxo; R₇ is —H; R is (C₁-C₈)alkyl, (C₃-C₈)cycloalkyl(C₁-C₈)alkyl, or halo(C₁-C₈)alkyl; R₉ and R₁₀ are independently (C₁-C₈)alkyl; X is N; and Y is C.

In another aspect, the invention provides compounds of Formula (I) wherein R₁ and R₂ are —H; R₃ is —F; R₄ and R₅ are —H; R₆ is oxetanyl, tetrahydrofuranyl, tetrahydropyran, piperidinyl, or azepanyl, wherein each is optionally substituted with 1 group that is (C₁-C₈)alkylcarbonyl or oxo; R₇ is —H; R₈ is methyl, ethyl, cyclopropylmethyl, or trifluoromethyl; R₉ and R₁₀ are methyl; X is N; and Y is C.

In another aspect, the invention provides compounds of Formula (I) wherein R₁ and R₂ are —H; R₃ is —F; R₄ and R₅ are —H; R₆ is piperidinyl substituted with 1, 2, 3, or 4 groups independently selected from (C₁-C₈)alkyl; R₇ is —H; R₈ is methyl, ethyl, cyclopropylmethyl, or trifluoromethyl; R₉ and R₁₀ are methyl; X is N; and Y is C.

In another aspect, the invention provides compounds of Formula (I) wherein R₁ and R₂ are —H; R₃ is —F; R₄ and R₅ are —H; R₆ is piperidinyl substituted with 1, 2, 3, or 4 methyl groups; R₇ is —H; R₈ is methyl, ethyl, cyclopropylmethyl, or trifluoromethyl; R₉ and R₁₀ are methyl; X is N; and Y is C.

In another aspect, the invention provides compounds of Formula (I) wherein R₁ and R₂ are —H; R₃ is —F; R₄ and R₅ are —H; R₆ is tetrahydrofuranyl, 2-oxotetrahydrofuranyl, tetrahydropyranyl, 2-oxoazepanyl, or 1-acetylpiperidinyl; R₇ is —H; R₈ is methyl, ethyl, cyclopropylmethyl, or trifluoromethyl; R₉ and R₁₀ are methyl; X is N; and Y is C.

In another aspect, the invention provides compounds of Formula (I) wherein R₁ and R₂ are —H; R₄ is —F; R₃ and R₅ are —H; R₆ is (C₁-C₁₄)alkyl, (C₃-C₈)cycloalkenyl, (C₃-C₈)cycloalkyl, (C₃-C₈)cycloalkyl(C₁-C₈)alkyl, heteroaryl(C₁-C₈)alkylthio(C₁-C₈)alkyl, or heterocyclyl, wherein the R₆ group is optionally substituted with 1 or 2 groups that are (C₁-C₈)alkoxy, (C₁-C₈)alkoxycarbonyl, (C₁-C₈)alkylcarbonyl, hydroxy, oxo, R₁₂CH(NH₂)C(O)O—, NH₂CH₂CH₂C(O)O— or —NR₁₁R₁₂; R₇ is —H; R₈ is (C₁-C₈)alkyl, (C₃-C₈)cycloalkyl(C₁-C₈)alkyl, or halo(C₁-C₈)alkyl; R₉ and R₁₀ are independently (C₁-C₈)alkyl; R₁₁ and R₁₂ are independently —H or (C₁-C₈)alkyl; X is N or CH; and Y is C.

In another aspect, the invention provides compounds of Formula (I) wherein R₁ and R₂ are —H; R₄ is —F; R₃ and R₅ are —H; R₆ is (C₁-C₁₄)alkyl substituted with 1 or 2 groups that are (C₁-C₈)alkoxy, (C₁-C₈)alkoxycarbonyl, or hydroxy; R₇ is —H; R₈ is (C₁-C₈)alkyl, (C₃-C₈)cycloalkyl(C₁-C₈)alkyl, or halo(C₁-C₈)alkyl; R₉ and R₁₀ are independently (C₁-C₈)alkyl; X is N; and Y is C.

In another aspect, the invention provides compounds of Formula (I) wherein R₁ and R₂ are —H; R₄ is —F; R₃ and R₅ are —H; R₆ is (C₁-C₁₄)alkyl substituted with 1 or 2 groups that are (C₁-C₈)alkoxy, (C₁-C₈)alkoxycarbonyl, or hydroxy; R₇ is —H; R₈ is methyl, ethyl, cyclopropyl, or trifluoromethyl; R₉ and R₁₀ are independently (C₁-C₈)alkyl; X is N; and Y is C.

In another aspect, the invention provides compounds of Formula (I) wherein R₁ and R₂ are —H; R₄ is —F; R₃ and R₅ are —H; R₆ is 2-hydroxyethyl, 1,3-dihydroxypropyl, 2,2-dimethoxyethyl, or 4-hydroxy-1-methoxy-1-oxobutan-2-yl; R₇ is —H; R₈ is methyl, ethyl, cyclopropylmethyl, or trifluoromethyl; R₉ and R₁₀ are methyl; X is N; and Y is C.

In another aspect, the invention provides compounds of Formula (I) wherein R₁ and R₂ are —H; R₄ is —F; R₃ and R₅ are —H; R₆ is (C₃-C₈)cycloalkenyl; R₇ is —H; R is (C₁-C₈)alkyl, (C₃-C₈)cycloalkyl(C₁-C₈)alkyl, or halo(C₁-C₈)alkyl; R₉ and R₁₀ are independently (C₁-C₈)alkyl; X is N; and Y is C.

In another aspect, the invention provides compounds of Formula (I) wherein R₁ and R₂ are —H; R₄ is —F; R₃ and R₅ are —H; R₆ is (C₃-C₈)cycloalkenyl; R₇ is —H; R₈ is methyl, ethyl, cyclopropylmethyl, or trifluoromethyl; R₉ and R₁₀ are methyl; X is N; and Y is C.

In another aspect, the invention provides compounds of Formula (I) wherein R₁ and R₂ are —H; R₄ is —F; R₃ and R₅ are —H; R₆ is cyclopentenyl; R₇ is —H; R₈ is methyl, ethyl, cyclopropylmethyl, or trifluoromethyl; R₉ and R₁₀ are methyl; X is N; and Y is C.

In another aspect, the invention provides compounds of Formula (I) wherein R₁ and R₂ are —H; R₄ is —F; R₃ and R₅ are —H; R₆ is (C₃-C₈)cycloalkyl optionally substituted with one group that is (C₁-C₈)alkoxy, hydroxy, R₁₂CH(NH₂)C(O)O—, NH₂CH₂CH₂C(O)O— or —NR₁₁R₁₂; R₇ is —H; R is (C₁-C₈)alkyl, (C₃-C₈)cycloalkyl(C₁-C₈)alkyl, or halo(C₁-C₈)alkyl; R₉ and R₁₀ are independently (C₁-C₈)alkyl; R₁₁ and R₁₂ are independently —H or (C₁-C₈)alkyl; X is N; and Y is C.

In another aspect, the invention provides compounds of Formula (I) wherein R₁ and R₂ are —H; R₄ is —F; R₃ and R₅ are —H; R₆ is cyclobutyl, cyclopentyl, or cyclohexyl, wherein each is optionally substituted with one group that is (C₁-C₈)alkoxy, hydroxy, R₁₂CH(NH₂)C(O)O—, NH₂CH₂CH₂C(O)O— or —NR₁₁R₁₂; R₇ is —H; R₈ is methyl, ethyl, cyclopropylmethyl, or trifluoromethyl; R₉ and R₁₀ are methyl; R₁₁ and R₁₂ are independently —H or (C₁-C₈)alkyl; X is N; and Y is C.

In another aspect, the invention provides compounds of Formula (I) wherein R₁ and R₂ are —H; R₄ is —F; R₃ and R₅ are —H; R₆ is cyclobutyl, 2-hydroxycyclopentyl, 2-aminocyclohexyl, 2-hydroxycyclohexyl, 4-hydroxycyclohexyl, 2-neopentylaminocyclohexyl, 4-neopentylaminocyclohexyl, or 4-methoxycyclohexyl; R₇ is —H; R₈ is methyl, ethyl, cyclopropylmethyl, or trifluoromethyl; R₉ and R₁₀ are methyl; X is N; and Y is C.

In another aspect, the invention provides compounds of Formula (I) wherein R₁ and R₂ are —H; R₄ is —F; R₃ and R₅ are —H; R₆ is cyclobutyl, trans-2-hydroxycyclopentyl, trans-2-aminocyclohexyl, trans-2-hydroxycyclohexyl, trans-4-hydroxycyclohexyl, trans-2-neopentylaminocyclohexyl, trans-4-neopentylaminocyclohexyl, or trans-4-methoxycyclohexyl; R₇ is —H; R₈ is methyl, ethyl, cyclopropylmethyl, or trifluoromethyl; R₉ and R₁₀ are methyl; X is N; and Y is C.

In another aspect, the invention provides compounds of Formula (I) wherein R₁ and R₂ are —H; R₄ is —F; R₃ and R₅ are —H; R₆ is (C₃-C₈)cycloalkyl(C₁-C₈)alkyl; R₇ is —H; R₈ is (C₁-C₈)alkyl, (C₃-C₈)cycloalkyl(C₁-C₈)alkyl, or halo(C₁-C₈)alkyl; R₉ and R₁₀ are independently (C₁-C₈)alkyl; X is N; and Y is C.

In another aspect, the invention provides compounds of Formula (I) wherein R₁ and R₂ are —H; R₄ is —F; R₃ and R₅ are —H; R₆ is cyclopropylmethyl; R₇ is —H; R₈ is methyl, ethyl, cyclopropylmethyl, or trifluormethyl; R₉ and R₁₀ are methyl; X is N; and Y is C.

In another aspect, the invention provides compounds of Formula (I) wherein R₁ and R₂ are —H; R₄ is —F; R₃ and R₅ are —H; R₆ is heteroaryl(C₁-C₈)alkylthio(C₁-C₈)alkyl; R₇ is —H; R₈ is (C₁-C₈)alkyl, (C₃-C₈)cycloalkyl(C₁-C₈)alkyl, or halo(C₁-C₈)alkyl; R₉ and R₁₀ are independently (C₁-C₈)alkyl; X is N; and Y is C.

In another aspect, the invention provides compounds of Formula (I) wherein R₁ and R₂ are —H; R₄ is —F; R₃ and R₅ are —H; R₆ is ethyl 2-((1H-imidazolyl)methylthio)ethyl; R₇ is —H; R₈ is methyl, ethyl, cyclopropylmethyl, or trifluoromethyl; R₉ and R₁₀ are methyl; X is N; and Y is C.

In another aspect, the invention provides compounds of Formula (I) wherein R₁ and R₂ are —H; R₄ is —F; R₃ and R₅ are —H; R₆ is heterocyclyl optionally substituted with 1 group that is (C₁-C₈)alkylcarbonyl or oxo; R₇ is —H; R₃ is (C₁-C₈)alkyl, (C₃-C₈)cycloalkyl(C₁-C₈)alkyl, or halo(C₁-C₈)alkyl; R₉ and R₁₀ are independently (C₁-C₈)alkyl; X is N; and Y is C.

In another aspect, the invention provides compounds of Formula (I) wherein R₁ and R₂ are —H; R₄ is —F; R₃ and R₅ are —H; R₆ is tetrahydrofuranyl, tetrahydropyran, piperidinyl, or azepanyl, wherein each is optionally substituted with 1 group that is (C₁-C₈)alkylcarbonyl or oxo; R₇ is —H; R₈ is methyl, ethyl, cyclopropylmethyl, or trifluoromethyl; R₉ and R₁₀ are methyl; X is N; and Y is C.

In another aspect, the invention provides compounds of Formula (I) wherein R₁ and R₂ are —H; R₄ is —F; R₃ and R₅ are —H; R₆ is tetrahydrofuranyl, 2-oxotetrahydrofuranyl, tetrahydropyranyl, 2-oxoazepanyl, or 1-acetylpiperidinyl; R₇ is —H; R₈ is methyl, ethyl, cyclopropylmethyl, or trifluoromethyl; R₉ and R₁₀ are methyl; X is N; and Y is C.

In another aspect, the invention provides compounds of Formula (I) wherein R₁ and R₂ are —H; R₃ and R₄ are —H; R₅ is —F; R₆ is (C₁-C₁₄)alkyl, (C₃-C₈)cycloalkenyl, (C₃-C₈)cycloalkyl, (C₃-C₈)cycloalkyl(C₁-C₈)alkyl, heteroaryl(C₁-C₈)alkylthio(C₁-C₈)alkyl, or heterocyclyl, wherein the R₆ group is optionally substituted with 1 or 2 groups that are (C₁-C₈)alkoxy, (C₁-C₈)alkoxycarbonyl, (C₁-C₈)alkylcarbonyl, hydroxy, oxo, R₁₂CH(NH₂)C(O)O—, NH₂CH₂CH₂C(O)O— or —NR₁₁R₁₂; R₇ is —H; R₈ is (C₁-C₈)alkyl, (C₃-C₈)cycloalkyl(C₁-C₈)alkyl, or halo(C₁-C₈)alkyl; R₉ and R₁₀ are independently (C₁-C₈)alkyl; R₁₁ and R₁₂ are independently —H or (C₁-C₈)alkyl; X is N or CH; and Y is C.

In another aspect, the invention provides compounds of Formula (I) wherein R₁ and R₂ are —H; R₃ and R₄ are —H; R₅ is —F; R₆ is (C₁-C₁₄)alkyl substituted with 1 or 2 groups that are (C₁-C₈)alkoxy, (C₁-C₈)alkoxycarbonyl, or hydroxy; R₇ is —H; R₈ is (C₁-C₈)alkyl, (C₃-C₈)cycloalkyl(C₁-C₈)alkyl, or halo(C₁-C₈)alkyl; R₉ and R₁₀ are independently (C₁-C₈)alkyl; X is N; and Y is C.

In another aspect, the invention provides compounds of Formula (I) wherein R₁ and R₂ are —H; R₃ and R₄ are —H; R₅ is —F; R₆ is (C₁-C₁₄)alkyl substituted with 1 or 2 groups that are (C₁-C₈)alkoxy, (C₁-C₈)alkoxycarbonyl, or hydroxy; R₇ is —H; R₈ is methyl, ethyl, cyclopropyl, or trifluoromethyl; R₉ and R₁₀ are independently (C₁-C₈)alkyl; X is N; and Y is C.

In another aspect, the invention provides compounds of Formula (I) wherein R₁ and R₂ are —H; R₃ and R₄ are —H; R₅ is —F; R₆ is 2-hydroxyethyl, 1,3-dihydroxypropyl, 2,2-dimethoxyethyl, or 4-hydroxy-1-methoxy-1-oxobutan-2-yl; R₇ is —H; R₈ is methyl, ethyl, cyclopropylmethyl, or trifluoromethyl; R₉ and R₁₀ are methyl; X is N; and Y is C.

In another aspect, the invention provides compounds of Formula (I) wherein R₁ and R₂ are —H; R₃ and R₄ are —H; R₅ is —F; R₆ is (C₃-C₈) cycloalkenyl; R₇ is —H; R₈ is (C₁-C₈) alkyl, (C₃-C₈) cycloalkyl (C₁-C₈) alkyl, or halo (C₁-C₈) alkyl; R₉ and R₁₀ are independently (C₁-C₈)alkyl; X is N; and Y is C.

In another aspect, the invention provides compounds of Formula (I) wherein R₁ and R₂ are —H; R₃ and R₄ are —H; R₅ is —F; R₆ is (C₃-C₈)cycloalkenyl; R₇ is —H; R is methyl, ethyl, cyclopropylmethyl, or trifluoromethyl; R₉ and R₁₀ are methyl; X is N; and Y is C.

In another aspect, the invention provides compounds of Formula (I) wherein R₁ and R₂ are —H; R₃ and R₄ are —H; R₅ is —F; R₆ is cyclopentenyl; R₇ is —H; R₈ is methyl, ethyl, cyclopropylmethyl, or trifluoromethyl; R₉ and R₁₀ are methyl; X is N; and Y is C.

In another aspect, the invention provides compounds of Formula (I) wherein R₁ and R₂ are —H; R₃ and R₄ are —H; R₅ is —F; R₆ is (C₃-C₈)cycloalkyl optionally substituted with one group that is (C₁-C₈)alkoxy, hydroxy, R₁₂CH(NH₂)C(O)O—, NH₂CH₂CH₂C(O)O— or —NR₁₁R₁₂; R₇ is —H; R₈ is (C₁-C₈)alkyl, (C₃-C₈)cycloalkyl(C₁-C₈)alkyl, or halo(C₁-C₈)alkyl; R₉ and R₁₀ are independently (C₁-C₈)alkyl; R₁₁ and R₁₂ are independently —H or (C₁-C₈)alkyl; X is N; and Y is C.

In another aspect, the invention provides compounds of Formula (I) wherein R₁ and R₂ are —H; R₃ and R₄ are —H; R₅ is —F; R₆ is cyclobutyl, cyclopentyl, or cyclohexyl, wherein each is optionally substituted with one group that is (C₁-C₈)alkoxy, hydroxy, R₁₂CH(NH₂)C(O)O—, NH₂CH₂CH₂C(O)O— or —NR₁₁R₁₂; R₇ is —H; R₈ is methyl, ethyl, cyclopropylmethyl, or trifluoromethyl; R₉ and R₁₀ are methyl; R₁₁ and R₁₂ are independently —H or (C₁-C₈)alkyl; X is N; and Y is C.

In another aspect, the invention provides compounds of Formula (I) wherein R₁ and R₂ are —H; R₃ and R₄ are —H; R₅ is —F; R₆ is cyclobutyl, 2-hydroxycyclopentyl, 2-aminocyclohexyl, 2-hydroxycyclohexyl, 4-hydroxycyclohexyl, 2-neopentylaminocyclohexyl, 4-neopentylaminocyclohexyl, or 4-methoxycyclohexyl; R₇ is —H; R₈ is methyl, ethyl, cyclopropylmethyl, or trifluoromethyl; R₉ and R₁₀ are methyl; X is N; and Y is C.

In another aspect, the invention provides compounds of Formula (I) wherein R₁ and R₂ are —H; R₃ and R₄ are —H; R₅ is —F; R₆ is cyclobutyl, trans-2-hydroxycyclopentyl, trans-2-aminocyclohexyl, trans-2-hydroxycyclohexyl, trans-4-hydroxycyclohexyl, trans-2-neopentylaminocyclohexyl, trans-4-neopentylaminocyclohexyl, or trans-4-methoxycyclohexyl; R₇ is —H; R₈ is methyl, ethyl, cyclopropylmethyl, or trifluoromethyl; R₉ and R₁₀ are methyl; X is N; and Y is C.

In another aspect, the invention provides compounds of Formula (I) wherein R₁ and R₂ are —H; R₃ and R₄ are —H; R₅ is —F; R₆ is (C₃-C₈)cycloalkyl(C₁-C₈)alkyl; R₇ is —H; R₈ is (C₁-C₈)alkyl, (C₃-C₈)cycloalkyl(C₁-C₈)alkyl, or halo(C₁-C₈)alkyl; R₉ and R₁₀ are independently (C₁-C₈)alkyl; X is N; and Y is C.

In another aspect, the invention provides compounds of Formula (I) wherein R₁ and R₂ are —H; R₃ and R₄ are —H; R₅ is —F; R₆ is cyclopropylmethyl; R₇ is —H; R₈ is methyl, ethyl, cyclopropylmethyl, or trifluormethyl; R₉ and R₁₀ are methyl; X is N; and Y is C.

In another aspect, the invention provides compounds of Formula (I) wherein R₁ and R₂ are —H; R₃ and R₄ are —H; R₅ is —F; R₆ is heteroaryl(C₁-C₈)alkylthio(C₁-C₈)alkyl; R₇ is —H; R₈ is (C₁-C₈)alkyl, (C₃-C₈)cycloalkyl(C₁-C₈)alkyl, or halo(C₁-C₈)alkyl; R₉ and R₁₀ are independently (C₁-C₈)alkyl; X is N; and Y is C.

In another aspect, the invention provides compounds of Formula (I) wherein R₁ and R₂ are —H; R₃ and R₄ are —H; R₅ is —F; R₆ is ethyl 2-((1H-imidazolyl)methylthio)ethyl; R₇ is —H; R₈ is methyl, ethyl, cyclopropylmethyl, or trifluoromethyl; R₉ and R₁₀ are methyl; X is N; and Y is C.

In another aspect, the invention provides compounds of Formula (I) wherein R₁ and R₂ are —H; R₃ and R₄ are —H; R₅ is —F; R₆ is heterocyclyl optionally substituted with 1 group that is (C₁-C₈)alkylcarbonyl or oxo; R₇ is —H; R₈ is (C₁-C₈)alkyl, (C₃-C₈) cycloalkyl(C₁-C₈)alkyl, or halo(C₁-C₈)alkyl; R₉ and R₁₀ are independently (C₁-C₈)alkyl; X is N; and Y is C.

In another aspect, the invention provides compounds of Formula (I) wherein R₁ and R₂ are —H; R₃ and R₄ are —H; R₅ is —F; R₆ is tetrahydrofuranyl, tetrahydropyran, piperidinyl, or azepanyl, wherein each is optionally substituted with 1 group that is (C₁-C₈)alkylcarbonyl or oxo; R₇ is —H; R₈ is methyl, ethyl, cyclopropylmethyl, or trifluoromethyl; R₉ and R₁₀ are methyl; X is N; and Y is C.

In another aspect, the invention provides compounds of Formula (I) wherein R₁ and R₂ are —H; R₃ and R₄ are —H; R₅ is —F; R₆ is tetrahydrofuranyl, 2-oxotetrahydrofuranyl, tetrahydropyranyl, 2-oxoazepanyl, or 1-acetylpiperidinyl; R₇ is —H; R₈ is methyl, ethyl, cyclopropylmethyl, or trifluoromethyl; R₉ and R₁₀ are methyl; X is N; and Y is C.

In another aspect, the invention provides compounds of Formula (I) wherein R₁ and R₂ are —H; R₃ is —H; R₄ and R₅ are —F; R₆ is (C₁-C₁₄)alkyl, (C₃-C₈)cycloalkenyl, (C₃-C₈)cycloalkyl, (C₃-C₈)cycloalkyl(C₁-C₈)alkyl, heteroaryl(C₁-C₈)alkylthio(C₁-C₈)alkyl, or heterocyclyl, wherein the R₆ group is optionally substituted with 1 or 2 groups that are (C₁-C₈)alkoxy, (C₁-C₈)alkoxycarbonyl, (C₁-C₈)alkylcarbonyl, hydroxy, oxo, R₁₂CH(NH₂)C(O)O—, NH₂CH₂CH₂C(O)O— or —NR₁₁R₁₂; R₇ is —H; R is (C₁-C₈)alkyl, (C₃-C₈)cycloalkyl(C₁-C₈)alkyl, or halo(C₁-C₈)alkyl; R₉ and R₁₀ are independently (C₁-C₈)alkyl; R₁₁ and R₁₂ are independently —H or (C₁-C₈)alkyl; X is N or CH; and Y is C.

In another aspect, the invention provides compounds of Formula (I) wherein R₁ and R₂ are —H; R₃ is —H; R₄ and R₅ are —F; R₆ is (C₁-C₁₄)alkyl substituted with 1 or 2 groups that are (C₁-C₈)alkoxy, (C₁-C₈)alkoxycarbonyl, or hydroxy; R₇ is —H; R₈ is (C₁-C₈)alkyl, (C₃-C₈)cycloalkyl(C₁-C₈)alkyl, or halo(C₁-C₈)alkyl; R₉ and R₁₀ are independently (C₁-C₈)alkyl; X is N; and Y is C.

In another aspect, the invention provides compounds of Formula (I) wherein R₁ and R₂ are —H; R₃ is —H; R₄ and R₅ are —F; R₆ is (C₁-C₁₄)alkyl substituted with 1 or 2 groups that are (C₁-C₈)alkoxy, (C₁-C₈)alkoxycarbonyl, or hydroxy; R₇ is —H; R₈ is methyl, ethyl, cyclopropyl, or trifluoromethyl; R₉ and R₁₀ are independently (C₁-C₈)alkyl; X is N; and Y is C.

In another aspect, the invention provides compounds of Formula (I) wherein R₁ and R₂ are —H; R₃ is —H; R₄ and R₅ are —F; R₆ is 2-hydroxyethyl, 1,3-dihydroxypropyl, 2,2-dimethoxyethyl, or 4-hydroxy-1-methoxy-1-oxobutan-2-yl; R₇ is —H; R₈ is methyl, ethyl, cyclopropylmethyl, or trifluoromethyl; R₉ and R₁₀ are methyl; X is N; and Y is C.

In another aspect, the invention provides compounds of Formula (I) wherein R₁ and R₂ are —H; R₃ is —H; R₄ and R₅ are —F; R₆ is (C₃-C₈) cycloalkenyl; R₇ is —H; R is (C₁-C₈) alkyl, (C₃-C₈) cycloalkyl (C₁-C₈) alkyl, or halo (C₁-C₈) alkyl; R₉ and R₁₀ are independently (C₁-C₈)alkyl; X is N; and Y is C.

In another aspect, the invention provides compounds of Formula (I) wherein R₁ and R₂ are —H; R₃ is —H; R₄ and R₅ are —F; R₆ is (C₃-C₈)cycloalkenyl; R₇ is —H; R is methyl, ethyl, cyclopropylmethyl, or trifluoromethyl; R₉ and R₁₀ are methyl; X is N; and Y is C.

In another aspect, the invention provides compounds of Formula (I) wherein R₁ and R₂ are —H; R₃ is —H; R₄ and R₅ are —F; R₆ is cyclopentenyl; R₇ is —H; R₈ is methyl, ethyl, cyclopropylmethyl, or trifluoromethyl; R₉ and R₁₀ are methyl; X is N; and Y is C.

In another aspect, the invention provides compounds of Formula (I) wherein R₁ and R₂ are —H; R₃ is —H; R₄ and R₅ are —F; R₆ is (C₃-C₈)cycloalkyl optionally substituted with one group that is (C₁-C₈)alkoxy, hydroxy, R₁₂CH(NH₂)C(O)O—, NH₂CH₂CH₂C(O)O— or —NR₁₁R₁₂; R₇ is —H; R₈ is (C₁-C₈)alkyl, (C₃-C₈)cycloalkyl(C₁-C₈)alkyl, or halo(C₁-C₈)alkyl; R₉ and R₁₀ are independently (C₁-C₈)alkyl; R₁₁ and R₁₂ are independently —H or (C₁-C₈)alkyl; X is N; and Y is C.

In another aspect, the invention provides compounds of Formula (I) wherein R₁ and R₂ are —H; R₃ is —H; R₄ and R₅ are —F; R₆ is cyclobutyl, cyclopentyl, or cyclohexyl, wherein each is optionally substituted with one group that is (C₁-C₈)alkoxy, hydroxy, R₁₂CH(NH₂)C(O)O—, NH₂CH₂CH₂C(O)O— or —NR₁₁R₁₂; R₇ is —H; R₈ is methyl, ethyl, cyclopropylmethyl, or trifluoromethyl; R₉ and R₁₀ are methyl; R₁₁ and R₁₂ are independently —H or (C₁-C₈)alkyl; X is N; and Y is C.

In another aspect, the invention provides compounds of Formula (I) wherein R₁ and R₂ are —H; R₃ is —H; R₄ and R₅ are —F; R₆ is cyclobutyl, 2-hydroxycyclopentyl, 2-aminocyclohexyl, 2-hydroxycyclohexyl, 4-hydroxycyclohexyl, 2-neopentylaminocyclohexyl, 4-neopentylaminocyclohexyl, or 4-methoxycyclohexyl; R₇ is —H; R₈ is methyl, ethyl, cyclopropylmethyl, or trifluoromethyl; R₉ and R₁₀ are methyl; X is N; and Y is C.

In another aspect, the invention provides compounds of Formula (I) wherein R₁ and R₂ are —H; R₃ is —H; R₄ and R₅ are —F; R₆ is cyclobutyl, trans-2-hydroxycyclopentyl, trans-2-aminocyclohexyl, trans-2-hydroxycyclohexyl, trans-4-hydroxycyclohexyl, trans-2-neopentylaminocyclohexyl, trans-4-neopentylaminocyclohexyl, or trans-4-methoxycyclohexyl; R₇ is —H; R₈ is methyl, ethyl, cyclopropylmethyl, or trifluoromethyl; R₉ and R₁₀ are methyl; X is N; and Y is C.

In another aspect, the invention provides compounds of Formula (I) wherein R₁ and R₂ are —H; R₃ is —H; R₄ and R₅ are —F; R₆ is (C₃-C₈)cycloalkyl(C₁-C₈)alkyl; R₇ is —H; R₈ is (C₁-C₈)alkyl, (C₃-C₈)cycloalkyl(C₁-C₈)alkyl, or halo(C₁-C₈)alkyl; R₉ and R₁₀ are independently (C₁-C₈)alkyl; X is N; and Y is C.

In another aspect, the invention provides compounds of Formula (I) wherein R₁ and R₂ are —H; R₃ is —H; R₄ and R₅ are —F; R₆ is cyclopropylmethyl; R₇ is —H; R₈ is methyl, ethyl, cyclopropylmethyl, or trifluormethyl; R₉ and R₁₀ are methyl; X is N; and Y is C.

In another aspect, the invention provides compounds of Formula (I) wherein R₁ and R₂ are —H; R₃ is —H; R₄ and R₅ are —F; R₆ is heteroaryl(C₁-C₈)alkylthio(C₁-C₈)alkyl; R₇ is —H; R₈ is (C₁-C₈)alkyl, (C₃-C₈)cycloalkyl(C₁-C₈)alkyl, or halo(C₁-C₈)alkyl; R₉ and R₁₀ are independently (C₁-C₈)alkyl; X is N; and Y is C.

In another aspect, the invention provides compounds of Formula (I) wherein R₁ and R₂ are —H; R₃ is —H; R₄ and R₅ are —F; R₆ is ethyl 2-((1H-imidazolyl)methylthio)ethyl; R₇ is —H; R₈ is methyl, ethyl, cyclopropylmethyl, or trifluoromethyl; R₉ and R₁₀ are methyl; X is N; and Y is C.

In another aspect, the invention provides compounds of Formula (I) wherein R₁ and R₂ are —H; R₃ is —H; R₄ and R₅ are —F; R₆ is heterocyclyl optionally substituted with 1 group that is (C₁-C₈)alkylcarbonyl or oxo; R₇ is —H; R₈ is (C₁-C₈)alkyl, (C₃-C₈)cycloalkyl(C₁-C₈)alkyl, or halo(C₁-C₈)alkyl; R₉ and R₁₀ are independently (C₁-C₈)alkyl; X is N; and Y is C.

In another aspect, the invention provides compounds of Formula (I) wherein R₁ and R₂ are —H; R₃ is —H; R₄ and R₅ are —F; R₆ is tetrahydrofuranyl, tetrahydropyran, piperidinyl, or azepanyl, wherein each is optionally substituted with 1 group that is (C₁-C₈)alkylcarbonyl or oxo; R₇ is —H; R₈ is methyl, ethyl, cyclopropylmethyl, or trifluoromethyl; R₉ and R₁₀ are methyl; X is N; and Y is C.

In another aspect, the invention provides compounds of Formula (I) wherein R₁ and R₂ are —H; R₃ is —H; R₄ and R₅ are —F; R₆ is tetrahydrofuranyl, 2-oxotetrahydrofuranyl, tetrahydropyranyl, 2-oxoazepanyl, or 1-acetylpiperidinyl; R₇ is —H; R₈ is methyl, ethyl, cyclopropylmethyl, or trifluoromethyl; R₉ and R₁₀ are methyl; X is N; and Y is C.

In another aspect, the invention provides compounds of Formula (I) wherein R₁ and R₂ are —H; R₃, R₄, and R₅ are —F; R₆ is (C₁-C₁₄) alkyl, (C₃-C₈)cycloalkenyl, (C₃-C₈)cycloalkyl, (C₃-C₈)cycloalkyl(C₁-C₈)alkyl, heteroaryl(C₁-C₈)alkylthio(C₁-C₈)alkyl, or heterocyclyl, wherein the R₆ group is optionally substituted with 1 or 2 groups that are (C₁-C₈)alkoxy, (C₁-C₈)alkoxycarbonyl, (C₁-C₈)alkylcarbonyl, hydroxy, oxo, R₁₂CH(NH₂)C(O)O—, NH₂CH₂CH₂C(O)O— or —NR₁₁R₁₂; R₇ is —H; R₈ is (C₁-C₈)alkyl, (C₃-C₈)cycloalkyl(C₁-C₈)alkyl, or halo(C₁-C₈)alkyl; R₉ and R₁₀ are independently (C₁-C₈)alkyl; R₁₁ and R₁₂ are independently —H or (C₁-C₈)alkyl; X is N or CH; and Y is C.

In another aspect, the invention provides compounds of Formula (I) wherein R₁ and R₂ are —H; R₃, R₄, and R₅ are —F; R₆ is (C₁-C₁₄)alkyl substituted with 1 or 2 groups that are (C₁-C₈)alkoxy, (C₁-C₈)alkoxycarbonyl, or hydroxy; R₇ is —H; R₈ is (C₁-C₈)alkyl, (C₃-C₈)cycloalkyl(C₁-C₈)alkyl, or halo(C₁-C₈)alkyl; R₉ and R₁₀ are independently (C₁-C₈)alkyl; X is N; and Y is C.

In another aspect, the invention provides compounds of Formula (I) wherein R₁ and R₂ are —H; R₃, R₄, and R₅ are —F; R₆ is (C₁-C₁₄)alkyl substituted with 1 or 2 groups that are (C₁-C₈)alkoxy, (C₁-C₈)alkoxycarbonyl, or hydroxy; R₇ is —H; R₈ is methyl, ethyl, cyclopropyl, or trifluoromethyl; R₉ and R₁₀ are independently (C₁-C₈)alkyl; X is N; and Y is C.

In another aspect, the invention provides compounds of Formula (I) wherein R₁ and R₂ are —H; R₃, R₄, and R₅ are —F; R₆ is 2-hydroxyethyl, 1,3-dihydroxypropyl, 2,2-dimethoxyethyl, or 4-hydroxy-1-methoxy-1-oxobutan-2-yl; R₇ is —H; R₈ is methyl, ethyl, cyclopropylmethyl, or trifluoromethyl; R₉ and R₁₀ are methyl; X is N; and Y is C.

In another aspect, the invention provides compounds of Formula (I) wherein R₁ and R₂ are —H; R₃, R₄, and R₅ are —F; R₆ is (C₃-C₈)cycloalkenyl; R₇ is —H; R₈ is (C₁-C₈)alkyl, (C₃-C₈)cycloalkyl(C₁-C₈)alkyl, or halo(C₁-C₈)alkyl; R₉ and R₁₀ are independently (C₁-C₈)alkyl; X is N; and Y is C.

In another aspect, the invention provides compounds of Formula (I) wherein R₁ and R₂ are —H; R₃, R₄, and R₅ are —F; R₆ is (C₃-C₈)cycloalkenyl; R₇ is —H; R₈ is methyl, ethyl, cyclopropylmethyl, or trifluoromethyl; R₉ and R₁₀ are methyl; X is N; and Y is C.

In another aspect, the invention provides compounds of Formula (I) wherein R₁ and R₂ are —H; R₃, R₄, and R₅ are —F; R₆ is cyclopentenyl; R₇ is —H; R₈ is methyl, ethyl, cyclopropylmethyl, or trifluoromethyl; R₉ and R₁₀ are methyl; X is N; and Y is C.

In another aspect, the invention provides compounds of Formula (I) wherein R₁ and R₂ are —H; R₃, R₄, and R₅ are —F; R₆ is (C₃-C₈)cycloalkyl optionally substituted with one group that is (C₁-C₈)alkoxy, hydroxy, R₁₂CH(NH₂)C(O)O—, NH₂CH₂CH₂C(O)O— or —NR₁₁R₁₂; R₇ is —H; R₈ is (C₁-C₈) alkyl, (C₃-C₈) cycloalkyl (C₁-C₈) alkyl, or halo (C₁-C₈) alkyl; R₉ and R₁₀ are independently (C₁-C₈)alkyl; R₁₁ and R₁₂ are independently —H or (C₁-C₈)alkyl; X is N; and Y is C.

In another aspect, the invention provides compounds of Formula (I) wherein R₁ and R₂ are —H; R₃, R₄, and R₅ are —F; R₆ is cyclobutyl, cyclopentyl, or cyclohexyl, wherein each is optionally substituted with one group that is (C₁-C₈)alkoxy, hydroxy, R₁₂CH(NH₂)C(O)O—, NH₂CH₂CH₂C(O)O— or —NR₁₁R₁₂; R₇ is —H; R₈ is methyl, ethyl, cyclopropylmethyl, or trifluoromethyl; R₉ and R₁₀ are methyl; R₁₁ and R₁₂ are independently —H or (C₁-C₈)alkyl; X is N; and Y is C.

In another aspect, the invention provides compounds of Formula (I) wherein R₁ and R₂ are —H; R₃, R₄, and R₅ are —F; R₆ is cyclobutyl, 2-hydroxycyclopentyl, 2-aminocyclohexyl, 2-hydroxycyclohexyl, 4-hydroxycyclohexyl, 2-neopentylaminocyclohexyl, 4-neopentylaminocyclohexyl, or 4-methoxycyclohexyl; R₇ is —H; R₈ is methyl, ethyl, cyclopropylmethyl, or trifluoromethyl; R₉ and R₁₀ are methyl; X is N; and Y is C.

In another aspect, the invention provides compounds of Formula (I) wherein R₁ and R₂ are —H; R₃, R₄, and R₅ are —F; R₆ is cyclobutyl, trans-2-hydroxycyclopentyl, trans-2-aminocyclohexyl, trans-2-hydroxycyclohexyl, trans-4-hydroxycyclohexyl, trans-2-neopentylaminocyclohexyl, trans-4-neopentylaminocyclohexyl, or trans-4-methoxycyclohexyl; R₇ is —H; R₈ is methyl, ethyl, cyclopropylmethyl, or trifluoromethyl; R₉ and R₁₀ are methyl; X is N; and Y is C.

In another aspect, the invention provides compounds of Formula (I) wherein R₁ and R₂ are —H; R₃, R₄, and R₅ are —F; R₆ is (C₃-C₈)cycloalkyl(C₁-C₈)alkyl; R₇ is —H; R₈ is (C₁-C₈)alkyl, (C₃-C₈)cycloalkyl(C₁-C₈)alkyl, or halo(C₁-C₈)alkyl; R₉ and R₁₀ are independently (C₁-C₈)alkyl; X is N; and Y is C.

In another aspect, the invention provides compounds of Formula (I) wherein R₁ and R₂ are —H; R₃, R₄, and R₅ are —F; R₆ is cyclopropylmethyl; R₇ is —H; R₈ is methyl, ethyl, cyclopropylmethyl, or trifluormethyl; R₉ and R₁₀ are methyl; X is N; and Y is C.

In another aspect, the invention provides compounds of Formula (I) wherein R₁ and R₂ are —H; R₃, R₄, and R₅ are —F; R₆ is heteroaryl(C₁-C₈)alkylthio(C₁-C₈)alkyl; R₇ is —H; R₈ is (C₁-C₈)alkyl, (C₃-C₈)cycloalkyl(C₁-C₈)alkyl, or halo(C₁-C₈)alkyl; R₉ and R₁₀ are independently (C₁-C₈)alkyl; X is N; and Y is C.

In another aspect, the invention provides compounds of Formula (I) wherein R₁ and R₂ are —H; R₃, R₄, and R₅ are —F; R₆ is ethyl 2-((1H-imidazolyl)methylthio)ethyl; R₇ is —H; R₈ is methyl, ethyl, cyclopropylmethyl, or trifluoromethyl; R₉ and R₁₀ are methyl; X is N; and Y is C.

In another aspect, the invention provides compounds of Formula (I) wherein R₁ and R₂ are —H; R₃, R₄, and R₅ are —F; R₆ is heterocyclyl optionally substituted with 1 group that is (C₁-C₈)alkylcarbonyl or oxo; R₇ is —H; R₈ is (C₁-C₈)alkyl, (C₃-C₈)cycloalkyl(C₁-C₈)alkyl, or halo(C₁-C₈)alkyl; R₉ and R₁₀ are independently (C₁-C₈)alkyl; X is N; and Y is C.

In another aspect, the invention provides compounds of Formula (I) wherein R₁ and R₂ are —H; R₃, R₄, and R₅ are —F; R₆ is tetrahydrofuranyl, tetrahydropyran, piperidinyl, or azepanyl, wherein each is optionally substituted with 1 group that is (C₁-C₈)alkylcarbonyl or oxo; R₇ is —H; R₈ is methyl, ethyl, cyclopropylmethyl, or trifluoromethyl; R₉ and R₁₀ are methyl; X is N; and Y is C.

In another aspect, the invention provides compounds of Formula (I) wherein R₁ and R₂ are —H; R₃, R₄, and R₅ are —F; R₆ is tetrahydrofuranyl, 2-oxotetrahydrofuranyl, tetrahydropyranyl, 2-oxoazepanyl, or 1-acetylpiperidinyl; R₇ is —H; R₈ is methyl, ethyl, cyclopropylmethyl, or trifluoromethyl; R₉ and R₁₀ are methyl; X is N; and Y is C.

In another aspect, the invention provides compounds of Formula (I) wherein R₁ and R₂ are —H; R₃ is —F; R₄ and R₅ are —H; R₆ is (C₁-C₁₄)alkyl, (C₃-C₈)cycloalkenyl, (C₃-C₈)cycloalkyl, (C₃-C₈)cycloalkyl(C₁-C₈)alkyl, heteroaryl(C₁-C₈)alkylthio(C₁-C₈)alkyl, or heterocyclyl, wherein the R₆ group is optionally substituted with 1 or 2 groups that are (C₁-C₈)alkoxy, (C₁-C₈)alkoxycarbonyl, (C₁-C₈)alkylcarbonyl, hydroxy, oxo, R₁₂CH(NH₂)C(O)O—, NH₂CH₂CH₂C(O)O— or —NR₁₁R₁₂; R₇ is —H; R₈ is (C₁-C₈)alkyl, (C₃-C₈)cycloalkyl(C₁-C₈)alkyl, or halo(C₁-C₈)alkyl; R₉ and R₁₀ are independently (C₁-C₈)alkyl; R₁₁ and R₁₂ are independently —H or (C₁-C₈)alkyl; X is CR₁₃; Y is C; and R₁₃ is —H or (C₁-C₈) alkyl.

In another aspect, the invention provides compounds of Formula (I) wherein R₁ and R₂ are —H; R₃ is —F; R₄ and R₅ are —H; R₆ is (C₁-C₁₄)alkyl substituted with 1 or 2 groups that are (C₁-C₈)alkoxy, (C₁-C₈)alkoxycarbonyl, or hydroxy; R₇ is —H; R₈ is (C₁-C₈)alkyl, (C₃-C₈)cycloalkyl(C₁-C₈)alkyl, or halo(C₁-C₈)alkyl; R₉ and R₁₀ are independently (C₁-C₈)alkyl; X is CR₁₃; Y is C; and R₁₃ is —H or (C₁-C₈)alkyl.

In another aspect, the invention provides compounds of Formula (I) wherein R₁ and R₂ are —H; R₃ is —F; R₄ and R₅ are —H; R₆ is (C₁-C₁₄)alkyl substituted with 1 or 2 groups that are (C₁-C₈) alkoxy, (C₁-C₈) alkoxycarbonyl, or hydroxy; R₇ is —H; R₈ is methyl, ethyl, cyclopropyl, or trifluoromethyl; R₉ and R₁₀ are independently (C₁-C₈)alkyl; X is CR₁₃; Y is C; and R₁₃ is —H or methyl.

In another aspect, the invention provides compounds of Formula (I) wherein R₁ and R₂ are —H; R₃ is —F; R₄ and R₅ are —H; R₆ is 2-hydroxyethyl, 1,3-dihydroxypropyl, 2,2-dimethoxyethyl, or 4-hydroxy-1-methoxy-1-oxobutan-2-yl; R₇ is —H; R₈ is methyl, ethyl, cyclopropylmethyl, or trifluoromethyl; R₉ and R₁₀ are methyl; X is CR₁₃; Y is C; and R₁₃ is —H or methyl.

In another aspect, the invention provides compounds of Formula (I) wherein R₁ and R₂ are —H; R₃ is —F; R₄ and R₅ are —H; R₆ is (C₃-C₈) cycloalkenyl; R₇ is —H; R₈ is (C₁-C₈) alkyl, (C₃-C₈) cycloalkyl (C₁-C₈) alkyl, or halo (C₁-C₈) alkyl; R₉ and R₁₀ are independently (C₁-C₈)alkyl; X is CR₁₃; Y is C; and R₁₃ is —H or (C₁-C₈) alkyl.

In another aspect, the invention provides compounds of Formula (I) wherein R₁ and R₂ are —H; R₃ is —F; R₄ and R₅ are —H; R₆ is (C₃-C₈)cycloalkenyl; R₇ is —H; R₈ is methyl, ethyl, cyclopropylmethyl, or trifluoromethyl; R₉ and R₁₀ are methyl; X is CR₁₃; Y is C; and R₁₃ is —H or methyl.

In another aspect, the invention provides compounds of Formula (I) wherein R₁ and R₂ are —H; R₃ is —F; R₄ and R₅ are —H; R₆ is cyclopentenyl; R₇ is —H; R₈ is methyl, ethyl, cyclopropylmethyl, or trifluoromethyl; R₉ and R₁₀ are methyl; X is CR₁₃; Y is C; and R₁₃ is —H or methyl.

In another aspect, the invention provides compounds of Formula (I) wherein R₁ and R₂ are —H; R₃ is —F; R₄ and R₅ are —H; R₆ is (C₃-C₈)cycloalkyl optionally substituted with one group that is (C₁-C₈)alkoxy, hydroxy, R₁₂CH(NH₂)C(O)O—, NH₂CH₂C(O)O—, NH₂CH₂CH₂C(O)O—, or —NR₁₁R₁₂; R₇ is —H; R₈ is (C₁-C₈)alkyl, (C₃-C₈)cycloalkyl(C₁-C₈)alkyl, or halo(C₁-C₈)alkyl; R₉ and R₁₀ are independently (C₁-C₈) alkyl; R₁₁ and R₁₂ are independently —H or (C₁-C₈)alkyl; Y is C; X is CR₁₃; and R₁₃ is —H or (C₁-C₈) alkyl.

In another aspect, the invention provides compounds of Formula (I) wherein R₁ and R₂ are —H; R₃ is —F; R₄ and R₅ are —H; R₆ is cyclobutyl, cyclopentyl, or cyclohexyl, wherein each is optionally substituted with one group that is (C₁-C₈)alkoxy, hydroxy, R₁₂CH(NH₂)C(O)O—, NH₂CH₂C(O)O—, NH₂CH₂CH₂C(O)O—, or —NR₁₁R₁₂; R₇ is —H; R₈ is methyl, ethyl, cyclopropylmethyl, or trifluoromethyl; R₉ and R₁₀ are methyl; R₁₁ and R₁₂ are independently —H or (C₁-C₈)alkyl; X is CR₁₃; Y is C; and R₁₃ is —H or methyl.

In another aspect, the invention provides compounds of Formula (I) wherein R₁ and R₂ are —H; R₃ is —F; R₄ and R₅ are —H; R₆ is cyclobutyl, 2-hydroxycyclopentyl, 2-aminocyclohexyl, 2-(2-aminoacetoxy)cyclopentyl, 2-(2-aminoacetoxy)cyclohexyl, 4-(2-aminoacetoxy)cyclohexyl, 2-hydroxycyclohexyl, 4-hydroxycyclohexyl, 2-neopentylaminocyclohexyl, 4-neopentylaminocyclohexyl, or 4-methoxycyclohexyl; R₇ is —H; R₈ is methyl, ethyl, cyclopropylmethyl, or trifluoromethyl; R₉ and R₁₀ are methyl; X is CR₁₃; Y is C; and R₁₃ is —H or methyl.

In another aspect, the invention provides compounds of Formula (I) wherein R₁ and R₂ are —H; R₃ is —F; R₄ and R₅ are —H; R₆ is cyclobutyl, trans-2-hydroxycyclopentyl, trans-2-(2-aminoacetoxy)cyclopentyl, trans-2-(2-aminoacetoxy)cyclohexyl, trans-4-(2-aminoacetoxy)cyclohexyl, trans-2-aminocyclohexyl, trans-2-hydroxycyclohexyl, trans-4-hydroxycyclohexyl, trans-2-neopentylaminocyclohexyl, trans-4-neopentylaminocyclohexyl, or trans-4-methoxycyclohexyl; R₇ is —H; R₈ is methyl, ethyl, cyclopropylmethyl, or trifluoromethyl; R₉ and R₁₀ are methyl; X is CR₁₃; Y is C; and R₁₃ is —H or methyl.

In another aspect, the invention provides compounds of Formula (I) wherein R₁ and R₂ are —H; R₃ is —F; R₄ and R₅ are —H; R₆ is (C₃-C₈)cycloalkyl(C₁-C₈)alkyl; R₇ is —H; R₈ is (C₁-C₈)alkyl, (C₃-C₈)cycloalkyl(C₁-C₈)alkyl, or halo(C₁-C₈)alkyl; R₉ and R₁₀ are independently (C₁-C₈)alkyl; X is CR₁₃; Y is C; and R₁₃ is —H or (C₁-C₈) alkyl.

In another aspect, the invention provides compounds of Formula (I) wherein R₁ and R₂ are —H; R₃ is —F; R₄ and R₅ are —H; R₆ is cyclopropylmethyl; R₇ is —H; R₈ is methyl, ethyl, cyclopropylmethyl, or trifluormethyl; R₉ and R₁₀ are methyl; X is CR₁₃; Y is C; and R₁₃ is —H or methyl.

In another aspect, the invention provides compounds of Formula (I) wherein R₁ and R₂ are —H; R₃ is —F; R₄ and R₅ are —H; R₆ is heteroaryl(C₁-C₈)alkylthio(C₁-C₈)alkyl; R₇ is —H; R₈ is (C₁-C₈) alkyl, (C₃-C₈)cycloalkyl(C₁-C₈)alkyl, or halo(C₁-C₈)alkyl; R₉ and R₁₀ are independently (C₁-C₈)alkyl; X is CR₁₃; Y is C; and R₁₃ is —H or (C₁-C₈) alkyl.

In another aspect, the invention provides compounds of Formula (I) wherein R₁ and R₂ are —H; R₃ is —F; R₄ and R₅ are —H; R₆ is ethyl 2-((1H-imidazolyl)methylthio)ethyl; R₇ is —H; R₈ is methyl, ethyl, cyclopropylmethyl, or trifluoromethyl; R₉ and R₁₀ are methyl; X is CR₁₃; Y is C; and R₁₃ is —H or methyl.

In another aspect, the invention provides compounds of Formula (I) wherein R₁ and R₂ are —H; R₃ is —F; R₄ and R₅ are —H; R₆ is heterocyclyl optionally substituted with 1 group that is (C₁-C₈)alkylcarbonyl or oxo; R₇ is —H; R₈ is (C₁-C₈)alkyl, (C₃-C₈)cycloalkyl(C₁-C₈)alkyl, or halo(C₁-C₈)alkyl; R₉ and R₁₀ are independently (C₁-C₈)alkyl; X is CR₁₃; Y is C; and R₁₃ is —H or (C₁-C₈) alkyl.

In another aspect, the invention provides compounds of Formula (I) wherein R₁ and R₂ are —H; R₃ is —F; R₄ and R₅ are —H; R₆ is tetrahydrofuranyl, tetrahydropyran, piperidinyl, or azepanyl, wherein each is optionally substituted with 1 group that is (C₁-C₈)alkylcarbonyl or oxo; R₇ is —H; R₈ is methyl, ethyl, cyclopropylmethyl, or trifluoromethyl; R₉ and R₁₀ are methyl; X is CR₁₃; Y is C; and R₁₃ is —H or methyl.

In another aspect, the invention provides compounds of Formula (I) wherein R₁ and R₂ are —H; R₃ is —F; R₄ and R₅ are —H; R₆ is tetrahydrofuranyl, 2-oxotetrahydrofuranyl, tetrahydropyranyl, 2-oxoazepanyl, or 1-acetylpiperidinyl; R₇ is —H; R₈ is methyl, ethyl, cyclopropylmethyl, or trifluoromethyl; R₉ and R₁₀ are methyl; X is CR₁₃; Y is C; and R₁₃ is —H or methyl.

In another aspect, the invention provides a pharmaceutical composition comprising a therapeutically effective amount of a compound of Formula (I) wherein at least one of R₃, R₄, and R₅ is —F, or a pharmaceutically acceptable salt thereof, and at least one pharmaceutically acceptable carrier, solvent, adjuvant or diluent.

In another aspect, the invention provides a method of treating a disease or disorder related to cell proliferation comprising administering a therapeutically effective amount of a compound of Formula (I) wherein at least one of R₃, R₄, and R₅ is —F, or a pharmaceutically acceptable salt thereof, to a patient in need of such treatment.

In another aspect, the invention provides a method of inhibiting cell proliferation comprising administering a therapeutically effective amount of a compound of Formula (I) wherein at least one of R₃, R₄, and R₅ is —F, or a pharmaceutically acceptable salt thereof, to a patient in need of such treatment.

In another aspect, the invention provides a method of treating a disease or disorder related to Heat-shock protein 90 comprising administering a therapeutically effective amount of a compound of Formula (I) wherein at least one of R₃, R₄, and R₅ is —F, or a pharmaceutically acceptable salt thereof, to a patient in need of such treatment.

In another aspect, the invention provides a method of inhibiting Heat-shock protein 90 comprising administering a therapeutically effective amount of a compound of Formula (I) wherein at least one of R₃, R₄, and R₅ is —F, or a pharmaceutically acceptable salt thereof, to a patient in need of such treatment.

In another aspect, the invention provides a method of treating cancer comprising administering a therapeutically effective amount of a compound of Formula (I) wherein at least one of R₃, R₄, and R₅ is —F, or a pharmaceutically acceptable salt thereof, to a patient in need of such treatment.

In another aspect, the invention provides a method of treating breast cancer comprising administering a therapeutically effective amount of a compound of Formula (I) wherein at least one of R₃, R₄, and R₅ is —F, or a pharmaceutically acceptable salt thereof, to a patient in need of such treatment.

In another aspect, the invention provides a method of treating uterine cancer comprising administering a therapeutically effective amount of a compound of Formula (I) wherein at least one of R₃, R₄, and R₅ is —F, or a pharmaceutically acceptable salt thereof, to a patient in need of such treatment.

In another aspect, the invention provides a pharmaceutical composition comprising a therapeutically effective amount of a compound of Formula (I) wherein the compound is Example 3, 6, 10, 17, 19, 20, 25, 28, 32, 39-43, 45, 48-99, or 100, or a pharmaceutically acceptable salt thereof, and at least one pharmaceutically acceptable carrier, solvent, adjuvant or diluent.

In another aspect, the invention provides a method of treating a disease or disorder related to cell proliferation comprising administering a therapeutically effective amount of a compound of Formula (I), wherein the compound is Example 3, 6, 10, 17, 19, 20, 25, 28, 32, 39-43, 45, 48-99, or 100, or a pharmaceutically acceptable salt thereof, to a patient in need of such treatment.

In another aspect, the invention provides a method of inhibiting cell proliferation comprising administering a therapeutically effective amount of a compound of Formula (I),

wherein the compound is Example 3, 6, 10, 17, 19, 20, 25, 28, 32, 39-43, 45, 48-99, or 100, or a pharmaceutically acceptable salt thereof, to a patient in need of such treatment.

In another aspect, the invention provides a method of treating a disease or disorder related to Heat-shock protein 90 comprising administering a therapeutically effective amount of a compound of Formula (I), wherein the compound is Example 3, 6, 10, 17, 19, 20, 25, 28, 32, 39-43, 45, 48-99, or 100, or a pharmaceutically acceptable salt thereof, to a patient in need of such treatment.

In another aspect, the invention provides a method of inhibiting Heat-shock protein 90 comprising administering a therapeutically effective amount of a compound of Formula (I),

wherein the compound is Example 3, 6, 10, 17, 19, 20, 25, 28, 32, 39-43, 45, 48-99, or 100, or a pharmaceutically acceptable salt thereof, to a patient in need of such treatment.

In another aspect, the invention provides a method of treating cancer comprising administering a therapeutically effective amount of a compound of Formula (I), wherein the compound is Example 3, 6, 10, 17, 19, 20, 25, 28, 32, 39-43, 45, 48-99, or 100, or a pharmaceutically acceptable salt thereof, to a patient in need of such treatment.

In another aspect, the invention provides a method of treating breast cancer comprising administering a therapeutically effective amount of a compound of Formula (I), wherein the compound is Example 3, 6, 10, 17, 19, 20, 25, 28, 32, 39-43, 45, 48-99, or 100, or a pharmaceutically acceptable salt thereof, to a patient in need of such treatment.

In another aspect, the invention provides a method of treating uterine cancer comprising administering a therapeutically effective amount of a compound of Formula (I), wherein the compound is Example 3, 6, 10, 17, 19, 20, 25, 28, 32, 39-43, 45, 48-99, or 100, or a pharmaceutically acceptable salt thereof, to a patient in need of such treatment.

In another aspect, the invention provides a pharmaceutical composition comprising a therapeutically effective amount of a compound of Formula (I) wherein the compound is selected from Examples 189-215, 228, or 231-250, or a pharmaceutically acceptable salt thereof, and at least one pharmaceutically acceptable carrier, solvent, adjuvant or diluent.

In another aspect, the invention provides a method of treating a disease or disorder related to cell proliferation comprising administering a therapeutically effective amount of a compound of Formula (I), wherein the compound is selected from Examples 189-215, 228, or 231-250, or a pharmaceutically acceptable salt thereof, to a patient in need of such treatment.

In another aspect, the invention provides a method of inhibiting cell proliferation comprising administering a therapeutically effective amount of a compound of Formula (I), wherein the compound is selected from Examples 189-215, 228, or 231-250, or a pharmaceutically acceptable salt thereof, to a patient in need of such treatment.

In another aspect, the invention provides a method of treating a disease or disorder related to Heat-shock protein 90 comprising administering a therapeutically effective amount of a compound of Formula (I), wherein the compound is selected from Example 189-215, 228, or 231-250, or a pharmaceutically acceptable salt thereof, to a patient in need of such treatment.

In another aspect, the invention provides a method of inhibiting Heat-shock protein 90 comprising administering a therapeutically effective amount of a compound of Formula (I), wherein the compound is selected from Example 189-215, 228, or 231-250, or a pharmaceutically acceptable salt thereof, to a patient in need of such treatment.

In another aspect, the invention provides a method of treating cancer comprising administering a therapeutically effective amount of a compound of Formula (I), wherein the compound is selected from Example 189-215, 228, or 231-250, or a pharmaceutically acceptable salt thereof, to a patient in need of such treatment.

In another aspect, the invention provides a method of treating breast cancer comprising administering a therapeutically effective amount of a compound of Formula (I), wherein the compound is selected from Example 189-215, 228, or 231-250, or a pharmaceutically acceptable salt thereof, to a patient in need of such treatment.

In another aspect, the invention provides a method of treating uterine cancer comprising administering a therapeutically effective amount of a compound of Formula (I), wherein the compound is selected from Example 189-215, 228, or 231-250, or a pharmaceutically acceptable salt thereof, to a patient in need of such treatment.

Compounds of the invention, or a salt thereof, where one or more basic nitrogens or amino groups exist at R₆ have improved aqueous solubility compared to their non-amino equivalents. For example, the mono hydrochloride salt of Example 121 has a solubility greater than 10 mg/mL in water as compared to the non-amino equivalent, which is essentially insoluble in water. The mono hydrochloride salts or mesylate salts of compounds of the invention, where R₆ contains one or more basic amino groups, have solubilities greater than 1 mg/mL. Therefore, compounds of the invention, where R₆ contains one or more amino groups, are more easily formulated into a solution for intravenous or related drug administration.

Further, compounds of the invention, where R₆ contains one or more basic amino groups, have increased HSP-90 inhibitory activity and greater potencies against cancer cell growth compared to similar analogs that lack amino groups at R₆. For Example, a compound of the invention, Example 106, inhibits growth of a PC-3 cell line 5-10 times more than compounds lacking a basic nitrogen or amino group at R₆.

Preferred R₆ amino groups for compounds of the invention include, but are not limited to, 2-(prop-2-ynylamino)ethyl, 2-(diprop-2-ynylamino)ethyl, 2-(2-aminoacetoxy)cyclopentyl, 2-(2-aminoacetoxy)cyclohexyl, 4-(2-aminoacetoxy)cyclohexyl, (S)-4-(2-amino-3-hydroxypropanoyloxy)cyclohexyl, 4-(2-(dimethylamino)acetoxy)cyclohexyl, trans-4-(prop-2-ynylamino)cyclohexyl, trans-2-(prop-2-ynylamino)cyclohexyl, trans-2-aminocyclohexyl, trans-3-aminocyclohexyl, trans-4-(cyclopropylmethylamino)cyclohexyl, trans-2-(cyclopropylmethylamino)cyclohexyl, trans-2-(dicyclopropylmethylamino)cyclohexyl, trans-4-(diprop-2-ynylamino)cyclohexyl, trans-2-(diethylamino)cyclohexyl, trans-2-(cyclopropylamino)cyclohexyl, trans-2-(neopentylamino)cyclohexyl, trans-4-(neopentylamino)cyclohexyl; pyridazinyl, pyrimidinyl, 2-(2-hydroxyethoxy)pyridinyl, 1-(2-(methylamino)ethyl)-1H-pyrazolyl, 1-(2-(isobutylamino)ethyl)-1H-pyrazolyl, 1-(2-(tert-butylamino)ethyl)-1H-pyrazolyl, 2-(pyrimidinylthio)ethyl, 2-(1H-imidazolylthio)ethyl, azetidinyl, piperidinyl, 2,2,6,6-tetramethylpiperidinyl, 1-(prop-2-ynyl)piperidinyl, 1-cyclopropylpiperidinyl, 1-cyclopropylmethylpiperidinyl, 1-(2-methoxyethyl)piperidinyl, 1-(prop-2-ynyl)pyrrolidinyl, 1-allylpiperidinyl, and 1-neopentylpiperidinyl.

Compounds of the invention with one or more basic nitrogens (or amino groups) at R₆ that have improved aqueous solubility and/or improved HSP-90 inhibitory activity and greater potency against cancer cell growth include, but are not limited to, Examples 4, 24, 37, 44, 38, 102-142, 216-219, and 251-254.

Accordingly, in another aspect, the invention provides compounds of Formula (I) wherein R₁, R₂, R₃, R₄, and R₅ are —H; R₆ is (C₁-C₁₄)alkyl, (C₃-C₈)cycloalkyl, heteroaryl, heteroarylthio(C₁-C₈)alkyl, or heterocyclyl, wherein the R₆ group is optionally substituted with 1 group that is (C₂-C₈)alkenyl, (C₁-C₈)alkoxy(C₁-C₈)alkyl, (C₁-C₈)alkyl, (C₂-C₈)alkynyl, (C₃-C₈)cycloalkyl, (C₃-C₈)cycloalkyl(C₁-C₈)alkyl, hydroxy(C₁-C₈)alkoxy, —NR₁₁R₁₂, (NR₁₁R₁₂) (C₁-C₈)alkyl, R₁₂CH(NH₂) C(O)O—, NH₂CH₂C(O)O—, NH₂CH₂CH₂C(O)O—, HOCH₂CH(NH₂)C(O)O— or (CH₃)₂NCH₂C(O)O—; R₇ is —H; R₈ is (C₁-C₈)alkyl, (C₃-C₈)cycloalkyl(C₁-C₈)alkyl, halo(C₁-C₈)alkyl; R₉ and R₁₀ are independently (C₁-C₈)alkyl; R₁₁ and R₁₂ are independently —H, (C₁-C₈)alkyl, (C₂-C₈)alkynyl, (C₃-C₈)cycloalkyl, or (C₃-C₈)cycloalkyl(C₁-C₈)alkyl; X is N or CH; and Y is C.

In another aspect, the invention provides compounds of Formula (I) wherein R₁, R₂, R₃, R₄, and R₅ are —H; R₆ is (C₁-C₁₄)alkyl substituted with —NR₁₁R₁₂; R₇ is —H; R₈ is (C₁-C₈)alkyl, (C₃-C₈)cycloalkyl(C₁-C₈)alkyl, halo(C₁-C₈)alkyl; R₉ and R₁₀ are independently (C₁-C₈)alkyl; R₁₁ and R₁₂ are independently —H or (C₂-C₈)alkynyl; X is N or CH; and Y is C.

In another aspect, the invention provides compounds of Formula (I) wherein R₁, R₂, R₃, R₄, and R₅ are —H; R₆ is 2-(prop-2-ynylamino)ethyl or 2-(diprop-2-ynylamino)ethyl; R₇ is —H; R₈ is methyl, difluoromethyl, trifluoromethyl; R₉ and R₁₀ are methyl; X is N; and Y is C.

In another aspect, the invention provides compounds of Formula (I) wherein R₁, R₂, R₃, R₄, and R₅ are —H; R₆ is (C₃-C₈)cycloalkyl substituted with 1 group that is —NR₁₁R₁₂, R₁₂CH(NH₂) C(O)O—, NH₂CH₂C(O)O—, NH₂CH₂CH₂C(O)O—, HOCH₂CH(NH₂)C(O)O— or (CH₃)₂NCH₂C(O)O—; R₇ is —H; R₈ is (C₁-C₈)alkyl or halo(C₁-C₈)alkyl; R₉ and R₁₀ are independently (C₁-C₈)alkyl; R₁₁ and R₁₂ are independently —H, (C₁-C₈) alkyl, (C₂-C₈) alkynyl, (C₃-C₈) cycloalkyl, or (C₃-C₈) cycloalkyl(C₁-C₈)alkyl; X is N or CH; and Y is C.

In another aspect, the invention provides compounds of Formula (I) wherein R₁, R₂, R₃, R₄, and R₅ are —H; R₆ is cyclopentyl or cyclohexyl substituted with 1 group that is —NR₁₁R₁₂, R₁₂CH(NH₂) C(O)O—, NH₂CH₂C(O)O—, NH₂CH₂CH₂C(O)O—, HOCH₂CH(NH₂)C(O)O— or (CH₃)₂NCH₂C(O)O—; R₇ is —H; R₈ is methyl, difluoromethyl, or trifluoromethyl; R₉ and R₁₀ are methyl; R₁₁ and R₁₂ are independently —H, (C₁-C₈) alkyl, (C₂-C₈)alkynyl, (C₃-C₈)cycloalkyl, or (C₃-C₈)cycloalkyl(C₁-C₈)alkyl; X is N or CH; and Y is C.

In another aspect, the invention provides compounds of Formula (I) wherein R₁, R₂, R₃, R₄, and R₅ are —H; R₆ is 2-(2-aminoacetoxy)cyclopentyl, (S)-4-(2-amino-3-hydroxypropanoyloxy)cyclohexyl, 2-(2-aminoacetoxy)cyclohexyl, 4-(2-aminoacetoxy)cyclohexyl, 4-(2-(dimethylamino)acetoxy)cyclohexyl, 4-(prop-2-ynylamino)cyclohexyl, 2-(prop-2-ynylamino)cyclohexyl, 2-aminocyclohexyl, 3-aminocyclohexyl, 4-(cyclopropylmethylamino)cyclohexyl, 2-(cyclopropylmethylamino)cyclohexyl, 2-(dicyclopropylmethylamino)cyclohexyl, 4-(diprop-2-ynylamino)cyclohexyl, 2-(diethylamino)cyclohexyl, 2-(cyclopropylamino)cyclohexyl, 2-(neopentylamino)cyclohexyl, or 4-(neopentylamino)cyclohexyl; R₇ is —H; R₈ is methyl, difluoromethyl, or trifluoromethyl; R₉ and R₁₀ are methyl; X is N; and Y is C.

In another aspect, the invention provides compounds of Formula (I) wherein R₁, R₂, R₃, R₄, and R₅ are —H; R₆ is trans-2-(2-aminoacetoxy)cyclopentyl, trans-2-(2-aminoacetoxy)cyclohexyl, trans-4-(2-aminoacetoxy)cyclohexyl, trans-4-(prop-2-ynylamino)cyclohexyl, trans-2-(prop-2-ynylamino)cyclohexyl, trans-2-aminocyclohexyl, trans-3-aminocyclohexyl, trans-4-(cyclopropylmethylamino)cyclohexyl, trans-2-(cyclopropylmethylamino)cyclohexyl, trans-2-(dicyclopropylmethylamino)cyclohexyl, trans-4-(diprop-2-ynylamino)cyclohexyl, trans-2-(diethylamino)cyclohexyl, trans-2-(cyclopropylamino)cyclohexyl, trans-2-(neopentylamino)cyclohexyl, or trans-4-(neopentylamino)cyclohexyl; R₇ is —H; R₈ is methyl, difluoromethyl, or trifluoromethyl; R₉ and R₁₀ are methyl; X is N; and Y is C.

In an aspect, the invention provides compounds of Formula (I) wherein R₁, R₂, R₃, R₄, and R₅ are —H; R₆ is heteroaryl optionally substituted with 1 group that is (C₁-C₈) alkoxy(C₁-C₈) alkyl or (NR₁₁R₁₂) (C₁-C₈) alkyl; R₇ is —H; R₈ is (C₁-C₈)alkyl, (C₃-C₈)cycloalkyl(C₁-C₈)alkyl, halo(C₁-C₈)alkyl; R₉ and R₁₀ are independently (C₁-C₈)alkyl; R₁ and R₁₂ are independently —H or (C₁-C₈)alkyl; X is N or CH; and Y is C.

In another aspect, the invention provides compounds of Formula (I) wherein R₁, R₂, R₃, R₄, and R₅ are —H; R₆ is pyrazolyl, pyridazinyl, pyrimidinyl, pyridinyl, wherein each is optionally substituted with 1 group that is (C₁-C₈)alkoxy(C₁-C₈)alkyl or (NR₁₁R₁₂) (C₁-C₈)alkyl; R₇ is —H; R₈ is methyl, difluoromethyl, or trifluoromethyl; R₉ and R₁₀ are methyl; R₁ and R₁₂ are independently —H or (C₁-C₈)alkyl; X is N; and Y is C.

In another aspect, the invention provides compounds of Formula (I) wherein R₁, R₂, R₃, R₄, and R₅ are —H; R₆ is pyridazinyl, pyrimidinyl, 2-(2-hydroxyethoxy)pyridinyl, 1-(2-(methylamino)ethyl)-1H-pyrazolyl, 1-(2-(isobutylamino)ethyl)-1H-pyrazolyl, 1-(2-(tert-butylamino)ethyl)-1H-pyrazolyl; R₇ is —H; R₈ is methyl, difluoromethyl, or trifluoromethyl; R₉ and R₁₀ are methyl; X is N; and Y is C.

In another aspect, the invention provides compounds of Formula (I) wherein R₁, R₂, R₃, R₄, and R₅ are —H; R₆ is heteroarylthio(C₁-C₈)alkyl; R₇ is —H; R is (C₁-C₈)alkyl, (C₃-C₈)cycloalkyl(C₁-C₈)alkyl, halo(C₁-C₈)alkyl; R₉ and R₁₀ are independently (C₁-C₈)alkyl; X is N or CH; and Y is C.

In another aspect, the invention provides compounds of Formula (I) wherein R₁, R₂, R₃, R₄, and R₅ are —H; R₆ is heteroarylthio(C₁-C₈)alkyl wherein the heteroaryl is pyrimidinyl or imidazolyl; R₇ is —H; R₈ is methyl, difluoromethyl, trifluoromethyl; R₉ and R₁₀ are methyl; X is N; and Y is C.

In another aspect, the invention provides compounds of Formula (I) wherein R₁, R₂, R₃, R₄, and R₅ are —H; R₆ is 2-(pyrimidinylthio)ethyl or 2-(1H-imidazolylthio)ethyl; R₇ is —H; R₈ is methyl, difluoromethyl, trifluoromethyl; R₉ and R₁₀ are methyl; X is N; and Y is C.

In another aspect, the invention provides compounds of Formula (I) wherein R₁, R₂, R₃, R₄, and R₅ are —H; R₆ is heterocyclyl optionally substituted with 1 group that is (C₂-C₈)alkenyl, (C₁-C₈)alkoxy(C₁-C₈)alkyl, (C₁-C₈)alkyl, (C₂-C₈) alkynyl, (C₃-C₈)cycloalkyl, or (C₃-C₈)cycloalkyl(C₁-C₈) alkyl; R₈ is (C₁-C₈) alkyl, (C₃-C₈)cycloalkyl(C₁-C₈)alkyl, halo(C₁-C₈)alkyl; R₉ and R₁₀ are independently (C₁-C₈)alkyl; X is N or CH; and Y is C.

In another aspect, the invention provides compounds of Formula (I) wherein R₁, R₂, R₃, R₄, and R₅ are —H; R₆ is azetidinyl, pyrrolidinyl, or piperidinyl, wherein each is optionally substituted with 1 group that is (C₂-C₈)alkenyl, (C₁-C₈)alkoxy(C₁-C₈)alkyl, (C₁-C₈)alkyl, (C₂-C₈)alkynyl, (C₃-C₈)cycloalkyl, or (C₃-C₈)cycloalkyl(C₁-C₈)alkyl; R₈ is methyl, difluormethyl, or trifluoromethyl; R₉ and R₁₀ are methyl; X is N; and Y is C.

In another aspect, the invention provides compounds of Formula (I) wherein R₁, R₂, R₃, R₄, and R₅ are —H; R₆ is azetidinyl, piperidinyl, 2,2,6,6-tetramethylpiperidinyl, 1-(prop-2-ynyl)piperidinyl, 1-cyclopropylpiperidinyl, 1-cyclopropylmethylpiperidinyl, 1-(2-methoxyethyl)piperidinyl, 1-(prop-2-ynyl)pyrrolidinyl, 1-allylpiperidinyl, or 1-neopentylpiperidinyl; R₈ is methyl, difluormethyl, or trifluoromethyl; R₉ and R₁₀ are methyl; X is N; and Y is C.

In another aspect, the invention provides compounds of Formula (I) wherein R₁, R₂, R₃, R₄, and R₅ are —H; R₆ is heterocyclyl substituted with 1, 2, 3, or 4 (C₁-C₈)alkyl groups; R₈ is (C₁-C₈)alkyl, (C₃-C₈)cycloalkyl(C₁-C₈)alkyl, halo(C₁-C₈)alkyl; R₉ and R₁₀ are independently (C₁-C₈)alkyl; X is N or CH; and Y is C.

In another aspect, the invention provides compounds of Formula (I) wherein R₁, R₂, R₃, R₄, and R₅ are —H; R₆ is piperidinyl substituted with 1, 2, 3, or 4 (C₁-C₈)alkyl groups; R₈ is methyl, difluormethyl, or trifluoromethyl; R₉ and R₁₀ are methyl; X is N; and Y is C.

In another aspect, the invention provides compounds of Formula (I) wherein R₁, R₂, R₃, R₄, and R₅ are —H; R₆ is piperidinyl substituted with 4 methyl groups; R₈ is methyl, difluormethyl, or trifluoromethyl; R₉ and R₁₀ are methyl; X is N; and Y is C.

In another aspect, the invention provides a pharmaceutical composition comprising a therapeutically effective amount of a compound of Formula (I) wherein the compound is Example 4, 24, 37, 44, 38, 47, 101-140, or 141, or a pharmaceutically acceptable salt thereof, and at least one pharmaceutically acceptable carrier, solvent, adjuvant or diluent.

In another aspect, the invention provides a method of treating a disease or disorder related to cell proliferation comprising administering a therapeutically effective amount of a compound of Formula (I), wherein the compound is Example 4, 24, 37, 44, 38, 47, 101-140, or 141, or a pharmaceutically acceptable salt thereof, to a patient in need of such treatment.

In another aspect, the invention provides a method of inhibiting cell proliferation comprising administering a therapeutically effective amount of a compound of Formula (I), wherein the compound is Example 4, 24, 37, 44, 38, 47, 101-140, or 141, or a pharmaceutically acceptable salt thereof, to a patient in need of such treatment.

In another aspect, the invention provides a method of treating a disease or disorder related to Heat-shock protein 90 comprising administering a therapeutically effective amount of a compound of Formula (I), wherein the compound is Example 4, 24, 37, 44, 38, 47, 101-140, or 141, or a pharmaceutically acceptable salt thereof, to a patient in need of such treatment.

In another aspect, the invention provides a method of inhibiting Heat-shock protein 90 comprising administering a therapeutically effective amount of a compound of Formula (I), wherein the compound is Example 4, 24, 37, 44, 38, 47, 101-140, or 141, or a pharmaceutically acceptable salt thereof, to a patient in need of such treatment.

In another aspect, the invention provides a method of treating cancer comprising administering a therapeutically effective amount of a compound of Formula (I), wherein the compound is Example 4, 24, 37, 44, 38, 47, 101-140, or 141, or a pharmaceutically acceptable salt thereof, to a patient in need of such treatment.

In another aspect, the invention provides a method of treating prostate cancer comprising administering a therapeutically effective amount of a compound of Formula (I), wherein the compound is Example 4, 24, 37, 44, 38, 47, 101-140, or 141, or a pharmaceutically acceptable salt thereof, to a patient in need of such treatment.

In another aspect, the invention provides a pharmaceutical composition comprising a therapeutically effective amount of a compound of Formula (I) wherein the compound is Example 216-219, or 251-254, or a pharmaceutically acceptable salt thereof, and at least one pharmaceutically acceptable carrier, solvent, adjuvant or diluent.

In another aspect, the invention provides a method of treating a disease or disorder related to cell proliferation comprising administering a therapeutically effective amount of a compound of Formula (I), wherein the compound is Example 216-219, or 251-254, or a pharmaceutically acceptable salt thereof, to a patient in need of such treatment.

In another aspect, the invention provides a method of inhibiting cell proliferation comprising administering a therapeutically effective amount of a compound of Formula (I), wherein the compound is Example 216-219, or 251-254, or a pharmaceutically acceptable salt thereof, to a patient in need of such treatment.

In another aspect, the invention provides a method of treating a disease or disorder related to Heat-shock protein 90 comprising administering a therapeutically effective amount of a compound of Formula (I), wherein the compound is Example 216-219, or 251-254, or a pharmaceutically acceptable salt thereof, to a patient in need of such treatment.

In another aspect, the invention provides a method of inhibiting Heat-shock protein 90 comprising administering a therapeutically effective amount of a compound of Formula (I), wherein the compound is Example 216-219, or 251-254, or a pharmaceutically acceptable salt thereof, to a patient in need of such treatment.

In another aspect, the invention provides a method of treating cancer comprising administering a therapeutically effective amount of a compound of Formula (I), wherein the compound is Example 216-219, or 251-254, or a pharmaceutically acceptable salt thereof, to a patient in need of such treatment.

In another aspect, the invention provides a method of treating prostate cancer comprising administering a therapeutically effective amount of a compound of Formula (I), wherein the compound is Example 216-219, or 251-254, or a pharmaceutically acceptable salt thereof, to a patient in need of such treatment.

Compounds of the invention where R₆ of Formula (I) is (1) a small ring such as cyclopropylmethyl or cyclobutyl, (2) a heterocycle containing an oxygen atom (oxetane, tetrahydrofuran or tetrahydropyran), (3) an oxygen substituted cyclopentane or cyclohexane ring, or (4) an alkyl chain substituted with oxygen, have unexpected properties.

Compounds of the invention where R₆ is cyclopropylmethyl or cyclobutyl have higher potencies against cancer cell growth as compared to open chain analogs. An example of (1) above is Example 148 which is 20-100 times more active in a PC-3 cell line than open chain butyl or alkyl analogs containing no oxygen.

Compounds of the invention where R₆ is an oxygen containing heterocycle are more active in a PC-3 cell line than cyclopentyl analogs that do not contain an oxygen atom. An example of (2) above is Example 181, where R₆ is tetrahydrofuran. Example 181 is 20-50 times more active in a PC-3 cell line than non-oxygen containing cyclopentyl analogs.

Compounds of the invention where R₆ is cyclopentyl or cyclohexyl substituted with an oxygen containing group have higher potencies against cancer cell growth than non-oxygen equivalents. An example of (3) above is Example 172, R₆ is 2-hydroxy cyclohexyl, which is 20-50 times more potent in a PC-3 cell line than cyclohexyl analogs not substituted with an oxygen atom.

Compounds of the invention where R₆ is an alkyl chain substituted with an oxygen atom have higher potency against cancer cell growth than non-oxygen alkyl chains. An example of (4) above is Example 150 where R₆ is 2-hydroxy ethyl. Example 150 is 10-30 times more potent in a PC-3 cell line than alkyl analogs without oxygen.

Compounds of the invention encompassed by (1)-(4) that have improved potency against cancer cell growth include, but are not limited to, Examples 14, 23, 33, 36, 46, and 142-183.

Accordingly, in another aspect, the invention provides compounds of Formula (I) wherein R₁, R₂, R₃, R₄, and R₅ are —H; R₆ is (C₃-C₈)cycloalkyl, heteroaryl, heterocyclyl, or hydroxy(C₁-C₈)alkyl, wherein the R₆ group is optionally substituted with 1 group that is hydroxy, hydroxy(C₁-C₈)alkoxy(C₁-C₈)alkoxy, hydroxy(C₁-C₈)alkyl, oxo, R₁₁ON═CH(CH₂)ₙO—, or HONHC(O) (CH₂)ₙO—; R₇ is —H; R₈ is (C₁-C₈)alkyl, (C₃-C₈)cycloalkyl(C₁-C₈)alkyl, aryl, aryl(C₁-C₈)alkyl, halo(C₁-C₈)alkyl, heteroaryl, heteroaryl(C₁-C₈)alkyl, or hydroxy(C₁-C₈)alkyl; R₉ and R₁₀ are independently (C₁-C₈)alkyl; R₁₁ is (C₁-C₈)alkyl or aryl; n is 1, 2, 3, 4, 5, or 6; X is N or CH; and Y is C.

In another aspect, the invention provides compounds of Formula (I) wherein R₁, R₂, R₃, R₄, and R₅ are —H; R₆ is (C₃-C₈)cycloalkyl optionally substituted with 1 group that is hydroxy, hydroxy(C₁-C₈)alkoxy(C₁-C₈)alkoxy, hydroxy(C₁-C₈)alkyl, R₁₁ON═CH(CH₂)ₙO—, or HONHC(O) (CH₂)ₙO—; R₇ is —H; R₈ is (C₁-C₈)alkyl, (C₃-C₈)cycloalkyl(C₁-C₈)alkyl, aryl, aryl(C₁-C₈)alkyl, halo(C₁-C₈)alkyl, heteroaryl, heteroaryl(C₁-C₈)alkyl, or hydroxy(C₁-C₈)alkyl; R₉ and R₁₀ are independently (C₁-C₈)alkyl; R₁₁ is (C₁-C₈)alkyl or aryl; n is 1, 2, 3, 4, 5, or 6; X is N or CH; and Y is C.

In another aspect, the invention provides compounds of Formula (I) wherein R₁, R₂, R₃, R₄, and R₅ are —H; R₆ is cyclobutyl, cyclopentyl, or cyclohexyl, wherein each is optionally substituted with 1 group that is hydroxy, hydroxy(C₁-C₈)alkoxy(C₁-C₈)alkoxy, hydroxy(C₁-C₈)alkyl, R₁₁ON═CH(CH₂)ₙO—, or HONHC(O) (CH₂)ₙO—; R₇ is —H; R₈ is (C₁-C₈)alkyl, (C₃-C₈)cycloalkyl(C₁-C₈)alkyl, aryl, aryl(C₁-C₈)alkyl, halo(C₁-C₈)alkyl, heteroaryl, heteroaryl (C₁-C₈) alkyl, or hydroxy (C₁-C₈) alkyl; R₉ and R₁₀ are methyl; R₁₁ is methyl or phenyl; n is 1; X is N or CH; and Y is C.

In another aspect, the invention provides compounds of Formula (I) wherein R₁, R₂, R₃, R₄, and R₅ are —H; R₆ is cyclobutyl, cyclopentyl, or cyclohexyl, wherein each is optionally substituted with 1 group that is hydroxy, hydroxy(C₁-C₈)alkoxy(C₁-C₈)alkoxy, hydroxy(C₁-C₈)alkyl, R₁₁ON═CH(CH₂)ₙO—, or HONHC(O) (CH₂)ₙO—; R₇ is —H; R₈ is benzyl, cyclopropylmethyl, methoxyphenyl, methyl, ethyl, hydroxymethyl, pyrazolyl, pyridinylmethyl, thienylmethyl, trifluoromethyl; R₉ and R₁₀ are methyl; R₁₁ is methyl or phenyl; n is 1; X is N or CH; and Y is C.

In another aspect, the invention provides compounds of Formula (I) wherein R₁, R₂, R₃, R₄, and R₅ are —H; R₆ is cyclobutyl, 2-hydroxycyclopentyl, 4-hydroxycyclohexyl, 2-hydroxycyclohexyl, 4-(2-(phenoxyimino)ethoxy)cyclohexyl, 4-(2-(methoxyimino)ethoxy)cyclohexyl, 4-(2-(hydroxyamino)-2-oxoethoxy)cyclohexyl, 4-(2-(2-hydroxyethoxy)ethoxy)cyclohexyl; R₇ is —H; R₈ is benzyl, cyclopropylmethyl, methoxyphenyl, methyl, ethyl, hydroxymethyl, pyrazolyl, pyridinylmethyl, thienylmethyl, trifluoromethyl; R₉ and R₁₀ are methyl; X is N; and Y is C.

In another aspect, the invention provides compounds of Formula (I) wherein R₁, R₂, R₃, R₄, and R₅ are —H; R₆ is cyclobutyl, trans-2-hydroxycyclopentyl, trans-4-hydroxycyclohexyl, trans-2-hydroxycyclohexyl, trans-4-(2-(phenoxyimino)ethoxy)cyclohexyl, trans-4-(2-(methoxyimino)ethoxy)cyclohexyl, trans-4-(2-(hydroxyamino)-2-oxoethoxy)cyclohexyl, trans-4-(2-(2-hydroxyethoxy)ethoxy)cyclohexyl; R₇ is —H; R₈ is benzyl, cyclopropylmethyl, methoxyphenyl, methyl, ethyl, hydroxymethyl, pyrazolyl, pyridinylmethyl, thienylmethyl, trifluoromethyl; R₉ and R₁₀ are methyl; X is N; and Y is C.

In another aspect, the invention provides compounds of Formula (I) wherein R₁, R₂, R₃, R₄, and R₅ are —H; R₆ is cyclobutyl, trans-2-hydroxycyclopentyl, trans-4-hydroxycyclohexyl, trans-2-hydroxycyclohexyl, trans-4-(2-(phenoxyimino)ethoxy)cyclohexyl, trans-4-(2-(methoxyimino)ethoxy)cyclohexyl, trans-4-(2-(hydroxyamino)-2-oxoethoxy)cyclohexyl, trans-4-(2-(2-hydroxyethoxy)ethoxy)cyclohexyl; R₇ is —H; R₈ is benzyl, cyclopropylmethyl, methoxyphenyl, methyl, ethyl, hydroxymethyl, pyrazolyl, pyridinylmethyl, thienylmethyl, trifluoromethyl; R₉ is methyl; R₁₀ is ethyl; X is N or CH; and Y is C.

In another aspect, the invention provides compounds of Formula (I) wherein R₁, R₂, R₃, R₄, and R₅ are —H; R₆ is trans-4-hydroxycyclohexyl; R₇ is —H; R₈ is benzyl, cyclopropylmethyl, methoxyphenyl, methyl, ethyl, hydroxymethyl, pyrazolyl, pyridinylmethyl, thienylmethyl, trifluoromethyl; R₉ is methyl; R₁₀ is ethyl; X is N; and Y is C.

In another aspect, the invention provides compounds of Formula (I) wherein R₁, R₂, R₃, R₄, and R₅ are —H; R₆ is trans-4-hydroxycyclohexyl; R₇ is —H; R₈ is methyl, ethyl, or trifluoromethyl; R₉ is methyl; R₁₀ is ethyl; X is N; and Y is C.

In another aspect, the invention provides compounds of Formula (I) wherein R₁, R₂, R₄, and R₅ are —H; R₃ is methoxy; R₆ is (C₃-C₈)cycloalkyl optionally substituted with 1 group that is hydroxy, hydroxy(C₁-C₈)alkoxy(C₁-C₈)alkoxy, hydroxy(C₁-C₈)alkyl, R₁₁ON═CH(CH₂)ₙO—, or HONHC(O) (CH₂)ₙO—; R₇ is —H; R₈ is (C₁-C₈)alkyl, (C₃-C₈)cycloalkyl(C₁-C₈)alkyl, aryl, aryl (C₁-C₈) alkyl, halo (C₁-C₈) alkyl, heteroaryl, heteroaryl (C₁-C₈) alkyl, or hydroxy (C₁-C₈)alkyl; R₉ and R₁₀ are independently (C₁-C₈) alkyl; R₁₁ is (C₁-C₈)alkyl or aryl; n is 1, 2, 3, 4, 5, or 6; X is N or CH; and Y is C.

In another aspect, the invention provides compounds of Formula (I) wherein R₁, R₂, R₄, and R₅ are —H; R₃ is methoxy; R₆ is cyclobutyl, cyclopentyl, or cyclohexyl, wherein each is optionally substituted with 1 group that is hydroxy, hydroxy(C₁-C₈)alkoxy(C₁-C₈)alkoxy, hydroxy(C₁-C₈)alkyl, R₁₁ON═CH(CH₂)ₙO—, or HONHC(O) (CH₂)ₙO—; R₇ is —H; R₈ is (C₁-C₈)alkyl, (C₃-C₈)cycloalkyl(C₁-C₈)alkyl, aryl, aryl(C₁-C₈)alkyl, halo(C₁-C₈)alkyl, heteroaryl, heteroaryl(C₁-C₈)alkyl, or hydroxy(C₁-C₈)alkyl; R₉ and R₁₀ are methyl; R₁₁ is methyl or phenyl; n is 1; X is N or CH; and Y is C.

In another aspect, the invention provides compounds of Formula (I) wherein R₁, R₂, R₄, and R₅ are —H; R₃ is methoxy; R₆ is cyclobutyl, cyclopentyl, or cyclohexyl, wherein each is optionally substituted with 1 group that is hydroxy, hydroxy(C₁-C₈)alkoxy(C₁-C₈)alkoxy, hydroxy(C₁-C₈)alkyl, R₁₁ON═CH(CH₂)ₙO—, or HONHC(O) (CH₂)ₙO—; R₇ is —H; R₈ is benzyl, cyclopropylmethyl, methoxyphenyl, methyl, ethyl, hydroxymethyl, pyrazolyl, pyridinylmethyl, thienylmethyl, trifluoromethyl; R₉ and R₁₀ are methyl; R₁₁ is methyl or phenyl; n is 1; X is N or CH; and Y is C.

In another aspect, the invention provides compounds of Formula (I) wherein R₁, R₂, R₄, and R₅ are —H; R₃ is methoxy; R₆ is cyclobutyl, 2-hydroxycyclopentyl, 4-hydroxycyclohexyl, 2-hydroxycyclohexyl, 4-(2-(phenoxyimino)ethoxy)cyclohexyl, 4-(2-(methoxyimino)ethoxy)cyclohexyl, 4-(2-(hydroxyamino)-2-oxoethoxy)cyclohexyl, 4-(2-(2-hydroxyethoxy)ethoxy)cyclohexyl; R₇ is —H; R₈ is benzyl, cyclopropylmethyl, methoxyphenyl, methyl, ethyl, hydroxymethyl, pyrazolyl, pyridinylmethyl, thienylmethyl, trifluoromethyl; R₉ and R₁₀ are methyl; X is N; and Y is C.

In another aspect, the invention provides compounds of Formula (I) wherein R₁, R₂, R₄, and R₅ are —H; R₃ is methoxy; R₆ is cyclobutyl, trans-2-hydroxycyclopentyl, trans-4-hydroxycyclohexyl, trans-2-hydroxycyclohexyl, trans-4-(2-(phenoxyimino)ethoxy)cyclohexyl, trans-4-(2-(methoxyimino)ethoxy)cyclohexyl, trans-4-(2-(hydroxyamino)-2-oxoethoxy)cyclohexyl, trans-4-(2-(2-hydroxyethoxy)ethoxy)cyclohexyl; R₇ is —H; R₈ is benzyl, cyclopropylmethyl, methoxyphenyl, methyl, ethyl, hydroxymethyl, pyrazolyl, pyridinylmethyl, thienylmethyl, trifluoromethyl; R₉ and R₁₀ are methyl; X is N; and Y is C.

In another aspect, the invention provides compounds of Formula (I) wherein R₁, R₂, R₄, and R₅ are —H; R₃ is methoxy; R₆ is trans-2-hydroxycyclopentyl; R₇ is —H; R₈ is benzyl, cyclopropylmethyl, methoxyphenyl, methyl, ethyl, hydroxymethyl, pyrazolyl, pyridinylmethyl, thienylmethyl, trifluoromethyl; R₉ and R₁₀ are methyl; X is N; and Y is C.

In another aspect, the invention provides compounds of Formula (I) wherein R₁, R₂, R₄, and R₅ are —H; R₃ is methoxy; R₆ is trans-2-hydroxycyclopentyl; R₇ is —H; R₈ is methyl, ethyl, or trifluoromethyl; R₉ and R₁₀ are methyl; X is N; and Y is C.

In another aspect, the invention provides compounds of Formula (I) wherein R₁, R₂, R₄, and R₅ are —H; R₃ is absent; R₆ is (C₃-C₈)cycloalkyl optionally substituted with 1 group that is hydroxy, hydroxy(C₁-C₈)alkoxy(C₁-C₈)alkoxy, hydroxy(C₁-C₈)alkyl, R₁₁ON═CH(CH₂)ₙO—, or HONHC(O) (CH₂)ₙO—; R₇ is —H; R₈ is (C₁-C₈)alkyl, (C₃-C₈)cycloalkyl(C₁-C₈)alkyl, aryl, aryl(C₁-C₈)alkyl, halo(C₁-C₈)alkyl, heteroaryl, heteroaryl(C₁-C₈)alkyl, or hydroxy(C₁-C₈)alkyl; R₉ and R₁₀ are independently (C₁-C₈) alkyl; R₁₁ is (C₁-C₈) alkyl or aryl; n is 1, 2, 3, 4, 5, or 6; X is N or CH; and Y is N.

In another aspect, the invention provides compounds of Formula (I) wherein R₁, R₂, R₄, and R₅ are —H; R₃ is absent; R₆ is cyclobutyl, cyclopentyl, or cyclohexyl, wherein each is optionally substituted with 1 group that is hydroxy, hydroxy(C₁-C₈)alkoxy(C₁-C₈)alkoxy, hydroxy(C₁-C₈)alkyl, R₁₁ON═CH(CH₂)ₙO—, or HONHC(O) (CH₂)ₙO—; R₇ is —H; R₈ is (C₁-C₈)alkyl, (C₃-C₈)cycloalkyl(C₁-C₈)alkyl, aryl, aryl(C₁-C₈)alkyl, halo(C₁-C₈)alkyl, heteroaryl, heteroaryl(C₁-C₈)alkyl, or hydroxy(C₁-C₈)alkyl; R₉ and R₁₀ are methyl; R₁₁ is methyl or phenyl; n is 1; X is N or CH; and Y is N.

In another aspect, the invention provides compounds of Formula (I) wherein R₁, R₂, R₄, and R₅ are —H; R₃ is absent; R₆ is cyclobutyl, cyclopentyl, or cyclohexyl, wherein each is optionally substituted with 1 group that is hydroxy, hydroxy(C₁-C₈)alkoxy(C₁-C₈)alkoxy, hydroxy(C₁-C₈)alkyl, R₁₁ON═CH(CH₂)ₙO—, or HONHC(O) (CH₂)ₙO—; R₇ is —H; R₈ is benzyl, cyclopropylmethyl, methoxyphenyl, methyl, ethyl, hydroxymethyl, pyrazolyl, pyridinylmethyl, thienylmethyl, trifluoromethyl; R₉ and R₁₀ are methyl; R₁₁ is methyl or phenyl; n is 1; X is N or CH; and Y is N.

In another aspect, the invention provides compounds of Formula (I) wherein R₁, R₂, R₄, and R₅ are —H; R₃ is absent; R₆ is cyclobutyl, 2-hydroxycyclopentyl, 4-hydroxycyclohexyl, 2-hydroxycyclohexyl, 4-(2-(phenoxyimino)ethoxy)cyclohexyl, 4-(2-(methoxyimino)ethoxy)cyclohexyl, 4-(2-(hydroxyamino)-2-oxoethoxy)cyclohexyl, 4-(2-(2-hydroxyethoxy)ethoxy)cyclohexyl; R₇ is —H; R₈ is benzyl, cyclopropylmethyl, methoxyphenyl, methyl, ethyl, hydroxymethyl, pyrazolyl, pyridinylmethyl, thienylmethyl, trifluoromethyl; R₉ and R₁₀ are methyl; X is N; and Y is N.

In another aspect, the invention provides compounds of Formula (I) wherein R₁, R₂, R₄, and R₅ are —H; R₃ is absent; R₆ is cyclobutyl, trans-2-hydroxycyclopentyl, trans-4-hydroxycyclohexyl, trans-2-hydroxycyclohexyl, trans-4-(2-(phenoxyimino)ethoxy)cyclohexyl, trans-4-(2-(methoxyimino)ethoxy)cyclohexyl, trans-4-(2-(hydroxyamino)-2-oxoethoxy)cyclohexyl, trans-4-(2-(2-hydroxyethoxy)ethoxy)cyclohexyl; R₇ is —H; R₈ is benzyl, cyclopropylmethyl, methoxyphenyl, methyl, ethyl, hydroxymethyl, pyrazolyl, pyridinylmethyl, thienylmethyl, trifluoromethyl; R₉ and R₁₀ are methyl; X is N; and Y is N.

In another aspect, the invention provides compounds of Formula (I) wherein R₁, R₂, R₄, and R₅ are —H; R₃ is absent; R₆ trans-4-hydroxycyclohexyl; R₇ is —H; R₈ is benzyl, cyclopropylmethyl, methoxyphenyl, methyl, ethyl, hydroxymethyl, pyrazolyl, pyridinylmethyl, thienylmethyl, trifluoromethyl; R₉ and R₁₀ are methyl; X is N; and Y is N.

In another aspect, the invention provides compounds of Formula (I) wherein R₁, R₂, R₄, and R₅ are —H; R₃ is absent; R₆ trans-4-hydroxycyclohexyl; R₇ is —H; R₈ is methyl, ethyl, or trifluoromethyl; R₉ and R₁₀ are methyl; X is N; and Y is N.

In another aspect, the invention provides compounds of Formula (I) wherein R₁, R₂, R₃, R₄, and R₅ are —H; R₆ is heteroaryl optionally substituted with 1 group that is hydroxy, hydroxy(C₁-C₈)alkoxy(C₁-C₈)alkoxy, hydroxy(C₁-C₈)alkyl, R₁₁ON═CH(CH₂)ₙO—, or HONHC(O) (CH₂)ₙO—; R₇ is —H; R₈ is (C₁-C₈)alkyl, (C₃-C₈)cycloalkyl(C₁-C₈)alkyl, aryl, aryl(C₁-C₈)alkyl, halo(C₁-C₈)alkyl, heteroaryl, heteroaryl(C₁-C₈)alkyl, or hydroxy(C₁-C₈)alkyl; R₉ and R₁₀ are independently (C₁-C₈)alkyl; R₁₁ is (C₁-C₈)alkyl or aryl; n is 1, 2, 3, 4, 5, or 6; X is N or CH; and Y is C.

In another aspect, the invention provides compounds of Formula (I) wherein R₁, R₂, R₃, R₄, and R₅ are —H; R₆ is pyrazolyl optionally substituted with hydroxy(C₁-C₈)alkyl; R₇ is —H; R₈ is (C₁-C₈) alkyl, (C₃-C₈) cycloalkyl (C₁-C₈) alkyl, aryl, aryl (C₁-C₈) alkyl, halo (C₁-C₈) alkyl, heteroaryl, heteroaryl (C₁-C₈) alkyl, or hydroxy (C₁-C₈) alkyl; R₉ and R₁₀ are methyl; X is N; and Y is C.

In another aspect, the invention provides compounds of Formula (I) wherein R₁, R₂, R₃, R₄, and R₅ are —H; R₆ is 1-(2-hydroxyethyl)-1H-pyrazolyl; R₇ is —H; R₈ is benzyl, cyclopropylmethyl, methoxyphenyl, methyl, ethyl, hydroxymethyl, pyrazolyl, pyridinylmethyl, thienylmethyl, trifluoromethyl; R₉ and R₁₀ are methyl; X is N; and Y is C.

In another aspect, the invention provides compounds of Formula (I) wherein R₁, R₂, R₃, R₄, and R₅ are —H; R₆ is heterocyclyl optionally substituted with oxo; R₇ is —H; R₈ is (C₁-C₈) alkyl, (C₃-C₈) cycloalkyl(C₁-C₈) alkyl, aryl, aryl(C₁-C₈)alkyl, halo(C₁-C₈)alkyl, heteroaryl, heteroaryl(C₁-C₈)alkyl, or hydroxy(C₁-C₈)alkyl; R₉ and R₁₀ are independently (C₁-C₈)alkyl; X is N or CH; and Y is C.

In another aspect, the invention provides compounds of Formula (I) wherein R₁, R₂, R₃, R₄, and R₅ are —H; R₆ is oxetanyl, tetrahydrofuranyl, or tetrahydropyranyl, wherein each is optionally substituted with 1 oxo group; R₇ is —H; R₈ is (C₁-C₈)alkyl, (C₃-C₈)cycloalkyl(C₁-C₈)alkyl, aryl, aryl(C₁-C₈)alkyl, halo(C₁-C₈)alkyl, heteroaryl, heteroaryl(C₁-C₈)alkyl, or hydroxy(C₁-C₈)alkyl; R₉ and R₁₀ are methyl; X is N; and Y is C.

In another aspect, the invention provides compounds of Formula (I) wherein R₁, R₂, R₃, R₄, and R₅ are —H; R₆ is oxetanyl, tetrahydrofuranyl, or tetrahydropyranyl; R₇ is —H; R₈ is (C₁-C₈)alkyl, (C₃-C₈)cycloalkyl(C₁-C₈)alkyl, aryl, aryl(C₁-C₈)alkyl, halo(C₁-C₈)alkyl, heteroaryl, heteroaryl(C₁-C₈)alkyl, or hydroxy(C₁-C₈)alkyl; R₉ and R₁₀ are methyl; X is N; and Y is C.

In another aspect, the invention provides compounds of Formula (I) wherein R₁, R₂, R₃, R₄, and R₅ are —H; R₆ is oxetanyl, tetrahydrofuranyl, or tetrahydropyranyl; R₇ is —H; R₈ is benzyl, cyclopropylmethyl, methoxyphenyl, methyl, ethyl, hydroxymethyl, pyrazolyl, pyridinylmethyl, thienylmethyl, trifluoromethyl; R₉ and R₁₀ are methyl; X is N; and Y is C.

In another aspect, the invention provides compounds of Formula (I) wherein R₁, R₂, R₃, R₄, and R₅ are —H; R₆ is hydroxy(C₁-C₈)alkyl; R₇ is —H; R is (C₁-C₈)alkyl, (C₃-C₈)cycloalkyl(C₁-C₈)alkyl, aryl, aryl(C₁-C₈)alkyl, halo(C₁-C₈)alkyl, heteroaryl, heteroaryl(C₁-C₈)alkyl, or hydroxy(C₁-C₈)alkyl; R₉ and R₁₀ are independently (C₁-C₈)alkyl; X is N or CH; and Y is C.

In another aspect, the invention provides compounds of Formula (I) wherein R₁, R₂, R₃, R₄, and R₅ are —H; R₆ is 2-hydroxyethyl, (S)-1-hydroxypropan-2-yl, (R)-1-hydroxypropan-2-yl, (S)-2-hydroxypropyl, or (R)-2-hydroxypropyl; R₇ is —H; R₈ is benzyl, cyclopropylmethyl, methoxyphenyl, methyl, ethyl, hydroxymethyl, pyrazolyl, pyridinylmethyl, thienylmethyl, trifluoromethyl; R₉ and R₁₀ are methyl; X is N; and Y is C.

In another aspect, the invention provides a pharmaceutical composition comprising a therapeutically effective amount of a compound of Formula (I) wherein the compound is Example 14, 23, 33, 36, 46, 142-182, or 183, or a pharmaceutically acceptable salt thereof, and at least one pharmaceutically acceptable carrier, solvent, adjuvant or diluent.

In another aspect, the invention provides a method of treating a disease or disorder related to cell proliferation comprising administering a therapeutically effective amount of a compound of Formula (I), wherein the compound is Example 14, 23, 33, 36, 46, 142-182, or 183, or a pharmaceutically acceptable salt thereof, to a patient in need of such treatment.

In another aspect, the invention provides a method of inhibiting cell proliferation comprising administering a therapeutically effective amount of a compound of Formula (I), wherein the compound is Example 14, 23, 33, 36, 46, 142-182, or 183, or a pharmaceutically acceptable salt thereof, to a patient in need of such treatment.

In another aspect, the invention provides a method of treating a disease or disorder related to Heat-shock protein 90 comprising administering a therapeutically effective amount of a compound of Formula (I), wherein the compound is Example 14, 23, 33, 36, 46, 142-182, or 183, or a pharmaceutically acceptable salt thereof, to a patient in need of such treatment.

In another aspect, the invention provides a method of inhibiting Heat-shock protein 90 comprising administering a therapeutically effective amount of a compound of Formula (I), wherein the compound is Example 14, 23, 33, 36, 46, 142-182, or 183, or a pharmaceutically acceptable salt thereof, to a patient in need of such treatment.

In another aspect, the invention provides a method of treating cancer comprising administering a therapeutically effective amount of a compound of Formula (I), wherein the compound is Example 14, 23, 33, 36, 46, 142-182, or 183, or a pharmaceutically acceptable salt thereof, to a patient in need of such treatment.

In another aspect, the invention provides a method of treating prostate cancer comprising administering a therapeutically effective amount of a compound of Formula (I), wherein the compound is Example 14, 23, 33, 36, 46, 142-182, or 183, or a pharmaceutically acceptable salt thereof, to a patient in need of such treatment.

In another aspect, the invention provides a pharmaceutical composition comprising a therapeutically effective amount of a compound of Formula (I) wherein the compound is Example 220-225, 229, 230, or 255-272, or a pharmaceutically acceptable salt thereof, and at least one pharmaceutically acceptable carrier, solvent, adjuvant or diluent.

In another aspect, the invention provides a method of treating a disease or disorder related to cell proliferation comprising administering a therapeutically effective amount of a compound of Formula (I), wherein the compound is Example 220-225, 229, 230, or 255-272, or a pharmaceutically acceptable salt thereof, to a patient in need of such treatment.

In another aspect, the invention provides a method of inhibiting cell proliferation comprising administering a therapeutically effective amount of a compound of Formula (I), wherein the compound is Example 220-225, 229, 230, or 255-272, or a pharmaceutically acceptable salt thereof, to a patient in need of such treatment.

In another aspect, the invention provides a method of treating a disease or disorder related to Heat-shock protein 90 comprising administering a therapeutically effective amount of a compound of Formula (I), wherein the compound is Example 220-225, 229, 230, or 255-272, or a pharmaceutically acceptable salt thereof, to a patient in need of such treatment.

In another aspect, the invention provides a method of inhibiting Heat-shock protein 90 comprising administering a therapeutically effective amount of a compound of Formula (I), wherein the compound is Example 220-225, 229, 230, or 255-272, or a pharmaceutically acceptable salt thereof, to a patient in need of such treatment.

In another aspect, the invention provides a method of treating cancer comprising administering a therapeutically effective amount of a compound of Formula (I), wherein the compound is Example 220-225, 229, 230, or 255-272, or a pharmaceutically acceptable salt thereof, to a patient in need of such treatment.

In another aspect, the invention provides a method of treating prostate cancer comprising administering a therapeutically effective amount of a compound of Formula (I), wherein the compound is Example 220-225, 229, 230, or 255-272, or a pharmaceutically acceptable salt thereof, to a patient in need of such treatment.

Compounds of the invention where R₅ and R₆ combine to form a 2-substituted imidazole show inhibitory activities against dual or multiple targets in addition to HSP-90. In cellular assays, these compounds show anti-proliferative activity via other pathways. For example, a compound of the invention where R₅ and R₆ form 2-methylimidazole, Example 186, inhibits a PC-3 cell line growth via HSP-90 plus other mechanism(s).

Compounds of the invention where R₅ and R₆ form a 2-imidazole and show improved potency against cancer cell growth via inhibition of HSP-90 and via other pathways include, but are not limited to, Examples 22 and 184-188.

Accordingly, in another aspect, the invention provides compounds of Formula (I) wherein R₁ and R₂ are independently —H or C₁-C₈ alkyl; R₃ and R₄ are independently —H or —F; R₅ and R₆ taken together with the nitrogen atom to which they are attached form a heteroaryl, wherein the heteroaryl is imidazolyl, oxazolyl, pyrazolyl, pyridazinyl, pyridinyl, pyrazinyl, pyrrolyl, or thiazolyl, wherein the heteroaryl is optionally substituted with (C₁-C₈)alkoxy, (C₁-C₈)alkoxycarbonyl, (C₁-C₈)alkyl, (C₁-C₈)alkylcarbonyl, (C₁-C₈)alkylcarbonyloxy, aryl, aryl(C₁-C₈)alkyl, cyano, cyano(C₁-C₈)alkyl, (C₃-C₈)cycloalkyl, (C₃-C₈)cycloalkyl(C₁-C₈)alkyl, halo (C₁-C₈)alkoxy, halo (C₁-C₈)alkyl, halogen, heteroaryl, heteroaryl (C₁-C₈) alkyl, heterocyclyl, heterocycle (C₁-C₈)alkyl, hydroxy, hydroxy(C₁-C₈)alkyl, mercapto, nitro, —NR₁₁R₁₂, (NR₁₁R₁₂) (C₁-C₈) alkyl, or (NR₁₁R₁₂) carbonyl; R₇ is —H or (C₁-C₈) alkyl; R₈ is (C₂-C₈) alkenyl, (C₁-C₈) alkyl, (C₂-C₈) alkynyl, (C₃-C₈) cycloalkenyl, (C₃-C₈) cycloalkenyl (C₁-C₈) alkyl, (C₃-C₈) cycloalkyl, (C₃-C₈) cycloalkyl (C₁-C₈) alkyl, aryl, aryl (C₁-C₈)alkyl, halo (C₁-C₈) alkyl, heteroaryl, heteroaryl (C₁-C₈)alkyl, heterocyclyl, heterocycle (C₁-C₈)alkyl, or hydroxy(C₁-C₈)alkyl; R₉ and R₁₀ are independently —H or (C₁-C₈) alkyl; or R₉ and R₁₀ taken together with the carbon atom to which they are attached form (C₃-C₈)cycloalkyl; R₁₁ and R₁₂ are independently —H, (C₂-C₈) alkenyl, (C₁-C₈) alkyl, (C₁-C₈) alkylcarbonyl, (C₂-C₈) alkynyl, aryl, aryl (C₁-C₈) alkyl, (C₃-C₈) cycloalkyl, (C₃-C₈)cycloalkyl (C₁-C₈)alkyl, formyl, heteroaryl, heteroaryl (C₁-C₈) alkyl, heterocyclyl, or heterocycle(C₁-C₈)alkyl; n is 1, 2, 3, 4, 5, or 6; X is N or CR₁₃; Y is C; and R₁₃ is —H or (C₁-C₈)alkyl.

In another aspect, the invention provides compounds of Formula (I) wherein R₁ and R₂ are —H; R₃ and R₄ are independently —H or —F; R₅ and R₆ taken together with the nitrogen atom to which they are attached form imidazolyl optionally substituted with (C₁-C₈)alkoxy, (C₁-C₈)alkoxycarbonyl, (C₁-C₈)alkyl, (C₁-C₈)alkylcarbonyl, (C₁-C₈)alkylcarbonyloxy, aryl, aryl(C₁-C₈)alkyl, cyano, cyano(C₁-C₈)alkyl, (C₃-C₈)cycloalkyl, (C₃-C₈)cycloalkyl(C₁-C₈)alkyl, halo (C₁-C₈)alkoxy, halo (C₁-C₈)alkyl, halogen, heteroaryl, heteroaryl(C₁-C₈)alkyl, heterocyclyl, heterocycle(C₁-C₈)alkyl, hydroxy, hydroxy(C₁-C₈)alkyl, mercapto, nitro, —NR₁₁R₁₂, (NR₁₁R₁₂) (C₁-C₈)alkyl, or (NR₁₁R₁₂)carbonyl; R₇ is —H or (C₁-C₈) alkyl; R₈ is (C₂-C₈) alkenyl, (C₁-C₈) alkyl, (C₂-C₈)alkynyl, (C₃-C₈)cycloalkenyl, (C₃-C₈)cycloalkenyl(C₁-C₈)alkyl, (C₃-C₈)cycloalkyl, (C₃-C₈) cycloalkyl (C₁-C₈) alkyl, aryl, aryl (C₁-C₈)alkyl, halo (C₁-C₈) alkyl, heteroaryl, heteroaryl (C₁-C₈)alkyl, heterocyclyl, heterocycle (C₁-C₈)alkyl, or hydroxy(C₁-C₈)alkyl; R₉ and R₁₀ are methyl; R₁₁ and R₁₂ are independently —H, (C₂-C₈) alkenyl, (C₁-C₈) alkyl, (C₁-C₈) alkylcarbonyl, (C₂-C₈) alkynyl, aryl, aryl (C₁-C₈) alkyl, (C₃-C₈) cycloalkyl, (C₃-C₈) cycloalkyl (C₁-C₈) alkyl, formyl, heteroaryl, heteroaryl (C₁-C₈) alkyl, heterocyclyl, or heterocycle(C₁-C₈)alkyl; n is 1, 2, 3, 4, 5, or 6; X is N; and Y is C.

In another aspect, the invention provides compounds of Formula (I) wherein R₁ and R₂ are —H; R₃ and R₄ are —H or —F; R₅ and R₆ taken together with the nitrogen atom to which they are attached form imidazolyl optionally substituted with (C₁-C₈) alkyl or aryl; R₇ is —H; R₈ is (C₁-C₈) alkyl, (C₃-C₈) cycloalkyl (C₁-C₈) alkyl, or halo (C₁-C₈) alkyl; R₉ and R₁₀ are methyl; X is N; and Y is C.

In another aspect, the invention provides compounds of Formula (I) wherein R₁ and R₂ are —H; R₃ and R₄ are —H or —F; R₅ and R₆ taken together with the nitrogen atom to which they are attached form imidazolyl optionally substituted with methyl or phenyl; R₇ is —H; R₈ is methyl, ethyl, cyclopropylmethyl, or trifluoromethyl; R₉ and R₁₀ are methyl; X is N; and Y is C.

In another aspect, the invention provides compounds of Formula (I) wherein R₁ and R₂ are —H; R₃ and R₄ are —H; R₅ and R₆ taken together with the nitrogen atom to which they are attached form imidazolyl optionally substituted with methyl or phenyl; R₇ is —H; R₈ is methyl, ethyl, cyclopropylmethyl, or trifluoromethyl; R₉ and R₁₀ are methyl; X is N; and Y is C.

In another aspect, the invention provides compounds of Formula (I) wherein R₁ and R₂ are —H; R₃ and R₄ are —H; R₅ and R₆ taken together with the nitrogen atom to which they are attached form imidazolyl optionally substituted with methyl or phenyl; R₇ is —H; R₈ is methyl or ethyl; R₉ and R₁₀ are methyl; X is N; and Y is C.

In another aspect, the invention provides compounds of Formula (I) wherein R₁ and R₂ are —H; R₃ and R₄ are independently —H or —F; R₅ and R₆ taken together with the nitrogen atom to which they are attached form imidazolyl optionally substituted with (C₁-C₈)alkoxy, (C₁-C₈)alkoxycarbonyl, (C₁-C₈)alkyl, (C₁-C₈)alkylcarbonyl, (C₁-C₈)alkylcarbonyloxy, aryl, aryl(C₁-C₈)alkyl, cyano, cyano(C₁-C₈)alkyl, (C₃-C₈)cycloalkyl, (C₃-C₈)cycloalkyl(C₁-C₈)alkyl, halo (C₁-C₈)alkoxy, halo (C₁-C₈)alkyl, halogen, heteroaryl, heteroaryl(C₁-C₈)alkyl, heterocyclyl, heterocycle(C₁-C₈)alkyl, hydroxy, hydroxy(C₁-C₈)alkyl, mercapto, nitro, —NR₁₁R₁₂, (NR₁₁R₁₂) (C₁-C₈)alkyl, or (NR₁₁R₁₂)carbonyl; R₇ is —H or (C₁-C₈) alkyl; R₈ is (C₂-C₈) alkenyl, (C₁-C₈) alkyl, (C₂-C₈)alkynyl, (C₃-C₈)cycloalkenyl, (C₃-C₈)cycloalkenyl(C₁-C₈)alkyl, (C₃-C₈)cycloalkyl, (C₃-C₈)cycloalkyl(C₁-C₈)alkyl, aryl, aryl(C₁-C₈)alkyl, halo(C₁-C₈)alkyl, heteroaryl, heteroaryl(C₁-C₈)alkyl, heterocyclyl, heterocycle(C₁-C₈)alkyl, or hydroxy(C₁-C₈)alkyl; R₉ and R₁₀ are methyl; R₁₁ and R₁₂ are independently —H, (C₂-C₈)alkenyl, (C₁-C₈)alkyl, (C₁-C₈)alkylcarbonyl, (C₂-C₈) alkynyl, aryl, aryl(C₁-C₈) alkyl, (C₃-C₈) cycloalkyl, (C₃-C₈)cycloalkyl(C₁-C₈) alkyl, formyl, heteroaryl, heteroaryl(C₁-C₈)alkyl, heterocyclyl, or heterocycle(C₁-C₈)alkyl; n is 1, 2, 3, 4, 5, or 6; X is CR₁₃; Y is C; and R₁₃ is —H or (C₁-C₈)alkyl.

In another aspect, the invention provides compounds of Formula (I) wherein R₁ and R₂ are —H; R₃ and R₄ are —H or —F; R₅ and R₆ taken together with the nitrogen atom to which they are attached form imidazolyl optionally substituted with (C₁-C₈) alkyl or aryl; R₇ is —H; R₈ is (C₁-C₈) alkyl, (C₃-C₈)cycloalkyl(C₁-C₈)alkyl, or halo(C₁-C₈)alkyl; R₉ and R₁₀ are methyl; X is CR₁₃; Y is C; and R₁₃ is —H or (C₁-C₈)alkyl.

In another aspect, the invention provides compounds of Formula (I) wherein R₁ and R₂ are —H; R₃ and R₄ are —H or —F; R₅ and R₆ taken together with the nitrogen atom to which they are attached form imidazolyl optionally substituted with methyl or phenyl; R₇ is —H; R₈ is methyl, ethyl, cyclopropylmethyl, or trifluoromethyl; R₉ and R₁₀ are methyl; X is CR₁₃; Y is C; and R₁₃ is —H, methyl, or ethyl.

In another aspect, the invention provides compounds of Formula (I) wherein R₁ and R₂ are —H; R₃ and R₄ are —H; R₅ and R₆ taken together with the nitrogen atom to which they are attached form imidazolyl optionally substituted with methyl or phenyl; R₇ is —H; R₈ is methyl, ethyl, cyclopropylmethyl, or trifluoromethyl; R₉ and R₁₀ are methyl; X is CR₁₃; Y is C; and R₁₃ is —H, methyl, or ethyl.

In another aspect, the invention provides compounds of Formula (I) wherein R₁ and R₂ are —H; R₃ and R₄ are —H; R₅ and R₆ taken together with the nitrogen atom to which they are attached form imidazolyl optionally substituted with methyl or phenyl; R₇ is —H; R₈ is methyl or ethyl; R₉ and R₁₀ are methyl; X is CR₁₃; Y is C; and R₁₃ is —H, methyl, or ethyl.

In another aspect, the invention provides a pharmaceutical composition comprising a therapeutically effective amount of a compound of Formula (I) wherein the compound is Example 22, 184-187, or 188, or a pharmaceutically acceptable salt thereof, and at least one pharmaceutically acceptable carrier, solvent, adjuvant or diluent.

In another aspect, the invention provides a method of treating a disease or disorder related to cell proliferation comprising administering a therapeutically effective amount of a compound of Formula (I), wherein the compound is Example 22, 184-187, or 188, or a pharmaceutically acceptable salt thereof, to a patient in need of such treatment.

In another aspect, the invention provides a method of inhibiting cell proliferation comprising administering a therapeutically effective amount of a compound of Formula (I), wherein the compound is Example 22, 184-187, or 188, or a pharmaceutically acceptable salt thereof, to a patient in need of such treatment.

In another aspect, the invention provides a method of treating a disease or disorder related to Heat-shock protein 90 comprising administering a therapeutically effective amount of a compound of Formula (I), wherein the compound is Example 22, 184-187, or 188, or a pharmaceutically acceptable salt thereof, to a patient in need of such treatment.

In another aspect, the invention provides a method of inhibiting Heat-shock protein 90 comprising administering a therapeutically effective amount of a compound of Formula (I), wherein the compound is Example 22, 184-187, or 188, or a pharmaceutically acceptable salt thereof, to a patient in need of such treatment.

In another aspect, the invention provides a method of treating cancer comprising administering a therapeutically effective amount of a compound of Formula (I), wherein the compound is Example 22, 184-187, or 188, or a pharmaceutically acceptable salt thereof, to a patient in need of such treatment.

In another aspect, the invention provides a method of treating prostate cancer comprising administering a therapeutically effective amount of a compound of Formula (I), wherein the compound is Example 22, 184-187, or 188, or a pharmaceutically acceptable salt thereof, to a patient in need of such treatment.

Compounds of the invention where R₆ is 4,4-difluorocyclohexyl and R₇ is hydrogen show high in vitro Hsp90 inhibitory activities. In addition, these compounds are more potent in a Her2 degradation assay. Compounds of the invention where R₆ is 4,4-difluorocyclohexyl and R₇ is hydrogen show improved potency against cancer cell growth via inhibition of HSP-90. Examples of such compounds herein include, but are not limited to, Examples 233, 243, 249, 265-267, 270, and 272.

In one aspect, the invention provides compounds of formula (I) wherein R₁ and R₂ are independently —H or C₁-C₈ alkyl; R₃ is absent or —H, —F, —OCH₃; R₄ and R₅ are independently —H, —F, —OCH₃; R₆ is (C₃-C₈)cycloalkyl optionally substituted with 1, 2, 3, 4, or 5 groups that are independently halogen; R₇ is —H or (C₁-C₈)alkyl; R₈ is (C₂-C₈)alkenyl, (C₁-C₈)alkyl, (C₂-C₈)alkynyl, (C₃-C₈)cycloalkenyl, (C₃-C₈)cycloalkenyl(C₁-C₈)alkyl, (C₃-C₈)cycloalkyl, (C₃-C₈)cycloalkyl(C₁-C₈)alkyl, aryl, aryl(C₁-C₈)alkyl, halo(C₁-C₈)alkyl, heteroaryl, heteroaryl(C₁-C₈)alkyl, heterocyclyl, heterocycle(C₁-C₈)alkyl, or hydroxy(C₁-C₈)alkyl; R₉ and R₁₀ are independently —H or (C₁-C₈)alkyl; or R₉ and R₁₀ taken together with the carbon atom to which they are attached form a (C₃-C₈)cycloalkyl group; X is N or CR₁₃; Y is C or N; and R₁₃ is —H or (C₁-C₈)alkyl.

In another aspect, the invention provides compounds of formula (I) wherein R₁ and R₂ are independently —H or C₁-C₈ alkyl; R₃ is absent or —H, —F, —OCH₃; R₄ and R₅ are independently —H, —F, —OCH₃; R₆ is cycloxehyl substituted with 1, 2, 3, 4, or 5 groups that are independently halogen; R₇ is —H or (C₁-C₈)alkyl; R₈ is (C₂-C₈)alkenyl, (C₁-C₈)alkyl, (C₂-C₈)alkynyl, (C₃-C₈)cycloalkenyl, (C₃-C₈)cycloalkenyl(C₁-C₈)alkyl, (C₃-C₈)cycloalkyl, (C₃-C₈)cycloalkyl(C₁-C₈)alkyl, aryl, aryl(C₁-C₈)alkyl, halo(C₁-C₈)alkyl, heteroaryl, heteroaryl(C₁-C₈)alkyl, heterocyclyl, heterocycle(C₁-C₈)alkyl, or hydroxy(C₁-C₈)alkyl; R₉ and R₁₀ are independently —H or (C₁-C₈) alkyl; or R₉ and R₁₀ taken together with the carbon atom to which they are attached form (C₃-C₈)cycloalkyl; X is N or CR₁₃; Y is C or N; and R₁₃ is —H or (C₁-C₈)alkyl.

In another aspect, the invention provides compounds of formula (I) wherein R₁ and R₂ are independently —H or C₁-C₈ alkyl; R₃ is absent or —H, —F, —OCH₃; R₄ and R₅ are independently —H, —F, —OCH₃; R₆ is a cycloxehyl group substituted with 1 or 2 groups that are independently fluoro or chloro; R₇ is —H or (C₁-C₈)alkyl; R₈ is (C₂-C₈)alkenyl, (C₁-C₈) alkyl, (C₂-C₈) alkynyl, (C₃-C₈) cycloalkenyl, (C₃-C₈) cycloalkenyl (C₁-C₈) alkyl, (C₃-C₈) cycloalkyl, (C₃-C₈) cycloalkyl (C₁-C₈) alkyl, aryl, aryl (C₁-C₈)alkyl, halo (C₁-C₈) alkyl, heteroaryl, heteroaryl (C₁-C₈)alkyl, heterocyclyl, heterocycle (C₁-C₈)alkyl, or hydroxy(C₁-C₈)alkyl; R₉ and R₁₀ are independently —H or (C₁-C₈) alkyl; or R₉ and R₁₀ taken together with the carbon atom to which they are attached form (C₃-C₈)cycloalkyl; X is N or CR₁₃; Y is C or N; and R₁₃ is —H or (C₁-C₈)alkyl.

In another aspect, the invention provides compounds of formula (I) wherein R₁ and R₂ are independently —H or C₁-C₈ alkyl; R₃ is absent or —H, —F, —OCH₃; R₄ and R₅ are independently —H, —F, —OCH₃; R₆ is 4,4-difluorocycloxehyl; R₇ is —H or (C₁-C₈) alkyl; R₈ is (C₂-C₈) alkenyl, (C₁-C₈) alkyl, (C₂-C₈) alkynyl, (C₃-C₈) cycloalkenyl, (C₃-C₈)cycloalkenyl(C₁-C₈)alkyl, (C₃-C₈)cycloalkyl, (C₃-C₈)cycloalkyl(C₁-C₈)alkyl, aryl, aryl(C₁-C₈)alkyl, halo(C₁-C₈)alkyl, heteroaryl, heteroaryl(C₁-C₈)alkyl, heterocyclyl, heterocycle(C₁-C₈)alkyl, or hydroxy(C₁-C₈)alkyl; R₉ and R₁₀ are independently —H or (C₁-C₈) alkyl; or R₉ and R₁₀ taken together with the carbon atom to which they are attached form (C₃-C₈)cycloalkyl; X is N or CR₁₃; Y is C or N; and R₁₃ is —H or (C₁-C₈)alkyl.

In another aspect, the invention provides compounds of formula (I) wherein R₁ and R₂ are independently —H or C₁-C₈ alkyl; R₃ is absent or —H, —F, —OCH₃; R₄ and R₅ are independently —H, —F, —OCH₃; R₆ is 4-fluorocycloxehyl; R₇ is —H or (C₁-C₈) alkyl; R₈ is (C₂-C₈) alkenyl, (C₁-C₈) alkyl, (C₂-C₈)alkynyl, (C₃-C₈)cycloalkenyl, (C₃-C₈)cycloalkenyl(C₁-C₈)alkyl, (C₃-C₈)cycloalkyl, (C₃-C₈)cycloalkyl(C₁-C₈)alkyl, aryl, aryl(C₁-C₈)alkyl, halo(C₁-C₈)alkyl, heteroaryl, heteroaryl(C₁-C₈)alkyl, heterocyclyl, heterocycle(C₁-C₈)alkyl, or hydroxy(C₁-C₈)alkyl; R₉ and R₁₀ are independently —H or (C₁-C₈) alkyl; or R₉ and R₁₀ taken together with the carbon atom to which they are attached form (C₃-C₈)cycloalkyl; X is N or CR₁₃; Y is C or N; and R₁₃ is —H or (C₁-C₈)alkyl. In another aspect, the invention provides compounds of formula (I) wherein R₁ and R₂ are independently —H or C₁-C₈ alkyl; R₃ is absent or —H, —F, —OCH₃; R₄ and R₅ are independently —H, —F, —OCH₃; R₆ is 4,4-dichlorocycloxehyl; R₇ is —H or (C₁-C₈) alkyl; R₈ is (C₂-C₈) alkenyl, (C₁-C₈) alkyl, (C₂-C₈)alkynyl, (C₃-C₈)cycloalkenyl, (C₃-C₈) cycloalkenyl (C₁-C₈) alkyl, (C₃-C₈) cycloalkyl, (C₃-C₈) cycloalkyl (C₁-C₈) alkyl, aryl, aryl (C₁-C₈)alkyl, halo (C₁-C₈) alkyl, heteroaryl, heteroaryl (C₁-C₈)alkyl, heterocyclyl, heterocycle (C₁-C₈)alkyl, or hydroxy(C₁-C₈)alkyl; R₉ and R₁₀ are independently —H or (C₁-C₈) alkyl; or R₉ and R₁₀ taken together with the carbon atom to which they are attached form (C₃-C₈)cycloalkyl; X is N or CR₁₃; Y is C or N; and R₁₃ is —H or (C₁-C₈)alkyl. In another aspect, the invention provides compounds of formula (I) wherein R₁ and R₂ are independently —H or C₁-C₈ alkyl; R₃ is absent or —H, —F, —OCH₃; R₄ and R₅ are independently —H, —F, —OCH₃; R₆ is 4-chlorocycloxehyl; R₇ is —H or (C₁-C₈) alkyl; R₈ is (C₂-C₈) alkenyl, (C₁-C₈) alkyl, (C₂-C₈)alkynyl, (C₃-C₈)cycloalkenyl, (C₃-C₈) cycloalkenyl (C₁-C₈) alkyl, (C₃-C₈) cycloalkyl, (C₃-C₈) cycloalkyl (C₁-C₈) alkyl, aryl, aryl (C₁-C₈)alkyl, halo (C₁-C₈) alkyl, heteroaryl, heteroaryl (C₁-C₈)alkyl, heterocyclyl, heterocycle (C₁-C₈)alkyl, or hydroxy(C₁-C₈)alkyl; R₉ and R₁₀ are independently —H or (C₁-C₈) alkyl; or R₉ and R₁₀ taken together with the carbon atom to which they are attached form (C₃-C₈)cycloalkyl; X is N or CR₁₃; Y is C or N; and R₁₃ is —H or (C₁-C₈)alkyl. Yet in another aspect, the invention provides compounds of formula (I) wherein R₁ and R₂ are independently —H; R₃ is absent or —H, or —F; R₄ and R₅ are independently —H; R₆ is 4,4-difluorocycloxehyl, 4,4-dichlorocycloxehyl, 4-difluorocycloxehyl, or 4-chlorocycloxehyl; R₇ is —H; R₈ is (C₁-C₈)alkyl or halo(C₁-C₈)alkyl; R₉ and R₁₀ are independently —H or (C₁-C₈)alkyl; X is N or CR₁₃; Y is C or N; and R₁₃ is —H or (C₁-C₈) alkyl.

Yet in another aspect, the invention provides compounds of formula (I) wherein R₁ and R₂ are independently —H; R₃ is absent or —H, or —F; R₄ and R₅ are independently —H; R₆ is 4,4-difluorocycloxehyl; R₇ is —H; R₈ is (C₁-C₈)alkyl or halo(C₁-C₈)alkyl; R₉ and R₁₀ are independently —H or (C₁-C₈)alkyl; X is N or CR₁₃; Y is C or N; and R₁₃ is —H or (C₁-C₈) alkyl.

In another aspect, the invention provides a pharmaceutical composition comprising a therapeutically effective amount of a compound of Formula (I) wherein the compound is Example 233, 243, 249, 265-267, 270, or 272, or a pharmaceutically acceptable salt thereof, and at least one pharmaceutically acceptable carrier, solvent, adjuvant or diluent.

In another aspect, the invention provides a method of treating a disease or disorder related to cell proliferation comprising administering a therapeutically effective amount of a compound of Formula (I), wherein the compound is Example 233, 243, 249, 265-267, 270, or 272, or a pharmaceutically acceptable salt thereof, to a patient in need of such treatment.

In another aspect, the invention provides a method of inhibiting cell proliferation comprising administering a therapeutically effective amount of a compound of Formula (I), wherein the compound is Example 233, 243, 249, 265-267, 270, or 272, or a pharmaceutically acceptable salt thereof, to a patient in need of such treatment.

In another aspect, the invention provides a method of treating a disease or disorder related to Heat-shock protein 90 comprising administering a therapeutically effective amount of a compound of Formula (I), wherein the compound is Example 233, 243, 249, 265-267, 270, or 272, or a pharmaceutically acceptable salt thereof, to a patient in need of such treatment.

In another aspect, the invention provides a method of inhibiting Heat-shock protein 90 comprising administering a therapeutically effective amount of a compound of Formula (I), wherein the compound is Example 233, 243, 249, 265-267, 270, or 272, or a pharmaceutically acceptable salt thereof, to a patient in need of such treatment.

In another aspect, the invention provides a method of treating cancer comprising administering a therapeutically effective amount of a compound of Formula (I), wherein the compound is Example 233, 243, 249, 265-267, 270, or 272, or a pharmaceutically acceptable salt thereof, to a patient in need of such treatment.

In another aspect, the invention provides a method of treating prostate cancer comprising administering a therapeutically effective amount of a compound of Formula (I), wherein the compound is Example 233, 243, 249, 265-267, 270, or 272, or a pharmaceutically acceptable salt thereof, to a patient in need of such treatment.

In one aspect, the invention provides compounds of formula (I) wherein R₁ and R₂ are independently —H or C₁-C₈ alkyl; R₃ is absent or —H, —F, or —OCH₃; R₄ and R₅ are independently —H, —F, or —OCH₃; R₆ is a (C₃-C₈)cycloalkyl group, which is optionally substituted with 1, 2, 3, 4, or 5 groups that are NH₂ (CH₂) C(O)O—, CH₃NH(CH₂)ₘ(O)O—, (CH₃)₂N(CH₂)ₘC(O)O—, NH₂(CH₂)ₜC(O)NH(CH₂)ₙC(O)O—, R₁₂CH(NH₂)C(O)O—, or NH₂(CH₂)ₘC(R₁₂)₂(CH₂)ₘC(O)O—; R₇ is —H or (C₁-C₈)alkyl; R₈ is (C₂-C₈) alkenyl, (C₁-C₈) alkyl, (C₂-C₈) alkynyl, (C₃-C₈)cycloalkenyl, (C₃-C₈)cycloalkenyl(C₁-C₈)alkyl, (C₃-C₈)cycloalkyl, (C₃-C₈)cycloalkyl(C₁-C₈)alkyl, aryl, aryl(C₁-C₈)alkyl, halo(C₁-C₈)alkyl, heteroaryl, heteroaryl(C₁-C₈)alkyl, heterocyclyl, heterocycle(C₁-C₈)alkyl, or hydroxy(C₁-C₈)alkyl; R₉ and R₁₀ are independently —H or (C₁-C₈)alkyl; or R₉ and R₁₀ taken together with the carbon atom to which they are attached form (C₃-C₈)cycloalkyl; R₁₂ is independently —H, (C₂-C₈) alkenyl, (C₁-C₈)alkyl, (C₁-C₈)alkylcarbonyl, (C₂-C₈)alkynyl, aryl, aryl(C₁-C₈)alkyl, (C₃-C₈) cycloalkyl, (C₃-C₈) cycloalkyl(C₁-C₈) alkyl, formyl, heteroaryl, heteroaryl(C₁-C₈)alkyl, heterocyclyl, or heterocycle(C₁-C₈)alkyl; or two R₁₂ groups together with the carbon to which they are attached form a (C₃-C₈)cycloalkyl group; each m is independently 1, 2, 3, or 4; each t is independently 1, 2, 3, or 4; X is N or CR₁₃; Y is C or N; and R₁₃ is —H or (C₁-C₈) alkyl.

In another aspect, the invention provides compounds of formula (I) wherein R₁ and R₂ are independently —H or C₁-C₈ alkyl; R₃ is absent or —H, —F, or —OCH₃; R₄ and R₅ are independently —H, —F, or —OCH₃; R₆ is cyclopentyl or cyclohexyl group, which is optionally substituted with 1, 2, 3, 4, or 5 groups that are NH₂(CH₂)ₘC(O)O—, CH₃NH(CH₂)ₘC(O)O—, (CH₃)₂N(CH₂)ₘC(O)O—, NH₂(CH₂)ₜC(O)NH(CH₂)ₘC(O)O—, R₁₂CH(NH₂)C(O)O—, or NH₂ (CH₂)ₘC(R₁₂)₂ (CH₂)ₘC(O)O—; R₇ is —H or (C₁-C₈)alkyl; R₈ is (C₂-C₈) alkenyl, (C₁-C₈) alkyl, (C₂-C₈) alkynyl, (C₃-C₈) cycloalkenyl, (C₃-C₈) cycloalkenyl (C₁-C₈) alkyl, (C₃-C₈) cycloalkyl, (C₃-C₈) cycloalkyl (C₁-C₈) alkyl, aryl, aryl (C₁-C₈) alkyl, halo (C₁-C₈) alkyl, heteroaryl, heteroaryl (C₁-C₈)alkyl, heterocyclyl, heterocycle (C₁-C₈)alkyl, or hydroxy(C₁-C₈)alkyl; R₉ and R₁₀ are independently —H or (C₁-C₈)alkyl; or R₉ and R₁₀ taken together with the carbon atom to which they are attached form (C₃-C₈)cycloalkyl; R₁₂ is independently —H, (C₂-C₈) alkenyl, (C₁-C₈) alkyl, (C₁-C₈) alkylcarbonyl, (C₂-C₈) alkynyl, aryl, aryl (C₁-C₈) alkyl, (C₃-C₈) cycloalkyl, (C₃-C₈) cycloalkyl (C₁-C₈) alkyl, formyl, heteroaryl, heteroaryl (C₁-C₈) alkyl, heterocyclyl, or heterocycle(C₁-C₈)alkyl; or two R₁₂ groups together with the carbon to which they are attached form a (C₃-C₈)cycloalkyl group; each m is independently 1, 2, 3, or 4; each t is independently 1, 2, 3, or 4; X is N or CR₁₃; Y is C or N; and R₁₃ is —H or (C₁-C₈) alkyl.

In yet another aspect, the invention provides compounds of formula (I) wherein R₁ and R₂ are independently —H; R₃ is absent or —H, or —F; R₄ and R₅ are independently —H; R₆ is a cyclopentyl or cyclohexyl group substituted with NH₂ (CH₂)ₘC(O)O—, CH₃NH(CH₂)ₘC(O)O—, (CH₃)₂N(CH₂)ₘC(O)O—, NH₂(CH₂)ₜC(O)NH(CH₂)ₘC(O)O—, R₁₂CH(NH₂)C(O)O—, or NH₂(CH₂)ₘC(R₁₂)₂(CH₂)ₘC(O)O—; R₇ is —H; R₈ is (C₁-C₈)alkyl, or halo(C₁-C₈)alkyl; R₉ and R₁₀ are independently —H or (C₁-C₈)alkyl; R₁₂ is independently —H or (C₁-C₈)alkyl; or two R₁₂ groups together with the carbon to which they are attached form a (C₃-C₈)cycloalkyl group; each m is independently 1 or 2; each t is independently 1 or 2; X is N or CR₁₃; Y is C or N; and R₁₃ is —H or (C₁-C₈)alkyl.

In yet another aspect, the invention provides compounds of formula (I) wherein R₁ and R₂ are independently —H; R₃ is absent or —H, or —F; R₄ and R₅ are independently —H; R₆ is a cyclopentyl or cyclohexyl group substituted with NH₂(CH₂)ₘC(O)O—, CH₃NH(CH₂)ₘC(O)O—, (CH₃)₂N(CH₂)ₘC(O)O—, NH₂(CH₂)ₜC(O)NH(CH₂)ₙC(O)O—, R₁₂CH(NH₂)C(O)O—, or NH₂(CH₂)ₘC(R₁₂)₂(CH₂)ₘC(O)O—; R₇ is —H; R₈ is (C₁-C₈)alkyl, or halo(C₁-C₈)alkyl; R₉ and R₁₀ are independently —H or (C₁-C₈)alkyl; R₁₂ is independently —H or (C₁-C₈)alkyl; or two R₁₂ groups together with the carbon to which they are attached form a (C₃-C₈)cycloalkyl group; each m is independently 1; each t is independently 1; X is N or CR₁₃; Y is C or N; and R₁₃ is —H.

In another aspect, the invention provides 5-(6,6-dimethyl-4-oxo-3-(trifluoromethyl)-4,5,6,7-tetrahydro-1H-indazol-1-yl)-3-((trans)-4-hydroxycyclohexylamino)picolinamide.

In another aspect, the invention provides a pharmaceutical composition comprising a therapeutically effective amount of 5-(6,6-dimethyl-4-oxo-3-(trifluoromethyl)-4,5,6,7-tetrahydro-1H-indazol-1-yl)-3-((trans)-4-hydroxycyclohexylamino)picolinamide, or a pharmaceutically acceptable salt thereof, and at least one pharmaceutically acceptable carrier, solvent, adjuvant or diluent.

In another aspect, the invention provides a method of treating a disease or disorder related to cell proliferation comprising administering a therapeutically effective amount of 5-(6,6-dimethyl-4-oxo-3-(trifluoromethyl)-4,5,6,7-tetrahydro-1H-indazol-1-yl)-3-((trans)-4-hydroxycyclohexylamino)picolinamide, or a pharmaceutically acceptable salt thereof, to a patient in need of such treatment.

In another aspect, the invention provides a method of inhibiting cell proliferation comprising administering a therapeutically effective amount of 5-(6,6-dimethyl-4-oxo-3-(trifluoromethyl)-4,5,6,7-tetrahydro-1H-indazol-1-yl)-3-((trans)-4-hydroxycyclohexylamino)picolinamide, or a pharmaceutically acceptable salt thereof, to a patient in need of such treatment.

In another aspect, the invention provides a method of treating a disease or disorder related to Heat-shock protein 90 comprising administering a therapeutically effective amount of 5-(6,6-dimethyl-4-oxo-3-(trifluoromethyl)-4,5,6,7-tetrahydro-1H-indazol-1-yl)-3-((trans)-4-hydroxycyclohexylamino)picolinamide, or a pharmaceutically acceptable salt thereof, to a patient in need of such treatment.

In another aspect, the invention provides a method of inhibiting Heat-shock protein 90 comprising administering a therapeutically effective amount of a compound of 5-(6,6-dimethyl-4-oxo-3-(trifluoromethyl)-4,5,6,7-tetrahydro-1H-indazol-1-yl)-3-((trans)-4-hydroxycyclohexylamino)picolinamide, or a pharmaceutically acceptable salt thereof, to a patient in need of such treatment.

In another aspect, the invention provides a method of treating cancer comprising administering a therapeutically effective amount of 5-(6,6-dimethyl-4-oxo-3-(trifluoromethyl)-4,5,6,7-tetrahydro-1H-indazol-1-yl)-3-((trans)-4-hydroxycyclohexylamino)picolinamide, or a pharmaceutically acceptable salt thereof, to a patient in need of such treatment.

In another aspect, the invention provides a method of treating prostate cancer comprising administering a therapeutically effective amount of 5-(6,6-dimethyl-4-oxo-3-(trifluoromethyl)-4,5,6,7-tetrahydro-1H-indazol-1-yl)-3-((trans)-4-hydroxycyclohexylamino)picolinamide, or a pharmaceutically acceptable salt thereof, to a patient in need of such treatment.

### DEFINITIONS

As used throughout this specification and the appended claims, the following terms have the following meanings:

The term “(C₂-C₁₄)alkenyl” as used herein, means a straight or branched chain hydrocarbon containing from 2 to 14 carbons and containing at least one carbon-carbon double bond. Representative examples of (C₂-C₁₄)alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, 3-decenyl, and tridec-12-en-2-yl.

The term “(C₂-C₈)alkenyl” as used herein, is a subset of (C₂-C₁₄)alkenyl and means a straight or branched chain hydrocarbon containing from 2 to 8 carbons and containing at least one carbon-carbon double bond. Representative examples of (C₂-C₈)alkenyl include, but are not limited to, ethenyl, 2-propenyl, 2-methyl-2-propenyl, 3-butenyl, 4-pentenyl, 5-hexenyl, 2-heptenyl, and 2-methyl-1-heptenyl. The term “(C₁-C₈)alkoxy” as used herein, means a (C₁-C₈)alkyl group, as defined herein, appended to the parent molecular moiety through an oxygen atom. Representative examples of (C₁-C₈)alkoxy include, but are not limited to, methoxy, ethoxy, propoxy, 2-propoxy, butoxy, tert-butoxy, isobutoxy, pentyloxy, hexyloxy, heptyloxy, and octyloxy. The term “(C₁-C₈)alkoxy(C₁₋C₈)alkoxy” as used herein, means a (C₁-C₈)alkoxy group, as defined herein, appended to the parent molecular moiety through another (C₁-C₈)alkoxy group, as defined herein. Representative examples of (C₁-C₈)alkoxy(C₁-C₈)alkoxy include, but are not limited to, tert-butoxymethoxy, 2-ethoxyethoxy, 2-methoxyethoxy, and methoxymethoxy.

The term “(C₁-C₈)alkoxy(C₁₋C₈)alkyl” as used herein, means a (C₁-C₈)alkoxy group, as defined herein, appended to the parent molecular moiety through a (C₁-C₈)alkyl group, as defined herein. Representative examples of (C₁-C₈)alkoxy(C₁-C₈)alkyl include, but are not limited to, tert-butoxymethyl, 2-ethoxyethyl, 2-methoxyethyl, and methoxymethyl.

The term “(C₁-C₈)alkoxycarbonyl” as used herein, means a (C₁-C₈)alkoxy group, as defined herein, appended to the parent molecular moiety through a carbonyl group, as defined herein. Representative examples of (C₁-C₈)alkoxycarbonyl include, but are not limited to, methoxycarbonyl, ethoxycarbonyl, tert-butoxycarbonyl, hexyloxycarbonyl, and octyloxycarbonyl.

The term “(C₁-C₁₄)alkyl” as used herein, means a straight or branched chain hydrocarbon containing from 1 to 14 carbon atoms. Representative examples of (C₁-C₁₄)alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, undecyl, dodecyl, and tridecyl.

The term “(C₁-C₈)alkyl” as used herein, is a subset of (C₁-C₁₄)alkyl and means a straight or branched chain hydrocarbon containing from 1 to 8 carbon atoms. Representative examples of (C₁-C₈)alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, and n-octyl.

The term “(C₁-C₈)alkylcarbonyl” as used herein, means a (C₁-C₈)alkyl group, as defined herein, appended to the parent molecular moiety through a carbonyl group, as defined herein. Representative examples of (C₁-C₈)alkylcarbonyl include, but are not limited to, acetyl, 1-oxopropyl, 2,2-dimethyl-1-oxopropyl, 1-oxobutyl, and 1-oxopentyl.

The term “(C₁-C₈)alkylcarbonyloxy” as used herein, means a (C₁-C₈)alkylcarbonyl group, as defined herein, appended to the parent molecular moiety through an oxygen atom. Representative examples of (C₁-C₈)alkylcarbonyloxy include, but are not limited to, acetyloxy, ethylcarbonyloxy, and tert-butylcarbonyloxy.

The term “(C₁-C₈)alkylsulfinyl” as used herein, means an (C₁-C₈)alkyl group, as defined herein, appended to the parent molecular moiety through a sulfinyl group, as defined herein. Representative examples of (C₁-C₈)alkylsulfinyl include, but are not limited to, methylsulfinyl and ethylsulfinyl.

The term “(C₁-C₈)alkylsulfinyl(C₁₋C₈)alkyl” as used herein, means a (C₁-C₈)alkylsulfinyl group, as defined herein, appended to the parent molecular moiety through a (C₁-C₈)alkyl group, as defined herein.

The term “(C₁-C₈)alkylsulfonyl” as used herein, means an (C₁-C₈)alkyl group, as defined herein, appended to the parent molecular moiety through a sulfonyl group, as defined herein. Representative examples of (C₁-C₈)alkylsulfonyl include, but are not limited to, methylsulfonyl and ethylsulfonyl.

The term “(C₁-C₈)alkylsulfonyl(C₁₋C₈)alkyl” as used herein, means a (C₁-C₈)alkylsulfonyl group, as defined herein, appended to the parent molecular moiety through a (C₁-C₈)alkyl group, as defined herein.

The term “(C₁-C₈)alkylthio” as used herein, means a (C₁-C₈)alkyl group, as defined herein, appended to the parent molecular moiety through a sulfur atom. Representative examples of (C₁-C₈)alkylthio include, but are not limited, methylthio, ethylthio, tert-butylthio, and hexylthio.

The term “(C₁-C₈)alkylthio(C₁₋C₈)alkyl” as used herein, means a (C₁-C₈)alkylthio group, as defined herein, appended to the parent molecular moiety through a (C₁-C₈)alkyl group, as defined herein. Representative examples of (C₁-C₈)alkylthio(C₁-C₈)alkyl include, but are not limited, methylthiomethyl and 2-(ethylthio)ethyl.

The term “(C₂-C₁₄)alkynyl” as used herein, means a straight or branched chain hydrocarbon group containing from 2 to 14 carbon atoms and containing at least one carbon-carbon triple bond. Representative examples of (C₂-C₁₄)alkynyl include, but are not limited, to acetylenyl, 1-propynyl, 2-propynyl, 3-butynyl, 2-pentynyl, 1-butynyl, and 2,10,10-trimethylundec-4-ynyl.

The term “(C₂-C₈)alkynyl” as used herein, is a subset of (C₂-C₁₄)alkynyl and means a straight or branched chain hydrocarbon group containing from 2 to 8 carbon atoms and containing at least one carbon-carbon triple bond. Representative examples of (C₂-C₈)alkynyl include, but are not limited, to acetylenyl, 1-propynyl, 2-propynyl, 3-butynyl, 2-pentynyl, and 1-butynyl.

The term “aryl,” as used herein, means phenyl or naphthyl group.

The aryl groups of the invention are optionally substituted with 1, 2, 3, 4, or 5 substituents independently selected from the group consisting of (C₂-C₈)alkenyl, (C₁-C₈)alkoxy, (C₁-C₈)alkoxy(C₁-C₈)alkoxy, (C₁-C₈)alkoxy(C₁-C₈)alkyl, (C₁-C₈)alkoxycarbonyl, (C₁-C₈)alkyl, (C₁-C₈)alkylcarbonyl, (C₁-C₈)alkylcarbonyloxy, (C₁-C₈)alkylsulfinyl, (C₁-C₈)alkylsulfonyl, (C₁-C₈)alkylthio, (C₂-C₈)alkynyl, carboxy, carboxy(C₁-C₈)alkyl, cyano, cyano(C₁-C₈)alkyl, (C₃-C₈)cycloalkyl, (C₃-C₈)cycloalkyl(C₁-C₈)alkyl, formyl, halo(C₁-C₈)alkoxy, halo(C₁-C₈)alkyl, halogen, hydroxy, hydroxy(C₁-C₈)alkyl, mercapto, nitro, phenyl, —NZ₁Z₂, and (NZ₁Z₂)carbonyl.

The term “aryl(C₁-C₈)alkoxy” as used herein, means an aryl group, as defined herein, appended to the parent molecular moiety through an (C₁-C₈)alkoxy group, as defined herein.

The term “aryl(C₁-C₈)alkyl” as used herein, means an aryl group, as defined herein, appended to the parent molecular moiety through an (C₁-C₈)alkyl group, as defined herein. Representative examples of aryl(C₁-C₈)alkyl include, but are not limited to, benzyl, 2-phenylethyl, 3-phenylpropyl, and 2-naphth-2-ylethyl.

The term “aryl(C₁-C₈)alkylthio” as used herein, means an aryl(C₁-C₈)alkyl group, as defined herein, appended to the parent molecular moiety through a sulfur atom.

The term “carbonyl” as used herein, means a —C(O)— group.

The term “carboxy” as used herein, means a —CO₂H group.

The term “carboxy(C₁-C₈)alkyl” as used herein, means a carboxy group, as defined herein, is attached to the parent molecular moiety through a (C₁-C₈)alkyl group.

The term “cyano” as used herein, means a —CN group.

The term “cyano(C₁-C₈)alkyl” as used herein, means a cyano group, as defined herein, appended to the parent molecular moiety through a (C₁-C₈)alkyl group, as defined herein. Representative examples of cyanoalkyl include, but are not limited to, cyanomethyl, 2-cyanoethyl, and 3-cyanopropyl.

The term “(C₃-C₈)cycloalkenyl” as used herein, means a (C₃-C₈)cycloalkyl group, as defined herein, containing at least one carbon-carbon double bond. Representative examples of (C₃-C₈)cycloalkenyl include, but are not limited to, cyclohexenyl, cyclohexadienyl and cyclopentenyl.

The term “(C₃-C₈)cycloalkenyl(C₁-C₈)alkoxy” as used herein, means a (C₃-C₈)cycloalkenyl group, as defined herein, appended to the parent molecular moiety through a (C₁-C₈)alkoxy group, as defined herein.

The term “(C₃-C₈)cycloalkenyl(C₁-C₈)alkyl” as used herein, means a (C₃-C₈)cycloalkenyl group, as defined herein, appended to the parent molecular moiety through a (C₁-C₈)alkyl group, as defined herein.

The term “(C₃-C₈)cycloalkenyl(C₁-C₈)alkylthio” as used herein, means a (C₃-C₈)cycloalkenyl(C₁-C₈)alkyl group, as defined herein, appended to the parent molecular moiety through a sulfur atom.

The term “(C₃-C₈)cycloalkyl” as used herein, means a saturated cyclic hydrocarbon group containing from 3 to 8 carbons, examples of (C₃-C₈)cycloalkyl include cyclopropyl, cyclobutyl, cyclopentyl, cyclohexyl, cycloheptyl, and cyclooctyl.

The term “(C₃-C₈)cycloalkyl(C₁-C₈)alkoxy” as used herein, means a (C₃-C₈)cycloalkyl group, as defined herein, appended to the parent molecular moiety through a (C₁-C₈)alkoxy group, as defined herein.

The term “(C₃-C₈)cycloalkyl(C₁-C₈)alkyl” as used herein, means a (C₃-C₈)cycloalkyl group, as defined herein, appended to the parent molecular moiety through a (C₁-C₈)alkyl group, as defined herein. Representative examples of (C₃-C₈)cycloalkyl(C₁-C₈)alkyl include, but are not limited to, cyclopropylmethyl, 2-cyclobutylethyl, cyclopentylmethyl, cyclohexylmethyl, and 4-cycloheptylbutyl.

The term “(C₃-C₈)cycloalkyl(C₁-C₈)alkylthio” as used herein, means a (C₃-C₈)cycloalkyl(C₁-C₈)alkyl group, as defined herein, appended to the parent molecular moiety through a sulfur atom.

The term “formyl” as used herein, means a —C(O)H group.

The term “halo” or “halogen” as used herein, means —Cl, —Br, —I or —F.

The term “halo(C₁-C₈)alkoxy” as used herein, means at least one halogen, as defined herein, appended to the parent molecular moiety through a (C₁-C₈)alkoxy group, as defined herein. Representative examples of halo(C₁-C₈)alkoxy include, but are not limited to, chloromethoxy, 2-fluoroethoxy, trifluoromethoxy, and pentafluoroethoxy.

The term “halo(C₁-C₈)alkyl” as used herein, means at least one halogen, as defined herein, appended to the parent molecular moiety through a (C₁-C₈)alkyl group, as defined herein. Representative examples of halo(C₁-C₈)alkyl include, but are not limited to, chloromethyl, difluoromethyl, 2-fluoroethyl, trifluoromethyl, pentafluoroethyl, and 2-chloro-3-fluoropentyl.

The term “heteroaryl,” as used herein, means a monocyclic heteroaryl or a bicyclic heteroaryl. The monocyclic heteroaryl is a 5 or 6 membered ring. The 5 membered ring consists of two double bonds and one, two, three or four nitrogen atoms and optionally one oxygen or sulfur atom. The 6 membered ring consists of three double bonds and one, two, three or four nitrogen atoms. The 5 or 6 membered heteroaryl is connected to the parent molecular moiety through any carbon atom or any nitrogen atom contained within the heteroaryl. Representative examples of monocyclic heteroaryl include, but are not limited to, furyl, imidazolyl, isoxazolyl, isothiazolyl, oxadiazolyl, oxazolyl, pyridinyl, pyridazinyl, pyrimidinyl, pyrazinyl, pyrazolyl, pyrrolyl, tetrazolyl, thiadiazolyl, thiazolyl, thienyl, triazolyl, and triazinyl. The bicyclic heteroaryl consists of a monocyclic heteroaryl fused to a phenyl, or a monocyclic heteroaryl fused to a cycloalkyl, or a monocyclic heteroaryl fused to a cycloalkenyl, or a monocyclic heteroaryl fused to a monocyclic heteroaryl. The bicyclic heteroaryl is connected to the parent molecular moiety through any carbon atom or any nitrogen atom contained within the bicyclic heteroaryl. Representative examples of bicyclic heteroaryl include, but are not limited to, benzimidazolyl, benzofuranyl, benzothienyl, benzoxadiazolyl, cinnolinyl, dihydroquinolinyl, dihydroisoquinolinyl, furopyridinyl, indazolyl, indolyl, isoquinolinyl, naphthyridinyl, quinolinyl, tetrahydroquinolinyl, and thienopyridinyl.

The heteroaryl groups of the invention are optionally substituted with 1, 2, 3, or 4 substituents independently selected from the group consisting of (C₂-C₈)alkenyl, (C₁-C₈)alkoxy, (C₁-C₈)alkoxy(C₁-C₈)alkoxy, (C₁-C₈)alkoxy(C₁-C₈)alkyl, (C₁-C₈)alkoxycarbonyl, (C₁-C₈)alkoxysulfonyl, (C₁-C₈)alkyl, (C₁-C₈)alkylcarbonyl, (C₁-C₈)alkylcarbonyloxy, (C₁-C₈)alkylthio, (C₂-C₈)alkynyl, carboxy, carboxy(C₁-C₈)alkyl, cyano, cyano(C₁-C₈)alkyl, formyl, halo (C₁-C₈)alkoxy, halo (C₁-C₈)alkyl, halogen, hydroxy, hydroxy(C₁-C₈)alkyl, mercapto, nitro, —NR₁₁R₁₂, (NR₁₁R₁₂) (C₁-C₈)alkyl, and (NR₁₁R₁₂)carbonyl. Heteroaryl groups of the invention that are substituted may be present as tautomers. The invention encompasses all tautomers including non-aromatic tautomers.

The term “heteroaryl(C₁-C₈)alkoxy” as used herein, means a heteroaryl group, as defined herein, appended to the parent molecular moiety through an (C₁-C₈)alkoxy group, as defined herein.

The term “heteroaryl(C₁-C₈)alkyl” as used herein, means a heteroaryl, as defined herein, appended to the parent molecular moiety through an (C₁-C₈)alkyl group, as defined herein.

The term “heteroaryl(C₁-C₈)alkylthio” as used herein, means a heteroaryl(C₁-C₈)alkyl group, as defined herein, appended to the parent molecular moiety through a sulfur atom.

The term “heteroaryl(C₁-C₈)alkylthio(C₁-C₈)alkyl” as used herein, means a heteroaryl(C₁-C₈)alkylthio group, as defined herein, appended to the parent molecular moiety through an (C₁-C₈)alkyl group, as defined herein.

The term “heteroarylthio” as used herein, means a heteroaryl group, as defined herein, appended to the parent molecular moiety through a sulfur atom.

The term “heteroarylthio(C₁-C₈)alkyl” as used herein, means a heteroarylthio group, as defined herein, appended to the parent molecular moiety through an (C₁-C₈)alkyl group, as defined herein.

The term “heterocycle” or “heterocyclyl” or “heterocyclic” as used herein, means a 3, 4, 5, 6 or 7 membered ring containing at least one heteroatom independently selected from the group consisting of O, N, and S. The 3 or 4 membered ring contains 1 heteroatom selected from the group consisting of O, N and S. The 5 membered ring contains zero or one double bond and one, two or three heteroatoms selected from the group consisting of O, N and S. The 6 or 7 membered ring contains zero, one or two double bonds and one, two or three heteroatoms selected from the group consisting of O, N and S. The heterocycle is connected to the parent molecular moiety through any carbon atom or any nitrogen atom contained within the heterocycle. Representative examples of heterocycle include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, oxetanyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl.

The heterocycle groups of the invention are optionally substituted with 1, 2, 3, 4, or 5 substituents independently selected from the group consisting of (C₂-C₈)alkenyl, (C₁-C₈)alkoxy, (C₁-C₈)alkoxy(C₁-C₈)alkoxy, (C₁-C₈)alkoxy(C₁-C₈)alkyl, (C₁-C₈)alkoxycarbonyl, (C₁-C₈)alkyl, (C₁-C₈)alkylcarbonyl, (C₁-C₈)alkylcarbonyloxy, (C₁-C₈)alkylthio, (C₂-C₈)alkynyl, carboxy, carboxy(C₁-C₈)alkyl, cyano, cyano(C₁-C₈)alkyl, formyl, halo(C₁-C₈)alkoxy, halo(C₁-C₈)alkyl, halogen, hydroxy, hydroxy(C₁-C₈)alkyl, mercapto, nitro, oxo, —NR₁₁R₁₂, (NR₁₁R₁₂)alkyl, and (NR₁₁R₁₂)carbonyl.

The term “heterocycle(C₁-C₈)alkoxy” as used herein, means a heterocycle group, as defined herein, appended to the parent molecular moiety through an (C₁-C₈)alkoxy group, as defined herein.

The term “heterocycle(C₁-C₈)alkyl” as used herein, means a heterocycle, as defined herein, appended to the parent molecular moiety through an (C₁-C₈)alkyl group, as defined herein.

The term “heterocycle(C₁-C₈)alkylthio” as used herein, means a heterocycle(C₁-C₈)alkyl group, as defined herein, appended to the parent molecular moiety through a sulfur atom.

The term “hydroxy” as used herein, means an —OH group.

The term “hydroxy(C₁-C₈)alkoxy” as used herein, means at least one hydroxy group, as defined herein, is appended to the parent molecular moiety through a (C₁-C₈)alkoxy group, as defined herein. Representative examples of hydroxy(C₁-C₈)alkoxy include, but are not limited to, hydroxymethoxy, 2-hydroxyethoxy, 3-hydroxypropoxy, 2,3-dihydroxypentyloxy, and 2-ethyl-4-hydroxyheptyloxy.

The term “hydroxy (C₁-C₈) alkoxy (C₁-C₈) alkoxy” as used herein, means a hydroxy(C₁-C₈)alkoxy group, as defined herein, is appended to the parent molecular moiety through a (C₁-C₈)alkoxy group, as defined herein. Representative examples of hydroxy(C₁-C₈)alkoxy(C₁-C₈)alkoxy include, but are not limited to, 2-(2-hydroxyethoxy)ethoxy.

The term “hydroxy(C₁-C₈)alkyl” as used herein, means at least one hydroxy group, as defined herein, is appended to the parent molecular moiety through a (C₁-C₈)alkyl group, as defined herein. Representative examples of hydroxy(C₁-C₈)alkyl include, but are not limited to, hydroxymethyl, 2-hydroxyethyl, 3-hydroxypropyl, 2,3-dihydroxypentyl, and 2-ethyl-4-hydroxyheptyl.

The term “mercapto” as used herein, means a —SH group.

The term “nitro” as used herein, means a —NO₂ group.

The term “—NR₁₁R₁₂” as used herein, means two groups, R₁₁ and R₁₂, which are appended to the parent molecular moiety through a nitrogen atom. R₁₁ and R₁₂ are each independently —H, (C₂-C₈)alkenyl, (C₁-C₈)alkyl, (C₁-C₈)alkylcarbonyl, (C₂-C₈) alkynyl, aryl, aryl(C₁-C₈) alkyl, (C₃-C₈) cycloalkyl, (C₃-C₈)cycloalkyl(C₁-C₈)alkyl, formyl, heteroaryl, heteroaryl(C₁-C₈)alkyl, heterocyclyl, or heterocycle(C₁-C₈)alkyl. Representative examples of —NR₁₁R₁₂ include, but are not limited to, amino, methylamino, ethylamino, diethylamino, neopentylamino, acetylamino, acetylmethylamino, cyclopropylamino, dicyclopropylamino, cyclopropylmethylamino, dicyclopropylmethylamino, propynylamino, and dipropynylamino.

The term “(NR₁₁R₁₂) (C₁₋C₈)alkyl” as used herein, means a —NR₁₁R₁₂ group, as defined herein, appended to the parent molecular moiety through a (C₁-C₈)alkyl group, as defined herein.

The term “(C₁-C₈)alkyl(NR₁₁) (C₁-C₈)alkyl” as used herein, means a (C₁-C₈)alkyl(NR₁₁)-group, as defined herein, appended to the parent molecular moiety through a (C₁-C₈)alkyl group, as defined herein. R₁₁ is —H, (C₂-C₈)alkenyl, (C₁-C₈)alkyl, (C₁-C₈)alkylcarbonyl, (C₂-C₈)alkynyl, aryl, aryl(C₁-C₈)alkyl, (C₃-C₈)cycloalkyl, (C₃-C₈)cycloalkyl(C₁-C₈)alkyl, formyl, heteroaryl, heteroaryl(C₁-C₈)alkyl, heterocycle, or heterocycle (C₁-C₈) alkyl.

The term “(NR₁₁R₁₂)carbonyl” as used herein, means a NZ₁Z₂ group, as defined herein, appended to the parent molecular moiety through a carbonyl group, as defined herein. Representative examples of (NR₁₁R₁₂)carbonyl include, but are not limited to, aminocarbonyl, (methylamino)carbonyl, (dimethylamino)carbonyl, and (ethylmethylamino)carbonyl.

The term “oxo” as used herein, means a ═O moiety.

The term “sulfinyl” as used herein, means a —S(O)— group.

The term “sulfonyl” as used herein, means a —SO₂— group.

Compounds of the invention can exist as stereoisomers, wherein asymmetric or chiral centers are present. Stereoisomers are designated “R” or “S,” depending on the configuration of substituents around the chiral carbon atom. The terms “R” and “S” used herein are configurations as defined in IUPAC 1974 Recommendations for Section E, Fundamental Stereochemistry, Pure Appl. Chem., (1976), 45: 13-30, hereby incorporated by reference. The invention contemplates various stereoisomers and mixtures thereof and are specifically included within the scope of this invention. Stereoisomers include enantiomers, diastereomers, and mixtures of enantiomers or diastereomers. Individual stereoisomers of compounds of the invention may be prepared synthetically from commercially available starting materials which contain asymmetric or chiral centers or by preparation of racemic mixtures followed by resolution well-known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary or (2) direct separation of the mixture of optical enantiomers on chiral chromatographic columns.

Cis and trans isomers and mixtures thereof may also exist in the compounds of the invention. Cis and trans isomers and mixtures thereof are specifically included within the scope of this invention. An example of cis and trans isomers of the invention includes, but is not limited to, compounds where R₆, of Formula (I), is a 4-substituted cyclohexyl group. The preferred isomer is trans for compounds of the invention where R₆ is a 4-substituted cyclohexyl group.

The term “pharmaceutically acceptable salt” or “salt,” as used herein, refers to salts that are well known in the art. For example, S. M Berge et al. describe pharmaceutically acceptable salts in detail in J. Pharmaceutical Sciences, 66:1-19 (1977), which is incorporated by reference. Preferred salts of the invention include, but are not limited to, acetate, monohydrochloride, dihydrochloride, and mesylate.

The invention encompasses the use of a therapeutically effective amount of compounds or salts of the invention for the preparation of a medicament for the treatment of cancer, inflammation, viral infection, or arthritis in a patient in need of such treatment.

In the methods for treating viral infections, particular viral infections include those resulting from HIV-1 and Hepatitis C virus.

### Pharmaceutical Compositions

The compounds of Formula (I) may be administered orally, topically, parenterally, by inhalation or spray or rectally in dosage unit formulations containing conventional non-toxic pharmaceutically acceptable carriers, adjuvants and vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a compound of Formula (I) and a pharmaceutically acceptable carrier. One or more compounds of Formula (I) may be present in association with one or more non-toxic pharmaceutically acceptable carriers and/or diluents and/or adjuvants, and if desired other active ingredients. The pharmaceutical compositions containing compounds of Formula (I) may be in a form suitable for oral use, for example, as tablets, troches, lozenges, aqueous or oily suspensions, dispersible powders or granules, emulsion, hard or soft capsules, or syrups or elixirs.

Compositions intended for oral use may be prepared according to any method known in the art for the manufacture of pharmaceutical compositions and such compositions may contain one or more agents selected from the group consisting of sweetening agents, flavoring agents, coloring agents and preservative agents in order to provide pharmaceutically elegant and palatable preparations. Tablets contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques. In some cases such coatings may be prepared by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatin capsules, wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Formulations for oral use may also be presented as lozenges.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, may also be present.

Pharmaceutical compositions of the invention may also be in the form of oil-in-water emulsions. The oily phase may be a vegetable oil or a mineral oil or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol, glucose or sucrose. Such formulations may also contain a demulcent, a preservative and flavoring and coloring agents. The pharmaceutical compositions may be in the form of a sterile injectable aqueous or oleaginous suspension. This suspension may be formulated according to the known art using those suitable dispersing or wetting agents and suspending agents that have been mentioned above. The sterile injectable preparation may also be a sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of Formula (I) may also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Compounds of Formula (I) may be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

For disorders of the eye or other external tissues, e.g., mouth and skin, the formulations are preferably applied as a topical gel, spray, ointment or cream, or as a suppository, containing the active ingredients in a total amount of, for example, 0.075 to 30% w/w, preferably 0.2 to 20% w/w and most preferably 0.4 to 15% w/w. When formulated in an ointment, the active ingredients may be employed with either paraffinic or a water-miscible ointment base.

Alternatively, the active ingredients may be formulated in a cream with an oil-in-water cream base. If desired, the aqueous phase of the cream base may include, for example at least 30% w/w of a polyhydric alcohol such as propylene glycol, butane-1,3-diol, mannitol, sorbitol, glycerol, polyethylene glycol and mixtures thereof. The topical formulation may desirably include a compound which enhances absorption or penetration of the active ingredient through the skin or other affected areas. Examples of such dermal penetration enhancers include dimethylsulfoxide and related analogs. The compounds of this invention can also be administered by a transdermal device. Preferably topical administration will be accomplished using a patch either of the reservoir and porous membrane type or of a solid matrix variety. In either case, the active agent is delivered continuously from the reservoir or microcapsules through a membrane into the active agent permeable adhesive, which is in contact with the skin or mucosa of the recipient. If the active agent is absorbed through the skin, a controlled and predetermined flow of the active agent is administered to the recipient. In the case of microcapsules, the encapsulating agent may also function as the membrane. The transdermal patch may include the compound in a suitable solvent system with an adhesive system, such as an acrylic emulsion, and a polyester patch. The oily phase of the emulsions of this invention may be constituted from known ingredients in a known manner. While the phase may comprise merely an emulsifier, it may comprise a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make-up the so-called emulsifying wax, and the wax together with the oil and fat make up the so-called emulsifying ointment base which forms the oily dispersed phase of the cream formulations. Emulsifiers and emulsion stabilizers suitable for use in the formulation of the invention include Tween 60, Span 80, cetostearyl alcohol, myristyl alcohol, glyceryl monostearate, and sodium lauryl sulfate, among others. The choice of suitable oils or fats for the formulation is based on achieving the desired cosmetic properties, since the solubility of the active compound in most oils likely to be used in pharmaceutical emulsion formulations is very low. Thus, the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters may be used. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredients are dissolved or suspended in suitable carrier, especially an aqueous solvent for the active ingredients. The antiinflammatory active ingredients are preferably present in such formulations in a concentration of 0.5 to 20%, advantageously 0.5 to 10% and particularly about 1.5% w/w. For therapeutic purposes, the active compounds of this combination invention are ordinarily combined with one or more adjuvants appropriate to the indicated route of administration. If administered per os, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose. Formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The compounds may be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Dosage levels of the order of from about 0.1 mg to about 140 mg per kilogram of body weight per day are useful in the treatment of the above-indicated conditions (about 0.5 mg to about 7 g per patient per day). The amount of active ingredient (at least one compound of Formula (I)) that may be combined with the carrier materials to produce a single dosage form will vary depending upon the host treated and the particular mode of administration. Dosage unit forms will generally contain between from about 1 mg to about 500 mg of an active ingredient. The daily dose can be administered in one to four doses per day. In the case of skin conditions, it may be preferable to apply a topical preparation of compounds of this invention to the affected area two to four times a day.

It will be understood, however, that the specific dose level for any particular patient will depend upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition may also be added to the animal feed or drinking water. It may be convenient to formulate the animal feed and drinking water compositions so that the animal takes in a therapeutically appropriate quantity of the composition along with its diet. It may also be convenient to present the composition as a premix for addition to the feed or drinking water. Preferred non-human animals include domesticated animals.

The compounds of the invention may be administered alone or in combination with at least one additional therapeutic agent or therapy, e.g., radiation therapy, to a patient in need of such treatment. The additional therapeutic agent or therapy may be administered at the same time, separately, or sequentially with respect to the administration of a compound of the invention. Such additional therapeutic agents included, but are not limited to, anti-cancer agents, anti-inflammatory agents, and the like.

The compounds of the invention may be prepared by use of known chemical reactions and procedures. Representative methods for synthesizing compounds of the invention are presented below. It is understood that the nature of the substituents required for the desired target compound often determines the preferred method of synthesis. All variable groups of these methods are as described in the generic description if they are not specifically defined below.

### Methods of Preparation

Representative synthetic procedures for the preparation of compounds of the invention are outlined below in Schemes 1-5.

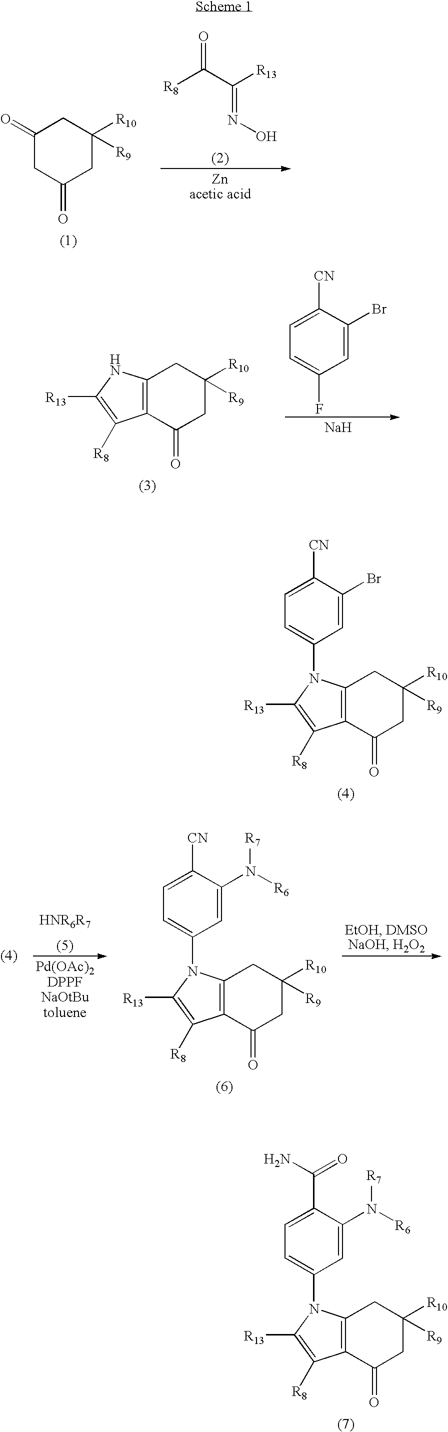

Compounds of formula (7), wherein R₆, R₇, R₈, R₉, R₁₀, and R₁₃ are as defined in Formula (I), are prepared as described in Scheme 1. A Dione of formula (I) is treated with an oxime of formula (2), zinc, and an acid in a solvent to provide a compound of formula (3). A compound of formula (3) is treated with 2-bromo-4-fluorobenzonitrile and a base in a solvent to provide a compound of formula (4). A compound of formula (4) is treated with an amine of formula (5), a metal, optionally a ligand such as 1,1′-bis(diphenylphosphino)ferrocene (DPPF), and a base in a solvent to provide a compound of formula (6). A compound of formula (6) is treated with aqueous base and a peroxide to provide a compound of formula (7).

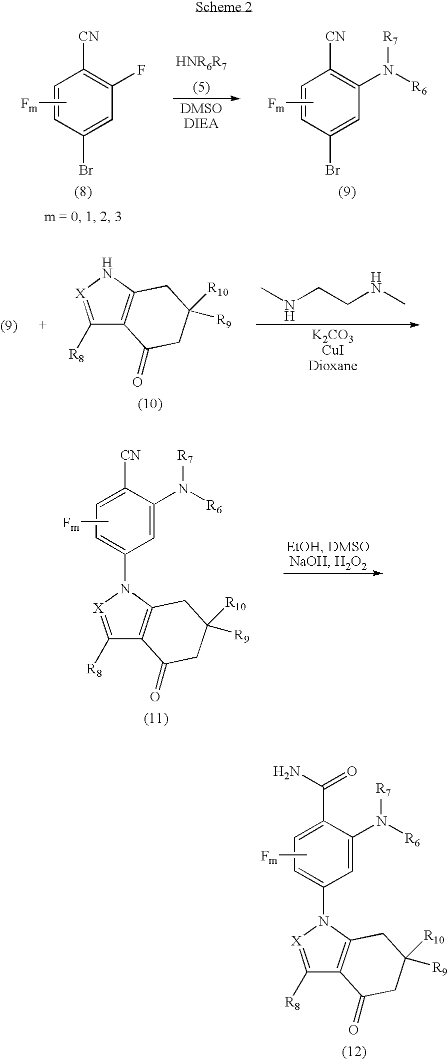

Compounds of formula (12), wherein R₆, R₇, R₈, R₉, R₁₀, and X are as defined in Formula (I), are prepared as described in Scheme 2. A benzonitrile of formula (8), wherein m=0, 1, 2, or 3, is treated with an amine of formula (5) and a base in a solvent to provide a benzonitrile of formula (9). A benzonitrile of formula (9) is treated with N,N′-dimethylethane-1,2-diamine, a base, and copper in the +1 oxidation state in a solvent to provide a compound of formula (11). A compound of formula (11) is treated with aqueous base and a peroxide to provide a compound of formula (12).

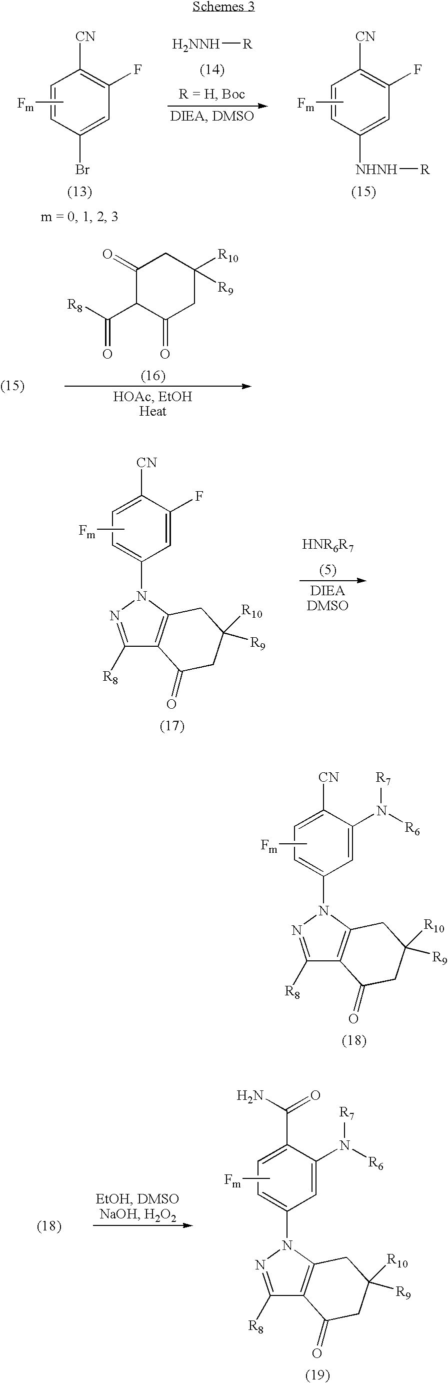

Compounds of formula (19), wherein R₆, R₇, R₈, R₉, and R₁₀ are as defined in Formula (I), are prepared as described in Scheme 3. A benzonitrile of formula (13) is treated with a base and a hydrazine of formula (14), wherein Boc is tert-butoxycarbonyl, in a solvent to provide a benzonitrile of formula (15). A benzonitrile of formula (15) is treated with an acid, a compound of formula (16), and heat in a solvent to provide a compound of formula (17). A compound of formula (17) is treated with a base and an amine of formula (5) in a solvent to provide a compound of formula (18). A compound of formula (18) is treated with aqueous base and a peroxide to provide a compound of formula (19).

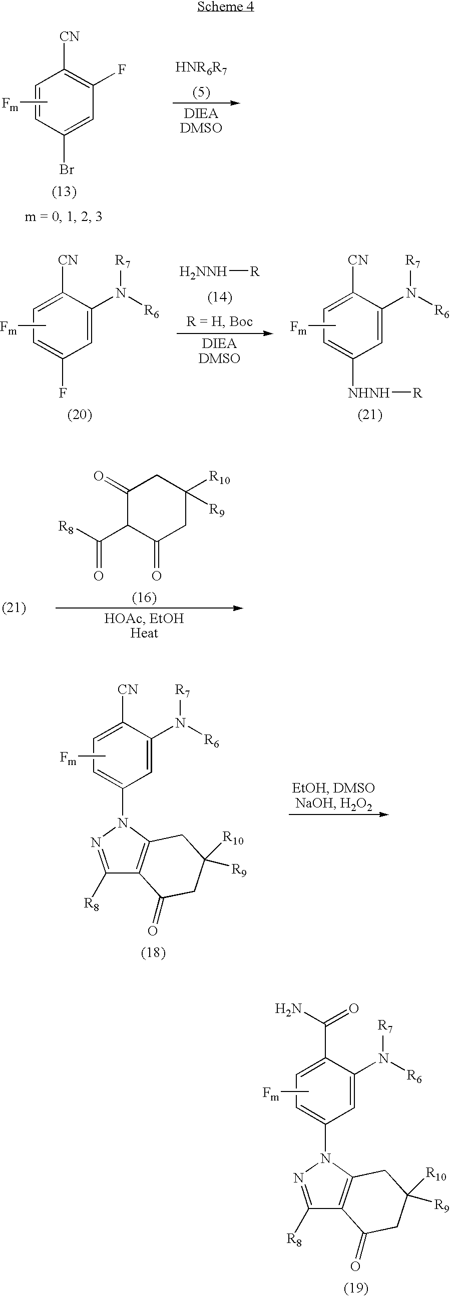

Alternatively, compounds of formula (19), wherein R₆, R₇, R₈, R₉, and R₁₀ are as defined in Formula (I), are prepared as described in Scheme 4. A benzonitrile of formula (13), wherein m=0, 1, 2, or 3, is treated with a base and a compound of formula (5) in a solvent to provide a compound of formula (20). A compound of formula (20) is treated with a base and a hydrazine of formula (14), wherein Boc is tert-butoxycarbonyl, in a solvent to provide a compound of formula (21). A compound of formula (21) is treated with an acid and a compound of formula (16) with heat in a solvent to provide a compound of formula (18). A compound of formula (18) is treated with aqueous base and a peroxide to provide a compound of formula (19).

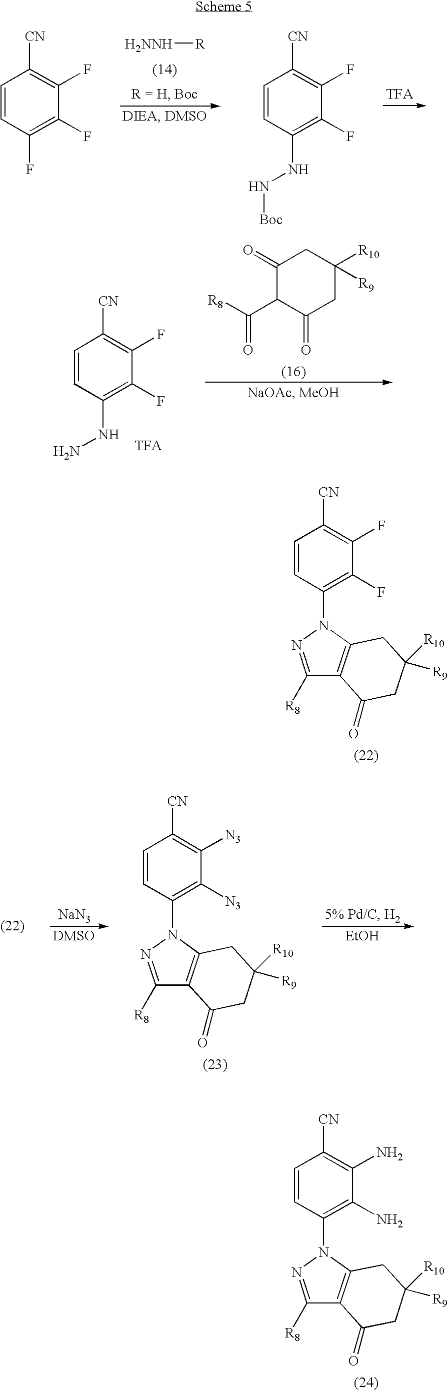

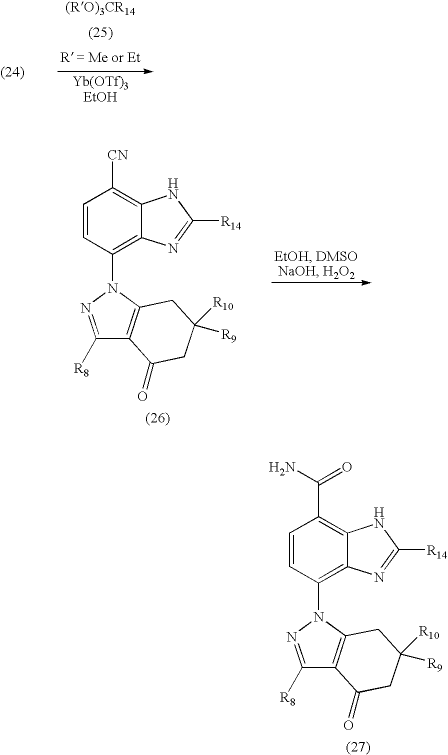

Compounds of formula (27), wherein R₈, R₉, and R₁₀ are as defined in Formula (I), and R₁₄ is H, (C₁-C₈)alkyl, (C₃-C₈)cycloalkyl, aryl, aryl(C₁-C₈)alkyl, heteroaryl, heteroaryl(C₁-C₈)alkyl, heterocyclyl, or heterocycle(C₁-C₈)alkyl, are prepared as described in Scheme 5. 2,3,4-Trifluorobenzonitrile is treated with a hydrazine of formula (14), wherein Boc is tert-butoxycarbonyl, in a solvent to provide tert-butyl 2-(4-cyano-2,3-difluorophenyl)hydrazinecarboxylate (or 2,3-difluoro-4-hydrazinylbenzonitrile). tert-Butyl 2-(4-cyano-2,3-difluorophenyl)hydrazinecarboxylate is treated with an acid in a solvent to provide 2,3-difluoro-4-hydrazinylbenzonitrile. 2,3-diFluoro-4-hydrazinylbenzonitrile is treated with a base and a compound of formula (16) in a solvent to provide a compound of formula (22). A compound of formula (22) is treated with sodium azide in a solvent to provide a compound of formula (23). A compound of formula (23) is treated with a metal catalyst under a hydrogen atmosphere (optionally with the pressure greater than atmospheric pressure) in a solvent to provide a compound of formula (24). A compound of formula (24) is treated with an ortho ester of formula (25) and ytterbium triflate in a solvent to provide a compound of formula (26). A compound of formula (26) is treated with aqueous base and a peroxide to provide a compound of formula (27).

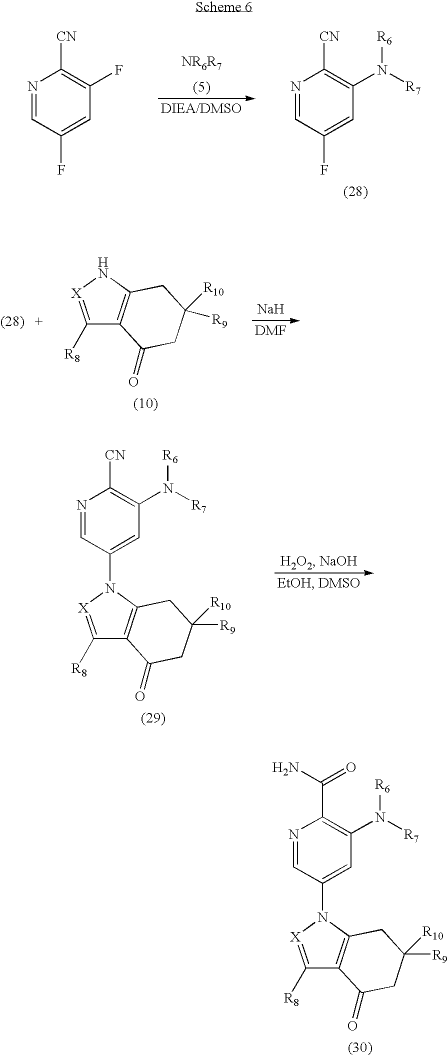

Compounds of formula (30), wherein X, R₆, R₇, R₈, R₉, and R₁₀ are as defined in Formula (I), are prepared as described in Scheme 6. 3,5-Difluoropicolinonitrile is treated with an amine of formula (5) in a solvent or cosolvent, such as diisopropylethylamine and dimethylsulfoxide, to provide a pyridine of formula (28). A pyridine of formula (28) is treated with a compound of formula (10) and a base in a solvent to provide a compound of formula (29). A compound of formula (29) is treated with aqueous base and a peroxide in a solvent or cosolvent, such as ethanol and dimethylsulfoxide, to provide a compound of formula (30).

Those having skill in the art will recognize that the starting materials and reaction conditions may be varied, the sequence of the reactions altered, and additional steps employed to produce compounds encompassed by the invention, as demonstrated by the following examples. In some cases, protection of certain reactive functionalities may be necessary to achieve some of the above transformations. In general, the need for such protecting groups as well as the conditions necessary to attach and remove such groups will be apparent to those skilled in the art of organic synthesis.

The disclosures of all articles and references mentioned in this application, including patents, are incorporated herein by reference in their entirety.

Compounds of the invention were named using Chemdraw version 10.0 (developed by CambridgeSoft available at cambridgesoft.com) or were given names which appeared to be consistent with Chemdraw version 10.0.

The invention is illustrated further by the following examples which are not to be construed as limiting the invention in scope or spirit to the specific procedures described in them. These examples illustrate the presently preferred methods for preparing the compounds of the invention.

### EXAMPLE 1

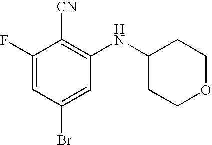

### 4-Bromo-2-fluoro-6-(tetrahydro-pyran-4-ylamino)-benzonitrile

To a solution of 0.8 g (3.67 mmol) of 4-Bromo-2,6-difluoro-benzonitrile in DMSO (15 mL) was added 0.497 g (3.85 mmol) DIEA and 0.371 g (3.67 mmol) of Tetrahydro-pyran-4-ylamine. This was stirred at 25° C. for 20 hours and then poured into 75 mL of water. The resulting solid was collected by filtration and washed with water (3×20 ml) and dried to give 0.925 g of 4-Bromo-2-fluoro-6-(tetrahydro-pyran-4-ylamino)-benzonitrile (84.2% yield). LCMS (m/z): M+H=298.9.

### EXAMPLE 2

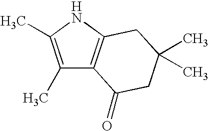

### 2,3,6,6-Tetramethyl-1,5,6,7-tetrahydro-indol-4-one

To a solution of 2,3-butanedione monoxime (10 g, 99 mmol) and dimedone (13.9 g, 99 mmol) in Acetic acid (96 mL) and water (41 mL) cooled at 0° C., was slowly added powder Zn (12.3 g, 188 mmol). The reaction mixture was refluxed overnight. The solvent was removed under reduced pressure, and the residue was partitioned between brine (100 mL) and CH₂Cl₂ (100 mL). The organic phase was evaporated under reduced pressure. The resulting solid was collected by vacuum filtration, and rinsed with CH₂Cl₂. The solid was then dried under vacuum to afford 5.6 g (30%) of 2,3,6,6-Tetramethyl-1,5,6,7-tetrahydro-indol-4-one as a yellow solid.

### EXAMPLE 3

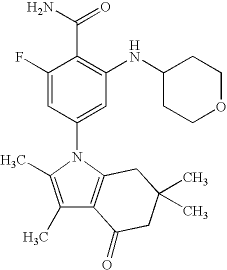

### 2-fluoro-6-(tetrahydro-2H-pyran-4-ylamino)-4-(2,3,6,6-tetramethyl-4-oxo-4,5,6,7-tetrahydro-1H-indol-1-yl)benzamide

4-Bromo-2-fluoro-6-(tetrahydro-pyran-4-ylamino)-benzonitrile (0.2 g, 0.67 mmol), 2,3,6,6-Tetramethyl-1,5,6,7-tetrahydro-indol-4-one (0.128 g, 0.558 mmol), and K₂CO₃ (0.463 g, 3.35 mmol) were dissolved/suspended in 5 mL Dioxane. The reaction mixture was degassed by 3 freeze (0° C.)/pump/thaw cycles under N₂. Then N,N′-Dimethylethylenediamine (0.086 g, 0.977 mmol) and CuI (191.4 mg, 1.005 mmol) were added and the reaction was again degassed by three more freeze/pump/thaw cycles. The reaction was then heated to 100° C. for 48 hours. After cooling the reaction mixture was filtered through celite, washed with EtOAc (3×20 mL), concentrated and purified via chromatography (50% EtOAc/Hex) to give 2-Fluoro-6-(tetrahydro-pyran-4-ylamino)-4-(2,3,6,6-tetramethyl-4-oxo-4,5,6,7-tetrahydro-indol-1-yl)-benzonitrile (0.01 g; 3.7% yield). LCMS (m/z): M+H=409.1.

2-Fluoro-6-(tetrahydro-pyran-4-ylamino)-4-(2,3,6,6-tetramethyl-4-oxo-4,5,6,7-tetrahydro-indol-1-yl)-benzonitrile (10 mg, 0.025 mmol) was dissolved in 8 mL of a 4:1 EtOH:DMSO solution. To this solution was added 0.4 mL 1M NaOH and 0.4 mL 30% H₂O₂ and the reaction mixture was stirred for 2 hours. The mixture was quenched by pouring into 50 ml 10% Na₂S₂O₃(aq) and extracted 3 times with EtOAc. The combined organic layers were dried over Na₂SO₄, concentrated and purified via chromatography (100% EtOAc) to give 2-fluoro-6-(tetrahydro-2H-pyran-4-ylamino)-4-(2,3,6,6-tetramethyl-4-oxo-4,5,6,7-tetrahydro-1H-indol-1-yl)benzamide (10 mg; 93.6% yield). LCMS (m/z): M+H=428.2.

### EXAMPLE 4

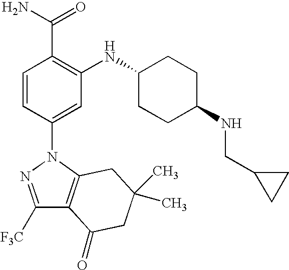

### 2-(trans-4-(cyclopropylmethylamino)cyclohexylamino)-4-(6,6-dimethyl-4-oxo-3-(trifluoromethyl)-4,5,6,7-tetrahydro-1H-indazol-1-yl)benzamide

2-Bromo-4-(6,6-dimethyl-4-oxo-3-trifluoromethyl-4,5,6,7-tetrahydro-indazol-1-yl)-benzonitrile (0.25 g, 0.672 mmol), Cyclohexane-1,4-diamine (0.338 g, 3.358 mmol), Pd(OAc)₂ (0.03 g, 0.134 mmol), DPPF (0.074 g, 0.134 mmol) and ^{t}BuONa (0.194 g, 2.016 mmol) were combined in a large microwave vial and dissolved/suspended in 6 mL of toluene. The vial was sealed and heated to 150° C. for 30 minutes in the microwave. The reaction mixture was purified directly (without workup) by chromatography (50% EtOAc/Hex) to give 2-(4-Amino-cyclohexylamino)-4-(6,6-dimethyl-4-oxo-3-trifluoromethyl-4,5,6,7-tetrahydro-indazol-1-yl)-benzonitrile (0.115 g; 40% yield). LCMS (m/z): M+H+MeCN=486.7.

To a solution of 2-(4-Amino-cyclohexylamino)-4-(6,6-dimethyl-4-oxo-3-trifluoromethyl-4,5,6,7-tetrahydro-indazol-1-yl)-benzonitrile (57.5 mg, 0.129 mmol) in 2 mL DMF in a medium microwave vial was added Bromomethyl-cyclopropane (17.4 mg, 0.129 mmol) and DIEA (16.7 mg, 0.129 mmol). The vial was sealed and the reaction was heated to 150° C. for 30 minutes in the microwave. The reaction mixture was cooled and then poured into 20 mL sat. NH₄Cl(aq) and extracted 3 times with EtOAc. The organic layers were combined, dried over Na₂SO₄, concentrated and purified by chromatography (75% EtOAc/Hex) to give 2-[4-(Cyclopropylmethyl-amino)-cyclohexylamino]-4-(6,6-dimethyl-4-oxo-3-trifluoromethyl-4,5,6,7-tetrahydro-indazol-1-yl)-benzonitrile (50 mg; 77.6% yield). LCMS (m/z): M+H=500.3.

2-[4-(Cyclopropylmethyl-amino)-cyclohexylamino]-4-(6,6-dimethyl-4-oxo-3-trifluoromethyl-4,5,6,7-tetrahydro-indazol-1-yl)-benzonitrile (50 mg, 0.1 mmol) was dissolved in 2 mL of a 4:1 EtOH:DMSO solution. To this solution was added 0.1 mL 1M NaOH and 0.1 mL 30% H₂O₂ and the reaction was stirred for 2 hours. The reaction was quenched by pouring into 10 mL 10% Na₂S₂O₃(aq) and extracted 3 times with CH₂Cl₂. The organics were dried over Na₂SO₄, concentrated, and purified by chromatography (100% EtOAc) to give 2-(trans-4-(cyclopropylmethylamino)cyclohexylamino)-4-(6,6-dimethyl-4-oxo-3-(trifluoromethyl)-4,5,6,7-tetrahydro-1H-indazol-1-yl)benzamide (10 mg; 20% yield). LCMS (m/z): M+H=518.3.

### EXAMPLE 5

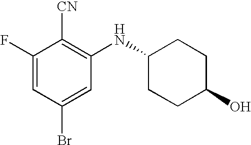

### 4-Bromo-2-fluoro-6-(trans-4-hydroxy-cyclohexylamino)-benzonitrile

To a solution of 0.5 g (2.29 mmol) of 4-Bromo-2,6-difluoro-benzonitrile in DMSO (10 mL) was added 0.31 g (2.4 mmol) DIEA and 0.42 g (2.29 mmol) of 4-Amino-cyclohexanol. This was stirred at 25° C. for 16 hours and then poured into 50 mL water. The resulting solid was collected by filtration and washed with water (3×20 mL) and dried to give 0.644 g of 4-Bromo-2-fluoro-6-(4-hydroxy-cyclohexylamino)-benzonitrile (90% yield). LCMS (m/z): M+H=313.0 and 315.0.

### EXAMPLE 6

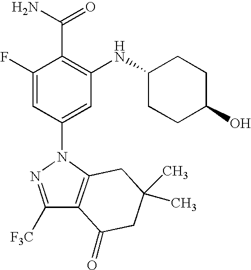

### 4-(6,6-dimethyl-4-oxo-3-(trifluoromethyl)-4,5,6,7-tetrahydro-1H-indazol-1-yl)-2-fluoro-6-(trans-4-hydroxycyclohexylamino)benzamide

4-Bromo-2-fluoro-6-(4-hydroxy-cyclohexylamino)-benzonitrile (0.563 g, 1.804 mmol), 6,6-Dimethyl-3-trifluoromethyl-1,5,6,7-tetrahydro-indazol-4-one (0.350 g, 1.504 mmol) and K₂CO₃ (1.243 g, 9 mmol) were dissolved/suspended in 7 mL Dioxane. The reaction mixture was degassed by 3 freeze (0° C.)/pump/thaw cycles under N₂. Then N,N′-Dimethylethylenediamine (0.232 g, 2.632 mmol) and CuI (516.1 mg, 2.71 mmol) were added and the reaction was again degassed by three more freeze/pump/thaw cycles. The reaction mixture was then heated to 100° C. for 48 hours. After cooling the reaction was filtered through celite, washed with EtOAc (3×20 mL), concentrated and purified via chromatography (50% EtOAc/Hex) to give 4-(6,6-Dimethyl-4-oxo-3-trifluoromethyl-4,5,6,7-tetrahydro-indazol-1-yl)-2-fluoro-6-(4-hydroxy-cyclohexylamino)-benzonitrile (0.368 g; 53% yield). LCMS (m/z): M+H=465.2.

4-(6,6-Dimethyl-4-oxo-3-trifluoromethyl-4,5,6,7-tetrahydro-indazol-1-yl)-2-fluoro-6-(4-hydroxy-cyclohexylamino)-benzonitrile (368 mg, 0.7931 mmol) was dissolved in 8 mL of a 4:1 EtOH:DMSO solution. To this solution was added 0.4 mL 1M NaOH and 0.4 mL 30% H₂O₂ and the reaction was stirred for 2 hours. The reaction mixture was quenched by pouring into 50 mL 10% Na₂S₂O₃(aq) and extracted 3 times with EtOAc. The organic layers were dried over Na₂SO₄, concentrated to approximately 10 mL and hexane added until solid formation began and the solution was then cooled to 0° C. for 48 hours and filtered to give 4-(6,6-dimethyl-4-oxo-3-(trifluoromethyl)-4,5,6,7-tetrahydro-1H-indazol-1-yl)-2-fluoro-6-(trans-4-hydroxycyclohexylamino)benzamide (300 mg; 78.4% yield). LCMS (m/z): M+H=483.2. ¹H NMR (DMSO, 20° C., 400 MHz) δ (ppm) 7.78 (b, 1H), 7.76 (b, 1H), 7.16 (d, 1H), 6.71 (d, 1H), 6.66 (dd, 1H), 4.54 (d, 1H), 3.49-3.40 (m, 1H), 2.97 (s, 2H), 2.42 (s, 2H), 1.99-1.91 (m, 2H), 1.82-1.75 (m, 2H), 1.35-1.13 (m, 4H), 1.02 (s, 6H).

### EXAMPLE 7

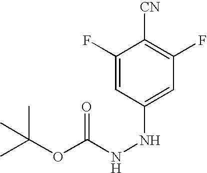

### tert-butyl 2-(4-cyano-3,5-difluorophenyl)hydrazinecarboxylate

To a solution of 2,4,6-trifluorobenzonitrile (5.62 g, 34.7 mmol) and tert-butylcarbazate (4.68 g, 34.7 mmol) in DMSO (70.0 mL) was added DIPEA (6.65 mL, 38.2 mmol) over 10 min. The resulting mixture was stirred at room temperature for 5 days. The mixture obtained was poured into brine solution and extracted with ethyl acetate (3×100 mL). The combined organic layer was washed with brine (3×30 mL), dried over Na₂SO₄, filtered and concentrated at reduced pressure to dryness. The residue obtained was purified by column chromatography (silica gel, 80:20 hexanes/ethyl acetate) to afford tert-butyl 2-(4-cyano-3,5-difluorophenyl)hydrazinecarboxylate (5.74 g, 61%) as a light yellow solid: ESI MS m/z=270 [M+H]⁺.

### EXAMPLE 8

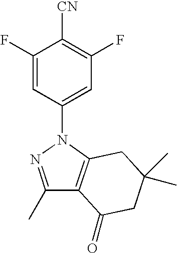

### 2,6-Difluoro-4-(3,6,6-trimethyl-4-oxo-4,5,6,7-tetrahydro-indazol-1-yl)-benzonitrile

A mixture of tert-butyl 2-(4-cyano-3,5-difluorophenyl)hydrazinecarboxylate (2.21 g, 8.20 mmol) and acetyldimedone (1.49 g, 8.20 mmol) in acetic acid (23 mL) and ethanol (0.5 mL) was stirred at 110° C. for 8 h. After cooling to room temperature, the reaction mixture was poured into water (300 mL) and extracted with ethyl acetate (3×100 mL). The combined organic layer was washed with brine (2×15 mL), saturated NaHCO₃ (3×15 mL) and brine (1×15 mL), dried over Na₂SO₄, filtered and concentrated at reduced pressure to dryness. The residue obtained was purified by column chromatography (silica gel, 80:20 hexanes/ethyl acetate) and triturated with hexanes/MTBE (2:1) to afford 2,6-Difluoro-4-(3,6,6-trimethyl-4-oxo-4,5,6,7-tetrahydro-indazol-1-yl)-benzonitrile (1.73 g, 67%) as a yellow solid: ESI MS m/z=316 [M+H]⁺.

### EXAMPLE 9

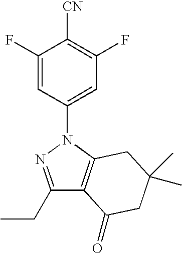

### 4-(3-Ethyl-6,6-dimethyl-4-oxo-4,5,6,7-tetrahydro-indazol-1-yl)-2,6-difluoro-benzonitrile

The title compound was prepared from tert-butyl 2-(4-cyano-3,5-difluorophenyl)hydrazinecarboxylate (22.09 g, 82.1 mmol) and acyl dimedone (16.1 g, 82.1 mmol) in a similar fashion as described in Example 8, as a yellow solid (14.25 g, 53%): ESI MS m/z=330 [M+H]⁺.

### EXAMPLE 10

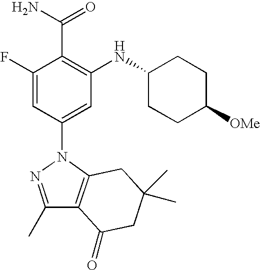

### 2-fluoro-6-(trans-4-methoxycyclohexylamino)-4-(3,6,6-trimethyl-4-oxo-4,5,6,7-tetrahydro-1H-indazol-1-yl)benzamide

4-Amino-cyclohexanol (2.627 g, 22.81 mmol) and 1,3-Dioxo-1,3-dihydro-isoindole-2-carboxylic acid ethyl ester (5 g, 22.81 mmol) were placed in a large test-tube and heated with a heat gun until both had melted and then for an additional 5 minutes. The resulting solution was allowed to cool and a solid formed. This solid was transferred to a flask, taken up in CH₂Cl₂ and purified via chromatography (75% EtOAc/Hex) to give 4.57 g of 2-(4-Hydroxy-cyclohexyl)-isoindole-1,3-dione (81% yield). LCMS (m/z): M+H+MeCN=287.1.

To a solution of 1.534 g (6.26 mmol) of 2-(4-Hydroxy-cyclohexyl)-isoindole-1,3-dione in 60 mL THF was added 0.553 g (13.8) NaH (60% in oil) and reaction mixture stirred under N₂ until gas formation ceased. The mixture was cooled to 0° C. and 3.56 g (25.1 mmol) MeI was added and the reaction was stirred at 25° C. for 24 hours. The mixture was then poured into 180 mL sat. NH₄Cl(aq) and extracted 3 times with DCM. The organic layers were dried over Na₂SO₄, concentrated and then purified via chromatography (50% EtOAc/Hex) to give 908 mg of 2-(4-Methoxy-cyclohexyl)-isoindole-1,3-dione (48.3% yield, LCMS (m/z): M+H=301.1). That was dissolved in 40 mL EtOH and 324 mg (8.76 mmol) Hydrazine was added. The reaction mixture was stirred at 65° C. for 3 hours. By the end of the 3 hours the solution was mostly white solid and a little liquid. The reaction was filtered and washed 3 times with 30 mL MeOH and concentrated to give 450 mg of crude product of 4-Methoxy-cyclohexylamine (ca. 100% yield). LCMS (m/z): M+H=130.2.

To a solution of 0.25 g (0.793 mmol) of 2,6-Difluoro-4-(3,6,6-trimethyl-4-oxo-4,5,6,7-tetrahydro-indazol-1-yl)-benzonitrile in DMSO (5 mL) was added 0.225 g (1.7446 mmol) DIEA and 0.186 g (1.438 mmol) of crude 4-Methoxy-cyclohexylamine. This was stirred at 100° C. for 18 hours and then poured into 25 mL water and extracted 3 times with CH₂Cl₂. The organic layers were dried over Na₂SO₄, concentrated and purified via chromatography (50% EtOAc/Hex) to give 0.163 g of 2-Fluoro-6-(4-methoxy-cyclohexylamino)-4-(3,6,6-trimethyl-4-oxo-4,5,6,7-tetrahydro-indazol-1-yl)-benzonitrile (48.5% yield). LCMS (m/z): M+H=425.2.

2-Fluoro-6-(4-methoxy-cyclohexylamino)-4-(3,6,6-trimethyl-4-oxo-4,5,6,7-tetrahydro-indazol-1-yl)-benzonitrile (163 mg, 0.384 mmol) was dissolved in 8 mL of a 4:1 EtOH:DMSO solution. To this solution was added 0.4 mL 1M NaOH and 0.4 mL 30% H₂O₂ and the reaction was stirred for 2 hours. The reaction mixture was quenched by pouring into 50 mL 10% Na₂S₂O₃(aq) and extracted 3 times with EtOAc. The organic layers were dried over Na₂SO₄, concentrated and purified via chromatography (50% EtOAc/Hex) to give 2-fluoro-6-(trans-4-methoxycyclohexylamino)-4-(3,6,6-trimethyl-4-oxo-4,5,6,7-tetrahydro-1H-indazol-1-yl)benzamide (168 mg; 98% yield). LCMS (m/z): M+H=443.2. ¹H NMR (DMSO, 20° C., 400 MHz) δ (ppm) 7.73-7.64 (m, 2H), 7.29 (d, 1H), 6.62-6.54 (m, 2H), 3.41-3.31 (m, 1H), 3.20 (s, 3H), 3.18-3.11 (m, 1H), 2.92 (s, 2H), 2.36 (s, 3H), 2.30 (s, 2H), 2.02-1.89 (m, 4H), 1.35-1.15 (m, 4H), 0.99 (s, 6H).

### EXAMPLE 11

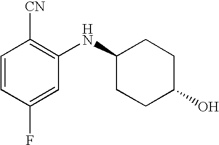

### 4-Fluoro-2-(4-hydroxy-cyclohexylamino)-benzonitrile

2,4-Difluorobenzonitrile (50.0 g, 0.359 mol), trans-4-aminocyclohexanol (41.4 g, 0.359 mol, 1 eq.), and N,N-diisopropylethylamine (62.6 mL, 0.359 mol, 1 eq.) were dissolved in 300 mL of DMSO. The reaction vessel was outfitted with a reflux condenser to avoid loss of N,N-diisopropylethylamine. The reaction mixture was then placed in an oil bath that had been pre-heated to 150° C., and was stirred at this temperature for 20 minutes. The solution was then cooled, poured into 750 mL of saturated aqueous NH₄Cl, and extracted with ethyl acetate (200 mL×3). The combined organics were washed with brine (150 mL×3), dried over Na₂SO₄, filtered, and concentrated in vacuo. The resultant residue was purified by column chromatography (1:1 ethyl acetate/hexane) to afford 20.9 g (25% yield) of the desired isomer as a white powder, and 36.1 g (43% yield) of the undesired isomer as a white powder.

### EXAMPLE 12

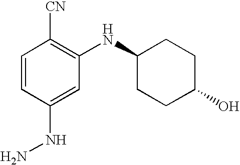

### 4-Hydrazino-2-(4-hydroxy-cyclohexylamino)-benzonitrile

4-Fluoro-2-(4-hydroxy-cyclohexylamino)-benzonitrile (537 mg, 2.29 mmol) was dissolved in hydrazine (2 g, 64 mmol) and heated to 60° C. and stirred for 30 min. The mixture was partially concentrated then partitioned between ethyl acetate (25 mL) and half saturated NaHCO₃ (25 mL). The organic layer was dried (MgSO₄) and concentrated to give 4-Hydrazino-2-(4-hydroxy-cyclohexylamino)-benzonitrile (400 mg, 70%) as an oil. LCMS (m/z): M+=246.7.

### EXAMPLE 13

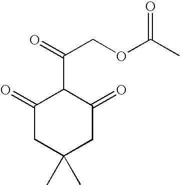

### Acetic acid 2-(4,4-dimethyl-2,6-dioxo-cyclohexyl)-2-oxo-ethyl ester

5,5-Dimethyl-1,3-cyclohexanedione (561 mg, 4 mmol) and triethylamine (810 mg, 8 mmol) and DMAP (1.37 mg) were dissolved in dichloroethane (10 mL) and treated dropwise with acetoxyacetyl chloride (601 mg, 4.4 mmol). The reaction mixture was treated with acetic acid (0.3 mL, 5 mmol), filtered and chromatographed (silica gel, 0 to 20% methanol in methylene chloride). Recrystallization with hexanes/ethyl acetate gave acetic acid 2-(4,4-dimethyl-2,6-dioxo-cyclohexyl)-2-oxo-ethyl ester (330 mg, 34%) as white crystals.

### EXAMPLE 14

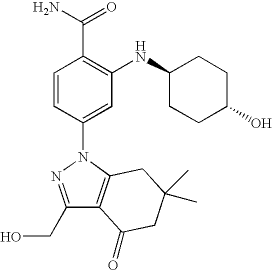

### 2-(trans-4-hydroxycyclohexylamino)-4-(3-(hydroxymethyl)-6,6-dimethyl-4-oxo-4,5,6,7-tetrahydro-1H-indazol-1-yl)benzamide

Acetic acid 2-(4,4-dimethyl-2,6-dioxo-cyclohexyl)-2-oxo-ethyl ester (206 mg, 0.86 mmol), 4-hydrazino-2-(4-hydroxy-cyclohexylamino)-benzonitrile (242 mg, 0.86 mmol) and sodium acetate (85 mg, 1 mmol) were combined in diochloroethane (2 mL), treated with methanol (0.3 mL) and stirred at RT overnight. The reaction mixture was concentrated and chromatographed (30 to 100% ethyl acetate in hexane) to give a crystalline solid. The product was triturated with EtOAc in hexanes and filtered off to give acetic acid 1-[4-cyano-3-(4-hydroxy-cyclohexylamino)-phenyl]-6,6-dimethyl-4-oxo-4,5,6,7-tetrahydro-1H-indazol-3-ylmethyl ester (236 mg, 61%) as a white solid. LCMS (m/z): M+H=451.2.

Acetic acid 1-[4-cyano-3-(4-hydroxy-cyclohexylamino)-phenyl]-6,6-dimethyl-4-oxo-4,5,6,7-tetrahydro-1H-indazol-3-ylmethyl ester (217 mg, 0.48 mmol) was dissolved in methanol (8 mL) and treated with 10% sodium hydroxide in methanol (0.29 mL of 10% solution in methanol, 30 mg, 0.72 mmol), 30% hydrogen peroxide (5 drops) and DMSO (9 drops) and stirred at RT. After 1 h, the reaction mixture was chromatographed (silica gel, 0 to 20% MeOH in DCM). The product was triturated with ethyl acetate and hexanes and filtered off to give 2-(trans-4-hydroxycyclohexylamino)-4-(3-(hydroxymethyl)-6,6-dimethyl-4-oxo-4,5,6,7-tetrahydro-1H-indazol-1-yl)benzamide (226 mg, 88% yield) as an off-white powder. LCMS (m/z): M+H=427.2. ¹H NMR (DMSO, 20° C., 400 MHz) δ (ppm) 8.31 (d, 1H), 7.89 (b, 1H), 7.71 (d, 1H), 7.20 (b, 1H), 6.77 (d, 1H), 6.66 (dd, 1H), 5.10 (t, 1H), 4.64 (d, 2H), 3.37-3.26 (m, 1H), 2.93 (s, 2H), 2.36 (s, 2H), 2.01-1.93 (m, 2H), 1.85-1.76 (m, 2H), 1.39-1.12 (m, 3H), 1.00 (s, 6H).

### EXAMPLE 15

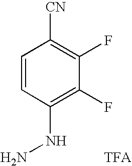

### 2,3-difluoro-4-hydrazino-benzonitrile, trifluoroacetate

2,3,4-trifluorobenzonitrile (3 g, 19 mmol), tert-butyl carbazate (4 g, 30 mmol) and Hunig's base were dissolved in dioxane (10 mL) and heated at 100° C. for 3 d. The mixture was concentrated and then partitioned between toluene (25 mL) and water (29 mL). The toluene layer was added to a column and chromatographed (silica gel, 10 to 40% ethyl acetate in hexanes) to give product as an oil. Recrystallization from ethyl acetate/hexanes gave N′-(4-cyano-2,3-difluoro-phenyl)-hydrazinecarboxylic acid tert-butyl ester (1.16 g, 22%) as a white powder. A second crop (0.5 g, 10%) was obtained as a slightly pinkish powder. LCMS (m/z): M+H=170.1 (loss of BOC).

N′-(4-cyano-2,3-difluoro-phenyl)-hydrazinecarboxylic acid tert-butyl ester (1.4 g, 5.2 mmol) was dissolved in dichloroethane (20 mL) and treated with TFA (16 mL) and stirred at RT for 3 d. The reaction mixture was concentrated and the solid residue was triturated with hexanes/ethyl acetate and filtered off to give 2,3-difluoro-4-hydrazino-benzonitrile, trifluoroacetate (1.058 g, 72%) as a light yellow crystalline solid. ¹H-NMR, DMSO-d₆, δ (ppm): 6.64 (2H, dd), 7.55 (2H, dd).

### EXAMPLE 16

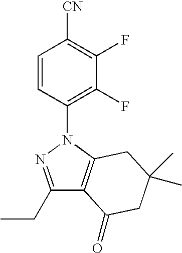

### 4-(3-Ethyl-6,6-dimethyl-4-oxo-4,5,6,7-tetrahydro-indazol-1-yl)-2,3-difluoro-benzonitrile

2,3-Difluoro-4-hydrazino-benzonitrile, trifluoroacetate (283 mg, 1 mmol) and 2-propionyldimedone (196 mg, 1 mmol) were dissolved in methanol (3 mL) and stirred at 40° C. for 3 d. The reaction mixture was concentrated and loaded onto a column with toluene and chromatographed (silica gel, 10 to 40% ethyl acetate in hexanes) to give 4-(3-ethyl-6,6-dimethyl-4-oxo-4,5,6,7-tetrahydro-indazol-1-yl)-2,3-difluoro-benzonitrile (254 mg, 77%) as a glass. LCMS (m/z): M+H=330.0.

### EXAMPLE 17

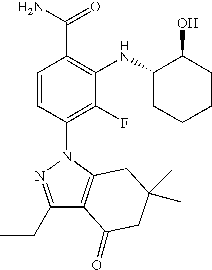

### 4-(3-ethyl-6,6-dimethyl-4-oxo-4,5,6,7-tetrahydro-1H-indazol-1-yl)-3-fluoro-2-((1S,2S)-2-hydroxycyclohexylamino)benzamide

4-(3-Ethyl-6,6-dimethyl-4-oxo-4,5,6,7-tetrahydro-indazol-1-yl)-2,3-difluoro-benzonitrile (85 mg, 0.258 mmol), (S,S)-2-benzyloxycyclohexylamine (80 mg, 0.387 mmol) and Hunig's base (33 mg, 0.258 mmol) were combined in DMSO (0.250 mL) and stirred at RT overnight. The mixture was diluted with methanol (1.5 mL) and treated with 10% NaOH in methanol (12 drops) and 30% hydrogen peroxide (10 drops). The reaction mixture was partitioned between ethyl acetate (5 mL) and water (1.5 mL). The organic layer was removed, concentrated and chromatographed (silica gel, 40 to 100% ethyl acetate in hexanes) to give 2-(S,S-2-Benzyloxy-cyclohexylamino)-4-(3-ethyl-6,6-dimethyl-4-oxo-4,5,6,7-tetrahydro-indazol-1-yl)-3-fluoro-benzamide (91 mg, 66%) as an oil. LCMS (m/z): M+H=533.2.

2-(S,S-2-Benzyloxy-cyclohexylamino)-4-(3-ethyl-6,6-dimethyl-4-oxo-4,5,6,7-tetrahydro-indazol-1-yl)-3-fluoro-benzamide was dissolved in ethanol (5 mL), treated with wet 5% Pd/C (˜10 mg), put under a H₂ balloon atmosphere and stirred for 16 h. The mixture was concentrated and chromatographed (silica gel, 50 to 100% ethyl acetate in hexanes) to give an oil. The oil was triturated with ethyl acetate/hexanes to give a crystalline solid which was filtered off to give 4-(3-ethyl-6,6-dimethyl-4-oxo-4,5,6,7-tetrahydro-1H-indazol-1-yl)-3-fluoro-2-((1S,2S)-2-hydroxycyclohexylamino)benzamide (41 mg, 54%) as a white powder. LCMS (m/z): M+H=443.1. ¹H NMR (DMSO, 20° C., 400 MHz) δ (ppm) 8.05 (b, 1H), 7.85 (d, 1H), 7.54-7.45 (m, 2H), 6.73 (dd, 1H), 4.71 (d, 1H), 3.40-3.32 (m, 1H), 3.28-3.21 (m, 1H), 2.78 (q, 2H), 2.66-2.54 (m, 2H), 2.32 (s, 2H), 1.95-1.87 (m, 1H), 1.83-1.74 (m, 1H), 1.63-1.50 (m, 2H), 1.28-1.07 (m, 7H), 0.99 (s, 6H).

### EXAMPLE 18

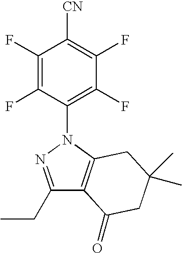

### 4-(3-Ethyl-6,6-dimethyl-4-oxo-4,5,6,7-tetrahydro-indazol-1-yl)-2,3,5,6-tetrafluoro-benzonitrile

A solution of 2,3,4,5,6-pentafluorobenzonitrile (386 mg, 2 mmol) in dioxane (1 mL) was treated slowly with a solution of hydrazine (380 mg) and Hunig's base (258 mg, 2 mmol) in dioxane (1 mL). The solution warmed slightly. The mixture was concentrated and added chromatographed (silica gel, 20 to 50% ethyl acetate in hexanes) to give 2,3,5,6-tetrafluoro-4-hydrazino-benzonitrile (315 mg, 76%) as a white solid. This was treated with propionyldimedone (392 mg, 2 mmol), dissolved in methanol (5 mL) and stirred at RT for 3 d. The mixture was concentrated, dissolved in toluene and heated at 70° C. for 16 h. The mixture was added to a column and chromatographed (silica gel, 5 to 50% ethyl acetate in hexanes) to give a glass. The glass was triturated with hexanes to give 4-(3-ethyl-6,6-dimethyl-4-oxo-4,5,6,7-tetrahydro-indazol-1-yl)-2,3,5,6-tetrafluoro-benzonitrile (253 mg, 35% overall) as slightly yellow solid. LCMS (m/z): M+H=366.0.

### EXAMPLE 19

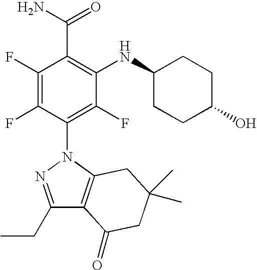

### 4-(3-ethyl-6,6-dimethyl-4-oxo-4,5,6,7-tetrahydro-1H-indazol-1-yl)-2,3,5-trifluoro-6-(trans-4-hydroxycyclohexylamino)benzamide

4-(3-Ethyl-6,6-dimethyl-4-oxo-4,5,6,7-tetrahydro-indazol-1-yl)-2,3,5,6-tetrafluoro-benzonitrile (195 mg, 0.533 mmol) and trans-4-hydroxycyclohexylamine (122 mg, 1.07 mmol) were combined in DMSO (2 mL) and treated with Hunig's base (69 mg, 0.533 mmol) and left at RT for about 1 h. The mixture was then diluted with methanol (4 mL) and treated with 10% NaOH in methanol (0.25 mL) and 30% hydrogen peroxide (7 drops). The reaction mixture was partitioned between ethyl acetate (45 mL) and water (15 mL). The aqueous layer was extracted with more ethyl acetate (20 mL) and the combined organic layers washed with brine (10 mL) and concentrated. The residue was chromatographed (silica gel, 40 to 100% ethyl acetate in hexanes) to give a glass (235 mg, 92%). Trituration with hexanes and ethyl acetate gave 4-(3-ethyl-6,6-dimethyl-4-oxo-4,5,6,7-tetrahydro-1H-indazol-1-yl)-2,3,5-trifluoro-6-(trans-4-hydroxycyclohexylamino)benzamide (157 mg, 61%) as a fluffy white powder. LCMS (m/z): M+H=479.2. ¹H NMR (DMSO, 20° C., 400 MHz) δ (ppm) 8.20 (s, 1H), 8.12 (s, 1H), 2.82-2.76 (m, 1H), 2.58 (s, 1H), 2.48-2.35 (m, 8H), 1.86-1.75 (m, 3H), 1.15 (t, 6H), 0.99 (s, 6H).

### EXAMPLE 20

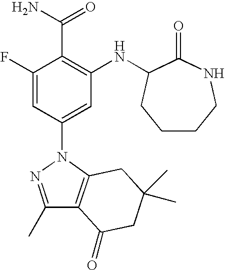

### 2-fluoro-6-(2-oxoazepan-3-ylamino)-4-(3,6,6-trimethyl-4-oxo-4,5,6,7-tetrahydro-1H-indazol-1-yl)benzamide

2,6-Difluoro-4-(3,6,6-trimethyl-4-oxo-4,5,6,7-tetrahydro-indazol-1-yl)-benzonitrile (200 mg, 0.634 mmol) and 2-aminocaprolactam (243 mg, 1.9 mmol) were combined in DMSO (1 mL) and heated at 60° C. for 2 h. The mixture was allowed to cool and was treated with methanol (1 mL), 10% NaOH in methanol (30 drops), and 30% hydrogen peroxide (10 drops) and stirred. The solution turned solid. The mixture was then heated gently with a heat gun until homogenous to effect complete reaction. The mixture was partitioned between ethyl acetate (40 mL) and 0.5 N HCl (10 mL). The aqueous layer was washed with more ethyl acetate (10 mL) and the combined organic layers were washed with brine (10 mL). The organic layer was dried (MgSO₄), filtered and concentrated to a solid. The solid was chromatographed (silica gel, 0 to 30% MeOH in DCM) to give the product as a glass which was triturated with ethyl acetate/methanol to give 2-fluoro-6-(2-oxoazepan-3-ylamino)-4-(3,6,6-trimethyl-4-oxo-4,5,6,7-tetrahydro-1H-indazol-1-yl)benzamide (169 mg, 60%) as a white solid. LCMS (m/z): M+H=442.2. ¹H NMR (DMSO, 20° C., 400 MHz) δ (ppm) 7.96-7.89 (m, 1H), 7.78 (d, 1H), 7.73-7.64 (m, 2H), 6.64 (dd, 1H), 6.44 (d, 1H), 4.35-4.28 (m, 1H), 3.36-3.24 (m, 1H), 3.17-2.79 (m, 3H), 2.45-2.20 (m, 5H), 1.98-1.71 (m, 4H), 1.43-1.31 (m, 1H), 1.30-1.18 (m, 1H), 1.08 (s, 3H), 0.95 (s, 3H).

### EXAMPLE 21

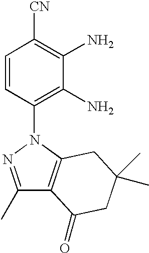

### 2,3-Diamino-4-(3,6,6-trimethyl-4-oxo-4,5,6,7-tetrahydro-indazol-1-yl)-benzonitrile

2,3-Difluoro-4-hydrazinobenzonitrile (995 mg, 3.51 mmol), acetyldimedone (640 mg, 3.51 mmol) and sodium acetate (288 mg, 3.51 mmol) were dissolved in methanol (6 mL) and heated to 65° C. and stirred for 3 h. The reaction mixture was concentrated and chromatographed (silica gel, 10 to 35% ethyl acetate in hexanes) to give a glass. The glass was triturated with hexanes/ethyl acetate to give 2,3-difluoro-4-(3,6,6-trimethyl-4-oxo-4,5,6,7-tetrahydro-indazol-1-yl)-benzonitrile (678 mg, 61%) as a white powder. LCMS (m/z): M+H=316.1.

2,3-Difluoro-4-(3,6,6-trimethyl-4-oxo-4,5,6,7-tetrahydro-indazol-1-yl)-benzonitrile (315 mg, 1 mmol) and sodium azide (325 mg, 5 mmol) were slurried in DMSO (1.5 mL) and stirred at 50° C. for 16 h. The mixture was treated with water (4 mL) and extracted twice with DCE (1 mL each). The organic layers were added to a column and chromatographed (silica gel, 20 to 40% ethyl acetate in hexanes) to give bis-azide (LCMS (m/z): M+H=362.1) intermediate in solution. The mixture was partially concentrated, then diluted with ethanol and partially concentrated again. Wet 5% Pd/C (34 mg) was slurried in ethanol and treated with the ethanol solution of the bis-azide and the entire mixture put under a H₂ atmosphere with a balloon and stirred. After 4 h, the mixture was purged with nitrogen, filtered through Celite, and concentrated to give 2,3-diamino-4-(3,6,6-trimethyl-4-oxo-4,5,6,7-tetrahydro-indazol-1-yl)-benzonitrile as a glass. LCMS (m/z): M+H=310.1. Half of this material was used in the next step.

### EXAMPLE 22

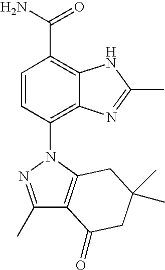

### 2-methyl-4-(3,6,6-trimethyl-4-oxo-4,5,6,7-tetrahydro-1H-indazol-1-yl)-1H-benzo[d]imidazole-7-carboxamide

Using the procedure of Wang et al., Syn. Comm. 2004, 34, 4265-4272, 2,3-diamino-4-(3,6,6-trimethyl-4-oxo-4,5,6,7-tetrahydro-indazol-1-yl)-benzonitrile (154 mg, 0.497 mmol) was dissolved in ethanol (1 mL) and treated with trimethyl orthoacetate (119 mg, 0.995 mmol) and ytterbium triflate (1 mg) and heated at 90° C. The mixture was concentrated and chromatographed (silica gel, 40 to 100% ethyl acetate in hexanes) to give 2-methyl-7-(3,6,6-trimethyl-4-oxo-4,5,6,7-tetrahydro-indazol-1-yl)-3H-benzoimidazole-4-carbonitrile as a glass which was used immediately for the next step.

2-Methyl-7-(3,6,6-trimethyl-4-oxo-4,5,6,7-tetrahydro-indazol-1-yl)-3H-benzoimidazole-4-carbonitrile was treated with methanol (1 mL), DMSO (0.1 mL), 10% NaOH in methanol (30 drops), and 30% hydrogen peroxide (10 drops) and stirred then heated gently with a heat gun until homogenous. The mixture was concentrated and chromatographed (silica gel, 0 to 20% methanol in DCM) to give an oil. The oil was triturated with ethyl acetate to give 2-methyl-4-(3,6,6-trimethyl-4-oxo-4,5,6,7-tetrahydro-1H-indazol-1-yl)-1H-benzo[d]imidazole-7-carboxamide as an off-white solid. LCMS (m/z): M+H=352.1.

### EXAMPLE 23

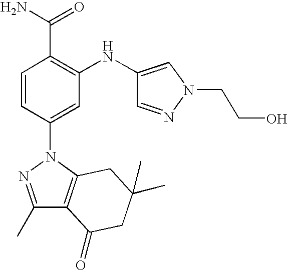

### 2-(1-(2-hydroxyethyl)-1H-pyrazol-4-ylamino)-4-(3,6,6-trimethyl-4-oxo-4,5,6,7-tetrahydro-1H-indazol-1-yl)benzamide

A suspension of 2-bromo-4-(3,6,6-trimethyl-4-oxo-4,5,6,7-tetrahydro-indazol-1-yl)-benzonitrile (0.19 g, 0.5 mmol), 4-amino-2-benxyloxyethylpyrazole (0.219 g, 1 mmol), Pd(OAc)₂ (0.006 g, 0.025 mmol), DPPF (0.028 g. 0.05 mmol) and NaOtBu (0.19 g, 2 mmol) in toluene (5 mL) was microwaved at 120° C. for 20 min. The reaction mixture was filtered through a pad of Celite. The filter pad was rinsed with EtOAc. The filtrate was evaporated under reduced pressure, and the residue was loaded onto a Biotage column that was eluted with 5-30% MeOH/CH₂Cl₂ to afford 0.25 g (100%) of 2-[1-(2-benzyloxy-ethyl)-1H-pyrazol-4-ylamino]-4-(3,6,6-trimethyl-4-oxo-4,5,6,7-tetrahydro-indazol-1-yl)-benzonitrile. LC/MS: m/z=164 [M+H]⁺.

A solution of 2-[1-(2-benzyloxy-ethyl)-1H-pyrazol-4-ylamino]-4-(3,6,6-trimethyl-4-oxo-4,5,6,7-tetrahydro-indazol-1-yl)-benzonitrile (0.25 g, 0.5 mmol) in EtOH/DMSO (4:1, 3 mL) was treated with 2 drops of 1 M NaOH and 4 drops of H₂O₂. The reaction mixture was stirred at RT for 1 h. Brine (20 mL) was added, and the aqueous phase was extracted with EtOAc (3×). The combined organic layers were dried over MgSO₄, and evaporated under reduced pressure. The residue was dried in vacuo to afford 0.262 g (100%) of 2-[1-(2-benzyloxy-ethyl)-1H-pyrazol-4-ylamino]-4-(3,6,6-trimethyl-4-oxo-4,5,6,7-tetrahydro-indazol-1-yl)-benzamide that was used without further purification. LC/MS: m/z=513 [M+H]⁺.

To a degassed solution of 2-[1-(2-benzyloxy-ethyl)-1H-pyrazol-4-ylamino]-4-(3,6,6-trimethyl-4-oxo-4,5,6,7-tetrahydro-indazol-1-yl)-benzamide (0.141 g, 0.275 mmol) and cyclohexene (0.28 mL, 2.75 mmol) in EtOH (10 mL) was added 10% Pd/C (0.07 g). Acetic acid (2 mL) was added, and the reaction mixture was refluxed for 1 day. The reaction mixture was filtered through a pad of Celite, and the filter pad was rinsed with EtOH. The solvent was removed under reduced pressure. Purification of the residue using a Biotage column eluted with 0-10% MeOH/CH₂Cl₂ afforded 0.115 g (100%) of 2-(1-(2-hydroxyethyl)-1H-pyrazol-4-ylamino)-4-(3,6,6-trimethyl-4-oxo-4,5,6,7-tetrahydro-1H-indazol-1-yl)benzamide. LC/MS: m/z=423 [M+H]⁺.

### EXAMPLE 24

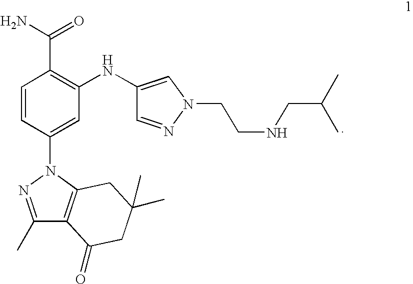

### 2-(1-(2-(isobutylamino)ethyl)-1H-pyrazol-4-ylamino)-4-(3,6,6-trimethyl-4-oxo-4,5,6,7-tetrahydro-1H-indazol-1-yl)benzamide

To a solution of 2-[1-(2-hydroxy-ethyl)-1H-pyrazol-4-ylamino]-4-(3,6,6-trimethyl-4-oxo-4,5,6,7-tetrahydro-indazol-1-yl)-benzamide (0.0659 g, 0.156 mmol) in CH₂Cl₂ (5 mL) were added diisopropylethyl amine (0.05 mL, 0.312 mmol) and methanesulfonyl chloride (0.02 mL, 0.312 mmol), and the reaction was stirred at RT for 3 h. After addition of water (5 mL), the reaction mixture was extracted with CH₂Cl₂. The combined organic layers were dried over MgSO₄, and evaporated under reduced pressure. The residue was dried in vacuo to afford 0.069 g (88%) of methanesulfonic acid 2-{4-[2-carbamoyl-5-(3,6,6-trimethyl-4-oxo-4,5,6,7-tetrahydro-indazol-1-yl)-phenylamino]-pyrazol-1-yl}-ethyl ester that was used without further purification. LC/MS: m/z=501 [M+H]⁺.

A solution of 2-{4-[2-carbamoyl-5-(3,6,6-trimethyl-4-oxo-4,5,6,7-tetrahydro-indazol-1-yl)-phenylamino]-pyrazol-1-yl}-ethyl ester (0.05 g, 0.14 mmol), and isopropyl amine (0.28 mL, 2.8 mmol) in THF (5 mL) was microwaved at 100° C. for 40 min. The solvent was evaporated under reduced pressure. Purification of the residue using a Biotage column eluted with 5-20% MeOH/CH₂Cl₂ afforded 0.06 g (24%) of 2-(1-(2-(isobutylamino)ethyl)-1H-pyrazol-4-ylamino)-4-(3,6,6-trimethyl-4-oxo-4,5,6,7-tetrahydro-1H-indazol-1-yl)benzamide as a clear oil. LC/MS: m/z=478 [M+H]⁺.

### EXAMPLE 25

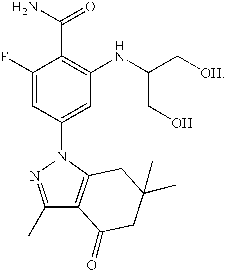

### 2-(1,3-dihydroxypropan-2-ylamino)-6-fluoro-4-(3,6,6-trimethyl-4-oxo-4,5,6,7-tetrahydro-1H-indazol-1-yl)benzamide

A solution of 2,6-difluoro-4-(3,6,6-trimethyl-4-oxo-4,5,6,7-tetrahydro-indazol-1-yl)-benzonitrile (0.0714 g, 0.226 mmol), serinol (0.025 g, 0.27 mmol) and diisopropylethyl amine (0.05 mL, 0.27 mmol) in DMSO (1 mL) was microwaved at 120° C. for 40 min. After addition of water (1 mL), the reaction mixture was extracted with EtOAc (3×). The combined organic layers were dried over MgSO₄, and evaporated under reduced pressure. Purification of the residue using a Biotage column eluted with 0-10% MeOH/CH₂Cl₂ afforded 0.025 g (28%) of 2-fluoro-6-(2-hydroxy-1-hydroxymethyl-ethylamino)-4-(3,6,6-trimethyl-4-oxo-4,5,6,7-tetrahydro-indazol-1-yl)-benzonitrile. LC/MS: m/z=387 [M+H]⁺.

A solution of 2-fluoro-6-(2-hydroxy-1-hydroxymethyl-ethylamino)-4-(3,6,6-trimethyl-4-oxo-4,5,6,7-tetrahydro-indazol-1-yl)-benzonitrile (0.018 g, 0.047 mmol) in EtOH/DMSO (4:1, 2 mL) was treated with 4 drops of 1M NaOH and 4 drops of H₂O₂. The reaction mixture was stirred at RT for 3 h. Brine (10 mL) was added, and the aqueous phase was extracted with EtOAc (3×). The combined organic layers were dried over MgSO₄, and evaporated under reduced pressure. Purification of the residue using a Biotage column eluted with 0-20% MeOH/CH₂Cl₂ afforded 0.012 g (62%) of 2-(1,3-dihydroxypropan-2-ylamino)-6-fluoro-4-(3,6,6-trimethyl-4-oxo-4,5,6,7-tetrahydro-1H-indazol-1-yl)benzamide as a white solid. LC/MS: m/z=405 [M+H]⁺. ¹H NMR (DMSO, 20° C., 400 MHz) δ (ppm) 7.67 (b, 2H), 7.33 (d, 1H), 6.55 (d, 1H), 4.72 (b, 1H), 3.50-3.47 (m, 1H), 2.91 (s, 3H), 2.49-2.31 (m, 8H), 1.00 (s, 6H).

### EXAMPLE 26

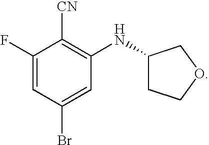

### (S)-4-bromo-2-fluoro-6-(tetrahydrofuran-3-ylamino)benzonitrile

A solution of 4-bromo-2,6-difluoro-benzonitrile (0.323 g, 1.48 mmol), (S)-3-amino-tetrahydrofuran (0.2209 g, 1.77 mmol) and diisopropylethayl amine (0.62 mL, 3.55 mmol) in DMSO (6 mL) was stirred at RT overnight. After addition of water (50 mL), some crystals started to precipitate. The white crystals were collected using vacuum filtration, and were dried under vacuum to afford 0.2541 g (60%) of (S)-4-bromo-2-fluoro-6-(tetrahydrofuran-3-ylamino)benzonitrile. LC/MS: m/z=284 and 286 [M+H]⁺.

### EXAMPLE 27

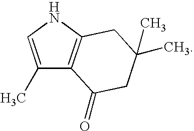

### 3,6,6-Trimethyl-1,5,6,7-tetrahydro-indol-4-one

To a solution of anti-pyruvic aldehyde-1-oxime (10 g, 1 eq) and 5,5-dimethyl-1,3-cyclohexanedione (16.1 g, 1 eq) in HOAc-H₂O (7:3, 200 mL), was added zinc powder (14.95 g, 2 eq) slowly cooling with a water bath at room temperature. The mixture was refluxed overnight, concentrated to dryness, partitioned between brine (300 mL) and dichloromethane (300 mL). The pH was adjusted to ca. 6 with saturated aqueous NaHCO₃, then extracted with dichloromethane (3×200 mL). The organic layers were combined, dried over Na₂SO₄, filtered, concentrated to give crude product. This was purified by flash chromatography, eluting with 5% ethyl acetate in dichloromethane to give expected product, which was triturated in ether-hexane (2:1) for 1 hour, then filtered, washed with hexane to give pure title compound (9 g, 45% yield) as a solid.

### EXAMPLE 28

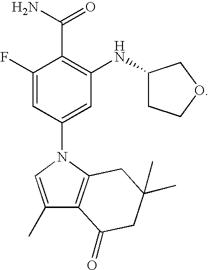

### 2(S)-2-fluoro-6-(tetrahydrofuran-3-ylamino)-4-(3,6,6-trimethyl-4-oxo-4,5,6,7-tetrahydro-1H-indol-1-yl)benzamide

Potassium carbonate (1.21 g, 8.75 mmol) was added to a solution of 4-bromo-2-fluoro-6-(S-tetrahydro-furan-3-ylamino)-benzonitrile (0.5 g, 1.75 mmol) and 3,6,6-Trimethyl-1,5,6,7-tetrahydro-indol-4-one (0.31 g, 1.75 mmol) in 1,4-dioxane (6 mL). The solution was degassed under N₂. The flask was then cooled at 0° C. and connected to a vacuum line. The vacuum was pulled until the solvent started bubbling. The degassing/vacuum cycle was repeated 2 times. Then N,N′-dimethylethylenediamine (0.27 mL, 2.54 mmol) and CuI (0.5 g, 2.63 mmol) were added. The degassing/vacuum cycle was performed 3 times. The rubber septum was replaced by a microwave cap. The solution was degassed one more time, and placed in an oil bath at 100° C. The reaction mixture was stirred at 100° C. for 4 days. The reaction mixture was filtered through a pad of Celite, and the filter pad was rinsed with EtOAc. The solvent was removed under reduced pressure. Purification of the residue using a Biotage column eluted with 0-50% MeOH/CH₂Cl₂ afforded 0.263 g (39%) of 2-fluoro-6-(S-tetrahydro-furan-3-ylamino)-4-(3,6,6-trimethyl-4-oxo-4,5,6,7-tetrahydro-indol-1-yl)-benzonitrile. LC/MS: m/z=382 [M+H]⁺. ¹H NMR (DMSO, 20° C., 400 MHz) δ (ppm) 7.73-7.69 (m, 2H), 7.44 (d, 1H), 6.55 (d, 1H), 6.46 (s, 1H), 4.46 (b, 1H), 3.87-3.70 (m, 7H), 2.97 (s, 3H), 2.74 (s, 2H), 2.49 (s, 2H), 0.98 (s, 6H)

A solution of 2-fluoro-6-(S-tetrahydro-furan-3-ylamino)-4-(3,6,6-trimethyl-4-oxo-4,5,6,7-tetrahydro-indol-1-yl)-benzonitrile (0.263 g, 0.69 mmol) in EtOH/DMSO (4:1, 10 mL) was treated with 1M NaOH (0.3 mL) and H₂O₂ (0.5 mL). The reaction mixture was stirred at RT for 1 h. Brine (20 mL) was added, and the aqueous phase was extracted with EtOAc (3×). The combined organic layers were dried over MgSO₄, and evaporated under reduced pressure. Purification of the residue using a Biotage column eluted with 0-10% MeOH/CH₂Cl₂ afforded 0.27 g (98%) of 2(S)-2-fluoro-6-(tetrahydrofuran-3-ylamino)-4-(3,6,6-trimethyl-4-oxo-4,5,6,7-tetrahydro-1H-indol-1-yl)benzamide as a white solid. LC/MS: m/z=400 [M+H]⁺.

### EXAMPLE 29

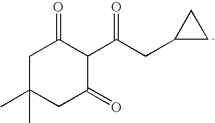

### 2-(2-Cyclopropylacetyl)-5,5-dimethylcyclohexane-1,3-dione

To 5,5-dimethyl-1,3-cyclohexanedione (6.93 g, 49.4 mmol), cyclopropylacetic acid (3.4 mL, 33 mmol), and 4-dimethylaminopyridine (6.04 g, 49.4 mmol) in CH₂Cl₂ (90 mL) at 0° C. was added a solution of N,N-dicyclohexylcarbodiimide (8.15 g, 39.5 mmol) in CH₂Cl₂ (90 mL) dropwise. The solution was allowed to warm to 25° C. and stirred for 14 hours. The crude mixture was filtered through celite and concentrated. It was taken up in EtOAc (200 mL) and washed with 2M aqueous HCl (2×200 mL). The aqueous layer was back-extracted with EtOAc (200 mL). The combined organic portions were washed with saturated aqueous NaCl (200 mL) and dried over Na₂SO₄. Purification by gradient flash chromatography, eluting with 0% to 25% EtOAc in hexanes provided 2-(2-cyclopropylacetyl)-5,5-dimethylcyclohexane-1,3-dione (6.2 g, 85%) as a pale yellow oil (LC/MS m/z=223.2 [M+H]⁺).

### EXAMPLE 30

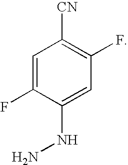

### 4-Hydrazino-2,5-difluorobenzonitrile

2,4,5-Trifluorobenzonitrile (1 g, 6.3 mmol) and hydrazine (610 mg, 19 mmol) were dissolved in dioxane (3 mL) and stirred for 16 h. The reaction mixture was partitioned between ethyl acetate (30 mL) and sat. NaHCO₃ (20 mL). The organic layer was washed with brine (10 mL), dried (MgSO₄), filtered and concentrated to give 4-hydrazino-2,5-difluorobenzonitrile (1.012 g, 93%) as a white solid. LC/MS: m/z (M+H)=170.0. ¹H-NMR (400 MHz, DMSO-d₆) δ (ppm): 4.41 (2H, br s), 6.93 (1H, dd), 7.51 (1H, dd), 8.22 (1H, br s).

### EXAMPLE 31

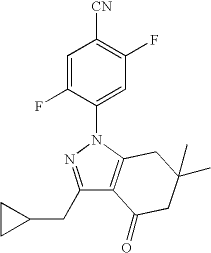

### 4-(3-Cyclopropylmethyl-6,6-dimethyl-4-oxo-4,5,6,7-tetrahydroindazol-1-yl)-2,5-difluorobenzonitrile

2-(2-Cyclopropylacetyl)-5,5-dimethylcyclohexane-1,3-dione (351 mg, 1.58 mol) and 4-hydrazino-2,5-difluorobenzonitrile (267 mg, 1.58 mmol) were combined with ACOH (0.4 mL) and EtOH (1.2 mL). The reaction was stirred at 25° C. for 2 hours. It was diluted with EtOAc (20 mL) and washed with sat. NaHCO₃ (20 mL) and sat. NaCl (20 mL). The organic layer was dried over Na₂SO₄ and concentrated. Purification by gradient flash chromatography eluting with 0% to 50% EtOAc in hexanes provided 4-(3-cyclopropylmethyl-6,6-dimethyl-4-oxo-4,5,6,7-tetrahydroindazol-1-yl)-2,5-difluorobenzonitrile (395 mg, 70%) as an orange solid (LC/MS m/z=356.0 [M+H]⁺).

### EXAMPLE 32

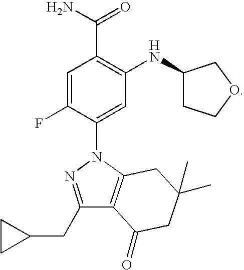

### (R)-4-(3-(cyclopropylmethyl)-6,6-dimethyl-4-oxo-4,5,6,7-tetrahydro-1H-indazol-1-yl)-5-fluoro-2-(tetrahydrofuran-3-ylamino)benzamide

4-(3-Cyclopropylmethyl-6,6-dimethyl-4-oxo-4,5,6,7-tetrahydroindazol-1-yl)-2,5-difluorobenzonitrile (95 mg, 0.27 mmol), R-(+)-3-aminotetrahydrofuran toluene-4-sulfonate (70 mg, 0.27 mmol), and diisopropylamine (0.1 mL, 0.6 mmol) were combined in DMSO (0.5 mL). The reaction was stirred at 100° C. for 48 hours and then cooled to 25° C., and EtOH (2 mL), 1 M NaOH (0.4 mL), and 30% aqueous H₂O₂ (0.4 mL) were added. The reaction was stirred at 25° C. for 1 hour. It was diluted with EtOAc (20 mL), and washed with 2 M HCl (2×20 mL). The organic portion was dried over Na₂SO₄ and concentrated. Purification by gradient flash chromatography provided (R)-4-(3-(cyclopropylmethyl)-6,6-dimethyl-4-oxo-4,5,6,7-tetrahydro-1H-indazol-1-yl)-5-fluoro-2-(tetrahydrofuran-3-ylamino)benzamide (43 mg, 36%) as a yellow solid (LC/MS m/z=441.1 [M+H]⁺). ¹H NMR (DMSO, 20° C., 400 MHz) δ (ppm) 8.13 (d, 1H), 7.87 (b, 1H), 7.59 (d, 1H), 7.32 (b, 1H), 6.61 (d, 1H), 3.98-3.91 (m, 1H), 3.70-3.60 (m, 2H), 3.59-3.51 (m, 1H), 3.37-3.30 (m, 1H), 2.54 (d, 2H), 2.50 (s, 2H), 2.16 (s, 2H), 2.10-1.99 (m, 1H), 1.58-1.50 (m, 1H), 1.00-0.90 (m, 1H), 0.83 (s, 6H), 0.24-0.18 (m, 2H), 0.03—0.02 (m, 2H).

### EXAMPLE 33

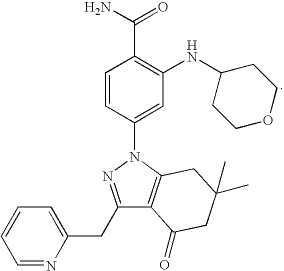

### 4-(6,6-dimethyl-4-oxo-3-(pyridin-2-ylmethyl)-4,5,6,7-tetrahydro-1H-indazol-1-yl)-2-(tetrahydro-2H-pyran-4-ylamino)benzamide

To 5,5-dimethyl-1,3-cyclohexanedione (1.57 g, 11.2 mmol), 2-pyridylacetic acid hydrochloride (1.29 g, 7.46 mmol), and 4-dimethylaminopyridine (2.28 g, 18.65 mmol) in CH₂Cl₂ (23 mL) at 0° C. was added a solution of N,N-dicyclohexylcarbodiimide (1.85 g, 8.95 mmol) in CH₂Cl₂ (23 mL) dropwise. The solution was allowed to warm to 25° C. and stirred for 14 hours. The crude mixture was filtered through celite and concentrated. Purification by gradient flash chromatography, eluting with 0% to 50% MeOH in CH₂Cl₂ provided 2-bromo-4-hydrazinobenzonitrile (2.39 g, 93%) (LC/MS m/z=259.7 [M+H]⁺).

Acetic acid (1.2 mL) and EtOH (3.6 mL) were added to 2-bromo-4-hydrazinobenzonitrile (1.02 g, 4.82 mmol) and 5,5-dimethyl-2-(2-pyridin-2-ylacetyl)-cyclohexane-3,3-dione (1.25 g, 4.82 mmol). The mixture was stirred at 150° C. for 15 minutes. The reaction was cooled to 25° C., diluted with EtOAc (50 mL), and washed with saturated aqueous NaHCO₃ (50 mL). The organic portion was dried over Na₂SO₄ and concentrated. Purification by gradient flash chromatography, eluting with 0% to 100% EtOAc in hexanes provided 2-bromo-4-(6,6-dimethyl-4-oxo-3-pyridin-2-ylmethyl-4,5,6,7-tetrahydroindazol-1-yl)-benzonitrile (300 mg, 14%) (LC/MS m/z=436.5 [M+H]⁺).

To 2-bromo-4-(6,6-dimethyl-4-oxo-3-pyridin-2-ylmethyl-4,5,6,7-tetrahydroindazol-1-yl)-benzonitrile (70 mg, 0.16 mmol) were added palladium acetate (4 mg, 0.02 mmol), 1,1′-bis(diphenylphosphino)ferrocene (9 mg, 0.02 mmol), and NaO^{t}Bu (46 mg, 0.48 mmol). The reagents were flushed with N₂ for 10 minutes. Toluene (1.1 mL) and 4-aminotetrahydropyran (65 mg, 0.64 mmol) were added, and the solution was stirred at 110° C. for 1 hour. The reaction was concentrated and purified by gradient flash chromatography, eluting with 0% to 100% EtOAc in hexanes to provide 4-(6,6-dimethyl-4-oxo-3-pyridin-2-ylmethyl-4,5,6,7-tetrahydroindazol-1-yl)-2-(tetrahydropyran-4-ylamino)-benzonitrile (33 mg, 45%) (LC/MS m/z=378.6 [M+H]⁺).

To 4-(6,6-dimethyl-4-oxo-3-pyridin-2-ylmethyl-4,5,6,7-tetrahydroindazol-1-yl)-2-(tetrahydropyran-4-ylamino)-benzonitrile in DMSO (0.2 mL) and EtOH (0.8 mL) were added 1 M aqueous NaOH (0.2 mL) and 30% aqueous H₂O₂ (0.2 mL) dropwise. The reaction was stirred for 2 hours at 25° C. It was diluted with EtOAc (20 mL) and extracted with H₂O (2×20 mL). The organic layer was dried over Na₂SO₄ and concentrated. Purification by gradient flash chromatography eluting with 0% to 50% MeOH in CH₂Cl₂ provided 4-(6,6-dimethyl-4-oxo-3-(pyridin-2-ylmethyl)-4,5,6,7-tetrahydro-1H-indazol-1-yl)-2-(tetrahydro-2H-pyran-4-ylamino)benzamide (29 mg, 85%) as a white solid (LC/MS m/z=473.5 [M+H]⁺). ¹H NMR (DMSO, 20° C., 400 MHz) δ (ppm) 8.47 (d, 1H), 8.42-8.40 (m, 1H), 7.92 (b, 1H), 7.73 (d, 1H), 7.69-7.63 (m, 1H), 7.29-7.22 (m, 2H), 7.19-7.15 (m, 1H), 6.83 (d, 1H), 6.67 (dd, 1H), 4.33 (s, 2H), 3.85-3.79 (m, 2H), 3.65-3.56 (m, 1H), 3.49-3.41 (m, 2H), 2.94 (s, 2H), 2.31 (s, 2H), 1.95-1.88 (m, 2H), 1.43-1.32 (m, 2H), 1.00 (s, 6H).

### EXAMPLE 34

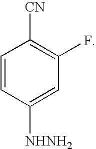

### 2-Fluoro-4-hydrazinobenzonitrile

1,4-Dioxane (40 mL) was added to 2,4-difluorobenzonitrile (2.78 g, 20.0 mmol). Hydrazine (3.2 mL, 100 mmol) was added via addition funnel. The reaction was stirred at 25° C. for 24 hours. It was diluted with EtOAc (100 mL) and 1 M NaOH (200 mL). The mixture was vigorously stirred for 10 minutes. The organic layer was removed and concentrated. Purification by gradient flash chromatography eluting with 0% to 50% EtOAc in hexanes gave 2-fluoro-4-hydrazinobenzonitrile (926 mg, 31%) as a pink solid (LC/MS m/z=193.1 [M+H+ MeCN]⁺).

### EXAMPLE 35

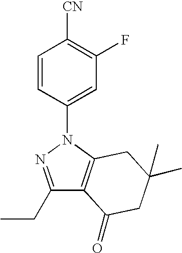

### 4-(3-Ethyl-6,6-dimethyl-4-oxo-4,5,6,7-tetrahydroindazol-1-yl)-2-fluorobenzonitrile

5,5-Dimethyl-2-propionylcyclohexane-1,3-dione (1.58 g, 8.07 mmol), 2-fluoro-4-hydrazinobenzonitrile (1.22 g, 8.07 mmol), ACOH (2 mL), and EtOH (6 mL) were combined. The mixture was stirred at 60° C. for 2.5 hours. The reaction was cooled to 25° C. and diluted with EtOAc (100 mL). It was washed with sat. NaHCO₃ (100 mL) and sat. NaCl (100 mL). Following concentration, the material was purified by gradient flash chromatography eluting with 0% to 25% EtOAc in hexanes to provide 4-(3-ethyl-6,6-dimethyl-4-oxo-4,5,6,7-tetrahydroindazol-1-yl)-2-fluorobenzonitrile (2.15 g, 85%) as an orange solid (LC/MS m/z=312.1 [M+H]⁺).

### EXAMPLE 36

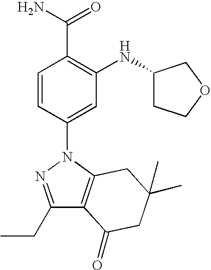

### (S)-4-(3-ethyl-6,6-dimethyl-4-oxo-4,5,6,7-tetrahydro-1H-indazol-1-yl)-2-(tetrahydrofuran-3-ylamino)benzamide

S-(−)-3-Aminotetrahydrofuran hydrochloride (149 mg, 1.20 mmol) and diisopropylamine (0.70 mL, 4.0 mmol) were added to 4-(3-ethyl-6,6-dimethyl-4-oxo-4,5,6,7-tetrahydroindazol-1-yl)-2-fluorobenzonitrile (311 mg, 1.00 mmol) in DMSO (0.7 mL). The mixture was stirred at 60° C. for 48 hours. It was diluted with EtOAc (10 mL) and washed with H₂O (10 mL) and sat. NaCl (10 mL). The material was concentrated and passed through a plug of silica eluting with EtOAc. After the solvent was removed, the white solid was dissolved in DMSO (0.4 mL) and EtOH (1.6 mL). To the solution were added 1 M NaOH (0.2 mL) and 30% H₂O₂ (0.2 mL). The reaction was stirred at 25° C. for 1 hour. It was diluted with EtOAc (10 mL) and washed with H₂O (10 mL) and sat. NaCl (10 mL). The organic layer was dried over Na₂SO₄ and concentrated. Purification by gradient flash chromatography eluting with 0% to 100% EtOAc in hexanes provided (S)-4-(3-ethyl-6,6-dimethyl-4-oxo-4,5,6,7-tetrahydro-1H-indazol-1-yl)-2-(tetrahydrofuran-3-ylamino)benzamide (251 mg, 63%) as a white solid (LC/MS m/z=397.1 [M+H]⁺). ¹H NMR (DMSO, 20° C., 400 MHz) δ (ppm) 8.56 (d, 1H), 7.75 (d, 2H), 6.72 (s, 1H), 4.18 (s, 1H), 4.12 (q, 1H), 4.02-3.53 (m, 8H), 2.48 (s, 4H), 1.17 (t, 3H), 1.00 (s, 6H).

### EXAMPLE 37

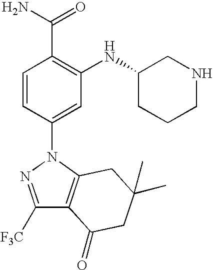

### (S)-4-(6,6-dimethyl-4-oxo-3-(trifluoromethyl)-4,5,6,7-tetrahydro-1H-indazol-1-yl)-2-(piperidin-3-ylamino)benzamide

2-Bromo-4-(6,6-dimethyl-4-oxo-3-trifluoromethyl-4,5,6,7-tetrahydro-indazol-1-yl)-benzonitrile (1.236 g), (S)-3-amino-piperidine-1-carboxylic acid tert-butyl ester (0.6 g), Pd(OAc)₂ (34.2 mg), DPPF (169.8 mg), and NaO^{t}Bu (588 mg) in toluene (8 mL) were mixed and microwaved at 120° C. for 15 min. Then the reaction mixture was concentrated and purified by Biotage column chromatography, eluted by 15% EtOAc in hexane to give 3-[2-Cyano-5-(6,6-dimethyl-4-oxo-3-trifluoromethyl-4,5,6,7-tetrahydro-indazol-1-yl)-phenylamino]-S-piperidine-1-carboxylic acid tert-butyl ester (1 g, 63%). LCMS (M+H) m/z=532.

3-[2-Cyano-5-(6,6-dimethyl-4-oxo-3-trifluoromethyl-4,5,6,7-tetrahydro-indazol-1-yl)-phenylamino]-S-piperidine-1-carboxylic acid tert-butyl ester (1 g), 1N NaOH aq. (10 mL), and H₂O₂ (3 mL) were dissolved in EtOH/DMSO (4:1, 20 mL) and stirred at RT for 2 h. Then the reaction mixture was concentrated and poured into Sat'd NH₄Cl aq. (150 mL), extracted by EtOAc (3×100 mL), dried over Na₂SO₄, filtered, concentrated to give 3-[2-Carbamoyl-5-(6,6-dimethyl-4-oxo-3-trifluoromethyl-4,5,6,7-tetrahydro-indazol-1-yl)-phenylamino]-S-piperidine-1-carboxylic acid tert-butyl ester (1 g, 96.7%). LCMS (M+H) m/z=549.3.

3-[2-Carbamoyl-5-(6,6-dimethyl-4-oxo-3-trifluoromethyl-4,5,6,7-tetrahydro-indazol-1-yl)-phenylamino]-S-piperidine-1-carboxylic acid tert-butyl ester (1 g) was dissolved in DCM (25 mL) and TFA (5 mL) and stirred at RT for 1 h. Then the reaction mixture was poured into Sat'd Na₂CO₃ aq. and extracted with DCM (3×100 mL), dried over Na₂SO₄, filtered, concentrated to give (S)-4-(6,6-dimethyl-4-oxo-3-(trifluoromethyl)-4,5,6,7-tetrahydro-1H-indazol-1-yl)-2-(piperidin-3-ylamino)benzamide (0.8 g, 97.8%). LCMS (M+H) m/z=449.3. ¹H NMR (DMSO, 20° C., 400 MHz) δ (ppm) 8.47 (d, 1H), 7.94 (b, 1H), 7.76 (d, 1H), 7.34 (b, 1H), 6.85 b, 1H), 6.70 (d, 1H), 3.64-3.29 (m, 1H), 3.11-3.02 (m, 1H), 2.95 (s, 2H), 2.85-2.76 (m, 1H), 2.62-2.52 (m, 1H), 2.46-2.37 (m, 4H), 1.94-1.85 (m, 1H), 1.68-1.57 (m, 1H), 1.53-1.35 (m, 2H), 1.01 (s, 6H).

### EXAMPLE 38

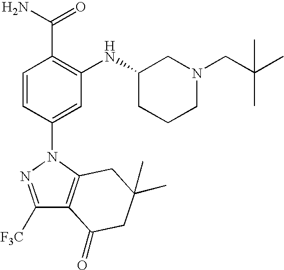

### (S)-4-(6,6-dimethyl-4-oxo-3-(trifluoromethyl)-4,5,6,7-tetrahydro-1H-indazol-1-yl)-2-(1-neopentylpiperidin-3-ylamino)benzamide

4-(6,6-Dimethyl-4-oxo-3-trifluoromethyl-4,5,6,7-tetrahydro-indazol-1-yl)-2-(S-piperidin-3-ylamino)-benzamide (80 mg), trimethylacetaldehyde (0.20 mL, 1 eq.), NaOAc (44.05 mg), and NaCNBH₃ (33.45 mg) were mixed and stirred in MeOH—H₂O(1:1, 4 mL) at RT overnight. Then the reaction mixture was concentrated and purified by biotech chromatography, eluted by 10% MeOH in DCM to give (S)-4-(6,6-dimethyl-4-oxo-3-(trifluoromethyl)-4,5,6,7-tetrahydro-1H-indazol-1-yl)-2-(1-neopentylpiperidin-3-ylamino)benzamide (60 mg, 65% yield). LCMS (M+H) m/z=520.2. ¹H NMR (DMSO, 20° C., 400 MHz) δ (ppm)8.74-8.66 (m, 1H), 7.92 (b, 1H), 7.76 (d, 1H), 7.34 (b, 1H), 7.82 (d, 1H), 6.68 (dd, 1H), 3.65-3.55 (m, 1H), 2.93 (s, 2H), 2.75-2.64 (m, 1H), 2.52-2.32 (m, 5H), 2.09-1.98 (m, 2H), 1.71-1.54 (m, 2H), 1.53-1.36 (m, 2H), 1.00 (s, 6H), 0.83 (s, 9H).

### EXAMPLE 39

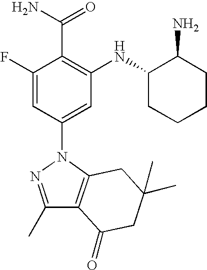

### 2-((1S,2S)-2-aminocyclohexylamino)-6-fluoro-4-(3,6,6-trimethyl-4-oxo-4,5,6,7-tetrahydro-1H-indazol-1-yl)benzamide

2,6-Difluoro-4-(3,6,6-trimethyl-4-oxo-4,5,6,7-tetrahydro-indazol-1-yl)-benzonitrile (250 mg), (1S,2S)-1,2-diaminocyclohexane (100 mg, 1.1 eq.), and DIPEA (0.3 mL) were stirred in DMSO (5 mL) at 120° C. for 2 h. LCMS showed product 2-(S,S-2-Amino-cyclohexylamino)-6-fluoro-4-(3,6,6-trimethyl-4-oxo-4,5,6,7-tetrahydro-indazol-1-yl)-benzonitrile was formed (M+H m/z=410.1). Then EtOH/DMSO (4:1, 20 mL), 1N NaOH aq. (5 mL), and H₂O₂ (3 mL) were added and the mixture stirred at RT overnight. The reaction mixture was concentrated and poured into Sat'd NH₄Cl aq. (150 mL), extracted by EA (3×100 mL), dried over Na₂SO₄, filtered, concentrated to give a crude product, which was purified by Biotage chromatography, eluted by 10% MeOH in DCM to give 2-((1S,2S)-2-aminocyclohexylamino)-6-fluoro-4-(3,6,6-trimethyl-4-oxo-4,5,6,7-tetrahydro-1H-indazol-1-yl)benzamide (278 g, 82% over 2 steps). LCMS (M+H) m/z=428.1. ¹H NMR (DMSO, 20° C., 400 MHz) δ (ppm) 7.79 (s, 1H), 7.00 (d, 1H), 6.80 (s, 1H), 6.64 (dd, 1H), 3.61-3.17 (m, 3H), 3.09-2.84 (m, 3H), 2.38 (s, 3H), 2.37-2.24 (m, 2H), 2.02-1.93 (m, 2H), 1.70-1.60 (m, 2H), 1.48-1.35 (m, 1H), 1.34-1.12 (m, 3H), 1.02 (s, 3H), 0.98 (s, 3H).

### EXAMPLE 40

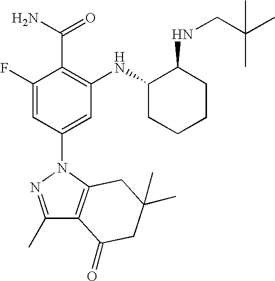

### 2-fluoro-6-((1S,2S)-2-(neopentylamino)cyclohexylamino)-4-(3,6,6-trimethyl-4-oxo-4,5,6,7-tetrahydro-1H-indazol-1-yl)benzamide

2-(S,S-2-Amino-cyclohexylamino)-6-fluoro-4-(3,6,6-trimethyl-4-oxo-4,5,6,7-tetrahydro-indazol-1-yl)-benzamide (90 mg), trimethylacetaldehyde (0.040 mL, 1 eq.), NaOAc (53 mg), and NaCNBH₃ (40 mg) were mixed and stirred in MeOH—H₂O (1:1, 4 mL) at RT overnight. Then the reaction mixture was concentrated and purified by Biotage chromatography, eluted by EtOAc to give 2-fluoro-6-((1S,2S)-2-(neopentylamino)cyclohexylamino)-4-(3,6,6-trimethyl-4-oxo-4,5,6,7-tetrahydro-1H-indazol-1-yl)benzamide (100 mg, 95% yield). LCMS (M+H) m/z=498.3.

### EXAMPLE 41

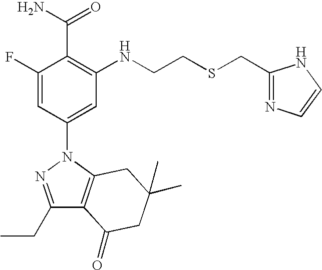

### 2-(2-((1H-imidazol-2-yl)methylthio)ethylamino)-4-(3-ethyl-6,6-dimethyl-4-oxo-4,5,6,7-tetrahydro-1H-indazol-1-yl)-6-fluorobenzamide

2,6-Difluoro-4-(3-ethyl-6,6-dimethyl-4-oxo-4,5,6,7-tetrahydro-indazol-1-yl)-benzonitrile (134 mg), 2-(1H-imidazol-2-ylmethylsulfanyl)-ethylamine (64 mg), and DIPEA (0.077 mL) were stirred in DMSO (2 mL) at 120° C. for 2 h. LCMS showed the product 4-(3-ethyl-6,6-dimethyl-4-oxo-4,5,6,7-tetrahydro-indazol-1-yl)-2-fluoro-6-[2-(1H-imidazol-2-ylmethylsulfany)-ethylamino]-benzonitrile was formed (M+H m/z=467.2). Then EtOH (8 mL), 1N NaOH aq. (2 mL), and H₂O₂ (1 mL) were added and the mixture stirred at RT overnight. The reaction mixture was concentrated and poured into Sat'd NH₄Cl aq. (150 mL), extracted by EA (3×100 mL), dried over Na₂SO₄, filtered, concentrated to give a crude product, which was purified by Biotage chromatography, eluted by 5-20% MeOH in DCM to give 2-(2-((1H-imidazol-2-yl)methylthio)ethylamino)-4-(3-ethyl-6,6-dimethyl-4-oxo-4,5,6,7-tetrahydro-1H-indazol-1-yl)-6-fluorobenzamide (11.4 mg, 6% over 2 steps). LCMS (M+H) m/z=485.1.

### EXAMPLE 42

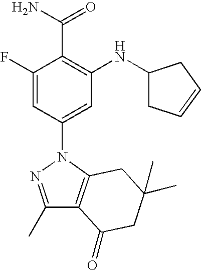

### 2-(cyclopent-3-enylamino)-6-fluoro-4-(3,6,6-trimethyl-4-oxo-4,5,6,7-tetrahydro-1H-indazol-1-yl)benzamide

2,6-Difluoro-4-(3,6,6-trimethyl-4-oxo-4,5,6,7-tetrahydro-indazol-1-yl)-benzonitrile (287 mg), cyclopent-3-enylamine (109 mg), and DIPEA (172.8 uL) were stirred in DMSO (2 mL) at 120° C. for 2 h. LCMS showed the product 2-(cyclopent-3-enylamino)-6-fluoro-4-(3,6,6-trimethyl-4-oxo-4,5,6,7-tetrahydro-indazol-1-yl)-benzonitrile was formed (M+H m/z=379.2). Then EtOH (8 mL), 2N NaOH aq. (1 mL), and H₂O₂ (1 mL) were added and the mixture stirred at RT for 3 h. The reaction mixture was concentrated and poured into Sat'd NH₄Cl aq. (150 mL), extracted by EtOAc (3×100 mL), dried over Na₂SO₄, filtered, concentrated to give a gum, which was purified by Biotage chromatography, eluted by 30-50% EtOAc in hexane to give 2-(cyclopent-3-enylamino)-6-fluoro-4-(3,6,6-trimethyl-4-oxo-4,5,6,7-tetrahydro-1H-indazol-1-yl)benzamide (323 mg, 90% over 2 steps). LCMS (M+H) m/z=397.1. ¹H NMR (DMSO, 20° C., 400 MHz) δ (ppm) 7.72 (s, 1H), 7.68 (s, 1H), 7.46 (d, 1H), 6.66-6.56 (m, 2H), 5.74 (s, 2H), 4.19-4.11 (m, 1H), 2.95 (s, 2H0, 2.84-2.74 (m, 2H), 2.38 (s, 3H), 2.31 (s, 2H), 2.22-2.18 (m, 1H), 2.18-2.13 (m, 1H), 1.01 (s, 6H).

### EXAMPLE 43

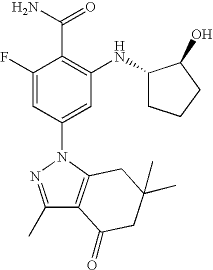

### 2-fluoro-6-((1S,2S)-2-hydroxycyclopentylamino)-4-(3,6,6-trimethyl-4-oxo-4,5,6,7-tetrahydro-1H-indol-1-yl)benzamide

4-Bromo-2,6-difluoro-bezonitrile (1 g), (1S,2S)-2-benzyloxy-cyclopentylamine (964 mg, 1.1 eq.), and DIPEA (0.882 mL, 1.1 eq.) were stirred in DMSO (5 mL) at 120° C. for 2 h. The reaction mixture was poured into Sat'd NH₄Cl aq. (150 mL), extracted by EtOAc (3×100 mL), dried over Na₂SO₄, filtered, concentrated to give 2-((1S,2S)-2-benzyloxy-cyclopentylamino)-4-bromo-6-fluoro-bezonitrile (1.8 g, 100% yield). LCMS (M+H) m/z=389, 391.

To a stirred solution of 2-((1S,2S)-2-benzyloxy-cyclopentylamino)-4-bromo-6-fluoro-bezonitrile (1.48 g) and 3,6,6-trimethyl-1,5,6,7-tetrahydro-indol-4-one (0.56 g) in dioxane (15 mL) were added K₂CO₃ (2.64 g). The suspension was freezed in ice-bath and degassed by pump/backfill of N₂ 3 times. Then CuI (1.1 g) and N,N′-dimethylethylenediamine (0.6 mL) were added. The reaction mixture was degassed again for 3 times with backfill of N₂. Then the sealed reaction mixture was stirred at 100° C. overnight. The reaction mixture was filtered through a celite pad, washed by EtOAc, concentrated to give a gum (LCMS m/z M+H=486.3). That was dissolved in EtOH-DMSO (4:1, 50 mL), then 1N NaOH aq (10 mL), and H₂O₂ (3 mL) were added and the mixture stirred at RT for 4 h. The reaction mixture was concentrated and poured into Sat'd NH₄Cl aq. (250 mL), extracted by EtOAc (3×150 mL), dried over Na₂SO₄, filtered, concentrated to give a gum, which was purified by Biotage chromatography, eluted by 30% EtOAc in hexane to give 2-fluoro-6-((1S,2S)-2-benzyloxy-cyclopentylamino)-4-(3,6,6-trimethyl-4-oxo-4,5,6,7-tetrahydro-indol-1-yl)-benzamide (1.2 g, 63% for 2 steps). LCMS (M+H) m/z=504.3.

2-fluoro-6-((1S,2S)-2-benzyloxy-cyclopentylamino)-4-(3,6,6-trimethyl-4-oxo-4,5,6,7-tetrahydro-indol-1-yl)-benzamide (1.2 g), 10% Pd/C (120 mg) and a few drops of TFA were dissolved in MeOH (30 mL) and hydrogenated under 55 psi of H₂ overnight. Then the reaction mixture was adjusted by TEA to pH 8˜9, filtered through a celite pad to remove Pd/C, concentrated and purified by Biotage chromatography, eluted by 50% EtOAc in hexane to give 2-fluoro-6-((1S,2S)-2-hydroxycyclopentylamino)-4-(3,6,6-trimethyl-4-oxo-4,5,6,7-tetrahydro-1H-indol-1-yl)benzamide (762 mg, 77% yield). LCMS (M+H) m/z=414.2. ¹H NMR (DMSO, 20° C., 400 MHz) δ (ppm) 7.69 (s, 1H), 7.65 (s, 1H), 7.37 (d, 1H), 6.82 (s, 1H0, 6.59 (s, 1H), 6.49 (dd, 1H), 4.92 (d, 1H), 3.86-3.80 (m, 1H), 3.55-3.47 (m, 1H), 3.31 (s, 1H), 2.74 (s, 2H), 2.25-2.09 (m, 5H), 1.83-1.56 (m, 3H), 1.56-1.46 (m, 1H), 1.40-1.30 (m, 1H), 0.99 (s, 3H), 0.98 (s, 3H).

### EXAMPLE 44

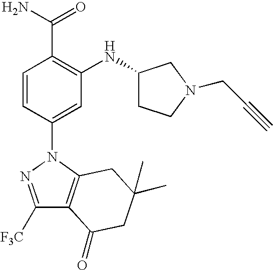

### (S)-4-(6,6-dimethyl-4-oxo-3-(trifluoromethyl)-4,5,6,7-tetrahydro-1H-indazol-1-yl)-2-(1-(prop-2-ynyl)pyrrolidin-3-ylamino)benzamide

2-Bromo-4-(6,6-dimethyl-4-oxo-3-trifluoromethyl-4,5,6,7-tetrahydro-indazol-1-yl)benzonitrile (0.412 g, 1 mmol), (S)-(−)-1-Boc-3-aminopyrrolidine (0.35 g, 2.0 mmol), PdOAc (29 mg, 0.13 mmol), DPPF (58 g, 0.1 mmol), and NaO^{t}Bu (192 mg, 2.0 mmol) were suspended in toluene (2 mL) and sealed in a microwave tube. The mixture was microwaved at 110° C. for 800 sec. The product was extracted (ethyl acetate 200 mL over water 100 mL). The organic layer was dried, concentrated, and subjected to chromatography, affording (S)-3-[2-Cyano-5-(6,6-dimethyl-4-oxo-3-trifluoromethyl-4,5,6,7-tetrahydro-indazol-1-yl)-phenylamino]-pyrrolidine-1-carboxylic acid tert-butyl ester (222 mg, 43%).

(S)-3-[2-Cyano-5-(6,6-dimethyl-4-oxo-3-trifluoromethyl-4,5,6,7-tetrahydro-indazol-1-yl)-phenylamino]-pyrrolidine-1-carboxylic acid tert-butyl ester (222 mg, 0.4 mmol) was diluted with dry methanol (5 mL) and acetyl chloride (0.5 mL) was added, dropwise. After stirring at ambient temperature for 1 h, the reaction mixture was brought to a brief boil twice using a heat gun. On cooling, the mixture was concentrated. The residue was azeotroped with acetonitrile and dried in vacuo to a tan solid. To this was added DMF (2 mL), diisopropylethylamine (0.16 mL, 0.9 mmol), and propargyl bromide (80% in toluene, 0.04 mL). The suspension was stirred 24 h at RT. Additional propargyl bromide solution (0.02 mL) was added and stirring was continued 20 more h. The mixture was extracted ethyl acetate (100 mL) over water (50 mL). The organic layer was dried over magnesium sulfate, filtered, concentrated, and chromatographed, affording 4-(6,6-Dimethyl-4-oxo-3-trifluoromethyl-4,5,6,7-tetrahydro-indazol-1-yl)-2-(1-prop-2-ynyl-5-pyrrolidin-3-ylamino)-benzonitrile (60 mg, 29%).

4-(6,6-Dimethyl-4-oxo-3-trifluoromethyl-4,5,6,7-tetrahydro-indazol-1-yl)-2-(1-prop-2-ynyl-5-pyrrolidin-3-ylamino)-benzonitrile (60 mg, 0.30 mmol) was combined with DMSO (6 drops), 95% ethanol (2 mL), KOH (127 mg, 2.3 mmol) at 50° C. Hydrogen peroxide (30% aqueous, 0.5 mL) was added, and the mixture was stirred for 30 min. The product was taken up in ethyl acetate (100 mL) and washed with water (50 mL). The organic layer was dried over magnesium sulfate. Concentration followed by silica gel chromatography afforded (S)-4-(6,6-dimethyl-4-oxo-3-(trifluoromethyl)-4,5,6,7-tetrahydro-1H-indazol-1-yl)-2-(1-(prop-2-ynyl)pyrrolidin-3-ylamino)benzamide as a foam (47 mg, 33%). LCMS (M+H) m/z=474. ¹H NMR (DMSO, 20° C., 400 MHz) δ (ppm) 8.53 (d, 1H), 7.98 (b, 1H), 7.78 (d, 1H), 7.33 (b, 1H), 6.78-6.73 (m, 2H), 4.06-3.97 (m, 1H), 3.40-3.36 (m, 2H), 3.13-3.09 (m, 1H), 2.88-2.81 (m, 1H), 2.78-2.69 (m, 1H), 2.53-2.45 (m, 4H), 2.43 (s, 2H), 2.36-2.25 (m, 1H), 1.60-1.50 (m, 1H), 1.02 (d, 6H).

### EXAMPLE 45

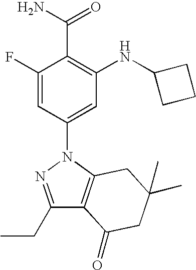

### 2-(cyclobutylamino)-4-(3-ethyl-6,6-dimethyl-4-oxo-4,5,6,7-tetrahydro-1H-indazol-1-yl)-6-fluorobenzamide

4-(3-Ethyl-6,6-dimethyl-4-oxo-4,5,6,7-tetrahydro-indazol-1-yl)-2,6-difluorobenzonitrile (0.33 g, 1.0 mmol), N,N-diisopropylethylamine (0.13 g, 1 mmol), cyclobutylamine (0.071 g, 1 mmol), and DMSO (4 mL) were subjected to microwave irradiation at 150° C. for 3000 sec. The mixture was extracted with ethyl acetate, washed with water, and dried over MgSO₄. Purification of the crude product by silica gel chromatography afforded 2-Cyclobutylamino-4-(3-ethyl-6,6-dimethyl-4-oxo-4,5,6,7-tetrahydro-indazol-1-yl)-6-fluoro-benzonitrile (0.31 g, 81%).

To a flask was added 2-Cyclobutylamino-4-(3-ethyl-6,6-dimethyl-4-oxo-4,5,6,7-tetrahydro-indazol-1-yl)-6-fluoro-benzonitrile (0.31 g, 0.8 mmol), 5 mL of Ethanol-DMSO (4:1), hydrogen peroxide (30%, ˜1 mL), and 1N NaOH (1 mL). The mixture was stirred at RT for 3 h. The product was extracted with ethyl acetate, washed with water, dried with MgSO₄. Solvent was removed, dried in vacuum to give 0.3 g of 2-(cyclobutylamino)-4-(3-ethyl-6,6-dimethyl-4-oxo-4,5,6,7-tetrahydro-1H-indazol-1-yl)-6-fluorobenzamide, yield 94%. LCMS (M+H) m/z=399. ¹H NMR (DMSO, 20° C., 400 MHz) δ (ppm) 7.73 (b, 1H), 7.69 (b, 1H), 7.44 (d, 1H), 6.64 (d, 1H), 6.45 (s, 1H), 3.97-3.87 (m, 1H), 2.92 (s, 2H), 2.79 (q, 2H), 2.42-2.29 (m, 4H), 1.90-1.67 (m, 4H), 1.18 (t, 3H), 1.00 (s, 6H).

### EXAMPLE 46

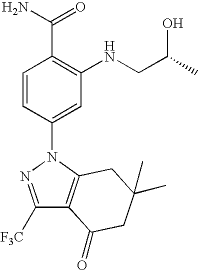

### (R)-4-(6,6-dimethyl-4-oxo-3-(trifluoromethyl)-4,5,6,7-tetrahydro-1H-indazol-1-yl)-2-(2-hydroxypropylamino)benzamide

2-Bromo-4-(6,6-dimethyl-4-oxo-3-trifluoromethyl-4,5,6,7-tetrahydro-indazol-1-yl)benzonitrile (0.20 g, 0.50 mmol), (R)-(−)-2-hydroxy-propylamine (0.11 g, 1.5 mmol), PdOAc (15 mg, 0.06 mmol), DPPF (28 mg, 0.05 mmol), and sodium t-butoxide (96 mg, 1.0 mmol) were suspended in toluene (3 mL) and sealed in a microwave tube. The mixture was microwaved at 110° C. for 1500 sec. The product was extracted with ethyl acetate/water. The organic layer was dried, concentrated, and subjected to chromatography, affording 4-(6,6-Dimethyl-4-oxo-3-trifluoromethyl-4,5,6,7-tetrahydroindazol-1-yl)-2-(R-2-hydroxy-propylamino)-benzonitrile (40 mg, 20%).

4-(6,6-Dimethyl-4-oxo-3-trifluoromethyl-4,5,6,7-tetrahydroindazol-1-yl)-2-(R-2-hydroxy-propylamino)-benzonitrile (40 mg) was stirred at ambient temperature in the presence of ethanol-DMSO (4:1, 10 mL); sodium hydroxide solution (1N, 0.5 mL); and hydrogen peroxide (30%, 0.5 mL) for 3 h. The product was then isolated by extraction with ethyl acetate/water followed by concentration and chromatography, affording 30 mg of (R)-4-(6,6-dimethyl-4-oxo-3-(trifluoromethyl)-4,5,6,7-tetrahydro-1H-indazol-1-yl)-2-(2-hydroxypropylamino)benzamide (71%). LCMS (M+H) m/z=425.

### EXAMPLE 47

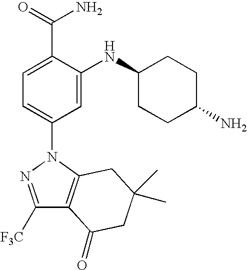

### 2-(trans-4-aminocyclohexylamino)-4-(6,6-dimethyl-4-oxo-3-(trifluoromethyl)-4,5,6,7-tetrahydro-1H-indazol-1-yl)benzamide

2-(4-Amino-cyclohexylamino)-4-(6,6-dimethyl-4-oxo-3-trifluoromethyl-4,5,6,7-tetrahydro-indazol-1-yl)-benzonitrile, from Example 4, was treated with 4:1 EtOH:DMSO, 1M NaOH, and 0.1 mL 30% H₂O₂ in a similar fashion as described in Example 4 to provide 2-(trans-4-aminocyclohexylamino)-4-(6,6-dimethyl-4-oxo-3-(trifluoromethyl)-4,5,6,7-tetrahydro-1H-indazol-1-yl)benzamide.

### EXAMPLE 49

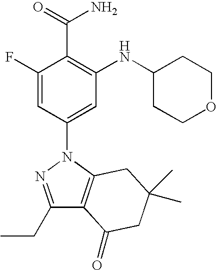

### 4-(3-ethyl-6,6-dimethyl-4-oxo-4,5,6,7-tetrahydro-1H-indazol-1-yl)-2-fluoro-6-(tetrahydro-2H-pyran-4-ylamino)benzamide

To a solution of 4-(3-ethyl-6,6-dimethyl-4-oxo-4,5,6,7-tetrahydro-indazol-1-yl)-2,6-difluoro-benzonitrile (88 mg, 0.27 mmol) in DMF (2 mL), 4-aminotetrahydropyran (27 mg, 0.27 mmol) is added, and the reaction mixture is stirred at 25° C. for 13 hours. Then, the mixture is diluted with EtOAc (10 mL), washed with H₂O (2×10 mL) and brine (1×10 mL), and the organic layers are dried over Na₂SO₄ and concentrated. Purification of the residue using a Biotage column (elution with 0-50% EtOAc in hexanes) affords 4-(3-ethyl-6,6-dimethyl-4-oxo-4,5,6,7-tetrahydro-1H-indazol-1-yl)-2-fluoro-6-(tetrahydro-2H-pyran-4-ylamino)benzonitrile (89 mg, 81% yield)as a white solid. LCMS: m/z=411 [M+H]⁺.

A solution of 4-(3-ethyl-6,6-dimethyl-4-oxo-4,5,6,7-tetrahydro-1H-indazol-1-yl)-2-fluoro-6-(tetrahydro-2H-pyran-4-ylamino)-benzonitrile (89 mg, 0.22 mmol) in EtOH/DMSO (4:1, 1 mL) is treated with 1M NaOH (10 drops) and 35% aqueous H₂O₂ (10 drops). The reaction is stirred for 13 h at 25° C. then diluted with EtOAc (10 mL), washed with H₂O (2×10 mL) and brine (1×10 mL). The combined organic layers are dried over Na₂SO₄, concentrated under reduced pressure, and the residue is purified using a Biotage column (elution with 0-50% EtOAc in hexanes) to give 4-(3-ethyl-6,6-dimethyl-4-oxo-4,5,6,7-tetrahydro-1H-indazol-1-yl)-2-fluoro-6-(tetrahydro-2H-pyran-4-ylamino)benzamide (64 mg, 69% yield) as a white solid. LCMS: m/z=429 [M+H]⁺. ¹H NMR (400 MHz, d₆ DMSO): δ 9.00 (b, 1H), 6.63-6.44 (m, 3H), 5.65 (b, 1H), 3.62-3.54 (m, 3H), 2.93 (q, 2H), 2.83 (s, 2H), 2.41 (s, 2H), 2.08-2.03 (m, 2H), 1.66-1.61 (m, 5H), 1.30 (t, 3H), 1.12 (s, 6H).

### EXAMPLE 50

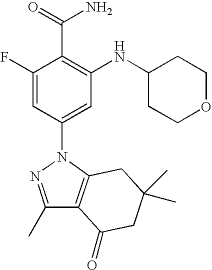

### 2-fluoro-6-(tetrahydro-2H-pyran-4-ylamino)-4-(3,6,6-trimethyl-4-oxo-4,5,6,7-tetrahydro-1H-indazol-1-yl)benzamide

2,6-Difluoro-4-(3,6,6-trimethyl-4-oxo-4,5,6,7-tetrahydro-indazol-1-yl)-benzonitrile (3 g, 9.51 mmol), 4-aminotetrahydropyran (1.44 g, 14.3 mmol) and diisopropylethylamine (1.84 g, 14.3 mmol) are combined in DMSO (12 mL) and stirred at 60° C. for 4 h. While still at 60° C., the mixture is treated with 25% NaOH (0.76 g, 19 mmol, 3 mL) solution and isopropanol (10 mL) followed by dropwise addition of 30% hydrogen peroxide (0.65 g, 19 mmol, 2.2 mL) solution. The reaction mixture is poured into water (100 mL) and extracted with EtOAc (2×100 mL). The combined organic layers are washed with brine (50 mL), dried (MgSO₄) and concentrated to give an off-white crystalline solid (3.75 g). The solid is purified via chromatography (silica, 50 to 100% EtOAc in hexanes) and the product triturated with hexanes and EtOAc to give 2-fluoro-6-(tetrahydro-2H-pyran-4-ylamino)-4-(3,6,6-trimethyl-4-oxo-4,5,6,7-tetrahydro-1H-indazol-1-yl)benzamide (2.92 g, 74%) as a white crystalline solid. LC/MS: m/z=415 [M+H]⁺. ¹H NMR (400 MHz, d₆ DMSO): δ 7.72 (d, 2H), 7.35 (d, 1H), 6.67 (s, 1H), 6.61 (d, 1H), 3.84-3.42 (m, 5H), 3.31 (s, 2H), 2.49 (s, 2H), 2.47 (s, 3H), 2.30 (s, 2H), 1.89 (d, 2H), 1.38 (q, 1H), 0.99 (s, 6H)

### EXAMPLE 52

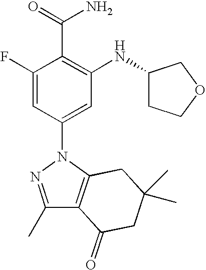

### (S)-2-fluoro-6-(tetrahydrofuran-3-ylamino)-4-(3,6,6-trimethyl-4-oxo-4,5,6,7-tetrahydro-1H-indazol-1-yl)benzamide

2,6-Difluoro-4-(3,6,6-trimethyl-4-oxo-4,5,6,7-tetrahydro-indazol-1-yl)-benzonitrile (3 g, 9.51 mmol), (S)-tetrahydrofuran-3-amine hydrochloride (1.76 g, 14.3 mmol) and diisopropylethylamine (2.46 g, 19 mmol) are combined in DMSO (12 mL) and stirred at 60° C. for 4 h. While still at 60° C., the mixture is treated with 25% NaOH (1.14 g, 28.5 mmol, 4.6 mL) and isopropanol (10 mL) followed by dropwise addition of 30% hydrogen peroxide (0.97 g, 28.5 mmol, 3.2 mL) solution. The reaction mixture is poured into water (100 mL) and extracted with EtOAc (2×100 mL). The combined organic layers are washed with brine (50 mL), dried over MgSO₄, and concentrate to a off-white crystalline solid. The solid is purified via chromatography (silica, 50 to 100% EtOAc in hexanes) to give a white crystalline solid. The solid is almost dissolved in EtOAc, diluted with hexanes and stirred overnight. The crystals are collected to give (S)-2-fluoro-6-(tetrahydrofuran-3-ylamino)-4-(3,6,6-trimethyl-4-oxo-4,5,6,7-tetrahydro-1H-indazol-1-yl)benzamide (2.75 g, 72%) as a white crystalline solid. LC/MS: m/z=401 [M+H]⁻. ¹H NMR (400 MHz, d₆ DMSO): δ 7.75 (d, 2H), 7.45 (d, 1H), 6.65 (d, 1H), 6.60 (s, 1H), 4.13-3.54 (m, 4H), 2.94 (s, 2H), 2.48 (s, 2H), 2.37 (s, 3H), 2.31 (s, 2H), 2.26-2.21 (m, 1H), 1.00 (s, 6H).

### EXAMPLE 60

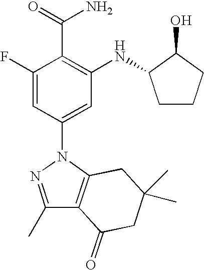

### 2-fluoro-6-((1S,2S)-2-hydroxycyclopentylamino)-4-(3,6,6-trimethyl-4-oxo-4,5,6,7-tetrahydro-1H-indazol-1-yl)benzamide

2,6-Difluoro-4-(3,6,6-trimethyl-4-oxo-4,5,6,7-tetrahydro-indazol-1-yl)-benzonitrile (3 g, 9.51 mmol), (1S,2S)-2-hydroxycyclopentylamine hydrochloride (1.96 g, 14.3 mmol), and diisopropylethylamine (2.46 g, 19 mmol) are combined in DMSO (12 mL) and stirred at 60° C. for 4 h to give 2-fluoro-6-((1S,2S)-2-hydroxy-cyclopentylamino)-4-(3,6,6-trimethyl-4-oxo-4,5,6,7-tetrahydro-indazol-1-yl)-benzonitrile. The mixture is then diluted with isopropanol (10 mL) and treated three times with 25% aqueous NaOH (1.5 mL) and 30% aqueous hydrogen peroxide (1 mL). The mixture is then partitioned between EtOAc (100 mL) and water (100 mL), and the aqueous layer is washed with additional EtOAc (100 mL). The combined organic layers are concentrated, and the oily residue is purified via chromatography (elution 30 to 100% EtOAc in hexanes). The clean fractions are combined, concentrated and recrystallized from EtOAc/hexanes to give 2-fluoro-6-((1S,2S)-2-hydroxycyclopentylamino)-4-(3,6,6-trimethyl-4-oxo-4,5,6,7-tetrahydro-1H-indazol-1-yl)benzamide (3.53 g, 89%) as a white crystalline solid. LCMS m/z=415 [M+H]⁻. ¹H NMR (400 MHz, d₆ DMSO): δ 7.72 (br s, 1H), 7.68 (br s, 1H), 7.38 (d, 1H), 6.72 (d, 1H), 6.61 (dd, 1H), 4.91 (d, 1H), 3.84 (m, 1H), 3.50 (m, 1H), 2.94 (2d, 2H), 2.38 (s, 3H), 2.31 (2 d, 2H), 2.15 (m, 1H), 1.57-1.82 (m, 3H), 1.36 (m, 1H), 1.02 (s, 3H), 1.00 (s, 3H).

### EXAMPLE 73

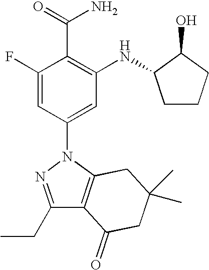

### 4-(3-ethyl-6,6-dimethyl-4-oxo-4,5,6,7-tetrahydro-1H-indazol-1-yl)-2-fluoro-6-((1S,2S)-2-hydroxycyclopentylamino)benzamide

4-(3-Ethyl-6,6-dimethyl-4-oxo-4,5,6,7-tetrahydro-indazol-1-yl)-2,6-difluoro-benzonitrile (800 mg, 2.43 mmol), (1S,2S)-2-hydroxycyclopentylamine hydrochloride (351 mg, 2.55 mmol) and diisopropylethylamine (471 mg, 3.6 mmol) are combined in DMSO (3 mL) and stirred at 50° C. After 2 h, additional amino alcohol (70 mg) and diisopropylethylamine (120 μL) are added and stirring is continued overnight. The mixture is then treated three times with 10% NaOH in methanol (1 mL) and 30% hydrogen peroxide (0.25 mL) and methanol (1 mL) and heated to 50° C. The reaction mixture is diluted with water (15 mL) and extracted with methylene chloride (2×10 mL). The organic layers are concentrated and purified via chromatography (silica, 0 to 15% methanol in DCM) to give a glass (1.4 g). The glass is re-purified via chromatography (silica, 30 to 100% EtOAc in hexanes over 16 cv), and is triturated with EtOAc and hexanes to give 4-(3-ethyl-6,6-dimethyl-4-oxo-4,5,6,7-tetrahydro-1H-indazol-1-yl)-2-fluoro-6-((1S,2S)-2-hydroxycyclopentylamino)benzamide (295 mg, 28%) as a crunchy solid. LC/MS: m/z=429 [M+H]⁺. ¹H NMR (400 MHz, d₆ DMSO): δ 7.72 (d, 2H), 7.39 (d, 1H), 6.63 (s, 1H), 6.61 (d, 1H), 2.94 (d, 2H), 2.80 (q, 2H), 2.47 (s, 2H), 2.30 (d, 2H), 2.15-2.13 (m, 1H), 1.76-1.35 (m, 5H), 1.18 (t, 3H), 1.01 (s, 3H), 0.99 (s, 3H).

### EXAMPLE 92

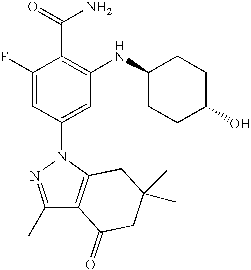

### 2-fluoro-6-(trans-4-hydroxycyclohexylamino)-4-(3,6,6-trimethyl-4-oxo-4,5,6,7-tetrahydro-1H-indazol-1-yl)benzamide

To a solution of 2,6-Difluoro-4-(3,6,6-trimethyl-4-oxo-4,5,6,7-tetrahydro-indazol-1-yl)-benzonitrile (1.26 g, 4.00 mmol) in DMSO (0.5 mL), DIEA (1.40 mL, 8.00 mmol) and trans-4-aminocyclohexanol (0.691 g, 6.00 mmol) are added, and the mixture is stirred at 100° C. for 13 hours. The reaction mixture is diluted with EtOAc (50 mL), washed with H₂O (2×50 mL) and brine (1×50 mL), and the organic layers are dried over Na₂SO₄ and concentrated to give 2-fluoro-6-(trans-4-hydroxycyclohexylamino)-4-(3,6,6-trimethyl-4-oxo-4,5,6,7-tetrahydro-1H-indazol-1-yl)benzonitrile (1.64 g, 100% yield) as a pink solid. LCMS m/z: =411 [M+H]⁺.

A solution of 2-fluoro-6-(trans-4-hydroxycyclohexylamino)-4-(3,6,6-trimethyl-4-oxo-4,5,6,7-tetrahydro-1H-indazol-1-yl)benzonitrile (1.64 g, 4.00 mmol) in EtOH/DMSO (4:1, 50 mL) is treated with 1M NaOH (10 mL) and 35% aqueous H₂O₂ (10 mL)

The reaction mixture is diluted with EtOAc (100 mL), washed with H₂O (2×50 mL) and brine (1×50 mL). The combined organic layers are dried over Na₂SO₄ and evaporated under reduced pressure. Purification of the residue using a Biotage column (elution with 0-60% EtOAc in hexanes) affords 2-fluoro-6-(trans-4-hydroxycyclohexylamino)-4-(3,6,6-trimethyl-4-oxo-4,5,6,7-tetrahydro-1H-indazol-1-yl)benzamide (1.71 g, 100% yield) as a white solid. LC/MS: m/z=429 [M+H]⁺. ¹H NMR (400 MHz, d₆ DMSO): δ 7.71-7.66 (m, 2H), 7.26 (d, 1H), 6.58 (d, 1H), 2.94 (s, 2H), 2.49 (s, 2H), 2.38 (s, 3H), 2.32 (s, 2H), 1.97-1.77 (m, 4H), 1.32-1.14 (m, 4H), 1.00 (s, 6H)

### EXAMPLE 98

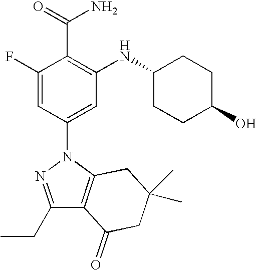

### 4-(3-ethyl-6,6-dimethyl-4-oxo-4,5,6,7-tetrahydro-1H-indazol-1-yl)-2-fluoro-6-(trans-4-hydroxycyclohexylamino)benzamide

4-(3-ethyl-6,6-dimethyl-4-oxo-4,5,6,7-tetrahydro-1H-indazol-1-yl)-2-fluoro-6-(trans-4-hydroxycyclohexylamino)-benzonitrile (1.5 g, 4.55 mmol) and trans-4-hydroxycyclohexylamine (1.05 g, 9.1 mmol) are combined in DMSO (6 mL) and stirred at 50° C. for 1 h. The mixture is then treated with 10% NaOH in methanol (2 mL) and 30% hydrogen peroxide (0.5 mL) and methanol (2 mL) and heated to 50° C. for 1 h. The reaction mixture is diluted with water (25 mL), extracted with dichloromethane (2×20 mL), and purified via chromatography (silica, 0 to 20% methanol in DCM over 16 cv) to give the product as a glass, which is triturated with EtOAc/hexanes, stirred overnight, filtered off and air dried to give 4-(3-ethyl-6,6-dimethyl-4-oxo-4,5,6,7-tetrahydro-1H-indazol-1-yl)-2-fluoro-6-(trans-4-hydroxycyclohexylamino)benzamide (1.72 g, 85.1%) as an off-white solid. LC/MS: m/z=443 [M+H]⁺. ¹H NMR (400 MHz, d₆ DMSO): δ 7.71 (br s, 1H), 7.65 (br s, 1 h), 7.28 (d, 1H), 6.61 (d, 1H), 6.59 (dd, 1H), 4.56 (d, 1H), 3.45 (m. 1H), 3.31 (m, 1H), 2.94 (s, 2H), 2.80 (s, 2H), 2.32 (s, 2H), 1.96 (m, 2H), 1.80 (m, 2H), 1.31 (m, 2H), 1.20 (m, 2H), 1.18 (t, 3H), 1.00 (s, 6H).

### EXAMPLE 99

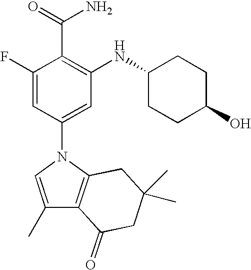

### 2-fluoro-6-(trans-4-hydroxycyclohexylamino)-4-(3,6,6-trimethyl-4-oxo-4,5,6,7-tetrahydro-1H-indol-1-yl)benzamide

4-Bromo-2-fluoro-6-(4-hydroxy-cyclohexylamino)-benzonitrile (1.74 g, 5.56 mmol), 3,6,6-trimethyl-1,5,6,7-tetrahydro-indol-4-one (1 g, 5.56 mmol), and K₂CO₃ (3.84 g, 27.8 mmol) are dissolved in 1,4-dioxane (16 mL). The reaction mixture is degassed by 3 freeze (0° C.)/pump/thaw cycles under N₂. Then, N,N′-dimethylethylenediamine (0.87 mL, 8.1 mmol) and CuI (1.6 g, 8.34 mmol) are added and the reaction is again degassed by three more freeze/pump/thaw cycles. The reaction mixture is then heated to 100° C. for 17 hours. After cooling, the reaction is filtered through Celite, washed with EtOAc (3×20 mL), concentrated and purified via Biotage column (0-50% EtOAc in Hex) to give 4-(3,6,6-trimethyl-4-oxo-4,5,6,7-tetrahydro-indol-1-yl)-2-fluoro-6-(4-hydroxy-cyclohexylamino)-benzonitrile (1.78 g; 78% yield). LC/MS: m/z=409 [M+H]⁺.

4-(3,6,6-Trimethyl-4-oxo-4,5,6,7-tetrahydro-indol-1-yl)-2-fluoro-6-(4-hydroxy-cyclohexylamino)-benzonitrile (1.78 g, 4.35 mmol) is dissolved in EtOH/DMSO (4:1, 15 mL). To this solution is added 1M NaOH (0.1 mL) and 30% H₂O₂ (0.2 mL) and the reaction is stirred for 2 hours. The reaction mixture is quenched by pouring into brine (30 mL) and extracted 3 times with EtOAc. The organic layers are dried over Na₂SO₄, concentrated, and purified using a Biotage column (elution with 0-10% MeOH/CH₂Cl₂) to afford 2-fluoro-6-(trans-4-hydroxycyclohexylamino)-4-(3,6,6-trimethyl-4-oxo-4,5,6,7-tetrahydro-1H-indol-1-yl)benzamide (0.8 g; 43% yield). LC/MS m/z=428 [M+H]. ¹H NMR (400 MHz, d₆ DMSO): δ 7.68 (d, 2H), 7.24 (d, 1H), 6.84 (s, 1H), 6.47 (t, 2H), 4.55 (d, 1H), 2.73 (s, 2H), 2.20 (d, 5H), 1.95 (br, 3H), 1.77 (d, 2H), 1.35-1.30 (m, 2H), 1.20-1.15 (m, 3H), 1.00 (s, 6H).

### EXAMPLE 172

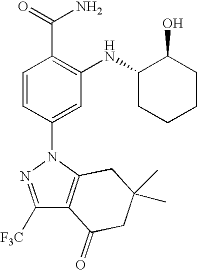

### 4-(6,6-dimethyl-4-oxo-3-(trifluoromethyl)-4,5,6,7-tetrahydro-1H-indazol-1-yl)-2-((1S,2S)-2-hydroxycyclohexylamino)benzamide

(1S,2S)-(+)-2-Benzyloxycyclohexylamine (5.00 g, 24.6 mmol), 2,4-difluorobenzonitrile (3.62 g, 21.4 mmol), and DIEA (3.73 mL, 21.4 mmol) are combined in DMSO (18 mL), and the reaction is stirred at 100° C. for 90 min. The reaction is poured into saturated NH₄Cl (100 mL) and extracted with EtOAc (2×100 mL).

The combined organic layers are dried over Na₂SO₄, concentrated, and the residue is purified by Biotage column chromatography (elution with 0-100% EtOAc in hexanes) to provide 2-((1S,2S)-2-(benzyloxy)cyclohexylamino)-4-fluorobenzonitrile (2.69 g, 39% yield) as an off-white solid. LCMS: m/z=325 [M+H]⁺.

DMF (26 mL) is added to 2-((1R,2R)-2-(benzyloxy)-cyclohexylamino)-4-fluorobenzonitrile (1.71 g, 5.27 mmol), 6,6-Dimethyl-3-trifluoromethyl-1,5,6,7-tetrahydro-indazol-4-one (1.22 g, 5.27 mmol), and NaH (60% dispersion of in mineral oil, 295 mg, 7.38 mmol). The reaction is stirred at 150° C. for 20 h then diluted with EtOAc (200 mL) and washed with H₂O (2×200 mL). The combined organic layers are dried over Na₂SO₄, concentrated, and the residue is purified by Biotage column chromatography (elution with 0-100% EtOAc in hexanes) to provide 2-((1S,2S)-2-(benzyloxy)cyclohexylamino)-4-(6,6-dimethyl-4-oxo-3-(trifluoromethyl)-4,5,6,7-tetrahydro-1H-indazol-1-yl)benzonitrile (1.29 g, 46% yield) LCMS: m/z=537 [M+H]⁺.

A solution of 2-((1S,2S)-2-(benzyloxy)cyclohexylamino)-4-(6,6-dimethyl-4-oxo-3-(trifluoromethyl)-4,5,6,7-tetrahydro-1H-indazol-1-yl)benzonitrile (1.29 g, 2.40 mmol) in DMSO (10 mL) is treated with 5M NaOH (2 mL) and 35% aqueous H₂O₂ (2 mL). The reaction is stirred for 45 min at 25° C. then diluted with EtOAc (100 mL), washed with H₂O (2×100 mL) and brine (1×100 mL). The combined organic layers are dried over Na₂SO₄, concentrated and the residue is purified using a Biotage column (elution with 0-100% EtOAc in hexanes) to yield 2-((1S,2S)-2-(benzyloxy)cyclohexylamino)-4-(6,6-dimethyl-4-oxo-3-(trifluoromethyl)-4,5,6,7-tetrahydro-1H-indazol-1-yl)benzamide (1.23 g, 92% yield) as a white solid. LC/MS: m/z=555 [M+H]⁺.

2-((1S,2S)-2-(benzyloxy)cyclohexylamino)-4-(6,6-dimethyl-4-oxo-3-(trifluoromethyl)-4,5,6,7-tetrahydro-1H-indazol-1-yl)benzamide (1.23 g, 2.22 mmol) is taken up in MeOH (5 mL) and 10% Pd/C (50 mg) is added. The reaction is stirred under H₂ at 25° C. for 24 h. The reaction mixture is filtered through celite, concentrated, and the residue is purified by chromatography (elution with 0-100% EtOAc in hexanes) to yield 4-(6,6-dimethyl-4-oxo-3-(trifluoromethyl)-4,5,6,7-tetrahydro-1H-indazol-1-yl)-2-((1S,2S)-2-hydroxycyclohexylamino)benzamide (972 mg, 55% yield) as an off-white solid. LC/MS: m/z=465 [M+H]⁺. ¹H NMR (400 MHz, d₆ DMSO): δ 8.51 (d, 1H), 7.93 (b, 1H), 7.73 (d, 1H), 7.26 (b, 1H), 6.91 (d, 1H), 6.66 (dd, 1H), 4.78 (d, 1H), 3.37-3.27 (m, 1H), 3.23-3.14 (m, 1H), 3.00-2.88 (m, 2H), 2.46-2.36 (m, 2H), 2.02-1.94 (m, 1H), 1.85-1.78 (m, 1H), 1.66-1.51 (m, 2H), 1.38-1.21 (m, 3H), 1.19-1.08 (m, 1H), 1.03 (s, 3H), 1.00 (s, 3H)

### EXAMPLE 176

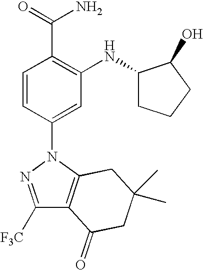

### 4-(6,6-dimethyl-4-oxo-3-(trifluoromethyl)-4,5,6,7-tetrahydro-1H-indazol-1-yl)-2-((1S,2S)-2-hydroxycyclopentylamino)benzamide

2-Bromo-4-(6,6-dimethyl-4-oxo-3-trifluoromethyl-4,5,6,7-tetrahydro-indazol-1-yl)-benzonitrile (12 mmol, 4.94 g), (1S, 2S)-2-benzyloxycyclopentylamine (17 mmol, 3.25 g) and dry toluene (30 mL) are combined and stirred under nitrogen. Pd(OAc)₂ (80 mg), 1,1′-Bis(diphenylphosphino)ferrocene (160 mg), and sodium t-butoxide (24 mmol, 2.30 g) are added, and the mixture is loared into a 110° C. bath and stirred for 15 min. The reaction mixture is extracted with EtOAc/water (600 mL/300 mL). The organic phase is dried over MgSO₄, concentrated and purified via chromatography to give the expected benzonitrile as a foam (6.39 g, quant.) The benzonitrile (12.2 mmol, 6.39 g) is combined with DMSO (2.5 mL), 95% ethanol (50 mL), and KOH (6.0 g). The reaction mixture is loared into a 50° C. oil bath and 32% hydrogen peroxide (˜4 mL) is introduced. After 20 min, the mixture is taken up in EtOAc/water (600 mL/300 mL). The organic layer is dried over MgSO₄, filtered, concentrated, and purified via chromatography to afford the desired (1S,2S)-2-(2-Benzyloxy-cyclopentylamino)-4-(6,6-dimethyl-4-oxo-3-trifluoromethyl-4,5,6,7-tetrahydro-indazol-1-yl)-benzamide as a white glass (5.71 g, 87%). LC/MS: m/z=541 [M+H]⁺.

The amide (5.71 g) is debenzylated using ca. 5 g 10% (wet) Pd/C in methanol (60 mL) under a hydrogen atmosphere. Upon complete reaction, the mixture is filtered through celite, concentrated, and the residue is purified via chromatography affording the desired 4-(6,6-dimethyl-4-oxo-3-(trifluoromethyl)-4,5,6,7-tetrahydro-1H-indazol-1-yl)-2-((1S,2S)-2-hydroxycyclopentylamino)benzamide as a white foam (4.27 g, 90%). LC/MS: m/z=451 [M+H]⁺. ¹H NMR (400 MHz, d₆ DMSO): δ 8.44 (d, 1H), 7.97 (b, 1H), 7.77 (d, 1H), 7.31 (b, 1H), 6.93 (d, 1H), 6.72 (dd, 1H), 4.88 (d, 1H), 3.88-3.82 (m, 1H), 3.55-3.48 (m, 1H), 2.96 (s, 2H), 2.46-2.37 (m, 2H), 2.21-2.08 (m, 1H), 1.85-1.57 (m, 3H), 1.56-1.47 (m, 1H), 1.42-1.32 (m, 1H), 1.03 (s, 3H), 1.01 (s, 3H).

### EXAMPLE 177

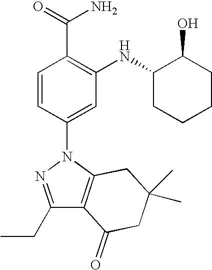

### 4-(3-ethyl-6,6-dimethyl-4-oxo-4,5,6,7-tetrahydro-1H-indazol-1-yl)-2-((1S,2S)-2-hydroxycyclohexylamino)benzamide

2-Bromo-4-(3-ethyl-6,6-dimethyl-4-oxo-4,5,6,7-tetrahydro-indazol-1-yl)-benzonitrile (0.5 mmol, 186 mg), (1S,2S)-2-(benzyloxy)cyclohexanamine (1 mmol, 205 mg) and dry toluene (1 mL) are combined and stirred under nitrogen. Pd(OAc)₂ (15 mg), 1,1′-Bis(diphenylphosphino)ferrocene (29 mg), and sodium t-butoxide (1 mmol, 96 mg) are added and the reaction mixture is irradiated in a sealed microwave vessel under nitrogen at 110° C. for 15 minutes at high absorbance. The reaction mixture is extracted with EtOAc/water (150 mL/100 mL), and the organic phase is dried over MgSO₄, concentrated and purified via chromatography to afford 2-((1S,2S)-2-(benzyloxy)cyclohexylamino)-4-(3-ethyl-6,6-dimethyl-4-oxo-4,5,6,7-tetrahydro-1H-indazol-1-yl)benzonitrile as a foam (192 mg, 77%). LCMS: m/z=497 [M+H]⁺.

2-((1S,2S)-2-(benzyloxy)cyclohexylamino)-4-(3-ethyl-6,6-dimethyl-4-oxo-4,5,6,7-tetrahydro-1H-indazol-1-yl)benzonitrile (0.38 mmol, 190 mg) is combined with DMSO (10 drops), 95% ethanol (2 mL), and KOH (206 mg), the mixture is warmed to 50° C., and 32% hydrogen peroxide (˜1 mL) is introduced. After 40 min, the mixture is taken up in EtOAc/water (150 mL/100 mL), the organic layer is dried over MgSO₄, filtered, concentrated, and purified via chromatography to afford 2-((1S,2S)-2-(benzyloxy)cyclohexylamino)-4-(3-ethyl-6,6-dimethyl-4-oxo-4,5,6,7-tetrahydro-1H-indazol-1-yl)benzamide as a white glass (153 g, 78%). LCMS: m/z=515 [M+H]⁺.

2-((1S,2S)-2-(benzyloxy)cyclohexylamino)-4-(3-ethyl-6,6-dimethyl-4-oxo-4,5,6,7-tetrahydro-1H-indazol-1-yl)benzamide (0.94 mmol, 483 mg) is debenzylated using ca. 700 mg 10% (wet) Pd/C in methanol (20 mL) under a hydrogen atmosphere. Upon complete reaction, the mixture is filtered through celite, concentrated, and the residue is purified via chromatography to yield 4-(3-ethyl-6,6-dimethyl-4-oxo-4,5,6,7-tetrahydro-1H-indazol-1-yl)-2-((1S,2S)-2-hydroxycyclohexylamino)benzamide as a white foam (361 mg, 90%). LCMS: m/z=425 [M+H]⁻. ¹H NMR (400 MHz, d₆ DMSO): δ 8.50 (d, 1H), 7.86 (b, 1H), 7.69 (d, 1H), 7.18 (b, 1H), 6.84 (d, 1H), 6.63 (dd, 1H), 4.77 (d, 1H), 3.38-3.29 (m, 1H), 3.23-3.12 (m, 1H), 2.98-2.83 (m, 2H), 2.83-2.76 (m, 2H), 2.38-2.25 (m, 2H), 2.04-1.95 (m, 1H), 1.86-1.78 (m, 1H), 1.66-1.52 (m, 2H), 1.38-1.22 (m, 3H), 1.22-1.11 (m, 4H), 1.01 (s, 3H), 0.98 (s, 3H).

### EXAMPLE 180

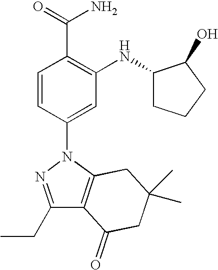

### 4-(3-ethyl-6,6-dimethyl-4-oxo-4,5,6,7-tetrahydro-1H-indazol-1-yl)-2-((1S,2S)-2-hydroxycyclopentylamino)benzamide

2-Bromo-4-(3-ethyl-6,6-dimethyl-4-oxo-4,5,6,7-tetrahydro-indazol-1-yl)-benzonitrile (1.5 mmol, 558 mg), (1S, 2S)-(+)-2-benzyloxycyclopentylamine (3 mmol, 573 mg) and dry toluene (4 mL) are combined and stirred under nitrogen, and Pd(OAc)₂ (29 mg), 1,1′-Bis(diphenylphosphino)ferrocene (58 mg), and sodium t-butoxide (3 mmol, 288 mg) are added and the reaction mixture is irradiated in a sealed microwave vessel under nitrogen at 110° C. for 15 minutes at high absorbance. The reaction mixture is extracted with EtOAc/water (2:1, 300 mL), the organic phase is dried over MgSO₄, concentrated and purified via chromatography to afford 2-((1S,2S)-2-(benzyloxy)cyclopentylamino)-4-(3-ethyl-6,6-dimethyl-4-oxo-4,5,6,7-tetrahydro-1H-indazol-1-yl)benzonitrile (573 mg, 67%) as a glass. LCMS: m/z=483 [M+H].

2-((1S,2S)-2-(benzyloxy)cyclopentylamino)-4-(3-ethyl-6,6-dimethyl-4-oxo-4,5,6,7-tetrahydro-1H-indazol-1-yl)benzonitrile (1.18 mmol, 570 mg) is combined with DMSO (15 drops), 95% ethanol (4 mL), and KOH (636 mg), and the reaction mixture is warmed to 45° C., and 32% hydrogen peroxide (˜1 mL) is introduced. After 30 min, the mixture is taken up in EtOAc/water (2:1, 600 mL), and the organic layer is dried over MgSO₄, filtered, concentrated, and purified via chromatography to give 2-((1S,2S)-2-(benzyloxy)cyclopentylamino)-4-(3-ethyl-6,6-dimethyl-4-oxo-4,5,6,7-tetrahydro-1H-indazol-1-yl)benzamide (513 g, 87%) as a white foam. LCMS: m/z=501 [M+H]⁺.

2-((1S,2S)-2-(benzyloxy)cyclopentylamino)-4-(3-ethyl-6,6-dimethyl-4-oxo-4,5,6,7-tetrahydro-1H-indazol-1-yl)benzamide (1.02 mmol, 510 mg) is debenzylated using ca. 600 mg 10% Pd/C (wet) in methanol (40 mL) under a hydrogen atmosphere. Upon complete reaction, the mixture is filtered through celite, concentrated, and the residue is purified via chromatography, affording 4-(3-ethyl-6,6-dimethyl-4-oxo-4,5,6,7-tetrahydro-1H-indazol-1-yl)-2-((1S,2S)-2-hydroxycyclopentylamino)benzamide (235 mg, 56%) as a white foam. LCMS: m/z=411 [M+H]⁺. ¹H NMR (400 MHz, d₆ DMSO): δ 7.94 (b, 1H), 7.39 (b, 1H), 7.37 (b, 1H), 7.18 (s, 1H), 4.81 (d, 1H), 3.81-3.77 (m, 1H), 3.62-3.54 (m, 1H), 2.81 (q, 2H), 2.64-2.54 (m, 2H), 2.29 (s, 2H), 2.14-2.05 (m, 1H), 1.74-1.63 (m, 2H), 1.56-1.42 (m, 2H), 1.33-1.23 (m, 1H), 1.18 (t, 3H), 0.98 (s, 6H).

### EXAMPLE 181

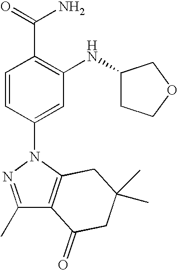

### (S)-2-(tetrahydrofuran-3-ylamino)-4-(3,6,6-trimethyl-4-oxo-4,5,6,7-tetrahydro-1H-indazol-1-yl)benzamide

To a solution of 2-fluoro-4-(3,6,6-trimethyl-4-oxo-4,5,6,7-tetrahydro-indazol-1-yl)-benzonitrile (491 mg, 1.65 mmol) in DMSO (0.7 mL), (S)-3-aminotetrahydrofuran hydrochloride (247 mg, 1.98 mmol) and DIEA (0.86 mL, 4.95 mmol) are added, and the reaction is stirred at 90° C. for 13 hours. The reaction mixture is then diluted with EtOAc (50 mL), washed with H₂O (2×50 mL) and brine (50 mL), and the organic layers are dried over Na₂SO₄ and concentrated. Purification of the residue using a Biotage column (elution with 0-100% EtOAc in hexanes) yields (S)-2-(tetrahydrofuran-3-ylamino)-4-(3,6,6-trimethyl-4-oxo-4,5,6,7-tetrahydro-1H-indazol-1-yl)benzonitrile (197 mg, 33% yield) as a light yellow solid. LCMS: m/z=365 [M+H]⁺.

A solution of (S)-2-(tetrahydrofuran-3-ylamino)-4-(3,6,6-trimethyl-4-oxo-4,5,6,7-tetrahydro-1H-indazol-1-yl)benzonitrile (197 mg, 0.54 mmol) in EtOH/DMSO (4:1, 0.5 mL) is treated with 1M NaOH (20 drops) and 35% aqueous H₂O₂ (20 drops), and the reaction is stirred for 13 h at 25° C. The reaction mixture is diluted with EtOAc (10 mL), washed with H₂O (2×10 mL) and brine (10 mL), and the combined organic layers are dried over Na₂SO₄ and concentrated under reduced pressure. Purification of the residue using a Biotage column (elution with 0-100% EtOAc in hexanes) affords (S)-2-(tetrahydrofuran-3-ylamino)-4-(3,6,6-trimethyl-4-oxo-4,5,6,7-tetrahydro-1H-indazol-1-yl)benzamide (160 mg, 77% yield) as a white solid. LCMS: m/z=383 [M+H]⁺. ¹H NMR (400 MHz, d₆ DMSO): δ 8.55 (d, 1H), 7.94 (b, 1H), 7.75 (d, 1H), 7.28 (b, 1H), 6.77-6.70 (m, 2H), 4.17-4.09 (m, 1H), 3.91-3.69 (m, 3H), 3.57-3.52 (m, 1H), 2.92 (s, 2H), 2.38 (s, 3H), 2.31 (s, 2H), 2.29-1.19 (m, 1H), 1.79-1.70 (m, 1H), 1.00 (s, 6H).

### EXAMPLE 182

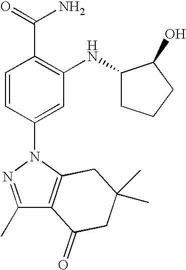

### 2-((1S,2S)-2-hydroxycyclopentylamino)-4-(3,6,6-trimethyl-4-oxo-4,5,6,7-tetrahydro-1H-indazol-1-yl)benzamide

To a solution of 200 mg (0.67 mmol) of 2-fluoro-4-(3,6,6-trimethyl-4-oxo-4,5,6,7-tetrahydro-indazol-1-yl)-benzonitrile in DMSO (0.5 mL), (1S,2S)-(+)-2-benzyloxycyclopentylamine (154 mg, 0.81 mmol) and DIEA (0.35 mL, 2.0 mmol) are added, and the reaction is stirred at 100° C. for 16 hours. Then, the mixture is diluted with EtOAc (10 mL), washed with H₂O (2×10 mL) and brine (1×10 mL), and the organic layers are dried over Na₂SO₄ and concentrated. Purification of the residue using a Biotage column (elution with 0-50% EtOAc in hexanes) yields of the 2-((1S,2S)-2-(benzyloxy)cyclopentylamino)-4-(3,6,6-trimethyl-4-oxo-4,5,6,7-tetrahydro-1H-indazol-1-yl)benzonitrile (269 mg, 85% yield) as an off-white solid. LCMS: m/z=469 [M+H]⁺.

A solution of 2-((1S,2S)-2-(benzyloxy)cyclopentylamino)-4-(3,6,6-trimethyl-4-oxo-4,5,6,7-tetrahydro-1H-indazol-1-yl)benzonitrile (262 mg, 0.56 mmol) in EtOH/DMSO (4:1, 5 mL) is treated with 1M NaOH (1 mL) and 35% aqueous H₂O₂ (1 mL), and the reaction is stirred for 15 min at 25° C. The reaction mixture is then diluted with EtOAc (50 mL), washed with H₂O (2×50 mL) and brine (1×10 mL), and the combined organic layers are dried over Na₂SO₄ and concentrated under reduced pressure. Purification of the residue using a Biotage column (elution with 0-100% EtOAc in hexanes) gives 2-((1S,2S)-2-(benzyloxy)cyclopentylamino)-4-(3,6,6-trimethyl-4-oxo-4,5,6,7-tetrahydro-1H-indazol-1-yl)benzamide (112 mg, 41% yield) as a white solid. LCMS: m/z=487 [M+H]⁺.

2-((1S,2S)-2-(benzyloxy)cyclopentylamino)-4-(3,6,6-trimethyl-4-oxo-4,5,6,7-tetrahydro-1H-indazol-1-yl)benzamide (112 mg, 0.23 mmol) is taken up in MeOH (10 mL), 10% Pd/C (20 mg) is added, and the reaction is stirred under H₂ at 25° C. for 2 h. The mixture is filtered through celite, concentrated under reduced pressure, and the residue is purified by chromatography (elution with 0-100% EtOAc in hexanes) to yield 2-((1S,2S)-2-hydroxycyclopentylamino)-4-(3,6,6-trimethyl-4-oxo-4,5,6,7-tetrahydro-1H-indazol-1-yl)benzamide (37 mg, 41% yield) as a white solid. LCMS: m/z=397 [M+H]⁺. ¹H NMR (400 MHz, d₆ DMSO) δ 8.41 (d, 1H), 7.90 (b, 1H), 7.71 (d, 1H), 7.21 (b, 1H), 6.87 (d, 1H), 6.67 (dd, 1H), 4,87 (d, 1H), 3.87-3.81 (m, 1H), 3.54-3.47 (m, 1H), 2.94-2.91 (m, 2H), 2.38 (s, 3H), 2.34-2.29 (m, 2H), 2.20-2.10 (m, 1H), 1.84-1.58 (m, 3H), 1.56-1.48 (m, 1H), 1.42-1.31 (m, 1H), 1.01 (s, 3H), 0.99 (s, 3H).

### EXAMPLE 183

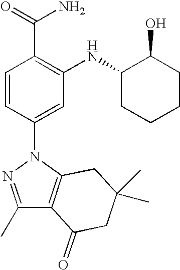

### 2-((1S,2S)-2-hydroxycyclohexylamino)-4-(3,6,6-trimethyl-4-oxo-4,5,6,7-tetrahydro-1H-indazol-1-yl)benzamide

To a solution of 2-fluoro-4-(3,6,6-trimethyl-4-oxo-4,5,6,7-tetrahydro-indazol-1-yl)-benzonitrile (200 mg, 0.67 mmol) in DMSO (0.5 mL) (1S,2S)-(+)-2-benzyloxycyclohexylamine (166 mg, 0.81 mmol) and DIEA (0.35 mL, 2.0 mmol) are added, and the reaction is stirred at 100° C. for 16 hours. The reaction mixture is then diluted with EtOAc (10 mL), washed with H₂O (2×10 mL) and brine (1×10 mL), and the organic layers are dried over Na₂SO₄ and concentrated. Purification of the residue using a Biotage column (elution with 0-50% EtOAc in hexanes) affords 2-((1S,2S)-2-(benzyloxy)cyclohexylamino)-4-(3,6,6-trimethyl-4-oxo-4,5,6,7-tetrahydro-1H-indazol-1-yl)benzonitrile (256 mg, 79% yield) as an off-white solid. LCMS: m/z=483 [M+H]⁺.

A solution of 2-((1S,2S)-2-(benzyloxy)cyclohexylamino)-4-(3,6,6-trimethyl-4-oxo-4,5,6,7-tetrahydro-1H-indazol-1-yl)benzonitrile (239 mg, 0.50 mmol) in EtOH/DMSO (4:1, 5 mL) is treated with 1M NaOH (1 mL) and 35% aqueous H₂O₂ (1 mL). The reaction is stirred for 60 min at 25° C. then diluted with EtOAc (50 mL), washed with H₂O (2×50 mL) and brine (1×10 mL). The combined organic layers are dried over Na₂SO₄ and concentrated under reduced pressure. Purification of the residue using a Biotage column (elution with 0-100% EtOAc in hexanes) yields 2-((1S,2S)-2-(benzyloxy)cyclohexylamino)-4-(3,6,6-trimethyl-4-oxo-4,5,6,7-tetrahydro-1H-indazol-1-yl)benzamide (95 mg, 38% yield) as a white solid. LC/MS: m/z=501 [M+H]⁺.

2-((1S,2S)-2-(benzyloxy)cyclohexylamino)-4-(3,6,6-trimethyl-4-oxo-4,5,6,7-tetrahydro-1H-indazol-1-yl)benzamide (95 mg, 0.19 mmol) is taken up in MeOH (20 mL), 10% Pd/C (20 mg) is added, and the reaction is stirred under H₂ at 25° C. for 2 h. The mixture is then filtered through celite, concentrated under reduced pressure, and the residue is purified by chromatography (elution with 0-100% EtOAc in hexanes) to yield 2-((1S,2S)-2-hydroxycyclohexylamino)-4-(3,6,6-trimethyl-4-oxo-4,5,6,7-tetrahydro-1H-indazol-1-yl)benzamide (43 mg, 55% yield) as a white solid. LCMS: m/z=411 [M+H]⁺. ¹H NMR (400 MHz, d₆ DMSO) δ 8.50 (d, 1H), 7.86 (b, 1H), 7.69 (d, 1H), 7.18 (b, 1H), 6.84 (d, 1H), 6.61 (dd, 1H), 4.76 (d, 1H), 3.37-3.26 (m, 1H), 3.23-3.13 (m, 1H), 2.98-2.82 (m, 2H), 2.38 (s, 3H), 2.37-2.25 (m, 2H), 2.03-1.94 (m, 1H), 1.85-1.78 (m, 1H), 1.66-1.52 (m, 2H), 1.38-1.21 (m, 3H), 1.20-1.09 (m, 1H), 1.01 (s, 3H), 0.98 (s, 3H).

### EXAMPLE 189

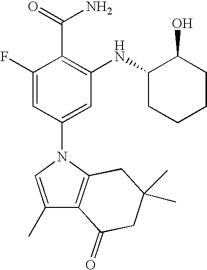

### 2-fluoro-6-((1S,2S)-2-hydroxycyclohexylamino)-4-(3,6,6-trimethyl-4-oxo-4,5,6,7-tetrahydro-1H-indol-1-yl)benzamide

4-Bromo-2,6-difluoro-bezonitrile (2 g, 1 eq), (1S,2S)-2-benzyloxy-cyclohexylamine (2.15 g, 1.1 eq), and DIPEA (1.76 mL, 1.1 eq) are dissolved in DMSO (10 mL) and stirred at room temperature overnight. Then the reaction mixture is poured into saturated aqueous NH₄Cl (250 mL), extracted with EtOAc (3×100 mL), dried over Na₂SO₄, filtered, and concentrated to give 2-((1S,2S)-2-(benzyloxy)cyclohexylamino)-4-bromo-6-fluoro-benzonitrile (3.4 g, 92% yield). LCMS: m/z=403, 405 [M+H]⁺.

To a stirred solution of 2-((1S,2S)-2-(benzyloxy)cyclohexylamino)-4-bromo-6-fluorobenzonitrile (4 g) and 3,6,6-trimethyl-1,5,6,7-tetrahydro-indol-4-one (1.48 g) in 1,4-dioxane (70 mL), K₂CO₃ (7 g) is added, and the suspension is frozen at ice-bath and degassed three times by pump/backfill N₂. Then, CuI (2.9 g) and N,N′-dimethylethylenediamine (1.58 mL) are added, the reaction mixture is degassed again three times and stirred at 100° C. overnight. The reaction mixture is filtered through celite, washed with EtOAc, and concentrated to give crude 2-((1S,2S)-2-(benzyloxy)cyclohexylamino)-6-fluoro-4-(3,6,6-trimethyl-4-oxo-4,5,6,7-tetrahydro-1H-indol-1-yl)benzonitrile. LCMS: m/z=500 [M+1]⁺.

2-((1S,2S)-2-(benzyloxy)cyclohexylamino)-6-fluoro-4-(3,6,6-trimethyl-4-oxo-4,5,6,7-tetrahydro-1H-indol-1-yl)benzonitrile is dissolved in EtOH/DMSO (4:1, 100 mL), 1N NaOH (20 mL) and H₂O₂ (3 mL) are added, and the reaction mixture is stirred at room temperature overnight. The reaction mixture is then concentrated, poured into saturated aqueous NH₄Cl (250 mL), extracted with EtOAc (3×150 mL), dried over Na₂SO₄, filtered, concentrated, and purified by Biotage chromatography (elution with 30% EA in hexane) to give 2-((1S,2S)-2-(benzyloxy)cyclohexylamino)-6-fluoro-4-(3,6,6-trimethyl-4-oxo-4,5,6,7-tetrahydro-1H-indol-1-yl)benzamide (2.67 g, 64% over 2 steps). LCMS: m/z=518 [M+H]⁺.

2-((1S,2S)-2-(benzyloxy)cyclohexylamino)-6-fluoro-4-(3,6,6-trimethyl-4-oxo-4,5,6,7-tetrahydro-1H-indol-1-yl)benzamide (2.67 g), 10% Pd/C (267 mg) and a few drops of TFA are dissolved in MeOH (30 mL) and hydrogenated at 50 psi overnight. Then the pH of the reaction mixture is adjusted by TEA to 8˜9, and filtered through celite to remove Pd/C, concentrated and purified by Biotage chromatography (elution with 50% EA in hexane) to give 2-fluoro-6-((1S,2S)-2-hydroxycyclohexylamino)-4-(3,6,6-trimethyl-4-oxo-4,5,6,7-tetrahydro-1H-indol-1-yl)benzamide (1.54 g, 70% yield). LCMS: m/z=428 [M+H]⁺. ¹H NMR (400 MHz, d₆ DMSO): δ 7.65 (d, 2H), 7.42 (d, 1H), 6.81 (s, 1H), 6.52 (s, 1H), 6.42 (d, 1H), 4.80 (d, 1H), 3.16 (br, 1H), 2.74 (q, 2H), 2.23 (m, 5H), 1.97 (br, 2H), 1.80 (br, 1H), 1.60 (br, 1H), 1.51 (br, 1H), 1.39-1.20 (m, 2H), 1.20-1.15 (m, 2H), 1.00-0.9 (d, 6H).

### EXAMPLE 192

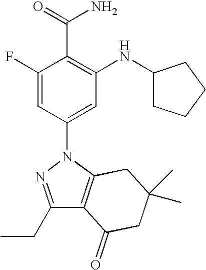

### 2-(cyclopentylamino)-4-(3-ethyl-6,6-dimethyl-4-oxo-4,5,6,7-tetrahydro-1H-indazol-1-yl)-6-fluorobenzamide

2,6-Difluoro-4-(3,6,6-trimethyl-4-oxo-4,5,6,7-tetrahydro-indazol-1-yl)-benzonitrile (1.5 mmol, 494 mg), aminocyclopentane (1.5 mmol, 0.15 mL), diisopropylethylamine (1.6 mmol, 0.29 mL) and DMSO (3 mL) are combined and heated by microwave irradiation in a sealed vessel to 150° C. for 20 minutes at high absorbance. The crude product is extracted with EtOAc/water (2:1, 300 mL), the organic phase is dried over MgSO₄, and filtered through a silica plug to afford the crude 2-cyclopentylamino-4-(3-ethyl-6,6-dimethyl-4-oxo-4,5,6,7-tetrahydro-indazol-1-yl)-6-fluorobenzonitrile as a white solid (281 mg, 47%). LCMS: m/z=395 [M+H]⁺.

2-Cyclopentylamino-4-(3-ethyl-6,6-dimethyl-4-oxo-4,5,6,7-tetrahydro-indazol-1-yl)-6-fluorobenzonitrile (280 mg) is combined with DMSO (0.2 mL), 95% ethanol (1 mL), and KOH (330 mg). The reaction mixture is warmed to 50° C. and 32% hydrogen peroxide (˜0.2 mL) is introduced. After 1 h, the mixture is taken up in EtOAc/water (300 mL/200 mL), and the organic layer is dried over MgSO₄, filtered, concentrated, and purified via chromatography to afford 2-(cyclopentylamino)-4-(3-ethyl-6,6-dimethyl-4-oxo-4,5,6,7-tetrahydro-1H-indazol-1-yl)-6-fluorobenzamide as a white glass (279 mg, 96%). LCMS m/z=413 [M+H]⁺. ¹H NMR (400 MHz, d₆ DMSO): δ 7.71 (br s, 1H), 7.66 (br s, 1H), 7.40 (d, 1H), 6.61 (d, 1H), 6.59 (s, 1H), 3.81 (m, 1H), 2.94 (s, 2H), 2.80 (q, 2H), 2.32 (s, 2H), 1.97 (m, 2H), 1.70-1.52 (m, 4H), 1.42 (m, 2H), 1.81 (t, 3H), 1.00 (s, 6H).

### EXAMPLE 193

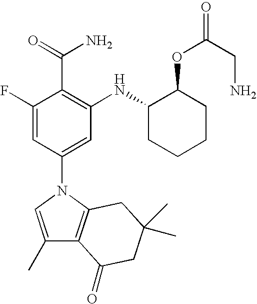

### (1S,2S)-2-(2-carbamoyl-3-fluoro-5-(3,6,6-trimethyl-4-oxo-4,5,6,7-tetrahydro-1H-indol-1-yl)phenylamino)cyclohexyl 2-aminoacetate

2-fluoro-6-((1S,2S)-2-hydroxycyclohexylamino)-4-(3,6,6-trimethyl-4-oxo-4,5,6,7-tetrahydro-1H-indol-1-yl)benzamide (350 mg, 1 eq), Boc-glycine (502 mg, 3.5 eq), EDC (549 mg, 3.5 eq), and DMAP (21 mg, 0.2 eq) are dissolved in dichloromethane (20 mL) and stirred at room temperature for 18 h. The mixture is concentrated and purified by Biotage chromatography (elution with 40% EA in hexane) to give (1S,2S)-2-(2-carbamoyl-3-fluoro-5-(3,6,6-trimethyl-4-oxo-4,5,6,7-tetrahydro-1H-indol-1-yl)phenylamino)cyclohexyl 2-(tert-butoxycarbonylamino)acetate (479 mg, 100% yield). LCMS: m/z=585 [M+H]⁺.

(1S,2S)-2-(2-carbamoyl-3-fluoro-5-(3,6,6-trimethyl-4-oxo-4,5,6,7-tetrahydro-1H-indol-1-yl)phenylamino)cyclohexyl 2-(tert-butoxycarbonylamino)acetate (479 mg, 1 eq) in acetic acid (3 mL) is heated to dissolution at 60° C. After 10 minutes of stirring, aqueous 12N HCl (80 μL, 1.15 eq) is added, and the reaction mixture is heated at 60° C. and stirred at this temperature for 4 h. The reaction mixture is then concentrated to 1.5 mL and MTBE (20 mL) is added and stirred at ambient temperature for 14 h. The resulting solids are filtered, washed with MTBE (2 mL), and dried completely to afford (1S,2S)-2-(2-carbamoyl-3-fluoro-5-(3,6,6-trimethyl-4-oxo-4,5,6,7-tetrahydro-1H-indol-1-yl)phenylamino)cyclohexyl 2-aminoacetate (426 mg, 100% yield). LCMS: m/z=485 [M+H]⁺. ¹H NMR (400 MHz, d₆ DMSO): δ 8.37 (br, 3H), 7.71 (s, 2H), 6.82 (s, 1H), 6.64 (s, 1H), 6.52 (d, 1H), 4.82 (t, 1H), 3.72 (br, 2H), 3.05 (s, 1H), 2.73 (s, 2H), 2.21 (d, 3H), 1.92 (s, 3H), 1.66 (br, 1H), 1.53 (br, 2H), 1.42 (br, 3H), 1.10 (s, 2H), 1.00 (s, 6H).

### EXAMPLE 194

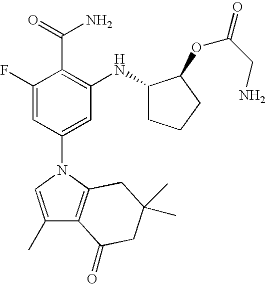

### (1S,2S)-2-(2-carbamoyl-3-fluoro-5-(3,6,6-trimethyl-4-oxo-4,5,6,7-tetrahydro-1H-indol-1-yl)phenylamino)cyclopentyl 2-aminoacetate

4-Bromo-2,6-difluoro-bezonitrile (1 g), ((1S,2S)-2-(benzyloxy)cyclopentanamine (964 mg, 1.1 eq), and DIPEA (0.882 mL, 1.1 eq) are stirred in DMSO (5 mL) at 120° C. for 2 h. The reaction mixture is poured to saturated aqueous NH₄Cl (150 mL), extracted with EtOAc (3×100 mL), dried over Na₂SO₄, filtered, and concentrated to give 2-((1S,2S)-2-(benzyloxy)-cyclopentylamino)-4-bromo-6-fluorobenzonitrile (1.8 g, 100% yield). LCMS: m/z=389, 391 [M+H]⁺.

To a stirred solution of 2-((1S,2S)-2-(benzyloxy)cyclopentylamino)-4-bromo-6-fluorobenzonitrile (1.48 g) and 3,6,6-trimethyl-1,5,6,7-tetrahydro-indol-4-one (0.56 g) in 1,4-dioxane (15 mL), K₂CO₃ (2.64 g) is added. The suspension is frozen at ice-bath and degassed three times by pump/backfill with N₂. CuI (1.1 g) and N,N′-dimethylethylenediamine (0.6 ml) are then added, and the reaction mixture is degassed again three times. The sealed reaction mixture is stirred at 100° C. overnight. The reaction mixture is then filtered through, washed with EtOAc, and concentrated to give crude 2-((1S,2S)-2-(benzyloxy)cyclopentylamino)-6-fluoro-4-(3,6,6-trimethyl-4-oxo-4,5,6,7-tetrahydro-1H-indol-1-yl)benzonitrile (LCMS: m/z=486 [M+H]⁺). This benzonitrile is dissolved in EtOH/DMSO (4:1, 50 mL), 1N NaOH (10 mL) and H₂O₂ (3 mL) are added, and the reaction is stirred at room temperature for 4 h. The mixture is concentrated, poured into saturated aqueous NH₄Cl (250 mL), extracted with EtOAc (3×150 mL), dried over Na₂SO₄, filtered, and concentrated. Purification using Biotage chromatography (elution with 30% EtOAc in hexane) yields 2-((1S,2S)-2-(benzyloxy)cyclopentylamino)-6-fluoro-4-(3,6,6-trimethyl-4-oxo-4,5,6,7-tetrahydro-1H-indol-1-yl)benzamide (1.2 g, 63% over 2 steps). LCMS: m/z=504 [M+H]⁺.

2-((1S,2S)-2-(benzyloxy)cyclopentylamino)-6-fluoro-4-(3,6,6-trimethyl-4-oxo-4,5,6,7-tetrahydro-1H-indol-1-yl)benzamide (1.2 g), 10% Pd/C (120 mg) and a few drops of TFA are dissolved in MeOH (30 mL) and hydrogenated at 55 psi overnight. The pH of the reaction mixture is adjusted by TEA to 8˜9, filtered through celite to remove Pd/C, concentrated and purified by Biotage chromatography (elution with 50% EA in hexane) to give 2-fluoro-6-((1S,2S)-2-hydroxycyclopentylamino)-4-(3,6,6-trimethyl-4-oxo-4,5,6,7-tetrahydro-1H-indol-1-yl)benzamide, Example 43 (762 mg, 77% yield). LCMS: m/z=414 [M+H]⁺.

2-fluoro-6-((1S,2S)-2-hydroxycyclopentylamino)-4-(3,6,6-trimethyl-4-oxo-4,5,6,7-tetrahydro-1H-indol-1-yl)benzamide (300 mg, 1 eq), Boc-glycine (453 mg, 2 eq), EDC (494.5 mg, 2 eq), and DMAP (18 mg, 0.1 eq) are dissolved in dichloromethane (20 mL) and stirred at room temperature for 18 h. The mixture is concentrated and purified by Biotage chromatography (elution with 40% EA in hexane) to give (1S,2S)-2-(2-carbamoyl-3-fluoro-5-(3,6,6-trimethyl-4-oxo-4,5,6,7-tetrahydro-1H-indol-1-yl)phenylamino)cyclopentyl 2-(tert-butoxycarbonylamino)acetate (414 mg, 98% yield). LCMS: m/z=571 [M+H]⁺.

1S,2S)-2-(2-carbamoyl-3-fluoro-5-(3,6,6-trimethyl-4-oxo-4,5,6,7-tetrahydro-1H-indol-1-yl)phenylamino)cyclopentyl 2-(tert-butoxycarbonylamino)acetate (414 mg, 1 eq) in acetic acid (3 mL) is heated to dissolution at 60° C. After 10 minutes of stirring, aqueous 12 N HCl (71 μL, 1.15 eq) is added. The reaction mixture is heated at 60° C. and stirred at this temperature for 4 h. The reaction mixture is concentrated to 1.5 mL and MTBE (20 mL) is added and stirred at ambient temperature for 14 h. The resulting solids are filtered, washed with MTBE (2 mL), and dried to afford (1S,2S)-2-(2-carbamoyl-3-fluoro-5-(3,6,6-trimethyl-4-oxo-4,5,6,7-tetrahydro-1H-indol-1-yl)phenylamino)cyclopentyl 2-aminoacetate (260 mg, 71% yield). LCMS: m/z=471 [M+H]⁺. ¹H NMR (400 MHz, d₆ DMSO): δ 8.40 (br, 3H), 7.73 (d, 2H), 6.85 (s, 1H), 6.65 (s, 1H), 6.55 (d, 1H), 5.03 (br, 1H), 3.91 (br, 1H), 3.75 (br, 2H), 3.05 (s, 1H), 2.70 (q, 1H), 2.25 (d, 3H), 2.01 (m, 1H), 1.89 (s, 1H), 1.75 (m, 3H), 1.51 (br, 1H), 1.10 (s, 3H), 1.00 (s, 6H).

### EXAMPLE 196-1

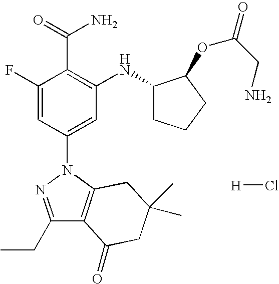

### (1S,2S)-2-(2-carbamoyl-5-(3-ethyl-6,6-dimethyl-4-oxo-4,5,6,7-tetrahydro-1H-indazol-1-yl)-3-fluorophenylamino)cyclopentyl 2-aminoacetate hydrochloride

4-(3-Ethyl-6,6-dimethyl-4-oxo-4,5,6,7-tetrahydro-indazol-1-yl)-2-fluoro-6-(2-hydroxy-cyclopentylamino)-benzamide (600 mg, 1.4 mmol), EDC (537 mg, 2.8 mmol), BOC-glycine (490 mg, 1.4 mmol) and DMAP (171 mg, 1.4 mmol) are dissolved in methylene chloride (12 mL) and stirred at room temperature under nitrogen. After 1 h, additional EDC (200 mg) is added and the mixture stirred for 16 h. The mixture is concentrated and purified via chromatography (silica, 10 to 80% EtOAc in hexanes) to give the intermediate, (1S,2S)-2-(2-carbamoyl-5-(3-ethyl-6,6-dimethyl-4-oxo-4,5,6,7-tetrahydro-1H-indazol-1-yl)-3-fluorophenylamino)-cyclopentyl 2-(tert-butoxycarbonylamino)acetate.

The intermediate is dissolved in 1,4-dioxane (8 mL), treated with 4M HCl in 1,4-dioxane (1.7 mL), heated to 60° C., and stirred for 16 h. The solution is cooled and added dropwise to a rapidly stirring solution of MTBE to effect solid formation. After stirring for 1 h, the solution is filtered off under nitrogen and left to dry under a nitrogen stream for 16 h to give (1S,2S)-2-(2-carbamoyl-5-(3-ethyl-6,6-dimethyl-4-oxo-4,5,6,7-tetrahydro-1H-indazol-1-yl)-3-fluorophenylamino)cyclopentyl 2-aminoacetate hydrochloride (567 mg, 77%) as a tan solid. LC/MS: m/z=486 [M+H]⁺. ¹H NMR (400 MHz, d₆ DMSO): δ 8.22 (br s, 1H), 7.75 (d, 1H), 7.46 (br s, 1H), 6.80 (d, 1H), 6.66 (dd, 1H), 5.01 (m, 1H), 3.91 (m, 1H), 3.78 (m, 2H), 3.66 (m, 1H), 2.93 (dd, 2H), 2.80, (q, 2H), 2.33 (s, 2H), 2.16 (m, 1H), 2.07 (m, 1H), 1.65-1.81 (m, 3H), 1.52 (m, 1H), 1.18 (t, 3H), 1.00 (2 s, 6H).

### EXAMPLE 197-1

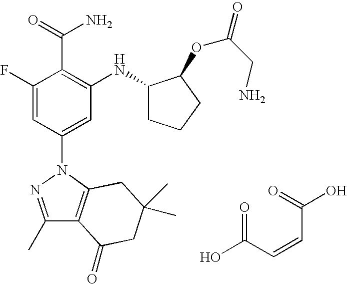

### (1S,2S)-2-(2-carbamoyl-3-fluoro-5-(3,6,6-trimethyl-4-oxo-4,5,6,7-tetrahydro-1H-indazol-1-yl)phenylamino)cyclopentyl 2-aminoacetate maleate

2-Fluoro-6-((1S,2S)-2-hydroxy-cyclopentylamino)-4-(3,6,6-trimethyl-4-oxo-4,5,6,7-tetrahydro-indazol-1-yl)-benzamide (20 g, 48.25 mmol), EDC (18.5 g, 96.5 mmol), BOC-glycine (16.9 g, 96.5 mmol) and DMAP (5.9 g, 48.25 mmol) are dissolved in methylene chloride (250 mL) and stirred for 16 h. The mixture is treated with water (250 mL) and the organic layer is washed with saturated NaHCO₃ solution (250 mL), 1N HCl (250 mL) and saturated NaCl solution (250 mL). The organic layer is dried over MgSO₄, filtered, concentrated, and purified via chromatography (30 to 100% EtOAc in hexanes). Recrystallization from EtOAc and hexanes gives (1S,2S)-2-(2-carbamoyl-3-fluoro-5-(3,6,6-trimethyl-4-oxo-4,5,6,7-tetrahydro-1H-indazol-1-yl)phenylamino)cyclopentyl 2-(tert-butoxycarbonylamino)acetate as a white powder. LCMS m/z=572 [M+H]⁺.

(1S,2S)-2-(2-carbamoyl-3-fluoro-5-(3,6,6-trimethyl-4-oxo-4,5,6,7-tetrahydro-1H-indazol-1-yl)phenylamino)cyclopentyl 2-(tert-butoxycarbonylamino)acetate (15 g, 26 mmol) is dissolved in 1,4-dioxane (100 mL), treated with methanesulfonic acid (3.03 g, 31.5 mmol) and heated to 100° C. for 4 h. The mixture is cooled and concentrated to a thick oil, which is partitioned between EtOAc (400 mL) and saturated NaHCO₃ solution (150 mL). The organic layer is washed with brine (150 mL), dried over MgSO₄, filtered and concentrated to a foam and put under vacuum for 1 h to give an amine, (1S,2S)-2-(2-carbamoyl-3-fluoro-5-(3,6,6-trimethyl-4-oxo-4,5,6,7-tetrahydro-1H-indazol-1-yl)phenylamino)cyclopentyl 2-aminoacetate (12.9 g, 86%) as a sticky solid (Example 197). The amine (125 mg, 0.26 mmol) and maleic acid (30.8 mg, 0.26 mmol) are dissolved in EtOH (5 mL) and concentrated to a glass. The glass is stirred for 20 h with EtOAc (5 mL) to give a crystalline solid. The solid is filtered off to give (1S,2S)-2-(2-carbamoyl-3-fluoro-5-(3,6,6-trimethyl-4-oxo-4,5,6,7-tetrahydro-1H-indazol-1-yl)phenylamino)cyclopentyl 2-aminoacetate maleate (101 mg) as a white crystalline solid. LCMS m/z=472 [M+H]⁺. ¹H NMR (400 MHz, d₆ DMSO): δ 8.12 (br s, 3H), 7.76 (2 s, 2H), 7.49 (d, 1H), 6.83 (d, 1H), 6.68 (dd, 1H), 6.01 (s, 2H), 5.03 (m, 1H), 3.93 (m, 1H), 3.86 (2 d, 2H), 2.97 (d, 1H), 2.91 (d, 1H), 2.41 (s, 3H), 2.33 (2 d, 2H), 2.14 (m, 2H), 1.78 (m, 2H), 1.70 (m, 1H), 1.55 (m, 1H), 1.03 (s, 3H), 1.02 (s, 3H).

### EXAMPLE 199

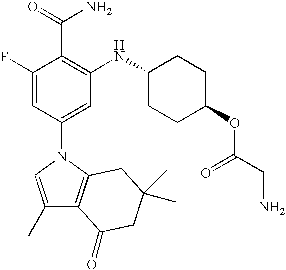

### (trans)-4-(2-carbamoyl-3-fluoro-5-(3,6,6-trimethyl-4-oxo-4,5,6,7-tetrahydro-1H-indol-1-yl)phenylamino)cyclohexyl 2-aminoacetate

To a solution of 4-(3,6,6-trimethyl-4-oxo-4,5,6,7-tetrahydro-1H-indol-1-yl)-2-fluoro-6-(trans-4-hydroxycyclohexylamino)benzamide (0.109 g, 0.25 mmol) in CH₂Cl₂ (2 mL) are added BOC-glycine (0.0875 g, 0.5 mmol), EDC (0.096 g, 0.5 mmol) and DMAP (3 mg, 0.025 mmol). The reaction mixture is stirred at 40° C. for 16 h. The solvent is removed, and the residue is purified using a Biotage column (elution with 0-50% EtOAc/hexanes) to afford (trans)-4-(2-carbamoyl-3-fluoro-5-(3,6,6-trimethyl-4-oxo-4,5,6,7-tetrahydro-1H-indol-1-yl)phenylamino)cyclohexyl 2-(tert-butoxycarbonylamino)acetate (0.0803 g, 55% yield). LC/MS: m/z=585 [M+H]⁺.

A solution of HCl in 1,4-dioxane (4 M) is added to a solution of (trans)-4-(2-carbamoyl-3-fluoro-5-(3,6,6-trimethyl-4-oxo-4,5,6,7-tetrahydro-1H-indol-1-yl)phenylamino)cyclohexyl 2-(tert-butoxycarbonylamino)acetate (0.0803 g, 0.138 mmol) in 1,4-dioxane (1 mL). The reaction mixture is stirred at 50° C. overnight, and then is poured onto TBME (1 mL). The white solid is isolated by vacuum filtration, and dried under vacuum to afford (trans)-4-(2-carbamoyl-3-fluoro-5-(3,6,6-trimethyl-4-oxo-4,5,6,7-tetrahydro-1H-indol-1-yl)phenylamino)cyclohexyl 2-aminoacetate (0.054 g, 27% yield). LC/MS: m/z=485 [M+H]⁺. ¹H NMR (400 MHz, d₆ DMSO): δ 8.23 (s, 3H), 7.67 (dd, 1H), 6.84 (d, 1H), 6.51 (s, 1H), 6.49 (dd, 1H), 4.80 (m, 1H), 3.79 (q, 2H), 3.49 (m, 1H), 2.74 (s, 2H), 2.24 (s, 2H), 2.19 (s, 3H), 2.03-1.90 (m, 4H), 1.57 (q, 2H), 1.34 (q, 2H), 0.98 (s, 6H) Example 200-1

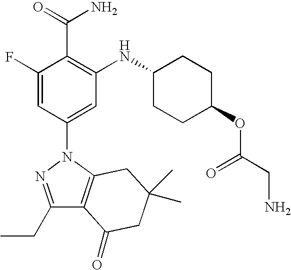

### (trans)-4-(2-carbamoyl-5-(3-ethyl-6,6-dimethyl-4-oxo-4,5,6,7-tetrahydro-1H-indazol-1-yl)-3-fluorophenylamino)cyclohexyl 2-aminoacetate hydrochloride

4-(3-Ethyl-6,6-dimethyl-4-oxo-4,5,6,7-tetrahydro-indazol-1-yl)-2-fluoro-6-(4-hydroxy-cyclohexylamino)-benzamide (350 mg, 0.79 mmol), EDC (303 mg, 1.6 mmol), BOC-glycine (277 mg, 1.6 mmol) and DMAP (97 mg, 0.79 mmol) are dissolved in methylene chloride (6 mL) and stirred at room temperature overnight. The mixture is purified via chromatography (silica, 40 to 90% EtOAc in hexanes) to give the intermediate, (trans)-4-(2-carbamoyl-5-(3-ethyl-6,6-dimethyl-4-oxo-4,5,6,7-tetrahydro-1H-indazol-1-yl)-3-fluorophenylamino)-cyclohexyl 2-(tert-butoxycarbonylamino)acetate.

The intermediate is dissolved in 1,4-dioxane (6 mL), treated with 4M HCl in 1,4-dioxane (58 mg, 1.58 mmol, 400 μL) and heated to 50° C. overnight forming a slurry. The slurry is diluted with MTBE (20 mL) and stirred rapidly. After 2 h, the solid is filtered off under nitrogen and left to dry under a nitrogen stream for 3 days to give (trans)-4-(2-carbamoyl-5-(3-ethyl-6,6-dimethyl-4-oxo-4,5,6,7-tetrahydro-1H-indazol-1-yl)-3-fluorophenylamino)cyclohexyl 2-aminoacetate hydrochloride (360 mg, 85%) as a white solid. LC/MS: m/z=500 [M+H]⁺. ¹H NMR (400 MHz, d₆ DMSO): δ 8.36 (b s, 4H), 7.74 (s, 1H), 7.68 (s, 1H), 6.66 (d, 1H), 6.61 (dd, 1H), 4.81 (m, 1H), 3.77 (m, 2H), 3.46 (m, 1H), 2.95 (s, 2H), 2.79 (q, 2H), 2.03 (m, 2H), 1.93 (m, 2H), 1.57 (m, 2H), 1.37 (m, 2H), 1.18 (t, 3H), 1.00 (s, 6H).

### EXAMPLE 201-1

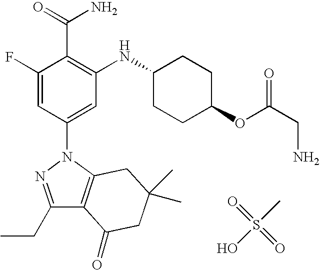

### trans-4-(2-carbamoyl-3-fluoro-5-(3,6,6-trimethyl-4-oxo-4,5,6,7-tetrahydro-1H-indazol-1-yl)phenylamino)cyclohexyl 2-aminoacetate methanesulfonate

To the suspension of trans-4-(2-carbamoyl-3-fluoro-5-(3,6,6-trimethyl-4-oxo-4,5,6,7-tetrahydro-1H-indazol-1-yl)phenylamino)cyclohexyl 2-(tert-butoxycarbonylamino)acetate (see Example 273) (5 g, 1 eq) in ACOH (40 mL), methanesulfonic acid (0.554 mL, 1 eq) are added and stirred at 60° C. for 3 h. The reaction mixture is concentrated completely to give sticky oil, then THF (150 mL) is added to form the clear solution at room temperature. The solution is concentrated at 45° C. to about 100 mL, then left at room temperature to precipitate out. The solid is collected, washed by cold THF and dried completely to give methanesulfonic acid salt (4.12 g, 83% yield). LCMS: m/z=486 [M+H]⁺. ¹H NMR (400 MHz, d₆ DMSO): δ 7.74 (b, 1H), 7.70 (b, 1H), 7.34-7.32 (m, 1H), 6.65 (m, 1H), 6.63-6.58 (m, 1H), 4.86-4.76 (m, 1H), 3.78 (b, 2H), 3.52-3.41 (m, 1H), 2.94 (s, 2H), 2.38 (s, 3H), 2.31 (s, 2H), 2.06-1.87 (m, 4H), 1.63-1.51 (m, 2H), 1.43-1.31 (m, 2H), 1.00 (s, 6H).

### EXAMPLE 207

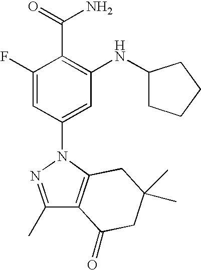

### 2-(cyclopentylamino)-6-fluoro-4-(3,6,6-trimethyl-4-oxo-4,5,6,7-tetrahydro-1H-indazol-1-yl)benzamide

To a solution of 2,6-Difluoro-4-(3,6,6-trimethyl-4-oxo-4,5,6,7-tetrahydro-indazol-1-yl)-benzonitrile (315 mg, 1.00 mmol) in DMSO (0.5 mL), cyclopentylamine (0.20 mL, 2.00 mmol) and DIEA (0.35 mL, 2.00 mmol) are added, and the reaction is stirred at 100° C. for 13 hours. The mixture is then diluted with EtOAc (50 mL), washed with H₂O (2×50 mL) and brine (1×50 mL), and the organic layers are dried over Na₂SO₄ and concentrated. Purification of the residue using a biotage column (elution with 0-100% EtOAc in hexanes) affords 2-(cyclopentylamino)-6-fluoro-4-(3,6,6-trimethyl-4-oxo-4,5,6,7-tetrahydro-1H-indazol-1-yl)benzonitrile (137 mg, 36% yield) as a tan solid. LCMS: m/z=381 [M+H]⁺.

A solution of 2-(cyclopentylamino)-6-fluoro-4-(3,6,6-trimethyl-4-oxo-4,5,6,7-tetrahydro-1H-indazol-1-yl)benzonitrile (137 mg, 0.36 mmol) in EtOH/DMSO (4:1, 4 mL) is treated with 5M NaOH (2 mL) and 35% aqueous H₂O₂ (2 mL), and the reaction is heated with a heat gun for 15 seconds. After cooling, the reaction is diluted with EtOAc (20 mL), washed with H₂O (2×20 mL) and brine (1×20 mL). The combined organic layers are dried over Na₂SO₄ and concentrated under reduced pressure. Purification of the residue using a Biotage column (elution with 0-100% EtOAc in hexanes) gives 2-(cyclopentylamino)-6-fluoro-4-(3,6,6-trimethyl-4-oxo-4,5,6,7-tetrahydro-1H-indazol-1-yl)benzamide (29 mg, 20% yield) as a clear colorless solid. LCMS: m/z=399 [M+H]⁺. ¹H NMR (400 MHz, d₆ DMSO): δ 7.71 (b, 1H), 7.67 (b, 1H), 7.38 (d, 1H), 6.63-6.60 (m, 1H), 6.66 (s, 1H), 3.85-3.76 (m, 1H), 2.94 (s, 2H), 2.38 (s, 3H), 2.31 (s, 2H), 2.01-1.91 (m, 2H), 1.70-1.52 (m, 4H), 1.47-1.37 (m, 2H), 1.00 (s, 6H).

### EXAMPLE 210

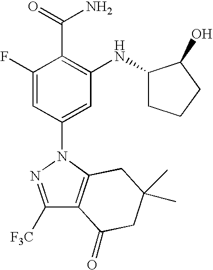

### 4-(6,6-dimethyl-4-oxo-3-(trifluoromethyl)-4,5,6,7-tetrahydro-1H-indazol-1-yl)-2-fluoro-6-((1S,2S)-2-hydroxycyclopentylamino)benzamide

4-Bromo-2,6-difluoro-bezonitrile (2.5 g, 1 eq), ((1S,2S)-2-aminocyclopentanol hydrochloride (1.74 g, 1.1 eq), and DIPEA (3.3 mL, 1.1 eq) are dissolved in DMSO (30 mL), and stirred at 120° C. for 2 h. The reaction mixture is poured to saturated aqueous NH₄Cl (150 mL), extracted with EtOAc (3×100 mL), dried over Na₂SO₄, filtered, and concentrated to give 4-bromo-2-fluoro-6-((1S,2S)-2-hydroxycyclopentylamino)benzonitrile (3.4 g, 100% yield). LCMS: m/z=299, 301 [M+H]⁺.

To a stirred solution of 4-bromo-2-fluoro-6-((1S,2S)-2-hydroxycyclopentylamino)benzonitrile (3.4 g) and 6,6-dimethyl-3-trifluoromethyl-1,5,6,7-tetrahydro-indazol-4-one (2.64 g) in 1,4-dioxane (100 mL) K₂CO₃ (6.8 g) is added, and the suspension is frozen at ice-bath and degassed three times by pump/backfill N₂. Then, CuI (2.77 g) and N,N′-dimethylethylenediamine (1.5 mL) are added, and the reaction mixture is degassed again three times. The sealed reaction mixture is stirred at 100° C. overnight. Then, the reaction mixture is filtered through, washed with EtOAc, and concentrated to give crude 4-(6,6-dimethyl-4-oxo-3-(trifluoromethyl)-4,5,6,7-tetrahydro-1H-indazol-1-yl)-2-fluoro-6-((1S,2S)-2-hydroxycyclopentylamino)benzonitrile. LCMS: m/z=451[M+H]⁺.

4-(6,6-dimethyl-4-oxo-3-(trifluoromethyl)-4,5,6,7-tetrahydro-1H-indazol-1-yl)-2-fluoro-6-((1S,2S)-2-hydroxycyclopentylamino)benzonitrile from the previous step is dissolved in EtOH/DMSO (4:1, 100 mL), 1N NaOH (20 mL) and H₂O₂ (5 mL) are added, and the mixture is stirred at room temperature for 4 h. Then the reaction mixture is concentrated and poured into saturated aqueous NH₄Cl (300 mL), extracted with EtOAc (3×150 mL), dried over Na₂SO₄, filtered, concentrated, and the residue is purified by Biotage chromatography (elution with 40% EtOAc in hexane) to give 4-(6,6-dimethyl-4-oxo-3-(trifluoromethyl)-4,5,6,7-tetrahydro-1H-indazol-1-yl)-2-fluoro-6-((1S,2S)-2-hydroxycyclopentylamino)benzamide (2.6 g, 45% 2-step-yield). LCMS: m/z=469 [M+H]⁺. ¹H NMR (400 MHz, d₆ DMSO): δ 7.78 (d, 2H), 7.29 (d, 1H), 6.79 (s, 1H), 6.70 (d, 1H), 4.92 (d, 1H), 3.84 (br, 1H), 3.50 (br, 1H), 2.97 (s, 3H), 2.42 (s, 2H), 2.14 (m, 1H), 2.18-2.16 (m, 2H), 1.51 (m, 1H), 1.35 (m, 1H), 1.02 (d, 6H).

### EXAMPLE 211

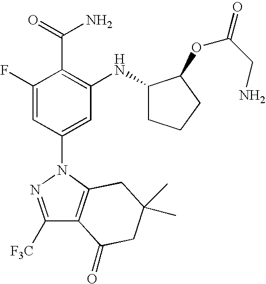

### (1S,2S)-2-(2-carbamoyl-5-(6,6-dimethyl-4-oxo-3-(trifluoromethyl)-4,5,6,7-tetrahydro-1H-indazol-1-yl)-3-fluorophenylamino)cyclopentyl 2-aminoacetate

4-(6,6-dimethyl-4-oxo-3-(trifluoromethyl)-4,5,6,7-tetrahydro-1H-indazol-1-yl)-2-fluoro-6-((1S,2S)-2-hydroxycyclopentylamino)benzamide (800 mg, 1 eq), Boc-glycine (600 mg, 2 eq), EDC (655 mg, 2 eq), and DMAP (21 mg, 0.1 eq) are dissolved in dichloromethane (20 mL) and stirred at room temperature for 18 h. The mixture is concentrated and purified by Biotage chromatography (elution with 300% EA in hexane) to give (1S,2S)-2-(2-carbamoyl-5-(6,6-dimethyl-4-oxo-3-(trifluoromethyl)-4,5,6,7-tetrahydro-1H-indazol-1-yl)-3-fluorophenylamino)cyclopentyl 2-(tert-butoxycarbonylamino)acetate (500 mg, 47% yield). LCMS: m/z=626 [M+H]⁺.

To the solution of (1S,2S)-2-(2-carbamoyl-5-(6,6-dimethyl-4-oxo-3-(trifluoromethyl)-4,5,6,7-tetrahydro-1H-indazol-1-yl)-3-fluorophenylamino)cyclopentyl 2-(tert-butoxycarbonylamino)acetate (500 mg, 1 eq) in acetic acid (5 mL), methanesulfonic acid (93 mg, 1.2 eq) is added over 10 min. The resulting mixture is heated to 60° C. and stirred at this temperature for 3 h. The slurry is cooled to 50° C. and MTBE (10 mL) is added. The mixture is further cooled to ambient temperature and stirred at ambient temperature for 14 h. The slurry is then filtered and washed with MTBE (10 mL). The solids are dried completely to give (1S,2S)-2-(2-carbamoyl-5-(6,6-dimethyl-4-oxo-3-(trifluoromethyl)-4,5,6,7-tetrahydro-1H-indazol-1-yl)-3-fluorophenylamino)cyclopentyl 2-aminoacetate (400 mg, 81% yield). LCMS: m/z=526 [M+H]⁻. ¹H NMR (400 MHz, d₆ DMSO): δ 8.14 (br, 3H), 7.83 (s, 2H), 7.36 (d, 1H), 6.84 (s, 1H), 7.77 (d, 1H), 3.78 (br, 2H), 2.95 (q, 2H), 2.44 (s, 2H), 2.28 (s, 3H), 2.16-2.05 (m, 2H), 1.76-1.66 (m, 3H), 1.53-1.50 (m, 1H), 1.34 (s, 2H), 1.02 (s, 6H).

### EXAMPLE 212

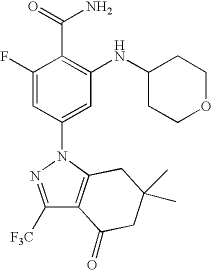

### 4-(6,6-dimethyl-4-oxo-3-(trifluoromethyl)-4,5,6,7-tetrahydro-1H-indazol-1-yl)-2-fluoro-6-(tetrahydro-2H-pyran-4-ylamino)benzamide

4-bromo-2-fluoro-6-(tetrahydro-2H-pyran-4-ylamino)benzonitrile (4.03 g, 13.5 mmol), CuI (465 mg, 2.4 mmol), K₂CO₃ (3.88 g, 28.1 mmol), and 6,6-dimethyl-3-(trifluoromethyl)-6,7-dihydro-1H-indazol-4(5H)-one (2.84 g, 12.2 mmol) are all suspended in 1,4-dioxane (40 mL). The reaction mixture is purged with N₂ gas for 1 h. N,N′-dimethylethylenediamine (0.53 mL, 4.9 mmol) is added and the solution is again purged for 1 h. The reaction mixture is then heated to approximately 100° C. and allowed to run overnight. The reaction mixture is cooled and quenched with 25% NH₄Cl/25% NH₄OH (v/v, 50 mL) solution. The aqueous layer is extracted EtOAc (3×85 mL), combined and washed H₂O (25 mL), brine (25 mL), and dried over Na₂SO₄. The solution is then concentrated to a dark oil and taken on as is to the next step. LC/MS: m/z=45136 [M+H]⁺.

3.0 g of the crude reaction mixture from the previous step is dissolved in EtOH/DMSO (4:1, 100 mL) solution. To this is added 1.0 M NaOH (6 mL) followed by 30% H₂O₂ (8 mL) solution. A precipitate is observed and the completion of the reaction is confirmed by LCMS analysis. The reaction is partitioned with brine and extracted EtOAc (3×150 mL). The organics are combined and washed with H₂O (2×75 mL) and brine (75 mL), dried over Na₂SO₄, concentrated, and purified using 40 mm Biotage column (gradient 40-60%, 80 mL/min flow rate for 20 CV). Pure fractions are combined and concentrated to give 4-(6,6-dimethyl-4-oxo-3-(trifluoromethyl)-4,5,6,7-tetrahydro-1H-indazol-1-yl)-2-fluoro-6-(tetrahydro-2H-pyran-4-ylamino)benzamide (1.05 g, 30% over two steps). LC/MS: m/z=469 [M+H]⁺.

### EXAMPLE 213

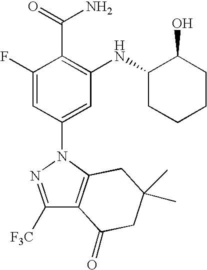

### 4-(6,6-dimethyl-4-oxo-3-(trifluoromethyl)-4,5,6,7-tetrahydro-1H-indazol-1-yl)-2-fluoro-6-((1S,2S)-2-hydroxycyclohexylamino)benzamide

4-Bromo-2,6-difluoro-benzonitrile (3 g, 13.8 mmol), (1S,2S)-2-hydroxycyclohexylamine (1.9 g, 16.5 mmol) and diisopropylethylamine base (3.56 g, 27.5 mmol) are dissolved in DMSO (10 mL) and heated to 80° C. for 16 h. The mixture is partitioned between EtOAc (100 mL) and 1 N HCl (50 mL). The organic layer is washed with brine (25 mL), dried over MgSO₄, filtered and concentrated to a glass. The glass is dissolved in toluene, added to a column and purified via chromatography (silica, 20 to 70% EtOAc in hexanes) to give 4-bromo-2-fluoro-6-((1S,2S)-2-hydroxycyclohexylamino)benzonitrile (3.9 g, 90%) as a fluffy tan solid.

6,6-Dimethyl-3-trifluoromethyl-1,5,6,7-tetrahydro-indazol-4-one (2 g, 8.6 mmol), 4-bromo-2-fluoro-6-((1S,2S)-2-hydroxycyclohexylamino)benzonitrile (2.97 g, 9.47 mmol), potassium carbonate (2.98 g, 21.5 mmol) and CuI (3.28 g, 17.2 mmol) are mixed in 1,4-dioxane (30 mL) and treated with N,N′-dimethylethylenediamine (1.44 g, 16.4 mmol). The mixture is bubbled with nitrogen at room temperature for 1 h, then heated under nitrogen at 100° C. for 23 h. The mixture is partitioned between EtOAc (100 mL) and 1N HCl (100 mL). The two phase mixture is filtered to remove approximately 2 g of solid. The organic layer is removed and the aqueous layer is again extracted with EtOAc (100 mL). The combined organic layers are washed with brine (50 mL), dried over MgSO₄, filtered, concentrated, and the residue is purified via chromatography (silica, 30 to 60% EtOAc in hexanes). Re-purification via chromatography (silica, 10 to 50% EtOAc in hexanes) affords 4-(6,6-dimethyl-4-oxo-3-(trifluoromethyl)-4,5,6,7-tetrahydro-1H-indazol-1-yl)-2-fluoro-6-((1S,2S)-2-hydroxycyclohexylamino)benzonitrile (2.96 g, 74%) as a light yellow solid.

4-(6,6-dimethyl-4-oxo-3-(trifluoromethyl)-4,5,6,7-tetrahydro-1H-indazol-1-yl)-2-fluoro-6-((1S,2S)-2-hydroxycyclohexylamino)benzonitrile is then dissolved in DMSO (8 mL) and treated with 25% NaOH (950 μL) and 30% hydrogen peroxide (680 μL). The mixture is heated gently with a heat gun to affect dissolution. After stirring for 1 h, the mixture is treated with water (30 mL), extracted with methylene chloride (2×30 mL), and the organic layers are purified via chromatography (silica, 30 to 100% EtOAc in hexanes). The product is concentrated, dissolved in methanol, precipitated with water to give a solid, which is filtered off, air dried, and vacuum dried, to give 4-(6,6-dimethyl-4-oxo-3-(trifluoromethyl)-4,5,6,7-tetrahydro-1H-indazol-1-yl)-2-fluoro-6-((1S,2S)-2-hydroxycyclohexylamino)benzamide (2.65 g, 64% overall) as an off-white solid. LC/MS: m/z=483 [M+H]⁺. ¹H NMR (400 MHz, d₆ DMSO): δ 7.75 (br s, 2H), 7.33 (d, 1H), 7.76 (d, 1H), 6.64 (dd, 1H), 4.84 (m, 1H), 3.15 (m, 1H), 2.96 (2 d, 2H), 2.42 (2 d, 2H), 1.98 (m, 1H), 1.82, (m, 1H), 1.62 (m, 1H), 1.54 (m, 1H), 1.20-1.36 (m, 3H), 1.12 (m, 1H), 1.03 (s, 3H), 1.01 (s, 3H).

### EXAMPLE 214-1

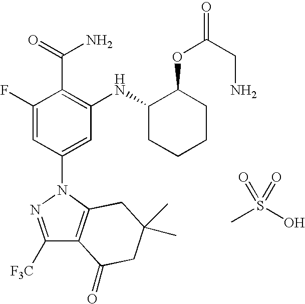

### (1S,2S)-2-(2-carbamoyl-5-(6,6-dimethyl-4-oxo-3-(trifluoromethyl)-4,5,6,7-tetrahydro-1H-indazol-1-yl)-3-fluorophenylamino)cyclohexyl 2-aminoacetate methanesulfonate

A mixture of 4-(6,6-dimethyl-4-oxo-3-(trifluoromethyl)-4,5,6,7-tetrahydro-1H-indazol-1-yl)-2-fluoro-6-((1S,2S)-2-hydroxycyclohexylamino)benzamide (1.00 g, 2.07 mmol), 1-(3-dimethylaminopropyl)-3-ethylcarbodiimide hydrochloride (795 mg, 4.15 mmol), 4-dimethylaminopyridine (253 mg, 2.07 mmol), N-(tert-butoxycarbonyl)glycine (726 mg, 4.15 mmol) in CH₂Cl₂ (20 mL) is stirred at room temperature 16 h. The crude reaction is concentrated on a rotary evaporator and loaded onto a Biotage column (gradient 0-100% EtOAc in hexanes) to provide 1.19 g of (1S,2S)-2-(2-carbamoyl-5-(6,6-dimethyl-4-oxo-3-(trifluoromethyl)-4,5,6,7-tetrahydro-1H-indazol-1-yl)-3-fluorophenylamino)cyclohexyl 2-(tert-butoxycarbonylamino)acetate (89% yield). LCMS: m/z=640 [M+H]⁺.

(1S,2S)-2-(2-carbamoyl-5-(6,6-dimethyl-4-oxo-3-(trifluoromethyl)-4,5,6,7-tetrahydro-1H-indazol-1-yl)-3-fluorophenylamino)cyclohexyl 2-(tert-butoxycarbonylamino)acetate (1.19 g, 1.86 mmol) is dissolved in acetic acid (12 mL), and methanesulfonic acid (0.13 mL, 2.04 mmol) is added and the reaction is heated to 60° C. After 18 h the reaction is cooled to room temperature, diluted with MeO^{t}Bu (100 mL) and stirred vigorously for 2 h. The resulting solid is filtered washing with MeO^{t}Bu (200 mL) to provide ((1S,2S)-2-(2-carbamoyl-5-(6,6-dimethyl-4-oxo-3-(trifluoromethyl)-4,5,6,7-tetrahydro-1H-indazol-1-yl)-3-fluorophenylamino)cyclohexyl 2-aminoacetate methanesulfonate (340 mg, 29% yield) as a gray solid. LCMS: m/z=540 [M+H]⁺. ¹H NMR (400 MHz, d₆ DMSO): δ 8.12 (b, 1H), 7.79 (b, 1H), 7.30 (d, 1H), 6.89 (b, 1H), 6.75 (dd, 1H), 4.86-4.78 (m, 1H), 3.82-3.52 (m, 3H), 2.97 (m, 2H), 2.43 (m, 2H), 2.03-1.88 (m, 2H), 1.73-1.57 (m, 2H), 1.44-1.21 (m, 4H), 1.05 (s, 3H), 1.01 (s, 3H).

### EXAMPLE 216

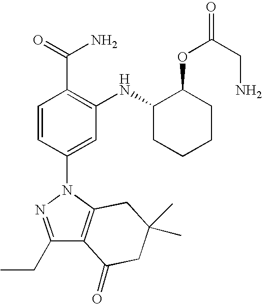

### (1S,2S)-2-(2-carbamoyl-5-(3-ethyl-6,6-dimethyl-4-oxo-4,5,6,7-tetrahydro-1H-indazol-1-yl)phenylamino)cyclohexyl 2-aminoacetate

4-(3-ethyl-6,6-dimethyl-4-oxo-4,5,6,7-tetrahydro-1H-indazol-1-yl)-2-((1S,2S)-2-hydroxycyclohexylamino)benzamide (0.82 mmol, 347 mg) is combined with BOC-glycine (1.3 mmol, 228 mg) and 4-(dimethylamino)pyridine (55 mg), and the mixture is suspended in dichloromethane (6 mL) and 1-ethyl-3-(3-dimethylaminopropyl) carbodiimide hydrochloride (1.3 mmol, 0.25 mg) is added with stirring. After 16 h, the mixture is concentrated and the residue is subjected to chromatography, affording (1S,2S)-2-(2-carbamoyl-5-(3-ethyl-6,6-dimethyl-4-oxo-4,5,6,7-tetrahydro-1H-indazol-1-yl)phenylamino)cyclohexyl 2-(tert-butoxycarbonyl-amino)acetate as a white foam (384 g, 80%). LCMS: m/z=582 [M+H]⁺.

(1S,2S)-2-(2-carbamoyl-5-(3-ethyl-6,6-dimethyl-4-oxo-4,5,6,7-tetrahydro-1H-indazol-1-yl)phenylamino)cyclohexyl 2-(tert-butoxycarbonylamino)acetate (0.65 mmol, 380 mg) is diluted with 1,4-dioxane (3 mL) and 4N HCl in 1,4-dioxane (0.3 mL) is added. After 1.5 h, reaction is incomplete, so it is left stirring at 45° C. and at room temperature overnight. Additional 4N HCl (0.04 mL) is added, and the reaction is stirred at 45° C. for 4 h and 60° C. for 1 h. Toluene (20 mL) is added, and the reaction is concentrated and dried in vacuo. Starting material is still present, so the residue is diluted with 1,4-dioxane (3 mL) and 4N HCL in 1,4-dioxane (0.3 mL), and heated at 60° C. for 4 h. Concentration in the presence of toluene (20 mL) affords (1S,2S)-2-(2-carbamoyl-5-(3-ethyl-6,6-dimethyl-4-oxo-4,5,6,7-tetrahydro-1H-indazol-1-yl)phenylamino)cyclohexyl 2-aminoacetate hydrochloride (334 mg, ˜quant.) as a white solid. LCMS: m/z=482 [M+H]⁺.

### EXAMPLE 218-1

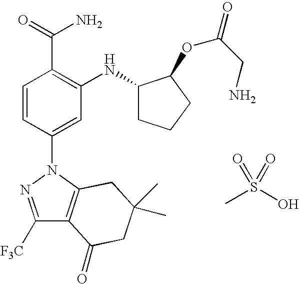

### (1S,2S)-2-(2-carbamoyl-5-(6,6-dimethyl-4-oxo-3-(trifluoromethyl)-4,5,6,7-tetrahydro-1H-indazol-1-yl)phenylamino)cyclopentyl 2-aminoacetate methanesulfonate

(1S,2S)-2-(2-carbamoyl-5-(6,6-dimethyl-4-oxo-3-(trifluoromethyl)-4,5,6,7-tetrahydro-1H-indazol-1-yl)phenylamino)cyclopentyl 2-(tert-butoxycarbonylamino)acetate (see Example 274) (3.38 mmol, 2.05 g) is dissolved in dichloromethane (30 mL) and cooled to 0° C. Trifluoroacetic acid (30 mL) is added over a period of 5 min. After 30 min, toluene (60 mL) is added, and the reaction is concentrated and dried in vacuo. The residue is diluted with dichloromethane (30 mL) and treated with methanesulfonic acid (3.4 mmol, 0.22 mL). The solution is concentrated in the presence of diethyl ether to a white solid. After drying in vacuo, the white solid is triturated with dry diethyl ether to afford (1S,2S)-2-(2-carbamoyl-5-(6,6-dimethyl-4-oxo-3-(trifluoromethyl)-4,5,6,7-tetrahydro-1H-indazol-1-yl)phenylamino)cyclopentyl 2-aminoacetate methanesulfonate (2.32 g, ˜quant.). LC/MS: m/z=508 [M+H]⁺. ¹H NMR (400 MHz, d₆ DMSO): δ 8.13 (s, 3H), 7.88 (s, H), 7.80 (d, 1H), 6.99 (d, 1H), 6.77 (q, 1H), 5.01 (m, 1H), 3.91 (s, 1H), 3.78 (m, 2H), 3.05 (s, 1H), 2.95 (m, 2H), 2.44 (d, 2H), 2.28 (s, 4H), 2.12 (m, 2H), 1.75 (m, 3H), 1.52 (m, 1H), 1.08 (s, 3H), 1.02 (s, 6H).

### EXAMPLE 219-1

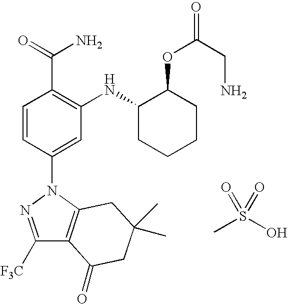

### (1S,2S)-2-(2-carbamoyl-5-(6,6-dimethyl-4-oxo-3-(trifluoromethyl)-4,5,6,7-tetrahydro-1H-indazol-1-yl)phenylamino)cyclohexyl 2-aminoacetate methanesulfonate

A mixture of 4-(6,6-dimethyl-4-oxo-3-(trifluoromethyl)-4,5,6,7-tetrahydro-1H-indazol-1-yl)-2-((1S,2S)-2-hydroxycyclohexylamino)benzamide (1.86 g, 4.00 mmol), 1-(3-dimethylaminopropyl)-3-ethylcarbodiimide hydrochloride (1.53 g, 8.00 mmol), 4-dimethylaminopyridine (488 mg, 4.00 mmol), N-(tert-butoxycarbonyl)glycine (1.40 g, 8.00 mmol) in CH₂Cl₂ (40 mL) is stirred at room temperature for 90 minutes. The crude reaction is concentrated on a rotary evaporator and purified using a Biotage column (gradient 0-100% EtOAc in hexanes) to provide (1S,2S)-2-(2-carbamoyl-5-(6,6-dimethyl-4-oxo-3-(trifluoromethyl)-4,5,6,7-tetrahydro-1H-indazol-1-yl)phenylamino)cyclohexyl 2-(tert-butoxycarbonylamino)acetate (2.34 g, 94% yield). LCMS: m/z=622 [M+H]⁺.

(1S,2S)-2-(2-carbamoyl-5-(6,6-dimethyl-4-oxo-3-(trifluoromethyl)-4,5,6,7-tetrahydro-1H-indazol-1-yl)phenylamino)cyclohexyl 2-(tert-butoxycarbonylamino)acetate (2.34 g, 3.76 mmol) is dissolved in AcOH (24 mL), methanesulfonic acid (0.27 mL, 4.14 mmol) is added, and the reaction is heated to 60° C. After 18 h the reaction is cooled to room temperature, diluted with MeO^{t}Bu (50 mL), and the resulting solid is filtered washing with MeO^{t}Bu (200 mL) to provide (1S,2S)-2-(2-carbamoyl-5-(6,6-dimethyl-4-oxo-3-(trifluoromethyl)-4,5,6,7-tetrahydro-1H-indazol-1-yl)phenylamino)cyclohexyl 2-aminoacetate methanesulfonate (2.25 g, 97% yield) as an off-white solid. LCMS: m/z=522 [M+H]⁺. ¹H NMR (400 MHz, d₆ DMSO): δ 8.13 (b, 3H), 8.01 (b, 1H), 7.78 (d, 1H), 7.33 (b, 1H), 7.03 (d, 1H), 6.74 (dd, 1H), 4.85-4.78 (m, 1H), 3.82-3.45 (m, 3H), 3.06 (s, 1H), 3.04-2.86 (m, 2H), 2.46-2.42 (m, 2H), 2.31 (s, 6H), 2.03-1.86 (m, 2H), 1.72-1.34 (m, 6H), 1.04 (s, 3H), 1.01 (s, 3H).

### EXAMPLE 220

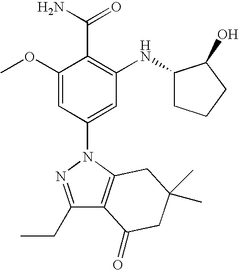

### 4-(3-ethyl-6,6-dimethyl-4-oxo-4,5,6,7-tetrahydro-1H-indazol-1-yl)-2-((1S,2S)-2-hydroxycyclopentylamino)-6-methoxybenzamide

4-(3-Ethyl-6,6-dimethyl-4-oxo-4,5,6,7-tetrahydro-indazol-1-yl)-2,6-difluoro-benzonitrile (4.55 g, 13.8 mmol), 1(S),2(S)-amino cyclopentanol HCl (2.28 g, 16.6 mmol) and diethylisopropylamine (5.36 g, 41 mmol) were combined in DMSO (25 mL), and stirred at 50° C. After 2 h additional amino alcohol (500 mg) and diethylisopropylamine (2.4 mL) were added and stirring continued for 16 h. The mixture was then treated with 1N NaOH (3 mL) and 30% hydrogen peroxide (1 mL) and heated to 50° C. After 2 h, sodium hydroxide (1 g) and peroxide solution (1 mL) were added followed after 1 h by methanol (10 mL). More sodium hydroxide (1 g) and peroxide solution (1 mL) were added and the temperature boosted to 70° C. for 1 h. The mixture was allowed to cool and partitioned between water (75 mL) and methylene chloride (75 mL). The methylene chloride layer was removed and the aqueous layer washed with more methylene chloride (50 mL). The combined organic layers were combined, concentrated and added to a column with toluene and chromatographed (silica gel, 20 to 100% ethyl acetate in hexanes) to give 2 separated products, a major product (Example 227) and a minor product (Example 220). Minor product: 4-(3-ethyl-6,6-dimethyl-4-oxo-4,5,6,7-tetrahydro-1H-indazol-1-yl)-2-((1S,2S)-2-hydroxycyclopentylamino)-6-methoxybenzamide (0.49 g, 8%), a white solid. LC/MS m/z=441 [M+H]⁺. ¹H NMR (400 MHz, DMSO): δ 8.10 (d, 1H), 7.67 (br s, 1H), 7.45 (br s, 1H), 6.51 (d, 1H), 6.43 (d, 1H), 4.84 (d, 1H), 3.84 (m, 1H), 3.83 (s, 3H), 3.48, (m, 1H), 2.94 (2 d, 2H), 2.80 (q, 2H), 2.32 (2 d, 2H), 2.14 (m, 1H), 1.67-1.81 (m, 2H), 1.52 (m, 1H), 1.35 (m, 1H), 1.19 (t, 3H), 1.02 (s, 3H), 0.99 (s, 3H).

### EXAMPLE 226

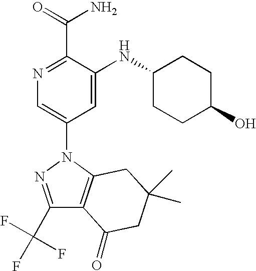

### 5-(6,6-dimethyl-4-oxo-3-(trifluoromethyl)-4,5,6,7-tetrahydro-1H-indazol-1-yl)-3-((trans)-4-hydroxycyclohexylamino)picolinamide

3,5-Difluoro-pyridine-2-carbonitrile (2 g, 14.27 mmol) and trans-1,4-aminocyclohexanol (1.644 g, 14.27 mmol) were dissolved in DMSO (12 mL). Diisopropyl-ethylamine (1.845 g, 14.27 mmol) was added and the reaction was sealed and heated to 150° C. for 30 minutes in the microwave. The reaction mixture was poured into saturated NH₄Cl aq. solution (200 mL) and extracted 3 times with EtOAc (100 mL). The organics were dried with Na₂SO₄ and concentrated to a brown oil and purified on a 40-M (100 g) Biotage silica gel column using a 0 to 75% EtOAc in Hexane gradient. 5-Fluoro-3-(4-hydroxy-cyclohexylamino)-pyridine-2-carbonitrile was isolated (1.214 g) as a white powder. LC/MS m/z=236 [M+H]⁺.

6,6-Dimethyl-3-trifluoromethyl-1,5,6,7-tetrahydroindazol-4-one (1.197 g, 5.16 mmol) and NaH (60% in oil, 289 mg, 7.23 mmol) were dissolved in DMF (25 mL) and when gas formation ceased 5-Fluoro-3-(4-hydroxy-cyclohexylamino)-pyridine-2-carbonitrile (1.214 g, 5.16 mmol) was added and the reaction was heated to 50° C. for 16 hours. The reaction mixture was poured into saturated NH₄Cl aq. solution (200 mL) and extracted 3 times with EtOAc (100 mL). The organics were dried with Na₂SO₄ and concentrated to a yellow solid and purified on a 40-M (100 g) Biotage silica gel column using a 0 to 100% EtOAc in Hexane gradient. 5-(6,6-Dimethyl-4-oxo-3-trifluoromethyl-4,5,6,7-tetrahydro-indazol-1-yl)-3-(4-hydroxy-cyclohexylamino)-pyridine-2-carbonitrile was isolated (2.30 g) as a yellow powder. LC/MS m/z=448 [M+H]⁺.

5-(6,6-Dimethyl-4-oxo-3-trifluoromethyl-4,5,6,7-tetrahydro-indazol-1-yl)-3-(4-hydroxy-cyclohexylamino)-pyridine-2-carbonitrile (2.30 g, 5.16 mmol) was dissolved in 20 mL of a 4:1 EtOH:DMSO solution. To this solution was added 0.2 ml 1M NaOH and 0.2 ml 30% H₂O₂ and the reaction was stirred for 3 hours. The reaction was quenched by pouring into 200 ml saturated NaCl aq. solution and extracted 3 times with CH₂Cl₂. The organics were dried over Na₂SO₄, concentrated and purified on a 40-M (100 g) Biotage silica gel column using a 0 to 100% EtOAc in Hexane gradient. The solids isolated were then triturated with CH₂Cl₂, filtered and the solids isolated to afford 400 mg of 5-(6,6-dimethyl-4-oxo-3-(trifluoromethyl)-4,5,6,7-tetrahydro-1H-indazol-1-yl)-3-((trans)-4-hydroxycyclohexylamino)picolinamide as a pale yellow powder. LC/MS m/z=466 [M+H]⁺. ¹H NMR (DMSO, 20° C., 400 MHz) δ (ppm) 8.68 (d, 1H), 8.09 (s, 1H), 7.95 (d, 1H), 7.56 (s, 1H), 7.42 (d, 1H), 4.55 (s, 1H), 2.97 (s, 2H), 2.43 (s, 2H), 1.97 (d, 2H), 1.80 (d, 2H), 1.27 (m, 4H), 1.02 (s, 6H).

### EXAMPLE 227

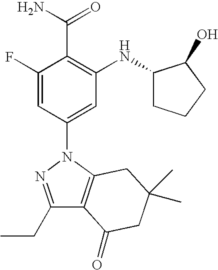

### 4-(3-ethyl-6,6-dimethyl-4-oxo-4,5,6,7-tetrahydro-1H-indazol-1-yl)-2-fluoro-6-((1S,2S)-2-hydroxycyclopentylamino)benzamide

The major product from Example 220 was recrystallized from ethyl acetate in hexanes to provide 4-(3-ethyl-6,6-dimethyl-4-oxo-4,5,6,7-tetrahydro-1H-indazol-1-yl)-2-fluoro-6-((1S,2S)-2-hydroxycyclopentylamino)benzamide (3.06 g, 51%) as a white solid. LC/MS m/z=429 [M+H]⁺. ¹H NMR (400 MHz, DMSO): δ 0.99 (3H, s), 1.02 (3H, s), 1.18 (3H, t), 1.36 (1H, m), 1.49-1.83 (4H, 4 m), 2.15 (1H, m), 2.32 (2H, 2d), 2.80 (2H, q), 2.95 (2H, 2d), 3.50 (1H, m), 3.84 (1H, m), 4.90 (1H, d), 6.62 (1H, dd, J=2,12 Hz), 6.73 (1H, br m), 7.40 (1H, d J=6 Hz), 7.67 (1H, br s), 7.72 (1H, br s).

### EXAMPLE 228

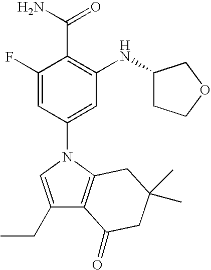

### (S)-4-(3-ethyl-6,6-dimethyl-4-oxo-4,5,6,7-tetrahydro-1H-indol-1-yl)-2-fluoro-6-(tetrahydrofuran-3-ylamino)benzamide

To a solution of NaOH (2.4% in H₂O, 60 mL) 3-oxo-pentanoic acid ethyl ester (4 g) is added, and the mixture is stirred at room temperature for 1 day. A solution of sodium nitrite (2.1 g) in H₂O (8 mL) is added, the mixture is cooled to 5° C., and treated with 20% sulfuric acid (17.2 mL) over a 1 h period. A solution of NaOH (40% in H₂O, 4 mL) is then added, and the mixture is extracted with Et₂O. The aqueous solution is acidified with 20% sulfuric acid at 5° C., and the reaction mixture is kept at that temperature for 1 h. The aqueous layer is extracted with EtOAc, and the combined organic layers are dried over Na₂SO₄, filtered, and evaporated to afford 2.4 g of 2-oxo-butyraldehyde oxime. The intermediate was used without further purification.

A suspension of 2-oxo-butyraldehyde oxime (2.44 g), 5,5-dimethyl-cyclohexane-1,3-dione (3.41 g), and Zn powder (3.15 g) in acetic acid (29.4 mL) and water (12.6 mL) is refluxed overnight. The reaction mixture is dried, dissolved in CH₂Cl₂, and poured into brine. The pH is adjusted to 6 by adding a saturated solution of Na₂CO₃. The aqueous layer is extracted with CH₂Cl₂, dried over Na₂SO₄, filtered, and concentrated. The residue is purified using a Biotage Column (elution with 20% EtOAc/hexanes) to afford 2.3 g (50%) of 3-ethyl-6,6-dimethyl-1,5,6,7-tetrahydro-indol-4-one. LC/MS: m/z=192 [M+H]⁺.

To a stirred solution of 4-bromo-2-fluoro-6-(S-tetrahydro-furan-3-ylamino)-benzonitrile (0.7 g) and 3-ethyl-6,6-dimethyl-1,5,6,7-tetrahydro-indol-4-one (0.5 g) in dioxane (20 mL), K₂CO₃ (1.45 g) is added. The suspension is cooled in ice-bath and degassed by pump/backfill of N₂ three times. Then, CuI (0.6 g) and N,N′-dimethylethylenediamine (0.324 ml) are added. The reaction mixture is degassed again three times. The sealed reaction mixture is stirred at 100° C. overnight. The reaction mixture is filtered through a celite pad, washed with EtOAc, and concentrated to give crude 4-(3-ethyl-6,6-dimethyl-4-oxo-4,5,6,7-tetrahydro-indol-1-yl)-2-fluoro-6-(tetrahydro-furan-3-ylamino)-benzonitrile (0.97 g, 100%), LCMS m/z=396 [M+H]⁺.

Crude 4-(3-ethyl-6,6-dimethyl-4-oxo-4,5,6,7-tetrahydro-indol-1-yl)-2-fluoro-6-(tetrahydro-furan-3-ylamino)-benzonitrile (0.97 g) is dissolved in EtOH/DMSO (4:1, 20 mL), and 1N NaOH aq. solution (5 mL) and H₂O₂ (3 mL) are added. Reaction is stirred at room temperature overnight. The reaction mixture is poured to saturated NH₄Cl solution (150 mL), extracted with EtOAc (3×50 mL), dried over Na₂SO₄, filtered, concentrated, and purified by biotech chromatography (elution with 50% EtOAc in hexane) to give (S)-4-(3-ethyl-6,6-dimethyl-4-oxo-4,5,6,7-tetrahydro-1H-indol-1-yl)-2-fluoro-6-(tetrahydrofuran-3-ylamino)benzamide (0.653 g, 64% yield). LCMS m/z=414 [M+H]⁺. ¹H NMR (400 MHz, d₆ DMSO): δ 7.72 (d, 2H), 7.46 (d, 1H), 6.85 (s, 1H), 6.58 (d, 1H), 6.47 (d, 1H), 3.90-3.70 (m, 4H), 3.55-3.45 (dd, 1H), 2.75 (s, 2H), 2.65 (q, 2H), 2.23 (s, 3H), 1.80-1.65 (m, 1H), 1.13 (t, 3H), 0.98 (s, 6H).

### EXAMPLE 229

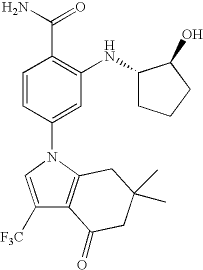

### 4-(6,6-dimethyl-4-oxo-3-(trifluoromethyl)-4,5,6,7-tetrahydro-1H-indol-1-yl)-2-((1S,2S)-2-hydroxycyclopentylamino)benzamide

2-Fluoro-4-Bromobenzonitrile (1.0 g, 5.0 mmol), (S,S)-trans-2-aminocyclopentanol (708 mg, 5.2 mmol), and N,N-diisopropylethylamine (2.18 mL, 7.5 mmol) are dissolved in DMSO (16 mL) in a “personal chemistry” microwave vial. This vial is placed in a microwave reactor and heated with microwave irradiation at 100° C. for 20 minutes. Analysis by LCMS showed complete conversion to product. The reaction is poured into H₂O and a white precipitate formed. The solids are isolated by vacuum filtration to give 4-bromo-2-((1S,2S)-2-hydroxycyclopentylamino)benzonitrile (660 mg, 47% yield). LC/MS: m/z=281 [M+H]⁺.

4-bromo-2-((1S,2S)-2-hydroxycyclopentylamino)benzonitrile (660 mg, 2.35 mmol) from the previous step, CuI (894 mg, 4.7 mmol), K₂CO₃ (812 mg, 5.88 mmol), and 6,6-dimethyl-3-trifluoromethyl-1,5,6,7-tetrahydro-indol-4-one (542 mg, 2.35 mmol) are suspended in 1,4-dioxane (16 mL). Nitrogen gas is bubbled through the mixture for 30 minutes. N,N′-dimethylethylenediamine (0.53 mL, 4.94 mmol) is added, the reaction vessel is sealed and heated to 100° C. and allowed to stir for 18 h. The reaction is cooled and quenched with 25% NH₄Cl/25% NH₄OH (v/v, 5 mL) solution. The aqueous layer is extracted with EtOAc (3×15 mL), washed with H₂O (5 mL) and brine (5 mL), and dried over Na₂SO₄. The solution is concentrated to an oil and purified using Biotage purification system (40 mm column, 80 ml/min flow rate, 0-60% Hex/EtOAc) to give 4-(6,6-dimethyl-4-oxo-3-(trifluoromethyl)-4,5,6,7-tetrahydro-1H-indol-1-yl)-2-((1R,2R)-2-hydroxycyclopentylamino)benzonitrile (323 mg, 32%). LC/MS: m/z=432 [M+H]⁺.

4-(6,6-dimethyl-4-oxo-3-(trifluoromethyl)-4,5,6,7-tetrahydro-1H-indol-1-yl)-2-((1R,2R)-2-hydroxycyclopentylamino)benzonitrile from the previous step (290 mg, 0.67 mmol) is dissolved in EtOH/DMSO (4:1, 11 mL) solution. To this 1.0 M NaOH (0.25 mL) is added, followed by 30% H₂O₂ (0.3 mL) solution. A precipitate is observed and the completion of the reaction is confirmed by LCMS analysis. The reaction is partitioned with brine and extracted with EtOAc (3×10 mL). The organic fractions are combined, washed with H₂O (2×5 mL) and brine (5 mL), and dried over Na₂SO₄. The crude mixture is concentrated and the oil is purified using a 40 mm Biotage column (gradient 40-60%, 80 mL/min flow rate for 20 CV). Pure fractions are combined and concentrated to give 4-(6,6-dimethyl-4-oxo-3-(trifluoromethyl)-4,5,6,7-tetrahydro-1H-indol-1-yl)-2-((1S,2S)-2-hydroxycyclopentylamino)benzamide (50 g, 17% yield). LC/MS: m/z=450 [M+H]⁺. ¹H NMR (400 MHz, d₆ DMSO): δ 8.42 (d, 1H), 7.93 (s, 1H), 7.72 (d, 1H), 7.65 (s, 1H), 7.25 (s, 1H), 6.82 (d, 1H), 6.60 (m, 1H), 4.90 (d, 1H), 3.84 (m, 1H), 3.54 (m, 1H), 2.73 (s, 2H), 2.33 (s, 2H), 2.14 (m, 1H), 1.60-1.81 (m, 2H), 1.51 (m, 1H), 1.36 (m, 1H), 0.99 (s, 6H).

### EXAMPLE 231

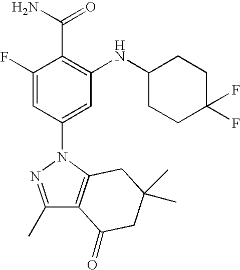

### 2-(4,4-Difluoro-cyclohexylamino)-6-fluoro-4-(3,6,6-trimethyl-4-oxo-4,5,6,7-tetrahydro-indazol-1-yl)-benzamide

2,6-Difluoro-4-(3,6,6-trimethyl-4-oxo-4,5,6,7-tetrahydro-indazol-1-yl)-benzonitrile (0.5 mmol, 158 mg), 4,4-difluorocyclohexyl-1-amine HCl (0.7 mmol, 120 mg), diisopropylethylamine (1.25 mmol, 0.27 mL) and DMSO (1 mL) are combined and heated by microwave irradiation in a sealed vessel to 150° C. for 20 min at high absorbance. The crude product is extracted with EtOAc/H₂O (125 mL/50 mL). The organic phase is dried over MgSO₄, filtered, and concentrated. Flash chromatography affords the desired 2-(4,4-Difluoro-cyclohexylamino)-6-fluoro-4-(3,6,6-trimethyl-4-oxo-4,5,6,7-tetrahydro-indazol-1-yl)-benzonitrile as a waxy solid (181 mg, 84%). LC/MS m/z=431 [M+H]⁺.

The benzonitrile (180 mg) is combined with DMSO (0.1 mL), 95% ethanol (2 mL), and KOH (190 mg). The reaction mixture is heated in a 45° C. oil bath and 32% H₂O₂ (˜0.5 mL) is introduced. After 1 h, the mixture is taken up in EtOAc/H₂O (150 mL/25 mL). The organic layer is dried over MgSO₄, filtered, concentrated, and chromatographed to afford the desired 2-(4,4-Difluoro-cyclohexylamino)-6-fluoro-4-(3,6,6-trimethyl-4-oxo-4,5,6,7-tetrahydro-indazol-1-yl)-benzamide as a white solid (146 mg, 77%). LC/MS m/z=449 [M+H]⁺.

### EXAMPLE 236

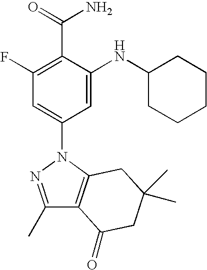

### 2-(cyclohexylamino)-6-fluoro-4-(3,6,6-trimethyl-4-oxo-4,5,6,7-tetrahydro-1H-indazol-1-yl)benzamide

2-(cyclohexylamino)-6-fluoro-4-(3,6,6-trimethyl-4-oxo-4,5,6,7-tetrahydro-1H-indazol-1-yl)benzonitrile (300 mg, 0.95 mmol) and cyclohexylamine (236 mg, 2.4 mmol) are combined in DMSO (2 mL) and stirred at 70° C. for 10 m. The mixture is diluted with ethanol (8 mL), and while still at 70° C., treated with 25% NaOH solution (100 uL), followed by dropwise addition of 30% hydrogen peroxide solution (100 μL). The mixture is concentrated, treated with 1 N HCl (5 mL), and extracted with toluene (2×1 mL). The residue is purified via chromatography (silica, 10 to 60% EtOAc in hexanes) to give a glass, which is recrystallized with EtOAc and hexanes to yield 2-(cyclohexylamino)-6-fluoro-4-(3,6,6-trimethyl-4-oxo-4,5,6,7-tetrahydro-1H-indazol-1-yl)benzamide (135 mg, 34%) as a white crystalline powder. LC/MS: m/z=413 [M+H]⁺. ¹H NMR (400 MHz, d₆ DMSO): δ 7.71 (br s, 1H), 7.67 (br s, 1H), 7.37 (d, 1H), 6.60 (d, 1H), 6.58 (dd, 1H), 3.38 (m, 1H), 2.93 (s, 2H), 2.38 (s, 3H), 2.31 (s, 2H), 1.90 (m, 2H), 1.65 (m, 2H), 1.55 (m, 1H), 1.37 (m, 2H), 1.23 (m, 3H), 1.00 (s, 6H).

### EXAMPLE 238

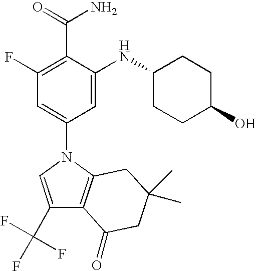

### 4-(6,6-dimethyl-4-oxo-3-(trifluoromethyl)-4,5,6,7-tetrahydro-1H-indol-1-yl)-2-fluoro-6-(trans-4-hydroxycyclohexylamino)benzamide

4-Bromo-2-fluoro-6-(4-hydroxy-cyclohexylamino)-benzonitrile (0.7 g, 2.24 mmol), 6,6-dimethyl-3-trifluoromethyl-1,5,6,7-tetrahydro-indol-4-one (1.55 g, 2.24 mmol), K₂CO₃ (1.55 g, 11.2 mmol), and CuI (0.64 g, 3.25 mmol) are suspended in 1,4-dioxane (5.5 mL) and the reaction mixture is degassed for 10 min under N₂. Then N,N′-dimethylethylenediamine (0.35 mL, 3.25 mmol) is added, and the reaction is again degassed for 10 min. The reaction mixture is then heated to 100° C. for 24 hours. After cooling, the reaction is filtered through Celite, washed with EtOAc (3×20 mL), concentrated and purified via Biotage column (elution with 10-50% EtOAc/Hex) to give 4-(6,6-dimethyl-4-oxo-3-trifluoromethyl-4,5,6,7-tetrahydro-indol-1-yl)-2-fluoro-6-(4-hydroxy-cyclohexylamino)-benzonitrile (0.4 g; 38% yield). LC/MS: m/z=464 [M+H]⁺.

4-(6,6-Dimethyl-4-oxo-3-trifluoromethyl-4,5,6,7-tetrahydro-indol-1-yl)-2-fluoro-6-(4-hydroxy-cyclohexylamino)-benzonitrile (0.4 g, 0.85 mmol) is dissolved in EtOH/DMSO solution (4:1, 2 mL). To this solution is added 1M NaOH (0.1 mL) and 30% H₂O₂ (0.1 mL) and the reaction is stirred for 1 hour. The reaction mixture is poured into brine (30 mL) and extracted 3 times with EtOAc. The organic layers are dried over Na₂SO₄, concentrated, and purified using a Biotage column (elution with 0-100% EtOAc/hexanes) to give 4-(6,6-dimethyl-4-oxo-3-(trifluoromethyl)-4,5,6,7-tetrahydro-1H-indol-1-yl)-2-fluoro-6-(trans-4-hydroxycyclohexylamino)benzamide (0.204 g; 50% yield). LC/MS: m/z=482 [M+H]⁺. ¹H NMR (400 MHz, d₆ DMSO): δ 7.74 (bs, 1H), 7.71 (bs, 1H), 7.69 (s, 1H), 7.17 (dd, 1H), 4.54 (d, 1H), 3.47-3.36 (m, 2H), 2.75 (s, 2H), 2.48-2.47 (m, 1H), 2.33 (s, 2H), 1.96-1.92 (m, 2H), 1.80-1.76 (m, 2H), 1.31 (q, 2H), 1.18 (q, 2H), 1.00 (s, 6H).

### EXAMPLE 242

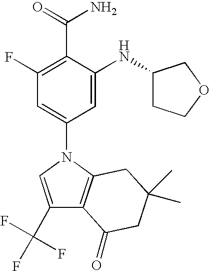

### (S)-4-(6,6-dimethyl-4-oxo-3-(trifluoromethyl)-4,5,6,7-tetrahydro-1H-indol-1-yl)-2-fluoro-6-(tetrahydrofuran-3-ylamino)benzamide

Potassium carbonate (1.73 g, 12.5 mmol) is added to a solution of 4-bromo-2-fluoro-6-(S-tetrahydro-furan-3-ylamino)-benzonitrile (0.71 g, 2.5 mmol) and 6,6-dimethyl-3-trifluoromethyl-1,5,6,7-tetrahydro-indol-4-one (0.58 g, 2.5 mmol) in 1,4-dioxane (6 mL). The solution is degassed under N₂, and the flask is then cooled at 0° C. and connected to a vacuum line. The vacuum is pulled until the solvent started bubbling. The degassing/vacuum cycle is repeated 2 times. Then N,N′-dimethylethylenediamine (0.4 mL, 3.63 mmol) and CuI (0.7 g, 3.75 mmol) are added. The degassing/vacuum cycle is performed 3 times. The rubber septum is replaced by a microwave cap. The solution is degassed one more time, and placed in an oil bath at 110° C. The reaction mixture is stirred at 110° C. overnight. The reaction mixture is filtered through a pad of Celite, and the filter pad is rinsed with EtOAc. The solvent is removed under reduced pressure. Purification of the residue using a Biotage column (elution with 0-70% EtOAc/hexanes) affords 2-fluoro-6-(S-tetrahydro-furan-3-ylamino)-4-(6,6-dimethyl-4-oxo-3-trifluoromethyl-4,5,6,7-tetrahydro-indol-1-yl)-benzonitrile (0.562 g, 52%). LC/MS m/z=436 [M+H]⁺.

A solution of 2-fluoro-6-(S-tetrahydro-furan-3-ylamino)-4-(6,6-dimethyl-4-oxo-3-trifluoromethyl-4,5,6,7-tetrahydro-indol-1-yl)-benzonitrile (0.562 g, 1.3 mmol) in EtOH/DMSO (4:1, 3 mL) is treated with 1M NaOH (0.1 mL) and H₂O₂ (0.1 mL). The reaction mixture is stirred at room temperature for 1 h, then brine (20 mL) is added, and the aqueous phase is extracted with EtOAc (3×). The combined organic layers are dried over MgSO₄, and evaporated under reduced pressure. Purification of the residue using a Biotage column (elution with 20-80% EtOAc/hexanes) yields (S)-4-(6,6-dimethyl-4-oxo-3-(trifluoromethyl)-4,5,6,7-tetrahydro-1H-indol-1-yl)-2-fluoro-6-(tetrahydrofuran-3-ylamino)benzamide (0.086 g, 15%) as a white solid. LC/MS: m/z=454 [M+H]. ¹H NMR (400 MHz, d₆ DMSO): δ 7.79 (b, 1H), 7.77 (b, 1H), 7.71 (d, 1H), 7.38 (dd, 1H), 6.70 (dd, 1H), 6.60 (b, 1H), 4.17 (b, 1H), 3.86-3.68 (m, 3H), 3.54 (dd, 1H), 2.76 (s, 2H), 2.31 (s, 2H), 2.26-2.18 (m, 1H), 1.77-1.70 (m, 1H), 1.00 (s, 6H)

### EXAMPLE 243

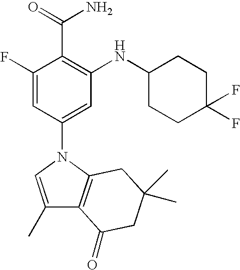

### 2-(4,4-difluorocyclohexylamino)-6-fluoro-4-(3,6,6-trimethyl-4-oxo-4,5,6,7-tetrahydro-1H-indol-1-yl)benzamide

2,6-Difluoro-4-(3,6,6-trimethyl-4-oxo-4,5,6,7-tetrahydro-indazol-1-yl)-benzonitrile (0.28 mmol, 89 mg), 4,4-difluorocyclohexyl-1-amine hydrochloride (0.5 mmol, 86 mg), diisopropylethylamine (1.5 mmol, 0.27 mL) and DMSO (1 mL) are combined and heated by microwave irradiation in a sealed vessel to 150° C. for 20 minutes at high absorbance. The crude product is extracted with EtOAc/water (2:1, 300 mL), and the organic phase is dried over MgSO₄, filtered, concentrated, and purified via chromatography to afford 2-(4,4-difluorocyclohexylamino)-6-fluoro-4-(3,6,6-trimethyl-4-oxo-4,5,6,7-tetrahydro-1H-indol-1-yl)benzonitrile as a waxy solid (66 mg, 55%). LCMS m/z=430 [M+H].

2-(4,4-difluorocyclohexylamino)-6-fluoro-4-(3,6,6-trimethyl-4-oxo-4,5,6,7-tetrahydro-1H-indol-1-yl)benzonitrile (0.15 mmol, 66 mg) is combined with DMSO (0.1 mL), 95% ethanol (3 mL), and KOH (96 mg), and the reaction mixture is warmed to 55° C. and 32% hydrogen peroxide (˜1 mL) is introduced. After 1 h, the mixture is taken up in EtOAc/water (2:1, 300 mL), the organic layers are dried over MgSO₄, filtered, concentrated, and purified via chromatography to afford 2-(4,4-difluorocyclohexylamino)-6-fluoro-4-(3,6,6-trimethyl-4-oxo-4,5,6,7-tetrahydro-1H-indol-1-yl)benzamide as a white solid (58 mg, 87%). LCMS: m/z=448 [M+H]⁺. ¹H NMR (400 MHz, d₆ DMSO): δ 7.73 (br s, 1H), 7.56 (br s, 1H), 7.70 (d, 1H), 6.85 (s, 1H), 6.56 (s, 1H), 6.52 (d, 1H), 3.67 (m, 1H), 2.74 (s, 2H), 2.23 (s, 2H), 2.20 (s, 3H), 2.06-1.91 (m, 6H), 1.46 (m, 2H), 0.98 (s, 6H).

### EXAMPLE 248

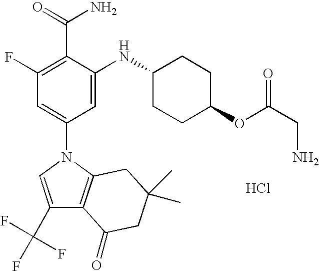

### trans-4-(2-carbamoyl-5-(6,6-dimethyl-4-oxo-3-(trifluoromethyl)-4,5,6,7-tetrahydro-1H-indol-1-yl)-3-fluorophenylamino)cyclohexyl 2-aminoacetate hydrochloride

To a solution of 4-(6,6-dimethyl-4-oxo-3-(trifluoromethyl)-4,5,6,7-tetrahydro-1H-indol-1-yl)-2-fluoro-6-(trans-4-hydroxycyclohexylamino)benzamide (0.0923 g, 0.19 mmol) in CH₂Cl₂ (2 mL) are added BOC-glycine (0.037 g, 0.38 mmol), EDC (0.073 g, 0.38 mmol) and DMAP (3 mg, 0.02 mmol). The reaction mixture is stirred at 40° C. for 2 h. The solvent is removed, and the residue is purified using a Biotage column (elution with 0-100% EtOAc/hexanes) to afford tert-butoxycarbonylamino-acetic acid 4-[2-carbamoyl-5-(6,6-dimethyl-4-oxo-3-trifluoromethyl-4,5,6,7-tetrahydro-indol-1-yl)-3-fluoro-phenylamino]-cyclohexyl ester (0.081 g, 67% yield). LC/MS: m/z=639 [M+H]⁺.

A solution of HCl in 1,4-dioxane (0.32 mL, 1.27 mmol, 4 M) is added to a solution of tert-butoxycarbonylamino-acetic acid 4-[2-carbamoyl-5-(6,6-dimethyl-4-oxo-3-trifluoromethyl-4,5,6,7-tetrahydro-indol-1-yl)-3-fluoro-phenylamino]-cyclohexyl ester (0.081 g, 0.127 mmol) in 1,4-dioxane (1 mL). The reaction mixture is stirred at 50° C. overnight. The reaction mixture is then poured onto TBME (1 mL). The white solid is isolated by vacuum filtration, and dried under vacuum to afford trans-4-(2-carbamoyl-5-(6,6-dimethyl-4-oxo-3-(trifluoromethyl)-4,5,6,7-tetrahydro-1H-indol-1-yl)-3-fluorophenylamino)cyclohexyl 2-aminoacetate hydrochloride (0.068 g, 66% yield). LC/MS: m/z=539 [M+H]⁺. ¹H NMR (400 MHz, d₆ DMSO): δ 8.22 (b, 3H), 7.76 (b, 1H), 7.74 (b, 1H), 7.70 (s, 1H), 6.65 (s, 1H), 6.64 (dd, 1H), 4.80 (b, 1H), 3.79 (q, 2H), 2.76 (s, 2H), 2.33 (s, 2H), 2.03-1.90 (m, 4H), 1.57 (q, 2H), 1.35 (q, 2H), 0.99 (s, 6H).

### EXAMPLE 253

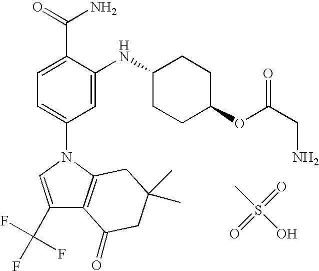

### trans-4-(2-carbamoyl-5-(6,6-dimethyl-4-oxo-3-(trifluoromethyl)-4,5,6,7-tetrahydro-1H-indol-1-yl)phenylamino)cyclohexyl 2-aminoacetate methanesulfonate

A mixture of 4-(6,6-dimethyl-4-oxo-3-(trifluoromethyl)-4,5,6,7-tetrahydro-1H-indol-1-yl)-2-(trans-4-hydroxycyclohexylamino)benzamide (385 mg, 0.83 mmol), 1-(3-dimethylaminopropyl)-3-ethylcarbodiimide hydrochloride (317 mg, 1.66 mmol), 4-dimethylaminopyridine (10 mg, 0.08 mmol), and N-(tert-butoxycarbonyl)glycine (291 mg, 1.66 mmol) in 10 mL CH₂Cl₂ is stirred at room temperature overnight. The crude reaction is concentrated and the crude is loaded onto a 25 mm Biotage column and purified (gradient 10-60% Hexanes/EtOAc). Pure fractions are combined and concentrated to give trans-4-(2-carbamoyl-5-(6,6-dimethyl-4-oxo-3-(trifluoromethyl)-4,5,6,7-tetrahydro-1H-indol-1-yl)phenylamino)cyclohexyl 2-(tert-butoxycarbonylamino)acetate. LC/MS m/z=621 [M+H]⁺.

trans-4-(2-carbamoyl-5-(6,6-dimethyl-4-oxo-3-(trifluoromethyl)-4,5,6,7-tetrahydro-1H-indol-1-yl)phenylamino)cyclohexyl 2-(tert-butoxycarbonylamino)acetate (190 mg, 0.31 mmol) is dissolved in 7 mL of dioxane, and methanesulfonic acid (24 μL, 0.36 mmol) is added. The reaction is heated to 100° C. After 1 h, the reaction is concentrated and placed on the high vacuum pump to give trans-4-(2-carbamoyl-5-(6,6-dimethyl-4-oxo-3-(trifluoromethyl)-4,5,6,7-tetrahydro-1H-indol-1-yl)phenylamino)cyclohexyl 2-aminoacetate methanesulfonate (120 mg, 75% yield) as a brownish solid. LC/MS m/z=521 [M+H]⁺.

### EXAMPLE 259

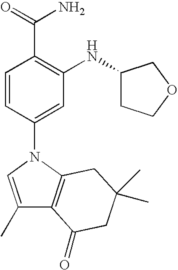

### (S)-2-(tetrahydrofuran-3-ylamino)-4-(3,6,6-trimethyl-4-oxo-4,5,6,7-tetrahydro-1H-indol-1-yl)benzamide

4-Bromo-2-fluoro-bezonitrile (1.25 g), (S)-tetrahydro-furan-3-ylamine hydrochloride (0.785 g), and DIPEA (2.4 mL) are dissolved in DMSO (20 mL) and stirred at room temperature overnight. The reaction mixture is poured into saturated aqueous NH₄Cl (150 mL), extracted with EtOAc (3×100 mL), dried over Na₂SO₄, filtered, and concentrated to give (S)-4-bromo-2-(tetrahydrofuran-3-ylamino)benzonitrile (1.6 g, 96% yield). LCMS: m/z=267, 269 [M+H]⁺.

To a stirred solution of (S)-4-bromo-2-(tetrahydrofuran-3-ylamino)benzonitrile (0.9 g) and 3,6,6-trimethyl-1,5,6,7-tetrahydro-indol-4-one IV (0.6 g) in 1,4-dioxane (20 mL), K₂CO₃ (1.82 g) is added and the suspension is frozen at ice-bath and degassed three times by pump/backfill N₂. Then, CuI (0.83 g) and N,N′-dimethylethylenediamine (0.44 mL) are added, the reaction mixture is degassed again three times, and stirred at 100° C. overnight. Then, the reaction mixture is filtered through celite, washed by EtOAc, and concentrated to give crude (S)-2-(tetrahydrofuran-3-ylamino)-4-(3,6,6-trimethyl-4-oxo-4,5,6,7-tetrahydro-1H-indol-1-yl)benzonitrile (1.2 g, 100%), LCMS: m/z=364 [M+H]⁺.

(S)-2-(tetrahydrofuran-3-ylamino)-4-(3,6,6-trimethyl-4-oxo-4,5,6,7-tetrahydro-1H-indol-1-yl)benzonitrile (1.2 g) is dissolved in EtOH/DMSO (4:1, 50 mL), 1N NaOH (8 mL) and H₂O₂ (5 mL) are added, and the reaction mixture is stirred at room temperature overnight. The reaction mixture is poured into saturated aqueous NH₄Cl (150 mL), extracted with EtOAc (3×50 mL), dried over Na₂SO₄, filtered, concentrated, and purified by Biotage chromatography (elution with 50% EA in hexane) to afford S)-2-(tetrahydrofuran-3-ylamino)-4-(3,6,6-trimethyl-4-oxo-4,5,6,7-tetrahydro-1H-indol-1-yl)benzamide (0.224 g, 18% over 2 steps). LCMS: m/z=382 [M+H]⁺. ¹H NMR (400 MHz, d₆ DMSO): δ 8.54 (d, 1H), 7.72 (d, 1H), 6.83 (s, 1H), 6.59 (s, 1H), 6.56 (d, 1H), 3.88-3.65 (m, 3H), 3.53 (dd, 1H), 2.97 (s, 2H), 2.71 (s, 2H), 2.23 (s, 3H), 2.20 (s, 4H), 1.80-1.70 (m, 1H), 0.97 (s, 6H)

### EXAMPLE 261

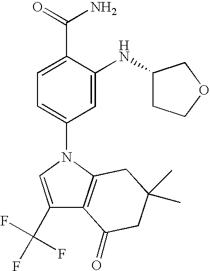

### (S)-4-(6,6-dimethyl-4-oxo-3-(trifluoromethyl)-4,5,6,7-tetrahydro-1H-indol-1-yl)-2-(tetrahydrofuran-3-ylamino)benzamide

6,6-dimethyl-3-trifluoromethyl-1,5,6,7-tetrahydro-indol-4-one (700 mg, 3.0 mmol) and 2-bromo-4-fluorobenzonitrile (900 mg, 4.5 mmol) are dissolved in anhydrous dimethylformamide (DMF, 20 mL). To this is added sodium hydride (60%, 70 mg, 3.0 mmol) and the reaction is stirred at 100° C. for 1 hour. The reaction mixture is cooled to room temperature and poured into water. The aqueous layer is extracted with EtOAc (3×60 mL), the organic layers are combined and washed H₂O (2×30 mL) and brine (30 mL), and dried over Na₂SO₄. The crude mixture is concentrated and the oil is purified using 40 mm Biotage column (gradient 0-60%, 80 mL/min flow rate for 20 CV). Pure fractions are combined and concentrated to give a tan solid. (1.02 g, 83%). LC/MS: m/z=411 [M+H]⁺.

A “Personal Chemistry” microwave vial is charged with the title compound of (300 mg, 0.73 mmol), (S)-3-aminotetrahydrofuran hydrochloride (360 mg, 2.9 mmol), palladium (II) acetate (8 mg, 5 mol %), 1,1′-bis(diphenylphosphino)ferrocene (DPPF) (40 mg, 10 mol %), and sodium tert-butoxide (210 mg, 2.19 mmol). To this is added toluene (5 mL) and the reaction is heated with microwave irradiation to 115° C. for 30 min. After allowing the reaction vessel to cool, a suspension formed and is filtered and the filtrate evaporated. The residue is purified by flash chromatography, and the intermediate product (162 mg, 53%) is hydrolyzed by dissolution in 25% dimethylsulfoxide/ethanol, adding 1 N NaOH (0.25 mL) and 30% aqueous hydrogen peroxide (0.25 mL), followed by stirring at room temperature for 1 hours. An off yellow precipitate formed and H₂O is added to precipitate more solids. The solids are collected by vacuum to yield of (S)-4-(6,6-dimethyl-4-oxo-3-(trifluoromethyl)-4,5,6,7-tetrahydro-1H-indol-1-yl)-2-(tetrahydrofuran-3-ylamino)benzamide (102 mg, 60% yield) as an off yellow powder. LC/MS: m/z=436 [M+H]⁻. ¹H NMR (400 MHz, d₆ DMSO): δ 8.56 (d, 1H), 7.97 (bs, 1H), 7.75 (d, 1H), 7.69 (s, 1H), 7.31 (bs, 1H), 6.73 (s, 1H), 6.65 (dd, 1H), 4.16 (m, 1H), 3.85 (m, 1H), 3.82 (m, 1H), 3.71 (m, 1H), 3.53 (dd, 1H), 2.74 (s, 2H), 2.33 (s, 2H), 1.73 (m, 1H), 0.99 (s, 6H).

### EXAMPLE 263

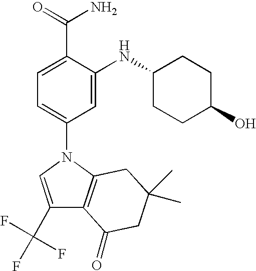

### 4-(6,6-dimethyl-4-oxo-3-(trifluoromethyl)-4,5,6,7-tetrahydro-1H-indol-1-yl)-2-(trans-4-hydroxycyclohexylamino)benzamide

4,4,4-Trifluoro-3-oxo-butyric acid ethyl ester (25 g, 136 mmol) is dissolved in 38 mL of AcOH and cooled to 0° C. in an ice water bath. Sodium nitrite (9.38 g, 136 mmol) is then added drop wise in 25 mL of water. The temperature is monitored and kept below 10° C. After the addition is complete the reaction is allowed to warm to room temperature. The reaction progress is monitored by LCMS and deemed complete after 3 h. 10 mL of water is added to the crude reaction mixture, and the crude is used in the next step.

5,5-dimethyl-1,3-cyclohexanedione (19.06 g, 136 mmol) is dissolved in 60 mL of AcOH and warmed to 70° C. When the target temperature is reached, Zn powder (17.79 g, 272 mmol) is combined with the crude mixture from the previous reaction. The reaction temperature is monitored and kept at a temperature between 70-80° C. and the exotherm is observed during the addition. After the addition of reactants is complete the reaction is heated to 80-90° C. and the reaction progress is monitored by LCMS. After 48 h no further reaction progress is observed. Water (600 mL) is then added to the reaction, and the aqueous layer is extracted with EtOAc (3×300 ml). The organics are combined and washed with water (2×200 mL), saturated aq. NaHCO₃ (150 mL) and brine (200 mL). The organics are dried over Na₂SO₄ and concentrated. The dark red crude oil is dissolved in EtOAc (200 mL) and passed through a short silica plug. The plug is then washed with EtOAc (550 mL) and concentrated under reduced pressure to give a red oil. This oil is loaded onto a 65 mm Biotage column and purified (gradient 0 to 60% EtOAc/hexanes). The product-containing fractions are combined and concentrated to a reduced volume, and hexane is added to precipitate pure 6,6-dimethyl-3-trifluoromethyl-1,5,6,7-tetrahydro-indol-4-one (4.39 g, 14% yield). LC/MS m/z=232 [M+H]⁻. ¹H NMR (400 MHz, d₆ DMSO): δ 11.86 (br s, 1H), 7.31 (s, 1H), 2.65 (s, 2H), 2.25 (s, 2H), 1.01 (s, 6H).

6,6-dimethyl-3-trifluoromethyl-1,5,6,7-tetrahydro-indol-4-one (1.0 g, 4.3 mmol) is dissolved in DMF (14 mL). To this NaH (170 mg, 8.6 mmol) is added and allowed to stir at room temperature for 15 minutes. After 15 minutes, 4-fluoro-2-(trans-4-hydroxycyclohexylamino)benzonitrile (1.0 g, 4.3 mmol) is added and the reaction vial is sealed and placed in a microwave reactor for 30 mins at 130° C. Upon completion the crude reaction mixture is loaded directly onto a 65 mm Biotage column and purified (gradient 10-60%; EtOAc/Hexanes). Pure fractions are combined to give 4-(6,6-dimethyl-4-oxo-3-(trifluoromethyl)-4,5,6,7-tetrahydro-1H-indol-1-yl)-2-(trans-4-hydroxycyclohexylamino)benzamide (250 mg, 13% yield). LC/MS m/z=464 [M+H]⁺. ¹H NMR (400 MHz, d₆ DMSO): δ (ppm) 8.35 (d, 1H), 7.91 (bs, 1H), 7.73 (s, 1H), 7.70 (s, 1H), 7.68 (s, 1H), 7.11 (bs, 1H), 6.74 (d, 1H), 6.56 (dd, 1H), 3.36-3.45 (m, 1H), 2.73 (s, 2H), 2.32 (s, 2H), 1.93 (m, 2H), 1.76 (m, 2H), 1.29 (m, 2H), 1.15 (m, 2H), 0.98 (s, 6H)

### EXAMPLE 273

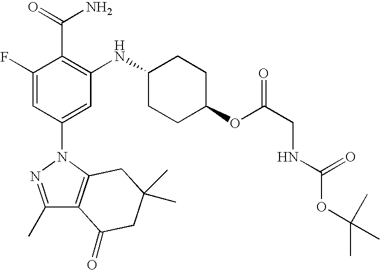

### trans-4-(2-carbamoyl-3-fluoro-5-(3,6,6-trimethyl-4-oxo-4,5,6,7-tetrahydro-1H-indazol-1-yl)phenylamino)cyclohexyl 2-(tert-butoxycarbonylamino)acetate

2-fluoro-6-(trans-4-hydroxycyclohexylamino)-4-(3,6,6-trimethyl-4-oxo-4,5,6,7-tetrahydro-1H-indazol-1-yl)benzamide (20 g, 34 mmol), Boc-glycine (16.4 g, 68 mmol), EDC (17.9 g, 68 mmol), and DMAP (0.57 g, 3.4 mmol) are dissolved in dichloromethane (435 mL) and stirred at room temperature for 18 h. The reaction mixture is quenched with water (450 mL), the layers are separated and the aqueous layer is extracted with dichloromethane (450 mL). The combined organic layers are washed successively with saturated NaHCO₃ (450 mL), 1 N HCl (450 mL), and brine (450 mL), and concentrated to approximately 70 mL. EtOAc (88 mL) is then added, and the mixture is further concentrated to remove residual methylene chloride. The resulting EtOAc solution is heated to 50° C. and hexane (180 mL) is added. After cooling to ambient temperature, the slurry is filtered and washed with EtOAc/hexane (1:2, 70 mL). The solids are collected and dried under vacuum to afford trans-4-(2-carbamoyl-3-fluoro-5-(3,6,6-trimethyl-4-oxo-4,5,6,7-tetrahydro-1H-indazol-1-yl)phenylamino)cyclohexyl 2-(tert-butoxycarbonylamino)acetate (27 g, 99% yield). LCMS: m/z=586 [M+H]⁺.

### EXAMPLE 274

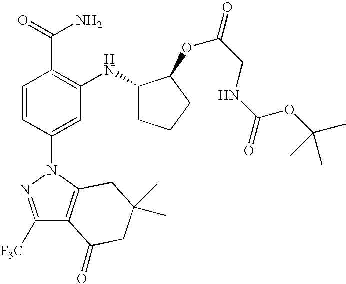

### (1S,2S)-2-(2-carbamoyl-5-(6,6-dimethyl-4-oxo-3-(trifluoromethyl)-4,5,6,7-tetrahydro-1H-indazol-1-yl)phenylamino)cyclopentyl 2-(tert-butoxycarbonylamino)acetate

4-(6,6-dimethyl-4-oxo-3-(trifluoromethyl)-4,5,6,7-tetrahydro-1H-indazol-1-yl)-2-((1S,2S)-2-hydroxycyclopentylamino)benzamide (4.92 mmol, 2.22 g) is combined with BOC-glycine (9.8 mmol, 1.71 g) and 4-(dimethylamino)pyridine (200 mg). The mixture is suspended in dichloromethane (50 mL), and 1-ethyl-3-(3-dimethylaminopropyl)carbodiimide hydrochloride (9.8 mmol, 1.88 g) is added with stirring. After 1.5 h, the mixture is concentrated and the residue is subjected to chromatography, 5 affording (1S,2S)-2-(2-carbamoyl-5-(6,6-dimethyl-4-oxo-3-(trifluoromethyl)-4,5,6,7-tetrahydro-1H-indazol-1-yl)phenylamino)cyclopentyl 2-(tert-butoxycarbonylamino)acetate as a white foam (2.80 g, 94%). LC/MS: m/z=408 [M+H]⁺.

Examples 48-100, 189-215, and 232-250 shown below in Table 1, were prepared essentially according to the synthetic methodology described herein, Examples 1-47 and Schemes 1-5, and/or by using methodology well known in the art.

TABLE 1

| | | Nomenclature | Mass |
|---|---|---|---|
| Ex. | Structure | NMR (400 MHz, DMSO) δ (ppm) | Spec |
| 48 | 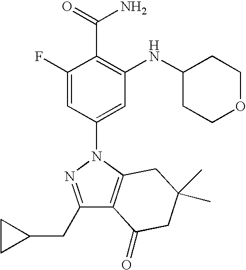 | 4-(3-(cyclopropylmethyl)-6,6-dimethyl-4-oxo-4,5,6,7-tetrahydro-1H-indazol-1-yl)-2-fluoro-6-(tetrahydro-2H-pyran-4-ylamino)benzamide7.59 (b, 1H), 7.55 (b, 1H),7.20 (d, 1H), 6.54 (d, 1H),6.46 (dd, 1H), 3.71-3.63 (m,2H), 3.52-3.43 (m, 1H), 3.34-3.25 (m, 2H), 2.79 (s, 2H),2.55 (d, 2H), 2.17 (s, 2H),1.79-1.72 (m, 2H), 1.29-1.18(m, 2H), 0.99-0.91 (m, 1H),0.84 (s, 6H), 0.25-0.19 (m,2H), 0.05-0.02 (m, 2H) | 455(M + H) |
| 49 | 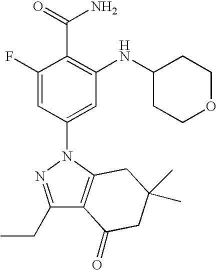 | 4-(3-ethyl-6,6-dimethyl-4-oxo-4,5,6,7-tetrahydro-1H-indazol-1-yl)-2-fluoro-6-(tetrahydro-2H-pyran-4-ylamino)benzamide9.00 (b, 1H), 6.63-6.44 (m,3H), 5.65 (b, 1H), 3.62-3.54(m, 3H), 2.93 (q, 2H), 2.83 (s,2H), 2.41 (s, 2H), 2.08-2.03(m, 2H), 1.66-1.61 (m, 5H),1.30 (t, 3H), 1.12 (s, 6H) | 429(M + H) |
| 50 | 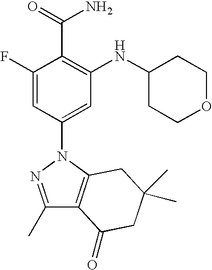 | 2-fluoro-6-(tetrahydro-2H-pyran-4-ylamino)-4-(3,6,6-trimethyl-4-oxo-4,5,6,7-tetrahydro-1H-indazol-1-yl)benzamide7.72 (d, 2H), 7.35 (d, 1H),6.67 (s, 1H), 6.61 (d, 1H),3.84-3.42 (m, 5H), 3.31 (s,2H), 2.49 (s, 2H), 2.47 (s,3H), 2.30 (s, 2H), 1.89 (d,2H), 1.38 (q, 1H), 0.99 (s, 6H) | 415(M + H) |
| 51 | 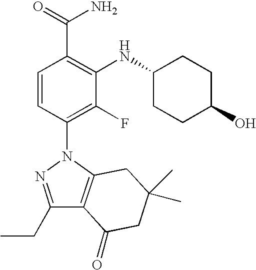 | 4-(3-ethyl-6,6-dimethyl-4-oxo-4,5,6,7-tetrahydro-1H-indazol-1-yl)-3-fluoro-2-(trans-4-hydroxycyclohexylamino)benzamide8.14 (s, 1H), 7.83 (d, 1H) 7.56(s, 1H), 6.81-6.78 (m, 1H),3.45 (b, 2H), 2.79 (q, 2H),2.60 (s, 2H), 2.49 (s, 3H),2.34 (s, 2H), 1.92-1.78 (m,5H), 1.21 (t, 3H), 1.00 (s, 6H) | 443(M + H) |
| 52 | 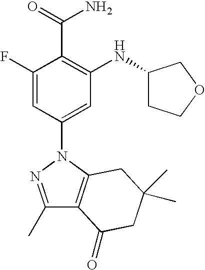 | (S)-2-fluoro-6-(tetrahydrofuran-3-ylamino)-4-(3,6,6-trimethyl-4-oxo-4,5,6,7-tetrahydro-1H-indazol-1-yl)benzamide7.75 (d, 2H), 7.45 (d, 1H),6.65 (d, 1H), 6.60 (s, 1H),4.13-3.54 (m, 4H), 2.94 (s,2H), 2.48 (s, 2H), 2.37 (s,3H), 2.31 (s, 2H), 2.26-2.21(m, 1H), 1.00 (s, 6H) | 401(M + H) |
| 53 | 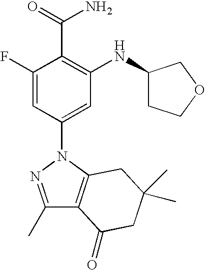 | (R)-2-fluoro-6-(tetrahydrofuran-3-ylamino)-4-(3,6,6-trimethyl-4-oxo-4,5,6,7-tetrahydro-1H-indazol-1-yl)benzamide7.73 (d, 2H), 7.46 (d, 1H),6.65 (dd, 1H), 6.60 (s, 1H,)4.14-3.54 (m, 4H), 2.94 (s,2H), 2.48 (s, 2H), 2.38 (s,3H), 2.30 (s, 2H), 2.26-2.21(m, 1H), 1.00 (s, 6H) | 401(M + H) |
| 54 | 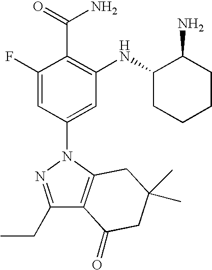 | 2-((1S,2S)-2-aminocyclohexylamino)-4-(3-ethyl-6,6-dimethyl-4-oxo-4,5,6,7-tetrahydro-1H-indazol-1-yl)-6-fluorobenzamide7.75 (d, 2H), 7.05 (d, 1H),6.65 (s, 1H), 6.63 (dd, 1H),3.36-2.76 (m, 7H), 2.47 (s,2H), 2.27 (q, 2H), 1.96-1.23(m, 5H), 1.18 (t, 3H), 1.00 (s,3H), 0.97 (s, 3H) | 442(M + H) |
| 55 | 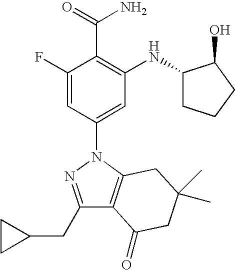 | 4-(3-(cyclopropylmethyl)-6,6-dimethyl-4-oxo-4,5,6,7-tetrahydro-1H-indazol-1-yl)-2-fluoro-6-((1S,2S)-2-hydroxycyclopentylamino)benzamide7.56 (b, 1H), 7.52 (b, 1H),6.58 (d, 1H), 6.47 (dd, 1H),4.75 (d, 1H), 3.71-3.66 (m,1H), 3.38-3.31 (m, 1H), 2.81(s, 1H), 2.80 (s, 1H), 2.58-2.53 (m, 2H), 2.19-2.13 (m,2H), 2.05-1.94 (m, 1H), 1.68-1.41 (m, 3H), 1.40-1.32 (m,1H), 1.26-1.15 (m, 1H), 1.00-0.91 (m, 1H), 0.86 (s, 3H),0.84 (s, 3H), 0.25-0.19 (m,2H), 0.04-0.01 (m, 2H) | 454(M + H) |
| 56 | 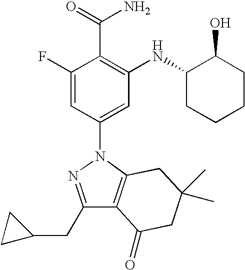 | 4-(3-(cyclopropylmethyl)-6,6-dimethyl-4-oxo-4,5,6,7-tetrahydro-1H-indazol-1-yl)-2-fluoro-6-((1S,2S)-2-hydroxycyclohexylamino)benzamide7.52 (b, 1H), 7.49 (b, 1H),7.28 (m, 1H), 6.54 (d, 1H),6.41 (dd, 1H), 4.66 (d, 1H),3.20-3.09 (m, 1H), 3.03-2.95(m, 1H), 2.88-2.70 (m, 2H),2.58-2.53 (m, 2H), 2.24-2.10(m, 2H), 1.89-1.81 (m, 1H),1.71-1.63 (m, 1H), 1.51-1.36(m, 2H), 1.22-1.03 (m, 3H),1.02-0.90 (m, 2H), 0.86 (s,3H), 0.83 (s, 3H), 0.25-0.20(m, 2H), 0.04-0.01 (m, 2H) | 469(M + H) |
| 57 | 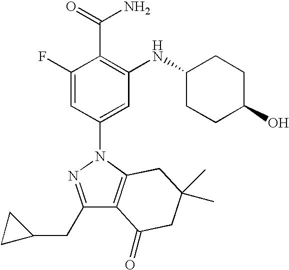 | 4-(3-(cyclopropylmethyl)-6,6-dimethyl-4-oxo-4,5,6,7-tetrahydro-1H-indazol-1-yl)-2-fluoro-6-(trans-4-hydroxycyclohexylamino)benzamide | 469(M + H) |
| 58 | 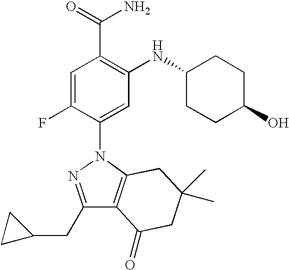 | 4-(3-(cyclopropylmethyl)-6,6-dimethyl-4-oxo-4,5,6,7-tetrahydro-1H-indazol-1-yl)-5-fluoro-2-(trans-4-hydroxycyclohexylamino)benzamide7.94 (d, 1H), 7.81 (b, 1H),7.55 (d, 1H), 7.25 (b, 1H),6.62 (d, 1H), 2.55 (d, 2H),2.50 (s, 3H), 2.33 (s, 2H),2.17 (s, 2H), 1.80-1.60 (m,4H), 1.18-0.95 (m, 4H), 0.84(s, 6H), 0.24-0.02 (m, 4H) | 469(M + H) |
| 59 | 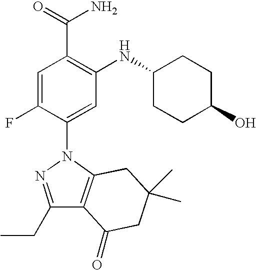 | 4-(3-ethyl-6,6-dimethyl-4-oxo-4,5,6,7-tetrahydro-1H-indazol-1-yl)-5-fluoro-2-(trans-4-hydroxycyclohexylamino)benzamide8.10 (d, 1H), 7.96 (b, 1H),7.70 (d, 1H), 7.40 (b, 1H),6.76 (d, 1H), 2.76 (q, 2H),2.64 (s, 2H), 2.47 (s, 3H),2.32 (s, 2H), 1.92-1.75 (m,5H), 1.30-1.24 (m, 2H), 1.18(t, 3H), 1.00 (s, 6H) | 442(M + H) |
| 60 | 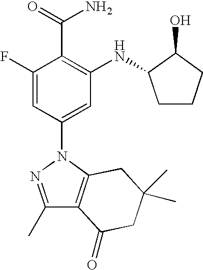 | 2-fluoro-6-((1S,2S)-2-hydroxycyclopentylamino)-4-(3,6,6-trimethyl-4-oxo-4,5,6,7-tetrahydro-1H-indazol-1-yl)benzamide7.71 (d, 2H), 7.38 (d, 1H),6.72 (s, 1H), 6.60 (dd,1H), 2.93 (s, 2H), 2.48 (s, 2H),2.47 (s, 3H), 2.33 (s, 2H),1.88-1.34 (m, 6H), 1.01 (s,3H), 0.99 (s, 3H) | 415(M + H) |
| 61 | 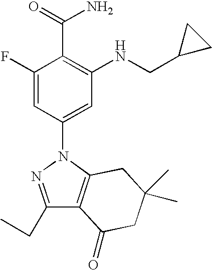 | 2-(cyclopropylmethylamino)-4-(3-ethyl-6,6-dimethyl-4-oxo-4,5,6,7-tetrahydro-1H-indazol-1-yl)-6-fluorobenzamide7.50 (b, 2H), 7.12 (t, 1H),6.40-6.37 (m, 2H), 2.79-2.76(m, 1H), 2.71 (s, 2H), 2.57 (q,2H), 2.27 (s, 2H), 2.09 (s,2H), 0.96 (t, 3H), 0.78 (s,6H), 0.29-0.01 (m, 4H) | 399(M + H) |
| 62 | 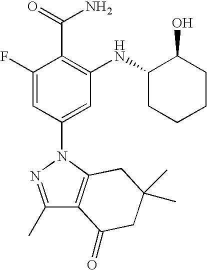 | 2-fluoro-6-((1S,2S)-2-hydroxycyclohexylamino)-4-(3,6,6-trimethyl-4-oxo-4,5,6,7-tetrahydro-1H-indazol-1-yl)benzamide7.67 (m, 2H), 7.43 (d, 1H),6.68 (s,1H), 6.56 (d, 1H), 4.83(d, 1H), 3.30 (m, 1H), 3.14 (m,1H), 2.99 (dd, 1H), 2.88 (dd,1H), 2.48 (s, 2H), 2.38 (s,3H), 2.30 (m, 2H), 2.00 (m,1H), 1.82 (m, 1H), 1.66-1.50(m, 2H), 1.37-1.07 (m, 4H),1.02 (s, 3H), 0.99 (s, 3H) | 429(M + H) |
| 63 | 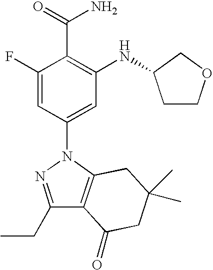 | (S)-4-(3-ethyl-6,6-dimethyl-4-oxo-4,5,6,7-tetrahydro-1H-indazol-1-yl)-2-fluoro-6-(tetrahydrofuran-3-ylamino)benzamide7.75 (d, 2H), 7.48 (d, 1H),6.66 (dd, 1H), 6.61 (s, 1H),4.14-3.54 (m, 4H), 2.97 (s,2H), 2.78 (q, 2H), 2.48 (s,3H), 2.31-2.21 (m, 3H), 1.17(t, 3H), 1.00 (s, 6H) | 415(M + H) |
| 64 | 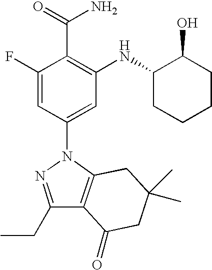 | 4-(3-ethyl-6,6-dimethyl-4-oxo-4,5,6,7-tetrahydro-1H-indazol-1-yl)-2-fluoro-6-((1S,2S)-2-hydroxycyclohexylamino)benzamide7.65 (d, 2H), 7.44 (d, 1H),6.68 (s, 1H), 6.56 (d, 1H),3.31-2.76 (m, 6H), 2.47 (s,2H), 2.26 (q, 2H), 1.96-1.61(m, 3H), 1.15 (t, 3H), 0.98 (s,3H), 0.92 (s, 3H) | 443(M + H) |
| 65 | 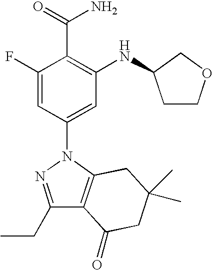 | (R)-4-(3-ethyl-6,6-dimethyl-4-oxo-4,5,6,7-tetrahydro-1H-indazol-1-yl)-2-fluoro-6-(tetrahydrofuran-3-ylamino)benzamide7.73 (d, 2H), 7.48 (d, 1H),6.66 (dd, 1H), 6.60 (s, 1H),4.14 (b, 1H), 3.87-3.54 (m,4H), 2.94 (s, 2H), 2.77 (q,2H), 2.47 (s, 2H), 2.31-2.21(m, 3H), 1.16 (t, 3H), 1.00 (s,6H) | 415(M + H) |
| 66 | 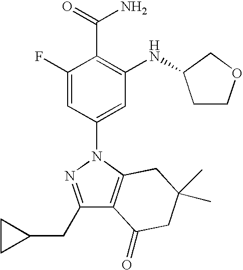 | (S)-4-(3-(cyclopropylmethyl)-6,6-dimethyl-4-oxo-4,5,6,7-tetrahydro-1H-indazol-1-yl)-2-fluoro-6-(tetrahydrofuran-3-ylamino)benzamide7.60 (s, 1H), 7.56 (s, 1H),7.33 (d, 1H), 6.50 (m, 1H), 6.46(s, 1H), 3.97 (m, 1H), 3.73-3.55 (m, 3H), 3.40 (m, 1H),2.80 (s, 2H), 2.55 (d, 2H),2.16 (s, 2H), 2.07 (m, 1H),0.85 (s, 6H) | 441(M + H) |
| 67 | 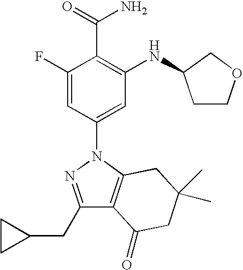 | (R)-4-(3-(cyclopropylmethyl)-6,6-dimethyl-4-oxo-4,5,6,7-tetrahydro-1H-indazol-1-yl)-2-fluoro-6-(tetrahydrofuran-3-ylamino)benzamide7.60 (d, 2H), 7.32 (d, 1H)6.51 (d, 1H), 6.46 (s, 1H),3.98-3.39 (m, 5H), 2.81 (s,3H), 2.55 (d, 2H), 2.32 (s,2H), 2.31-2.06 (m, 3H), 1.59-1.57 (m, 1H), 0.85 (s, 6H, 0.24(d, 2H), 0.02 (d, 2H) | 441(M + H) |
| 68 | 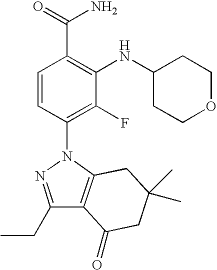 | 4-(3-ethyl-6,6-dimethyl-4-oxo-4,5,6,7-tetrahydro-1H-indazol-1-yl)-3-fluoro-2-(tetrahydro-2H-pyran-4-ylamino)benzamide8.12 (s, 1H), 7.93 (dd, 1H),7.56 (dd, 2H), 6.82 (q, 1H),3.82-3.32 (m, 4H), 2.77 (q,2H), 2.60 (s, 2H), 2.47 (s,1H), 2.32 (s, 2H), 1.84-1.82(m, 2H), 1.37-1.9 (m, 2H), 1.15(t, 3H), 0.98 (s, 6H) | 429(M + H) |
| 69 | 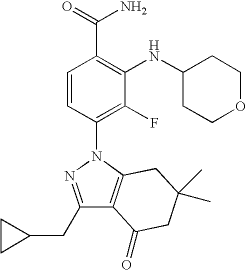 | 4-(3-(cyclopropylmethyl)-6,6-dimethyl-4-oxo-4,5,6,7-tetrahydro-1H-indazol-1-yl)-3-fluoro-2-(tetrahydro-2H-pyran-4-ylamino)benzamide7.97 (s, 1H), 7.78 (d, 1H),7.41 (d, 2H), 6.67 (dd, 1H),3.67-3.20 (m, 4H), 2.82 (s,2H), 2.55 (d, 2H), 2.53 (s,2H), 2.32 (s, 2H), 2.17 (s,2H), 1.70-1.68 (m, 1H), 1.191.17 (m, 1H), 0.83 (s, 6H),0.24-0.01 (m, 4H) | 455(M + H) |
| 70 | 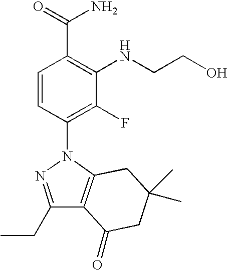 | 4-(3-ethyl-6,6-dimethyl-4-oxo-4,5,6,7-tetrahydro-1H-indazol-1-yl)-3-fluoro-2-(2-hydroxyethylamino)benzamide8.03 (s, 1H), 7.92-7.89 (m,1H), 7.50 (dd, 2H), 6.73 (dd,1H), 4.73 (t, 1H), 3.53-3.33(m, 4H), 2.78 9q, 2H), 2.76 (s,2H), 2.48 (s, 1H), 2.32 (s,2H), 1.18 (t, 3H), 0.99 (s, 6H) | 389(M + H) |
| 71 | 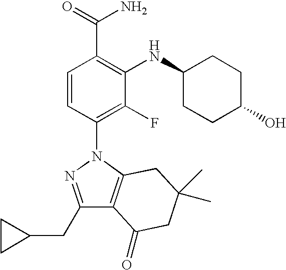 | 4-(3-(cyclopropylmethyl)-6,6-dimethyl-4-oxo-4,5,6,7-tetrahydro-1H-indazol-1-yl)-3-fluoro-2-(trans-4-hydroxycyclohexylamino)benzamide7.93 (b, 1H), 7.66 (d, 1H),7.39 (d, 2H), 6.64 (dd, 1H),3.28 (b, 1H), 2.55 (d, 2H),2.46 (s, 2H), 2.33 (s, 1H),2.19 (s, 2H), 1.77-1.62 (m,4H), 1.08-0.94 (m, 4H), 0.84(s, 6H), 0.24-0.01 (m, 4H) | 469(M + H) |
| 72 | 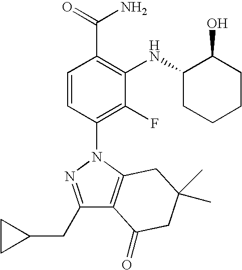 | 4-(3-(cyclopropylmethyl)-6,6-dimethyl-4-oxo-4,5,6,7-tetrahydro-1H-indazol-1-yl)-3-fluoro-2-((1S,2S)-2-hydroxycyclohexylamino)benzamide7.91 (b, 1H), 7.70 (d, 1H),7.34 (dd, 2H), 6.59 (dd, 1H),2.56 (d, 2H), 2.47 (s, 2H),2.33 (s, 1H), 2.18 (s, 2H),1.79-0.93 (m, 8H), 0.092 (s,7H), 0.24-0.01 (m, 4H) | 469(M + H) |
| 73 | 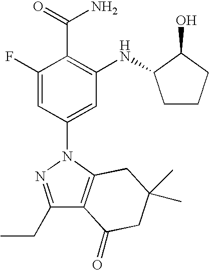 | 4-(3-ethyl-6,6-dimethyl-4-oxo-4,5,6,7-tetrahydro-1H-indazol-1-yl)-2-fluoro-6-((1S,2S)-2-hydroxycyclopentylamino)benzamide7.72 (d, 2H), 7.39 (d, 1H),6.63 (s, 1H), 6.61 (d, 1H),2.94 (d, 2H), 2.80 (q, 2H),2.47 (s, 2H), 2.30 (d, 2H),2.15-2.13 (m, 1H), 1.76-1.35(m, 5H), 1.18 (t, 3H), 1.01 (s,3H), 0.99 (s, 3H) | 429(M + H) |
| 74 | 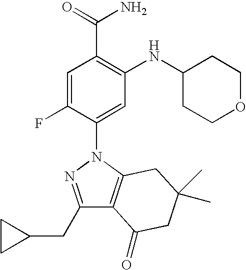 | 4-(3-(cyclopropylmethyl)-6,6-dimethyl-4-oxo-4,5,6,7-tetrahydro-1H-indazol-1-yl)-5-fluoro-2-(tetrahydro-2H-pyran-4-ylamino)benzamide8.06 (d, 1H), 7.86 (b, 1H),7.58 (d, 1H), 7.30 (b, 1H),6.71 (d, 1H), 3.67-3.17 (m,5H), 2.55 (d, 2H), 2.50 (s,2H), 2.33 (s, 1H), 2.17 (s,2H), 1.71 (b, 2H), 1.26-1.15(m, 2H), 0.84 (s, 6H), 0.24-0.01 (m, 4H) | 455(M + H) |
| 75 | 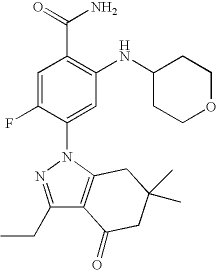 | 4-(3-ethyl-6,6-dimethyl-4-oxo-4,5,6,7-tetrahydro-1H-indazol-1-yl)-5-fluoro-2-(tetrahydro-2H-pyran-4-ylamino)benzamide8.20 (d, 1H), 8.07 (b, 1H),7.76 (d, 1H), 7.45 (b, 1H),6.84 (d, 1H), 3.80-3.45 (m,5H), 2.75 (q, 2H), 2.62 (s,2H), 2.48 (s, 1H), 2.31 (s,2H), 1.96 (b, 1H), 1.37-1.32(m, 2H), 1.17 (t, 3H), 0.98 (s,6H) | 429(M + H) |
| 76 | 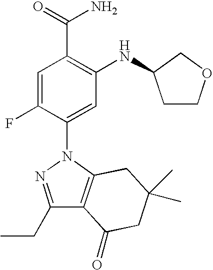 | (R)-4-(3-ethyl-6,6-dimethyl-4-oxo-4,5,6,7-tetrahydro-1H-indazol-1-yl)-5-fluoro-2-(tetrahydrofuran-3-ylamino)benzamide8.29 (d, 1H), 8.02 (b, 1H),7.75 (d, 1H), 7.47 (b, 1H),6.76 (d, 1H), 4.10-3.49 (m,4H), 2.77 (q, 2H), 2.65 (s,2H), 2.32 (s, 1H), 2.22 (s,2H), 2.21-2.19 (m, 1H), 1.71-1.68 (s, 1H), 1.18 (t, 3H),0.99 (s, 6H) | 415(M + H) |
| 77 | 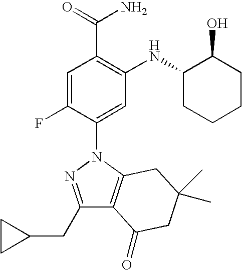 | 4-(3-(cyclopropylmethyl)-6,6-dimethyl-4-oxo-4,5,6,7-tetrahydro-1H-indazol-1-yl)-5-fluoro-2-((1S,25)-2-hydroxycyclohexylamino)benzamide8.10 (d, 1H), 7.79 (b, 1H),7.53 (d, 1H), 7.22 (b, 1H),6.69 (d, 1H), 2.56 (d, 2H),2.49 (s, 2H), 2.33 (s, 2H),2.17 (d, 2H), 1.77-0.92 (m,8H), 0.85 (s, 3H), 0.83 (s,3H), 0.25-0.01 (m, 4H) | 469(M + H) |
| 78 | 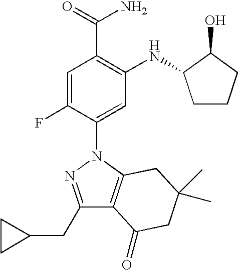 | 4-(3-(cyclopropylmethyl)-6,6-dimethyl-4-oxo-4,5,6,7-tetrahydro-1H-indazol-1-yl)-5-fluoro-2-((1S,2S)-2-hydroxycyclopentylamino)benzamide8.01 (d, 1H), 7.84 (b, 1H),7.57 (d, 1H), 7.27 (b, 1H),6.72 (d, 1H), 2.55 (d, 2H),2.51 (s, 2H), 2.33 (s, 2H),2.17 (s, 2H), 1.98-0.83 (m,6H), 0.84 (s, 6H), 0.25-0.01(m, 4H) | 455(M + H) |
| 79 | 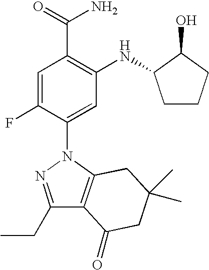 | 4-(3-ethyl-6,6-dimethyl-4-oxo-4,5,6,7-tetrahydro-1H-indazol-1-yl)-5-fluoro-2-((1S,2S)-2-hydroxycyclopentylamino)benzamide8.16 (d, 1H), 7.99 (s, 1H),7.72 (d, 1H), 7.42 (s, 1H),6.87 (d, 1H), 3.85-3.80 (m,1H), 2.76 (q, 2H), 2.64 (s,2H), 2.47 (s, 1H), 2.32 (s,2H), 2.12-1.33 (m, 5H), 1.13(t, 3H), 0.99 (s, 6H) | 429(M + H) |
| 80 | 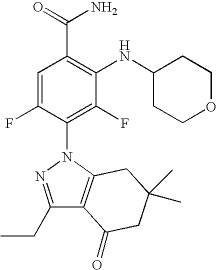 | 4-(3-ethyl-6,6-dimethyl-4-oxo-4,5,6,7-tetrahydro-1H-indazol-1-yl)-3,5-difluoro-2-(tetrahydro-2H-pyran-4-ylamino)benzamide7.91 (s, 1H), 7.77 (s, 1H),6.87 (dd, 1H), 5.01 (dd, 1H),3.74-3.68 (m, 2H), 3.01 (q,2H), 2.87-2.24 (m, 8H), 1.68-1.41 (m, 3H), 1.18 (t, 3H),1.01 (s, 3H), 0.98 (s, 3H) | 447(M + H) |
| 81 | 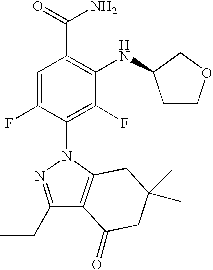 | 4-(3-ethyl-6,6-dimethyl-4-oxo-4,5,6,7-tetrahydro-1H-indazol-1-yl)-3,5-difluoro-2-((R)-tetrahydrofuran-3-ylamino)benzamide7.91 (s, 1H), 7.78 (s, 1H),6.91-6.87 (m, 1H), 5.18-5.07(m, 1H), 3.71-3.40 (m, 6H),2.97-1.71 (m, 9H), 1.16 (t,3H), 1.01 (s, 3H), 0.98 (s, 3H) | 433(M + H) |
| 82 | 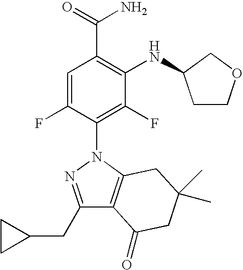 | 4-(3-(cyclopropylmethyl)-6,6-dimethyl-4-oxo-4,5,6,7-tetrahydro-1H-indazol-1-yl)-3,5-difluoro-2-((R)-tetrahydrofuran-3-ylamino)benzamide7.78 (s, 1H), 7.65 (s, 1H),6.79-6.75 (m, 1H), 5.07-4.97(m, 1H), 3.57-3.29 (m, 4H),2.71-1.51 (m, 9H), 0.88-0.80(m, 7H), 0.25-0.01 (m, 4H) | 459(M + H) |
| 83 | 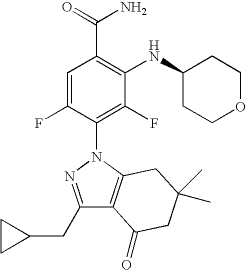 | 4-(3-(cyclopropylmethyl)-6,6-dimethyl-4-oxo-4,5,6,7-tetrahydro-1H-indazol-1-yl)-3,5-difluoro-2-(tetrahydro-2H-pyran-4-ylamino)benzamide7.75 (s, 1H), 7.62 (s, 1H),6.73 (dd, 1H), 4.84 (dd, 1H),3.58-3.51 (m, 1H), 3.91-0.93(m, 13H), 0.84 (s, 3H), 0.82(s, 3H), 0.24-0.01 (m, 4H) | 473(M + H) |
| 84 | 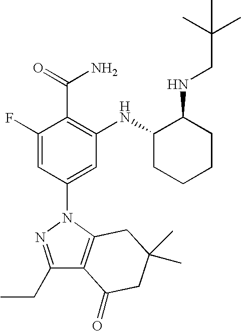 | 4-(3-ethyl-6,6-dimethyl-4-oxo-4,5,6,7-tetrahydro-1H-indazol-1-yl)-2-fluoro-6-((1S,2S)-2-(neopentylamino)cyclohexylamino)benzamide7.80 (d, 2H), 7.53 (d, 1H),6.83 (s, 1H), 6.72 (d, 1H),6.65 (br, 1H), 3.14-3.03 (q,2H), 2.99-2.90 (q, 2H), 2.62(s, 2H), 2.50-2.40 (m, 4H),2.28 (d, 1H), 2.17 (d, 1H),2.05 (d, 1H), 1.85-1.65 (m,2H), 1.32 (t, 3H), 1.13 (d,4H), 0.92 (s, 6H), 0.66 (s,2H), 0.44 (s, 2H), 0.22 (s,2H), 0.00 (s, 2H) | 512(M + H) |
| 85 | 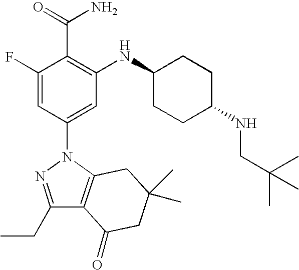 | 4-(3-ethyl-6,6-dimethyl-4-oxo-4,5,6,7-tetrahydro-1H-indazol-1-yl)-2-fluoro-6-(trans-4-(neopentylamino)cyclohexylamino)benzamide7.71-7.67 (m, 2H), 7.26 (d,1H), 6.63-6.57 (m, 2H), 2.93(s, 2H), 2.79 (q, 2H), 2.482.45 (m, 2H), 2.31 (s, 2H),2.00-1.87 (m, 4H), 1.37-1.15(m, 7H), 1.00 (s, 6H), 0.88 (s,9H) | 512(M + H) |
| 86 | 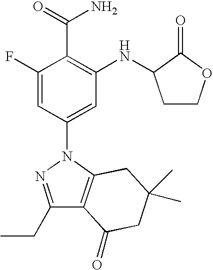 | 4-(3-ethyl-6,6-dimethyl-4-oxo-4,5,6,7-tetrahydro-1H-indazol-1-yl)-2-fluoro-6-(2-oxotetrahydrofuran-3-ylamino)benzamide7.81 (s, 2H), 7.49 (d, 1H),6.74-6.70 (m, 2H), 4.69-4.22(m, 3H), 2.99-2.14 (m, 8H),1.18 (t, 3H), 1.01 (s, 3H),0.98 (s, 3H) | 429(M + H) |
| 87 | 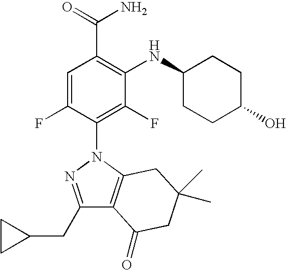 | 4-(3-(cyclopropylmethyl)-6,6-dimethyl-4-oxo-4,5,6,7-tetrahydro-1H-indazol-1-yl)-3,5-difluoro-2-(trans-4-hydroxycyclohexylamino)benzamide7.74 (s, 1H), 7.60 (s, 1H),6.68 (q, 1H), 4.70 (q, 1H),2.64-0.90 (m, 16H), 0.84 (s,3H), 0.82 (s, 3H), 0.27-0.01(m, 4H) | 487(M + H) |
| 88 | 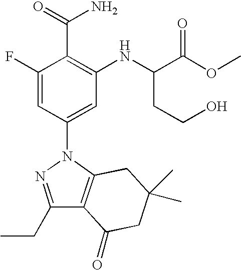 | methyl 2-(2-carbamoyl-5-(3-ethyl-6,6-dimethyl-4-oxo-4,5,6,7-tetrahydro-1H-indazol-1-yl)-3-fluorophenylamino)-4-hydroxybutanoate7.79 (s, 1H), 7.75 (s, 1H),7.70 (d, 1H), 6.69 (d, 1H),6.68 (s, 1H), 4.00 (s, 3H),3.66-3.46 (m, 2H), 2.99-2.76(m, 4H), 2.47 (s, 2H), 2.31 (q,2H), 1.97-1.84 (m, 2H), 1.17(t, 3H), 1.03 (s, 3H), 0.97 (s,3H) | 461(M + H) |
| 89 | 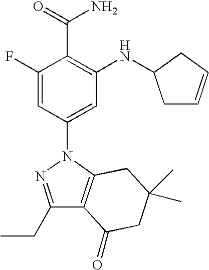 | 2-(cyclopent-3-enylamino)-4-(3-ethyl-6,6-dimethyl-4-oxo-4,5,6,7-tetrahydro-1H-indazol-1-yl)-6-fluorobenzamide7.71 (s, 1H), 7.69 (s, 1H),7.46 (d, 1H), 6.66-6.60 (m,2H), 5.75 (d, 2H), 2.96 (s,2H), 2.79 (q, 2H), 2.48 (s,2H), 2.32 (s, 2H), 2.24-2.16(m, 4H), 1.18 (t, 3H), 1.00 (s,6H) | 411(M + H) |
| 90 | 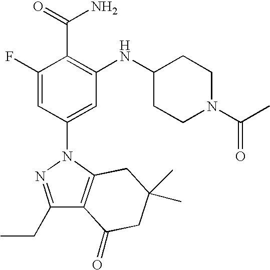 | 2-(1-acetylpiperidin-4-ylamino)-4-(3-ethyl-6,6-dimethyl-4-oxo-4,5,6,7-tetrahydro-1H-indazol-1-yl)-6-fluorobenzamide7.74 (s, 1H), 7.72 (s, 1H),7.36 (d, 1H), 6.70 (s, 1H),6.61 (d, 1H), 4.14-3.66 (m,3H), 2.95 (s, 3H), 2.78 (q,2H), 2.48 (s, 2H), 2.31 (s,3H), 1.99-1.39 (m, 6H), 1.17(t, 3H), 1.00 (s, 6H) | 470(M + H) |
| 91 | 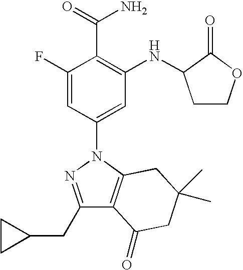 | 4-(3-(cyclopropylmethyl)-6,6-dimethyl-4-oxo-4,5,6,7-tetrahydro-1H-indazol-1-yl)-2-fluoro-6-(2-oxotetrahydrofuran-3-ylamino)benzamide7.65 (s, 2H), 7.32 (d, 1H),6.59-6.56 (m, 2H), 4.53-4.46(m, 1H), 4.23 (t, 1H), 4.134.06 (m, 1H), 2.77 (q, 1H),2.52 (s, 2H), 2.32 (s, 2H),2.08-1.92 (m, 2H), 1.81 (d,2H), 0.86 (s, 3H), 0.82 (s,3H), 0.24-0.02 (m, 4H) | 455(M + H) |
| 92 | 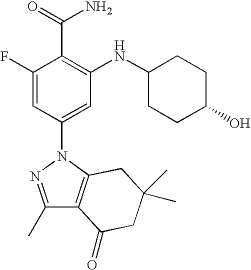 | 2-fluoro-6-(4-hydroxycyclohexylamino)-4-(3,6,6-trimethyl-4-oxo-4,5,6,7-tetrahydro-1H-indazol-1-yl)benzamide7.71-7.66 (m, 2H), 7.26 (d,1H), 6.58 (d, 1H), 2.94 (s,2H), 2.49 (s, 2H), 2.38 (s,3H), 2.32 (s, 2H), 1.97-1.77(m, 4H), 1.32-1.14 (m, 4H),1.00 (s, 6H) | 429(M + H) |
| 93 | 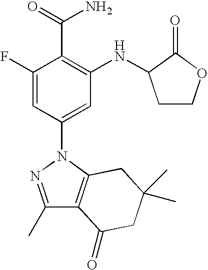 | 2-fluoro-6-(2-oxotetrahydrofuran-3-ylamino)-4-(3,6,6-trimethyl-4-oxo-4,5,6,7-tetrahydro-1H-indazol-1-yl)benzamide7.81 (s, 2H), 7.46 (d, 1H),6.70 (d, 1H), 4.67-4.00 (m,4H), 2.66-2.37 (m, 8H), 0.99(s, 6H) | 415(M + H) |
| 94 | 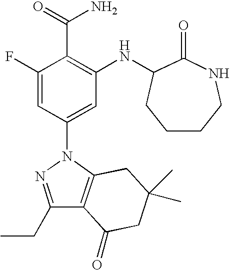 | 4-(3-ethyl-6,6-dimethyl-4-oxo-4,5,6,7-tetrahydro-1H-indazol-1-yl)-2-fluoro-6-(2-oxoazepan-3-ylamino)benzamide7.93 (t, 1H), 7.91 (d, 1H), 7.68(s, 1H), 6.63 (dd, 1H), 6.46(s, 1H), 4.33-4.30 (m, 1H),3.17-1.13 (m, 15H), 1.07 (s,6H), 0.93 (s, 2H) | 456(M + H) |
| 95 | 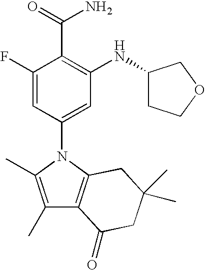 | (S)-2-fluoro-6-(tetrahydrofuran-3-ylamino)-4-(2,3,6,6-tetramethyl-4-oxo-4,5,6,7-tetrahydro-1H-indol-1-yl)benzamide7.77 (s, 2H), 7.26 (d, 1H),6.50 (d, 1H), 6.41 (s, 1H),4.11 (b, 1H), 3.84-3.67 (m,6H), 2.46 (s, 2H), 2.19 (s,2H), 2.14 (s, 3H), 1.96 (s, 6H) | 414(M + H) |
| 96 | 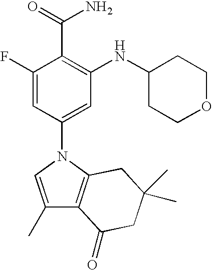 | 2-fluoro-6-(tetrahydro-2H-pyran-4-ylamino)-4-(3,6,6-trimethyl-4-oxo-4,5,6,7-tetrahydro-1H-indol-1-yl)benzamide7.71 (s, 1H), 7.67 (s, 1H),7.32 (s, 1H), 6.83 (s, 1H),6.51-6.48 (m, 1H), 3.78-3.42(m, 5H), 2.72 (s, 2H), 2.48 (s,1H), 2.22-1.15 (m, 9H), 0.97(s, 6H) | 414(M + H) |
| 97 | 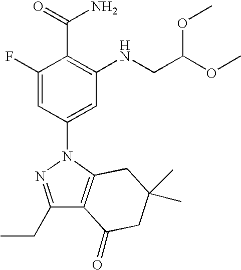 | 2-(2,2-dimethoxyethylamino)-4-(3-ethyl-6,6-dimethyl-4-oxo-4,5,6,7-tetrahydro-1H-indazol-1-yl)-6-fluorobenzamide7.73 (s, 1H), 7.71 (s, 1H),7.33 (d, 1H), 6.67-6.62 (m,2H), 4.53 (t, 1H), 3.31 (s,6H), 3.27 (t, 1H), 2.95 (s,2H), 2.79 (q, 2H), 2.31 (s,2H), 1.18 (t, 3H), 1.00 (s, 6H) | 433(M + H) |
| 98 | 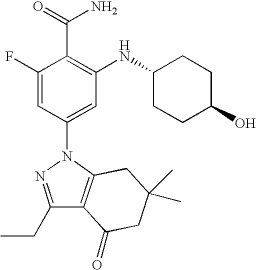 | 4-(3-ethyl-6,6-dimethyl-4-oxo-4,5,6,7-tetrahydro-1H-indazol-1-yl)-2-fluoro-6-(trans-4-hydroxycyclohexylamino)benzamide7.70 (b, 1H), 7.65 (b, 1H),7.27 (d, 1H), 6.63-6.56 (m,2H), 4.56 (d, 1H), 3.50-3.41(m, 1H), 3.36-3.23 (m, 1H),2.94 (s, 2H), 2.83-2.76 (q,2H), 2.32 (s, 2H), 2.00-1.91(m, 2H), 1.84-1.76 (m, 2H),1.37-1.14 (m, 7H), 1.00 (s, 6H) | 443(M + H) |
| 99 | 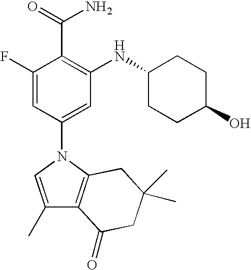 | 2-fluoro-6-(trans-4-hydroxycyclohexylamino)-4-(3,6,6-trimethyl-4-oxo-4,5,6,7-tetrahydro-1H-indol-1-yl)benzamide7.68 (d, 2H), 7.24 (d, 1H),6.84 (s, 1H), 6.47 (t, 2H),4.55 (d, 1H), 2.73 (s, 2H),2.20 (d, 5H), 1.95 (br, 3H),1.77 (d, 2H), 1.35-1.30 (m,2H), 1.20-1.15 (m, 3H), 1.00(s, 6H) | 428(M + H) |
| 100 | 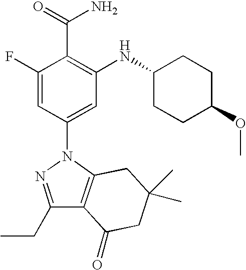 | 4-(3-ethyl-6,6-dimethyl-4-oxo-4,5,6,7-tetrahydro-1H-indazol-1-yl)-2-fluoro-6-(trans-4-methoxycyclohexylamino)benzamide7.71 (s, 1H), 7.66 (s, 1H),7.31 (d, 1H), 6.62-6.57 (m,2H), 3.21 (s, 3H), 2.97-2.93(m, 6H), 2.80 (q, 2H), 2.48(s, 2H), 2.31 (s, 2H), 1.96-1.93 (m, 4H), 1.31-1.23 (m,3H), 1.00 (s, 6H) | 457(M + H) |
| 189 | 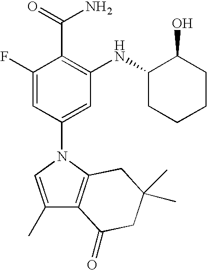 | 2-fluoro-6-((1S,2S)-2-hydroxycyclohexylamino)-4-(3,6,6-trimethyl-4-oxo-4,5,6,7-tetrahydro-1H-indol-1-yl)benzamide7.65 (d, 2H), 7.42 (d, 1H),6.81 (s, 1H), 6.52 (s, 1H),6.42 (d, 1H), 4.80 (d, 1H),3.16 (br, 1H), 2.74 (q, 2H),2.23 (m, 5H), 1.97 (br, 2H),1.80 (br, 1H), 1.60 (br, 1H),1.51 (br, 1H), 1.39-1.20 (m,2H), 1.20-1.15 (m, 2H), 1.00-0.9 (d, 6H) | 428(M + H) |
| 190 | 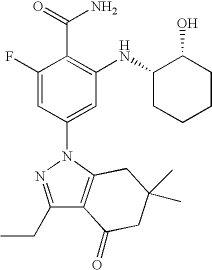 | 4-(3-ethyl-6,6-dimethyl-4-oxo-4,5,6,7-tetrahydro-1H-indazol-1-yl)-2-fluoro-6-((1S,2R)-2-hydroxycyclohexylamino)benzamide7.68-7.61 (m, 1H), 7.40 (d,1H), 6.60 (b, 1H), 6.56-6.52(dd, 1H), 4.78 (d, 1H), 3.82-3.77 (m, 1H), 3.47-3.39 (m,1H), 3.00-2.84 (m, 3H), 2.83-2.75 (m, 2H), 2.37-2.26 (m,2H), 1.69-1.44 (m, 6H), 1.37-1.25 (m, 2H), 1.18 (t, 3H),1.01 (s, 3H), 0.98 (s, 3H) | 443(M + H) |
| 191 | 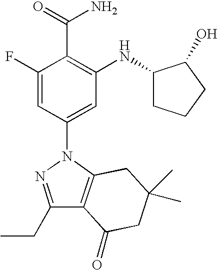 | 4-(3-ethyl-6,6-dimethyl-4-oxo-4,5,6,7-tetrahydro-1H-indazol-1-yl)-2-fluoro-6-((1S,2R)-2-hydroxycyclopentylamino)benzamide7.65 (s, 1H), 7.41 (d, 1H),6.54 (d, 2H), 4.98 (d, 1H), 4.05(m, 1H), 3.59 (m, 1H), 2.94 (s,1H), 2.89 (s, 1H), 2.78 (q,2H), 2.31 (q, 2H), 2.00 (m,1H), 1.75 (m, 2H), 1.61-1.35 (m,3H), 1.17 (t, 3H), 1.01 (s, 3H),0.98 (s, 3H) | 429(M + H) |
| 192 | 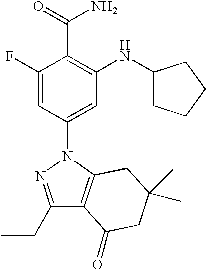 | 2-(cyclopentylamino)-4-(3-ethyl-6,6-dimethyl-4-oxo-4,5,6,7-tetrahydro-1H-indazol-1-yl)-6-fluorobenzamide7.71 (br s, 1H), 7.66 (br s,1H), 7.40 (d, 1H), 6.61 (d,1H), 6.59 (s, 1H), 3.81 (m,1H), 2.94 (s, 2H), 2.80 (q,2H), 2.32 (s, 2H), 1.97 (m,2H), 1.70-1.52 (m, 4H), 1.42(m, 2H), 1.81 (t, 3H), 1.00 (s,6H) | 413(M + H) |
| 193 | 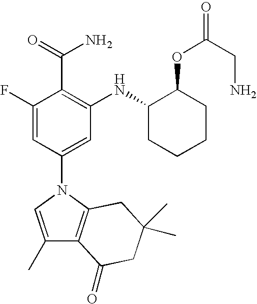 | (1S,2S)-2-(2-carbamoyl-3-fluoro-5-(3,6,6-trimethyl-4-oxo-4,5,6,7-tetrahydro-1H-indol-1-yl)phenylamino)cyclohexyl 2-aminoacetate8.37 (br, 3H), 7.71 (s, 2H),6.82 (s, 1H), 6.64 (s, 1H),6.52 (d, 1H), 4.82 (t, 1H),3.72 (br, 2H), 3.05 (s, 1H),2.73 (s, 2H), 2.21 (d, 3H),1.92 (s, 3H), 1.66 (br, 1H),1.53 (br, 2H), 1.42 (br, 3H),1.10 (s, 2H), 1.00 (s, 6H) | 485(M + H) |
| 194 | 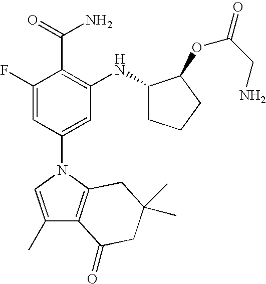 | (1S,2S)-2-(2-carbamoyl-3-fluoro-5-(3,6,6-trimethyl-4-oxo-4,5,6,7-tetrahydro-1H-indol-1-yl)phenylamino)cyclopentyl 2-aminoacetate8.40 (br, 3H), 7.73 (d, 2H),6.85 (s, 1H), 6.65 (s, 1H),6.55 (d, 1H), 5.03 (br, 1H),3.91 (br, 1H), 3.75 (br, 2H),2.25 (d, 3H), 2.01 (m, 1H),1.89 (s, 1H), 1.75 (m, 3H),1.51 (br, 1H), 1.10 (s, 3H),1.00 (s, 6H) | 471(M + H) |
| 195 | 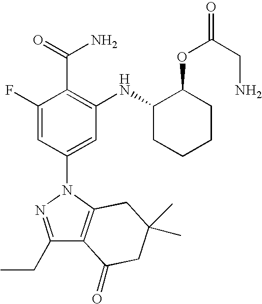 | (1S,2S)-2-(2-carbamoyl-5-(3-ethyl-6,6-dimethyl-4-oxo-4,5,6,7-tetrahydro-1H-indazol-1-yl)-3-fluorophenylamino)cyclohexyl 2-aminoacetate8.35 (s, 3H), 7.14 (s, 2H),7.35 (d, 1H), 6.82 (s,1H), 6.63(m, 1H), 5.81 (m, 1H), 3.77-3.51 (m, 3H), 2.94 (q, 2H),2.80 (q, 2H), 2.32 (q, 2H),2.03-1.85 (m, 2H), 1.71-1.48(m, 3H), 1.38 (m, 3H), 1.91(t, 2H), 1.02 (s, 3H), 0.99 (s,3H) | 500(M + H) |
| 196 | 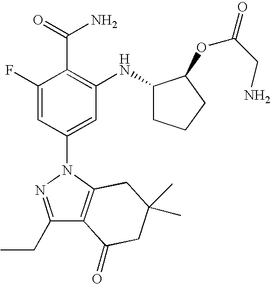 | (1S,2S)-2-(2-carbamoyl-5-(3-ethyl-6,6-dimethyl-4-oxo-4,5,6,7-tetrahydro-1H-indazol-1-yl)-3-fluorophenylamino)cyclopentyl2-aminoacetate8.22 (brs, 1H), 7.75 (d, 1H),7.46 (brs, 1H), 6.80 (d, 1H),6.66 (dd, 1H), 5.01 (m, 1H),3.91 (m, 1H), 3.78 (m, 2H),3.66 (m, 1H), 2.93 (dd, 2H),2.80, (q, 2H), 2.33 (s, 2H),2.16 (m, 1H), 2.07 (m, 1H),1.65-1.81 (m, 3H), 1.52 (m,1H), 1.18 (t, 3H), 1.00 (2 s,6H) | 486(M + H) |
| 197 | 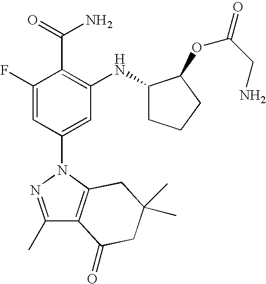 | (1S,2S)-2-(2-carbamoyl-3-fluoro-5-(3,6,6-trimethyl-4-oxo-4,5,6,7-tetrahydro-1H-indazol-1-yl)phenylamino)cyclopentyl 2-aminoacetate8.21 (br s, 1H), 7.75 (d, 1H),7.45 (br s, 1H), 6.81 (d, 1H),6.66 (dd, 1H), 4.99 (m, 1H),3.92 (m, 1H), 3.81 (m, 2H),2.93 (dd, 2H), 2.39 (s, 3H),2.33 (s, 2H), 2.16 (m, 1H),2.07 (m, 1H), 1.63-1.80 (m,3H), 1.52 (m, 1H), 1.18 (t,3H), 1.01 (s, 3H), 1.00 (s, 3H) | 472(M + H) |
| 198 | 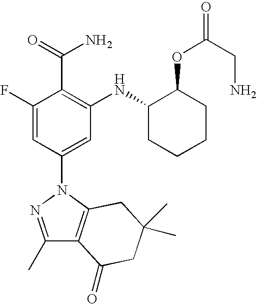 | (1S,2S)-2-(2-carbamoyl-3-fluoro-5-(3,6,6-trimethyl-4-oxo-4,5,6,7-tetrahydro-1H-indazol-1-yl)phenylamino)cyclohexyl 2-aminoacetate8.23 (s, 3H), 7.72 (s, 2H),7.38 (d, 1H), 6.83 (s, 1H), 6.61(d, 1H), 2.98 (d, 1H), 2.87 (s,2H), 2.39 (s, 4H), 2.32 (d,2H), 1.90-2.01 (m, 2H), 1.55-1.60 (m, 2H), 1.38 (m, 2H),1.02 (s, 3H), 0.99 (s, 6H) | 486(M + H) |
| 199 | 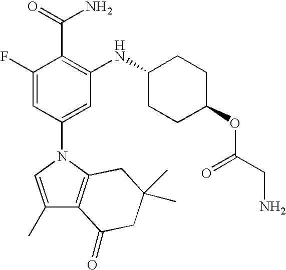 | (trans)-4-(2-carbamoyl-3-fluoro-5-(3,6,6-trimethyl-4-oxo-4,5,6,7-tetrahydro-1H-indol-1-yl)phenylamino)cyclohexyl 2-aminoacetate8.23 (s, 3H), 7.67 (dd, 1H),6.84 (d, 1H), 6.51 (s, 1H),6.49 (dd, 1H), 4.80 (m, 1H),3.79 (q, 2H), 3.49 (m, 1H),2.74 (s, 2H), 2.24 (s, 2H),2.19 (s, 3H), 2.03-1.90 (m,4H), 1.57 (q, 2H), 1.34 (q,2H), 0.98 (s, 6H) | 485(M + H) |
| 200 | 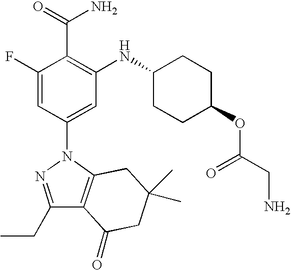 | (trans)-4-(2-carbamoyl-5-(3-ethyl-6,6-dimethyl-4-oxo-4,5,6,7-tetrahydro-1H-indazol-1-yl)-3-fluorophenylamino)cyclohexyl 2-aminoacetate8.36 (b s, 4H), 7.74 (s, 1H),7.68 (s, 1H), 6.66 (d, 1H),6.61 (dd, 1H), 4.81 (m, 1H),3.77 (m, 2H), 3.46 (m, 1H),2.95 (s, 2H), 2.79 (q, 2H),2.03 (m, 2H), 1.93 (m, 2H),1.57 (m, 2H), 1.37 (m, 2H),1.18 (t, 3H), 1.00 (s, 6H) | 500(M + H) |
| 201 | 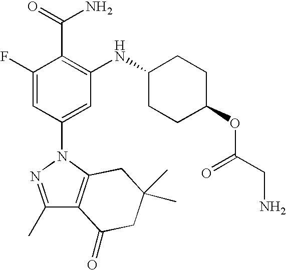 | (trans)-4-(2-carbamoyl-3-fluoro-5-(3,6,6-trimethyl-4-oxo-4,5,6,7-tetrahydro-1H-indazol-1-yl)phenylamino)cyclohexyl 2-aminoacetate7.74 (b, 1H), 7.70 (b, 1H),7.34-7.32 (m, 1H), 6.65 (m,1H), 6.63-6.58 (m, 1H), 4.86-4.76 (m, 1H), 3.78 (b, 2H),3.52-3.41 (m, 1H), 2.94 (s,2H), 2.38 (s, 3H), 2.31 (s,2H), 2.06-1.87 (m, 4H), 1.63-1.51 (m, 2H), 1.43-1.31 (m,2H), 1.00 (s, 6H) | 486(M + H) |
| 202 | 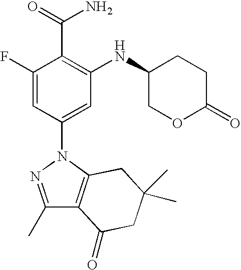 | (S)-2-fluoro-6-(6-oxotetrahydro-2H-pyran-3-ylamino)-4-(3,6,6-trimethyl-4-oxo-4,5,6,7-tetrahydro-1H-indazol-1-yl)benzamide7.90 (br s, 1h), 7.69 (br s,1H), 7.61 (s, 1H), 7.10 (m,2H), 4.10 (dd, 1H), 3.98 (dd,1H), 3.86 (m, 1H), 2.92 (s,2H), 2.39 (s, 3H), 2.31 (s,2H), 2.30 (m, 1H), 2.01-2.16(m, 2H), 1.88 (m, 1H), 1.01 (s,6H) | 429(M + H) |
| 203 | 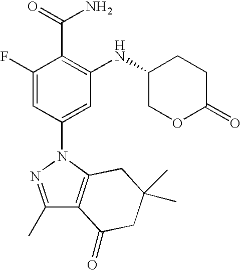 | (R)-2-fluoro-6-(6-oxotetrahydro-2H-pyran-3-ylamino)-4-(3,6,6-trimethyl-4-oxo-4,5,6,7-tetrahydro-1H-indazol-1-yl)benzamide7.9 (br s, 1H), 7.69 (br s,1H), 7.61 (s, 1H), 7.10 (m,3H), 4.10 (dd, 1H), 3.98 (dd,1H), 3.86 (m, 1H), 2.92 (s,2H), 2.39 (s, 3H), 2.31 (s,2H), 2.25-2.34 (m, 1H), 2.02-2.17 (m, 2H), 1.88 (m, 1H),1.01 (s, 6H) | 429(M + H) |
| 204 | 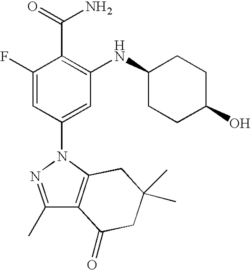 | 2-fluoro-6-((cis)-4-hydroxycyclohexylamino)-4-(3,6,6-trimethyl-4-oxo-4,5,6,7-tetrahydro-1H-indazol-1-yl)benzamide7.73 (s, 1H), 7.69 (s, 1H),7.53 (d, 1H), 6.60 (s, 1H), 6.57(m, 1H), 4.48 (d, 1H), 2.92 (s,2H), 2.37 (s, 3H), 2.30 (s,2H), 1.47-1.65 (m, 8H), 0.99 (s,6H) | 429(M + H) |
| 205 | 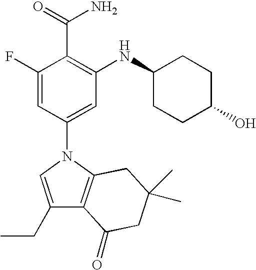 | 4-(3-ethyl-6,6-dimethyl-4-oxo-4,5,6,7-tetrahydro-1H-indol-1-yl)-2-fluoro-6-((trans)-4-hydroxycyclohexylamino)benzamide7.65 (d, 2H), 7.25 (d, 1H),6.84 (s, 1H), 6.48 (d, 2H),4.54 (d, 1H), 2.97 (s, 5H),2.74 (s, 2H), 2.65 (q, 2H),2.24 (s, 2H), 1.96 (br, 2H),1.80 (d, 2H), 1.30 (m, 2H),1.16 (m, 2H), 1.00 (s, 6H) | 442(M + H) |
| 206 | 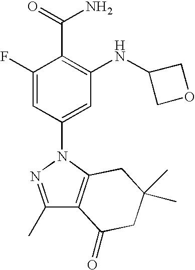 | 2-fluoro-6-(oxetan-3-ylamino)-4-(3,6,6-trimethyl-4-oxo-4,5,6,7-tetrahydro-1H-indazol-1-yl)benzamide7.80 (br s, 2H), 7.64 (d, 1H),6.71 (dd, 1H), 6.29 (d, 1h),4.85 (ps t, 2H), 4.63 (m, 1H),4.42 (ps t, 2H), 2.90 (s, 2H),2.37 (s, 3H), 2.31, (s, 2H),1.00 (s, 6H) | 387(M + H) |
| 207 | 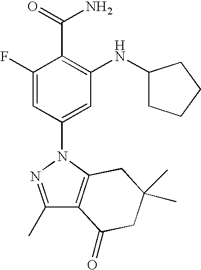 | 2-(cyclopentylamino)-6-fluoro-4-(3,6,6-trimethyl-4-oxo-4,5,6,7-tetrahydro-1H-indazol-1-yl)benzamide7.71 (b, 1H), 7.67 (b, 1H),7.38 (d, 1H), 6.63-6.60 (m,1H), 6.66 (s, 1H), 3.85-3.76(m, 1H), 2.94 (s, 2H), 2.38 (s,3H), 2.31 (s, 2H), 2.01-1.91(m, 2H), 1.70-1.52 (m, 4H),1.47-1.37 (m, 2H), 1.00 (s, 6H) | 399(M + H) |
| 208 | 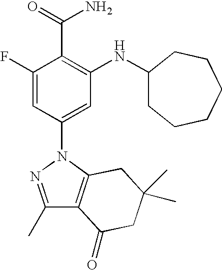 | 2-(cycloheptylamino)-6-fluoro-4-(3,6,6-trimethyl-4-oxo-4,5,6,7-tetrahydro-1H-indazol-1-yl)benzamide7.71 (b, 1H), 7.66 (b, 1H),6.60 (d, 1H), 6.58 (dd, 1H),6.52 (b, 1H), 3.59-3.49 (m,1H), 2.94 (s, 2H), 2.38 (s,3H), 2.31 (s, 2H), 1.93-1.83(m, 2H), 1.64-1.41 (m, 10H),1.00 (s, 6H) | 427(M + H) |
| 209 | 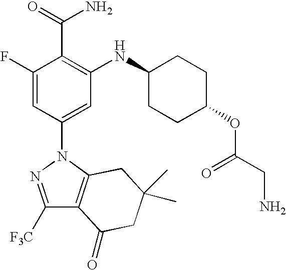 | (trans)-4-(2-carbamoyl-5-(6,6-dimethyl-4-oxo-3-(trifluoromethyl)-4,5,6,7-tetrahydro-1H-indazol-1-yl)-3-fluorophenylamino)cyclohexyl 2-aminoacetate8.16 (m, 3H), 7.80 (d, 2H),6.74 (s, 1H), 6.68 (m, 1H), 4.80(m, 1H), 3.80 (q, 2H), 3.47 (m,1H), 2.42 (s, 2H), 2.35 (s,8H), 1.97 (m, 4H), 1.88 (s,4H), 1.55 (m, 2H), 1.37 (m,2H), 1.02 (s, 6H) | 540(M + H) |
| 210 | 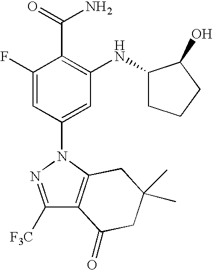 | 4-(6,6-dimethyl-4-oxo-3-(trifluoromethyl)-4,5,6,7-tetrahydro-1H-indazol-1-yl)-2-fluoro-6-((1S,2S)-2-hydroxycyclopentylamino)benzamide7.78 (d, 2H), 7.29 (d, 1H),6.79 (s, 1H), 6.70 (d, 1H),4.92 (d, 1H), 3.84 (br, 1H),3.50 (br, 1H), 2.97 (s, 3H),2.42 (s, 2H), 2.14 (m, 1H),2.18-2.16 (m, 2H), 1.51 (m,1H), 1.35 (m, 1H), 1.02 (d, 6H) | 469(M + H) |
| 211 | 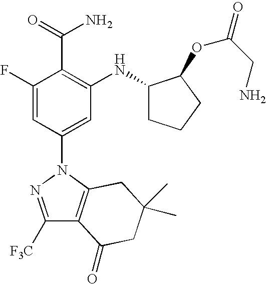 | (1S,2S)-2-(2-carbamoyl-5-(6,6-dimethyl-4-oxo-3-(trifluoromethyl)-4,5,6,7-tetrahydro-1H-indazol-1-yl)-3-fluorophenylamino)cyclopentyl2-aminoacetate8.14 (br, 3H), 7.83 (s, 2H),7.36 (d, 1H), 6.84 (s, 1H),7.77 (d, 1H), 3.78 (br, 2H),2.95 (q, 2H), 2.44 (s, 2H),2.28 (s, 3H), 2.16-2.05 (m,2H), 1.76-1.66 (m, 3H), 1.53-1.50 (m, 1H), 1.34 (s, 2H),1.02 (s, 6H) | 526(M + H) |
| 212 | 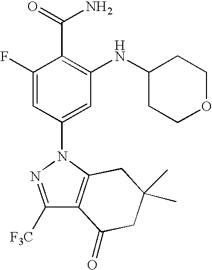 | 4-(6,6-dimethyl-4-oxo-3-(trifluoromethyl)-4,5,6,7-tetrahydro-1H-indazol-1-yl)-2-fluoro-6-(tetrahydro-2H-pyran-4-ylamino)benzamide | 469(M + H) |
| 213 | 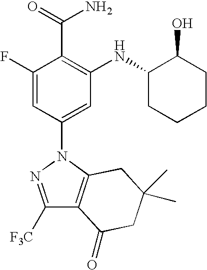 | 4-(6,6-dimethyl-4-oxo-3-(trifluoromethyl)-4,5,6,7-tetrahydro-1H-indazol-1-yl)-2-fluoro-6-((1S,2S)-2-hydroxycyclohexylamino)benzamide7.75 (br s, 2H), 7.33 (d, 1H),7.76 (d, 1H), 6.64 (dd, 1H),4.84 (m, 1H), 3.15 (m, 1H),2.96 (2 d, 2H), 2.42 (2 d, 2H),1.98 (m, 1H), 1.82, (m, 1H),1.62 (m, 1H), 1.54 (m, 1H),1.20-1.36 (m, 3H), 1.12 (m,1H), 1.03 (s, 3H), 1.01 (s, 3H) | 483(M + H) |
| 214 | 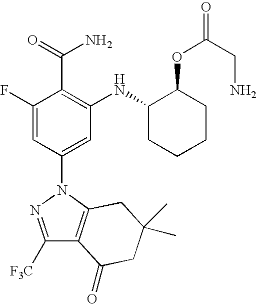 | (1S,2S)-2-(2-carbamoyl-5-(6,6-dimethyl-4-oxo-3-(trifluoromethyl)-4,5,6,7-tetrahydro-1H-indazol-1-yl)-3-fluorophenylamino)cyclohexyl 2-aminoacetate8.12 (b, 1H), 7.79 (b, 1H),7.30 (d, 1H), 6.89 (b, 1H),6.75 (dd, 1H), 4.86-4.78 (m,1H), 3.82-3.52 (m, 3H), 2.97(m, 2H), 2.43 (m, 2H), 2.03-1.88 (m, 2H), 1.73-1.57 (m,2H), 1.44-1.21 (m, 4H), 1.05(s, 3H), 1.01 (s, 3H) | 540(M + H) |
| 215 | 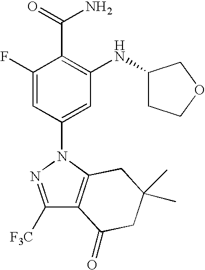 | (S)-4-(6,6-dimethyl-4-oxo-3-(trifluoromethyl)-4,5,6,7-tetrahydro-1H-indazol-1-yl)-2-fluoro-6-(tetrahydrofuran-3-ylamino)benzamide7.82 (d, 2H), 7.35 (d, 1H),6.76 (q, 1H), 6.69 (s, 1H), 4.13(s, 1H), 3.82 (m, 2H), 3.71 (m,1H), 3.55 (m, 1H), 2.42 (s,2H), 2.22 (m, 1H), 1.75 (m,1H), 1.02 (s, 6H) | 454(M + H) |
| 232 | 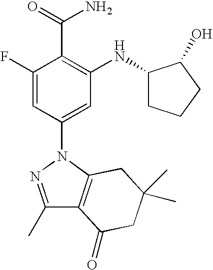 | 2-fluoro-6-((1S,2R)-2-hydroxycyclopentylamino)-4-(3,6,6-trimethyl-4-oxo-4,5,6,7-tetrahydro-1H-indazol-1-yl)benzamide7.65 (s br s, 2H), 7.40 (d,1H), 6.57 (d, 1H), 6.55 (dd,1H), 4.99 (d, 1H), 4.05 (m,1H), 3.59 (m, 1H), 2.95 (d,1H), 2.88 (d, 1H), 2.38 (s,3H), 2.31 (2 d, 2H), 1.99(m, 1H), 1.68-1.83 (m, 2H),1.46-1.61 (m, 2H), 1.40 (m,1H), 1.01 (s, 3H), 0.98 (s,3H) | 415(M + H) |
| 233 | 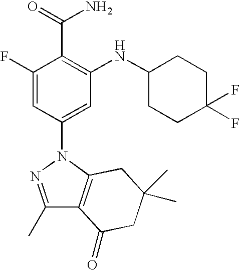 | 2-(4,4-difluorocyclohexylamino)-6-fluoro-4-(3,6,6-trimethyl-4-oxo-4,5,6,7-tetrahydro-1H-indazol-1-yl)benzamide7.76 (br s, 1H), 7.71 (br s,1H), 7.39 (d, 1H), 6.68 (s,1H), 6.62 (d, 1H), 3.64 (m,1H), 2.94 (s, 2H), 2.38 (s,3H), 2.31 (s, 2H), 1.99 (m,6H), 1.49 (m, 2H), 1.00 (s,6H) | 449(M + H) |
| 234 | 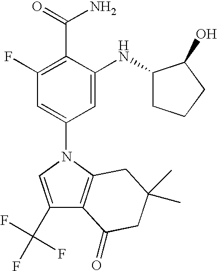 | 4-(6,6-dimethyl-4-oxo-3-(trifluoromethyl)-4,5,6,7-tetrahydro-1H-indol-1-yl)-2-fluoro-6-((1S,2S)-2-hydroxycyclopentylamino)benzamide7.75 (d, 2H), 7.67 (s, 1H),7.29 (d, 1H), 6.68 (s, 1H),6.63 (d, 1H), 4.92 (d, 1H),3.84 (m, 1H), 3.52 (m, 1H),2.75 (s, 2H), 2.32 (s, 2H),2.15 (m, 1H), 1.60-1.81 (m,2H), 1.50 (m, 1H), 1.35 (m,1H), 0.99 (s, 6H) | 468(M + H) |
| 235 | 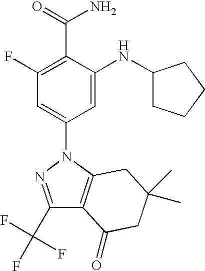 | 2-(cyclopentylamino)-4-(6,6-dimethyl-4-oxo-3-(trifluoromethyl)-4,5,6,7-tetrahydro-1H-indazol-1-yl)-6-fluorobenzamide7.78 (2 br s, 2H), 7.28 (d,1H), 6.69 (dd, 2H), 6.67 (d,1H), 3.81 (m, 1H), 2.97 (s,2H), 2.4s (s, 2H), 1.97 (m,2H), 1.51-1.70 (m, 4H), 1.41(m, 2H), 1.02 (s, 6H) | 453(M + H) |
| 236 | 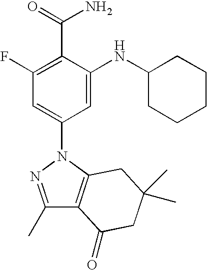 | 2-(cyclohexylamino)-6-fluoro-4-(3,6,6-trimethyl-4-oxo-4,5,6,7-tetrahydro-1H-indazol-1-yl)benzamide7.71 (br s, 1H), 7.67 (br s,1H), 7.37 (d, 1H), 6.60 (d,1H), 6.58 (dd, 1H), 3.38 (m,1H), 2.93 (s, 2H), 2.38 (s,3H), 2.31 (s, 2H), 1.90 (m,2H), 1.65 (m, 2H), 1.55 (m,1H), 1.37 (m, 2H), 1.23 (m,3H), 1.00 (s, 6H) | 413(M + H) |
| 237 | 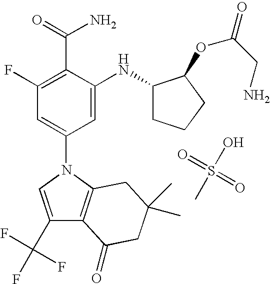 | (1S,2S)-2-(2-carbamoyl-5-(6,6-dimethyl-4-oxo-3-(trifluoromethyl)-4,5,6,7-tetrahydro-1H-indol-1-yl)-3-fluorophenylamino)cyclopentyl2-aminoacetatemethanesulfonate8.12 (s, 3H), 7.78 (s, 2H),7.67 (s, 1H), 7.41 (d, 1H),6.73 (d, 2H), 5.04 (m, 1H),3.92 (m, 1H), 3.77 (m, 2H),3.54 (s, 1H), 2.76 (q, 2H),2.34 (s, 2H), 2.27 (s, 5H),2.17 (m, 1H), 2.07 (m, 1H),1.76 (m, 2H), 1.68 (m, 1H),1.50 (m, 1H), 0.99 (d, 7H) | 525(M + H) |
| 238 | 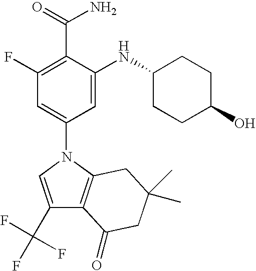 | 4-(6,6-dimethyl-4-oxo-3-(trifluoromethyl)-4,5,6,7-tetrahydro-1H-indol-1-yl)-2-fluoro-6-(trans-4-hydroxycyclohexylamino)benzamide7.74 (bs, 1H), 7.71 (bs, 1H),7.69 (s, 1H), 7.17 (dd, 1H),4.54 (d, 1H), 3.47-3.36 (m,2H), 2.75 (s, 2H), 2.48-2.47(m, 1H), 2.33 (s, 2H), 1.96-1.92 (m, 2H), 1.80-1.76 (m,2H), 1.31 (q, 2H), 1.18 (q,2H), 1.00 (s, 6H) | 482(M + H) |
| 239 | 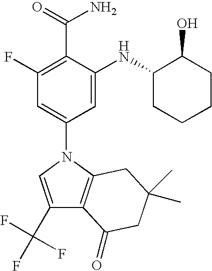 | 4-(6,6-dimethyl-4-oxo-3-(trifluoromethyl)-4,5,6,7-tetrahydro-1H-indol-1-yl)-2-fluoro-6-((1S,2S)-2-hydroxycyclohexylamino)benzamide7.71 (d, 2H), 7.66 (s, 1H),7.35 (d, 1H), 6.64 (s, 1H),6.57 (d, 1H), 3.28 (m, 1H),3.20 (m, 1H), 2.74 (m, 2H),2.32 (m, 2H), 1.98 (m, 1H),1.80 (m, 1H), 1.61 (m, 1H),1.52 (m, 1H), 1.28 (m, 3H),0.99 (d, 6H) | 482(M + H) |
| 240 | 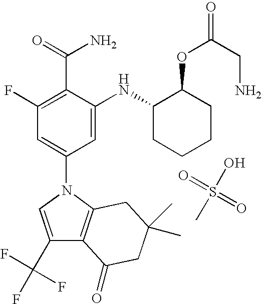 | (1S,2S)-2-(2-carbamoyl-5-(6,6-dimethyl-4-oxo-3-(trifluoromethyl)-4,5,6,7-tetrahydro-1H-indol-1-yl)-3-fluorophenylamino)cyclohexyl2-aminoacetatemethanesulfonate8.13 (s, 3H), 7.75 (s, 2H),7.66 (s, 1H), 7.34 (d, 1H),6.74 (s, 1H), 6.67 (m, 1H),4.82 (m, 1H), 3.92 (m, 1H),3.73 (m, 1H), 3.56 (m, 1H),2.74 (m, 2H), 2.33 (s, 2H),2.28 (s, 3H), 2.00 (m, 1H),1.90 (m, 1H), 1.68 (m, 1H),1.35-1.60 (m, 5H), 1.00 (d,6H) | 539(M + H) |
| 241 | 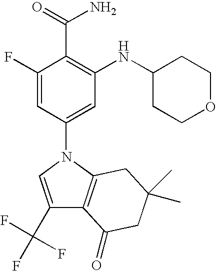 | 4-(6,6-dimethyl-4-oxo-3-(trifluoromethyl)-4,5,6,7-tetrahydro-1H-indol-1-yl)-2-fluoro-6-(tetrahydro-2H-pyran-4-ylamino)benzamide7.76 (b, 2H), 7.68 (s, 1H),7.24 (d, 1H), 6.68 (s, 1H),6.62 (d, 1H), 3.80 (d, 2H),3.66 (b, 1H), 3.46 (b, 2H),2.73 (s, 2H), 2.31 (s, 2H),1.90 (d, 2H), 1.35 (qd, 2H),0.99 (s, 6H) | 468(M + H) |
| 242 | 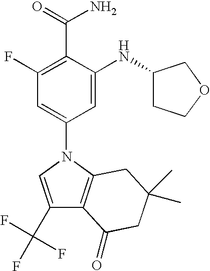 | (S)-4-(6,6-dimethyl-4-oxo-3-(trifluoromethyl)-4,5,6,7-tetrahydro-1H-indol-1-yl)-2-fluoro-6-(tetrahydrofuran-3-ylamino)benzamide7.79 (b, 1H), 7.77 (b, 1H),7.71 (d, 1H), 7.38 (dd, 1H),6.70 (dd, 1H), 6.60 (b, 1H),4.17 (b, 1H), 3.86-3.68 (m,3H), 3.54 (dd, 1H), 2.76 (s,2H), 2.31 (s, 2H), 2.26-2.18(m, 1H), 1.77-1.70 (m, 1H),1.00 (s, 6H) | 454(M + H) |
| 243 | 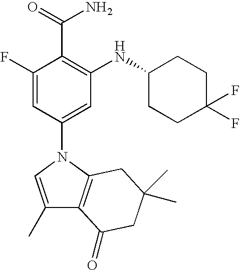 | 2-(4,4-difluorocyclohexylamino)-6-fluoro-4-(3,6,6-trimethyl-4-oxo-4,5,6,7-tetrahydro-1H-indol-1-yl)benzamide7.73 (br s, 1H), 7.56 (br s,1H), 7.70 (d, 1H), 6.85 (s,1H), 6.56 (s, 1H), 6.52 (d,1H), 3.67 (m, 1H), 2.74 (s,2H), 2.23 (s, 2H), 2.20 (s,3H), 2.06-1.91 (m, 6H), 1.46(m, 2H), 0.98 (s, 6H) | 448(M + H) |
| 244 | 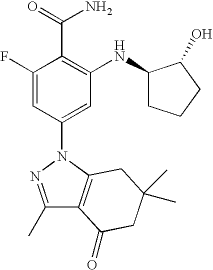 | 2-fluoro-6-((1R,2R)-2-hydroxycyclopentylamino)-4-(3,6,6-trimethyl-4-oxo-4,5,6,7-tetrahydro-1H-indazol-1-yl)benzamide7.81 (b, 1H), 7.78 (b, 1H),6.82 (d, 1H), 6.71 (dd, 1H),3.93 (b, 1H), 3.04 (m, 2H),2.59-2.55 (m, 2H), 2.47 (s,3H), 2.42-2.38 (m, 2H), 2.27-2.20 (m, 1H), 2.06 (dd, 1H),1.92-1.58 (m, 2H), 1.50-1.40(m, 1H), 1.28-1.22 (m, 1H),1.10 (s, 6H) | 415(M + H) |
| 245 | 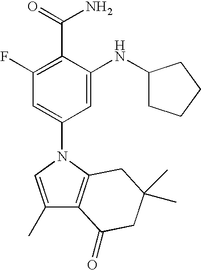 | 2-(cyclopentylamino)-6-fluoro-4-(3,6,6-trimethyl-4-oxo-4,5,6,7-tetrahydro-1H-indol-1-yl)benzamide7.67 (d, 2H), 7.37 (d, 1H),6.83 (s, 1H), 6.50 (d, 1H),6.42 (s, 1H), 3.83 (q, 1H),2.73 (s, 2H), 2.23 (s, 2H),2.19 (s, 2H), 2.11 (d, 1H),1.96 (s, 2H), 1.70-1.50 (m,4H), 1.45-1.35 (m, 2H), 0.99(s, 6H) | 398(M + H) |
| 246 | 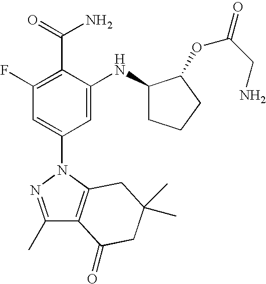 | (1R,2R)-2-(2-carbamoyl-3-fluoro-5-(3,6,6-trimethyl-4-oxo-4,5,6,7-tetrahydro-1H-indazol-1-yl)phenylamino)cyclopentyl 2-aminoacetate7.76 (s, 1H), 7.72 (s, 1H),7.40 (d, 1H), 6.80 (s, 1H),6.65 (dd, 1H), 4.88-4.85 (m,1H), 3.83 (b, 1H), 2.93 (s,3H), 2.38 (s, 4H), 2.31 (b,1H), 2.16-1.98 (m, 3H), 1.76-1.46 (m, 6H), 1.10 (s, 3H),0.99 (s, 3H) | 472(M + H) |
| 247 | 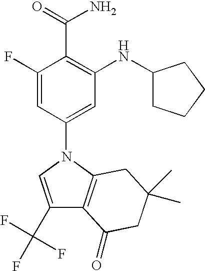 | 2-(cyclopentylamino)-4-(6,6-dimethyl-4-oxo-3-(trifluoromethyl)-4,5,6,7-tetrahydro-1H-indol-1-yl)-6-fluorobenzamide7.73 (d, 2H), 7.69 (s, 1H),7.31 (d, 1H), 6.64 (d, 1H),6.55 (s, 1H), 3.83 (q, 1H),2.75 (s, 2H), 2.33 (s, 2H),1.96 (m, 2H), 1.65-1.55 (m,4H), 1.40-1.35 (m, 2H), 0.99(s, 6H) | 452(M + H) |
| 248 | 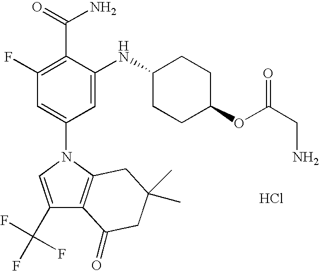 | trans-4-(2-carbamoyl-5-(6,6-dimethyl-4-oxo-3-(trifluoromethyl)-4,5,6,7-tetrahydro-1H-indol-1-yl)-3-fluorophenylamino)cyclohexyl2-aminoacetate hydrochloride8.22 (b, 3H), 7.76 (b, 1H),7.74 (b, 1H), 7.70 (s, 1H),6.65 (s, 1H), 6.64 (dd, 1H),4.80 (b, 1H), 3.79 (q, 2H),2.76 (s, 2H), 2.33 (s, 2H),2.03-1.90 (m, 4H), 1.57 (q,2H), 1.35 (q, 2H), 0.99 (s,6H) | 539(M + H) |
| 249 | 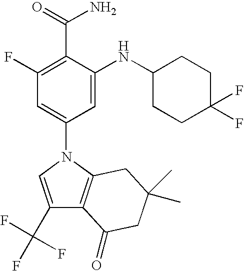 | 2-(4,4-difluorocyclohexylamino)-4-(6,6-dimethyl-4-oxo-3-(trifluoromethyl)-4,5,6,7-tetrahydro-1H-indol-1-yl)-6-fluorobenzamide | 502(M + H) |
| 250 | 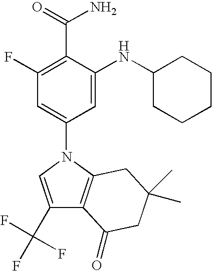 | 2-(cyclohexylamino)-4-(6,6-dimethyl-4-oxo-3-(trifluoromethyl)-4,5,6,7-tetrahydro-1H-indol-1-yl)-6-fluorobenzamide | 466(M + H) |

Examples 101-141, 216-219, 251, 252, and 254 shown below in Table 2, were prepared essentially according to the synthetic methodology described herein, Examples 1-47 and Schemes 1-5, and/or by using methodology well known in the art.

TABLE 2

| | | Nomenclature | |
|---|---|---|---|
| Ex. | Structure | NMR (400 MHz, DMSO) δ (ppm) | MS |
| 101 | 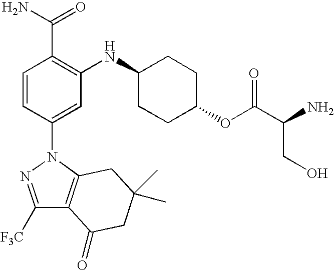 | (S)-(trans-4-(2-carbamoyl-5-(6,6-dimethyl-4-oxo-3-(trifluoromethyl)-4,5,6,7-tetrahydro-1H-indazol-1-yl)phenylamino)cyclohexyl) 2-amino-3-hydroxypropanoate | 552(M + H) |
| 102 | 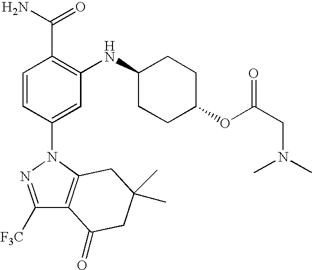 | trans-4-(2-carbamoyl-5-(6,6-dimethyl-4-oxo-3-(trifluoromethyl)-4,5,6,7-tetrahydro-1H-indazol-1-yl)phenylamino)cyclohexyl 2-(dimethylamino) acetate | 550(M + H) |
| 103 | 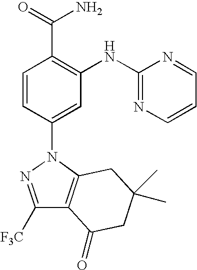 | 4-(6,6-dimethyl-4-oxo-3-(trifluoromethyl)-4,5,6,7-tetrahydro-1H-indazol-1-yl)-2-(pyrimidin-2-ylamino)benzamide9.05 (d, 1H), 8.53 (d, 2H),8.37 (b, 1H), 7.96 (d, 2H),2.27 (dd, 2H), 6.97 (t, 1H),3.37 (s, 2H), 2.47 (s, 2H),1.04 (s, 6H) | 445(M + H) |
| 104 | 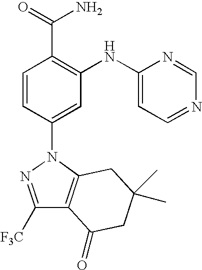 | 4-(6,6-dimethyl-4-oxo-3-(trifluoromethyl)-4,5,6,7-tetrahydro-1H-indazol-1-yl)-2-(pyrimidin-4-ylamino)benzamide8.90 (s, 1H), 8.70 (b, 1H),8.38 (b, 1H), 7.97 (d, 1H),7.86 (b, 1H), 7.57-7.50 (m,1H), 7.37 (d, 1H), 7.01-6.96(m, 1H), 6.50 (s, 1H), 3.21(s, 2H), 2.97 (s, 2H), 1.06(s, 6H) | 445(M + H) |
| 105 | 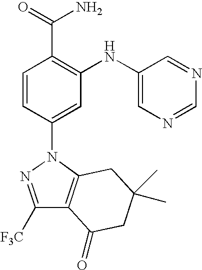 | 4-(6,6-dimethyl-4-oxo-3-(trifluoromethyl)-4,5,6,7-tetrahydro-1H-indazol-1-yl)-2-(pyrimidin-5-ylamino)benzamide8.82 (s, 1H), 8.77 (s, 2H),8.26 (b, 1H), 7.91 (d, 1H),7.72 (b, 1H), 7.39 (d, 1H),7.15 (dd, 1H), 2.94 (s, 2H),2.35 (s, 2H), 1.02 (s, 6H) | 445(M + H) |
| 106 | 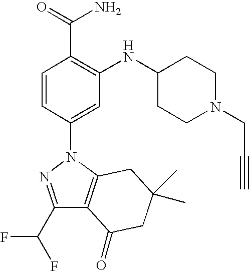 | 4-(3-(difluoromethyl)-6,6-dimethyl-4-oxo-4,5,6,7-tetrahydro-1H-indazol-1-yl)-2-(1-(prop-2-ynyl)piperidin-4-ylamino)benzamide8.45 (d, 1H), 7.96 (b, 1H),7.76 (d, 1H), 7.29 (b, 1H),6.84 (d, 1H), 6.70 (dd, 1H),3.44-3.28 (m, 1H), 3.26 (d,2H), 3.11-3.10 (m, 1H), 2.97(s, 1H), 2.94 (s, 2H), 2.74-2.65 (m, 2H), 2.40 (s, 2H),2.37-2.29 (m, 2H), 2.00-1.99(m, 2H), 1.46-1.35 (m, 2H),1.01 (s, 6H) | 470(M + H) |
| 107 | 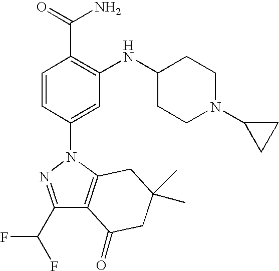 | 2-(1-cyclopropylpiperidin-4-ylamino)-4-(3-(difluoromethyl)-6,6-dimethyl-4-oxo-4,5,6,7-tetrahydro-1H-indazol-1-yl)benzamide8.23 (d, 1H), 7.71 (b, 1H),7.52 (d, 1H), 7.04 (b, 1H),6.58 (d, 1H), 6.45 (dd, 1H),3.22-3.13 (m, 1H), 2.73 (s,1H), 2.70 (s, 2H), 2.60-2.50(m, 1H), 2.15 (s, 2H), 2.14-2.06 (m, 2H), 1.69-1.60 (m,2H), 1.36-1.30 (m, 1H), 1.15-1.04 (m, 2H), 0.76 (s, 6H),0.17-0.11 (m, 2H), 0.05-0.02(m, 2H) | 472(M + H) |
| 108 | 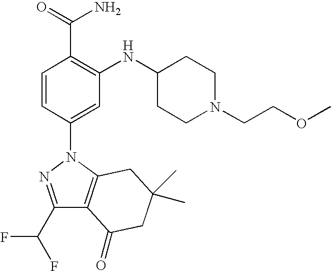 | 4-(3-(difluoromethyl)-6,6-dimethyl-4-oxo-4,5,6,7-tetrahydro-1H-indazol-1-yl)-2-(1-(2-methoxyethyl)piperidin-4-ylamino)benzamide | 490(M + H) |
| 109 | 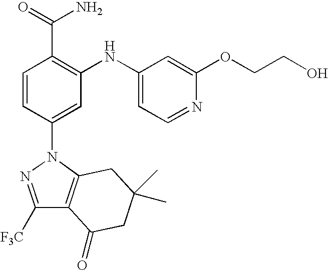 | 4-(6,6-dimethyl-4-oxo-3-(trifluoromethyl)-4,5,6,7-tetrahydro-1H-indazol-1-yl)-2-(2-(2-hydroxyethoxy)pyridin-4-ylamino)benzamide8.20 (b, 1H), 8.12-8.09 (m,1H), 7.89 (d, 1H), 7.73-7.68(m, 1H), 7.61 (b, 1H), 6.99-6.91 (m, 2H), 6.90-6.80 (m,1H), 4.23 (t, 2H), 3.73-3.66(m, 2H), 2.87 (s, 2H), 2.39(s, 2H), 1.00 (s, 6H) | 504(M + H) |
| 110 | 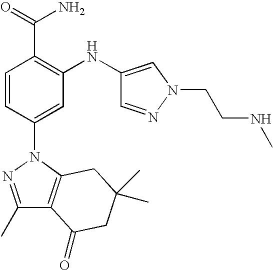 | 2-(1-(2-(methylamino)ethyl)-1H-pyrazol-4-ylamino)-4-(3,6,6-trimethyl-4-oxo-4,5,6,7-tetrahydro-1H-indazol-1-yl)benzamide7.83 (b, 1H), 7.92 (s, 1H),7.84 (d, 1H), 7.57 (s, 1H),7.46 (b, 1H), 6.94 (d, 1H),6.82 (dd, 1H), 4.42 (t, 2H),3.52-3.31 (m, 2H), 2.83 (s,2H), 2.52 (s, 3H), 2.34 (s,3H), 2.29 (s, 3H), 0.99 (s,6H) | 436(M + H) |
| 111 | 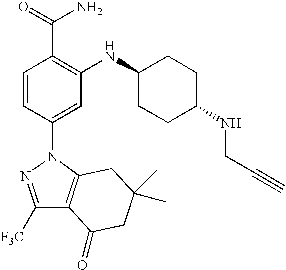 | 4-(6,6-dimethyl-4-oxo-3-(trifluoromethyl)-4,5,6,7-tetrahydro-1H-indazol-1-yl)-2-(trans-4-(prop-2-ynylamino)cyclohexylamino)benzamide8.36 (d, 1H), 7.75 (s, 1H),6.84 (d, 1H), 6.68 (m, 1H),2.98 (t, 1H), (s, 1H), 2.95(s, 2H), 2.42 (s, 2H), 1.99(d, 2H), 1.86 (d, 2H), 1.17(m, 5H), 1.01 (s, 6H) | 502(M + H) |
| 112 | 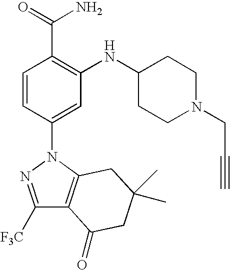 | 4-(6,6-dimethyl-4-oxo-3-(trifluoromethyl)-4,5,6,7-tetrahydro-1H-indazol-1-yl)-2-(1-(prop-2-ynyl)piperidin-4-ylamino)benzamide8.44 (d, 1H), 7.98 (b, 1H),7.77 (d, 1H), 7.32 (b, 1H),6.88 (d, 1H), 6.70 (dd, 1H),3.25 (d, 2H), 3.11-3.10 (m,1H), 2.94 (s, 2H), 2.73-2.65(m, 2H), 2.52 (s, 1H), 2.42(s, 2H), 2.37-2.29 (m, 2H),2.00-1.90 (m, 2H), 1.46-1.35(m, 2H), 1.01 (s, 6H) | 488(M + H) |
| 113 | 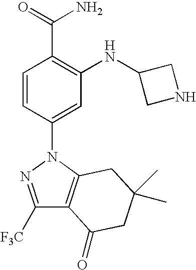 | 2-(azetidin-3-ylamino)-4-(6,6-dimethyl-4-oxo-3-(trifluoromethyl)-4,5,6,7-tetrahydro-1H-indazol-1-yl)benzamide | 422(M + H) |
| 114 | 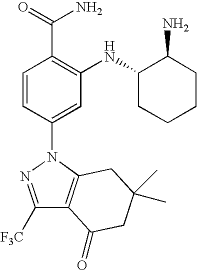 | 2-((1S,2S)-2-aminocyclohexylamino)-4-(6,6-dimethyl-4-oxo-3-(trifluoromethyl)-4,5,6,7-tetrahydro-1H-indazol-1-yl)benzamide8.20 (d, 1H), 8.08 (b, 1H),7.79 (d, 1H), 7.40 (b, 1H),7.06 (d, 1H), 6.78 (dd, 1H),3.05-2.91 (m, 3H), 2.43 (s,1H), 2.40 (s, 1H), 2.06-1.94(m, 2H), 1.70-1.57 (m, 2H),1.53-1.42 (m, 1H), 1.34-1.15(m, 4H), 1.02 (s, 3H), 0.99(s, 3H) | 464(M + H) |
| 115 | 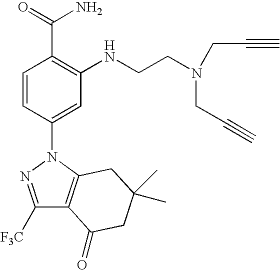 | 4-(6,6-dimethyl-4-oxo-3-(trifluoromethyl)-4,5,6,7-tetrahydro-1H-indazol-1-yl)-2-(2-(diprop-2-ynylamino)ethylamino)benzamide | 486(M + H) |
| 116 | 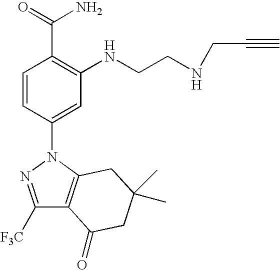 | 4-(6,6-dimethyl-4-oxo-3-(trifluoromethyl)-4,5,6,7-tetrahydro-1H-indazol-1-yl)-2-(2-(prop-2-ynylamino)ethylamino)benzamide | 448(M + H) |
| 117 | 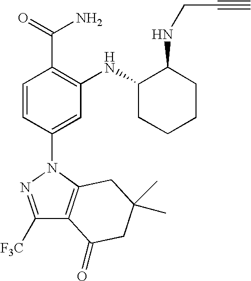 | 4-(6,6-dimethyl-4-oxo-3-(trifluoromethyl)-4,5,6,7-tetrahydro-1H-indazol-1-yl)-2-((1S,2S)-2-(prop-2-ynylamino)cyclohexylamino)benzamide8.49 (d, 1H), 7.93 (b, 1H),7.76 (d, 1H), 7.32 (b, 1H),6.94 (d, 1H), 6.70 (dd, 1H),3.43-3.28 (m, 1H), 2.95 (s,2H), 2.86 (s, 1H), 2.71 (s,2H), 2.64-2.59 (m, 1H), 2.42(s, 2H), 1.99-1.86 (m, 2H),1.67-1.54 (m, 2H), 1.37-1.11(m, 4H), 1.01 (s, 6H) | 502(M + H) |
| 118 | 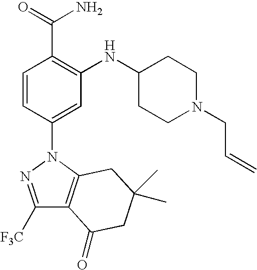 | 2-(1-allylpiperidin-4-ylamino)-4-(6,6-dimethyl-4-oxo-3-(trifluoromethyl)-4,5,6,7-tetrahydro-1H-indazol-1-yl)benzamide8.49 (d, 1H), 7.99 (b, 1H),7.78 (d, 1H), 7.32 (b, 1H),6.86 (d, 1H), 6.70 (dd, 1H),5.84-5.72 (m, 1H), 5.18-5.04(m, 2H), 2.94 (s, 2H), 2.91(d, 2H), 2.71-2.63 (m, 2H),2.52 (s, 1H), 2.41 (s, 2H),2.19-2.09 (m, 2H), 1.96-1.87(m, 2H), 1.48-1.37 (m, 2H),1.01 (s, 6H) | 490(M + H) |
| 119 | 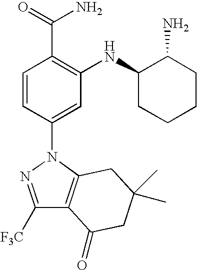 | 2-((1R,2R)-2-aminocyclohexylamino)-4-(6,6-dimethyl-4-oxo-3-(trifluoromethyl)-4,5,6,7-tetrahydro-1H-indazol-1-yl)benzamide | 464(M + H) |
| 120 | 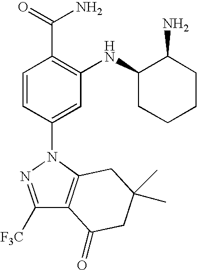 | 2-((1R,2S)-2-aminocyclohexylamino)-4-(6,6-dimethyl-4-oxo-3-(trifluoromethyl)-4,5,6,7-tetrahydro-1H-indazol-1-yl)benzamide | 464(M + H) |
| 121 | 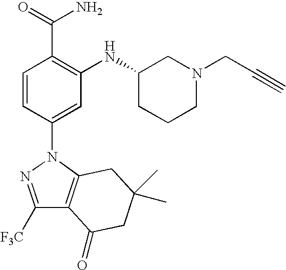 | (S)-4-(6,6-dimethyl-4-oxo-3-(trifluoromethyl)-4,5,6,7-tetrahydro-1H-indazol-1-yl)-2-(1-(prop-2-ynyl)piperidin-3-ylamino)benzamide8.50 (d, 1H), 7.97 (b, 1H),7.76 (d, 1H), 7.29 (b, 1H),6.85 (d, 1H), 6.70 (dd, 1H),3.16-3.06 (m, 1H), 2.96 (d,2H), 2.87 (s, 2H), 2.71 (s,2H), 2.42 (s, 2H), 1.78-1.20(m, 6H), 1.01 (s, 6H) | 488(M + H) |
| 122 | 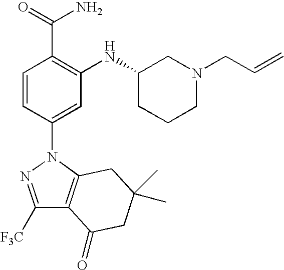 | (S)-2-(1-allylpiperidin-3-ylamino)-4-(6,6-dimethyl-4-oxo-3-(trifluoromethyl)-4,5,6,7-tetrahydro-1H-indazol-1-yl)benzamide8.53 (d, 1H), 7.95 (b, 1H),7.75 (d, 1H), 7.29 (b, 1H),6.83 (d, 1H), 6.69 (dd, 1H),5.38-5.73 (m, 1H), 5.18 (dd,1H), 5.07 (dd, 1H), 3.63-3.55(m, 1H), 2.94 (s, 2H), 2.72-2.62 (m, 1H), 2.46-2.37 (m,3H), 2.31-2.22 (m, 1H), 2.21-2.11 (m, 1H), 1.76-1.59 (m,2H), 1.55-1.44 (m, 1H), 1.42-1.32 (m, 1H), 1.02 (s, 3H),1.00 (s, 3H) | 490(M + H) |
| 123 | 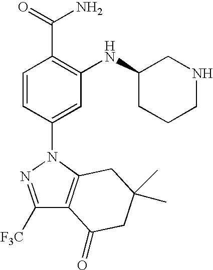 | (R)-4-(6,6-dimethyl-4-oxo-3-(trifluoromethyl)-4,5,6,7-tetrahydro-1H-indazol-1-yl)-2-(piperidin-3-ylamino)benzamide8.46 (d, 1H), 8.01 (b, 1H),7.78 (d, 1H), 7.34 (b, 1H),6.87 (d, 1H), 6.72 (dd, 1H),3.18-3.10 (m, 1H), 2.99 (s,2H), 3.01-2.83 (m, 2H), 2.68-2.59 (m, 1H), 2.42 (s, 2H),2.45-2.36 (m, 1H), 1.97-1.88(m, 1H), 1.77-1.38 (m, 3H),1.01 (s, 6H) | 450(M + H) |
| 124 | 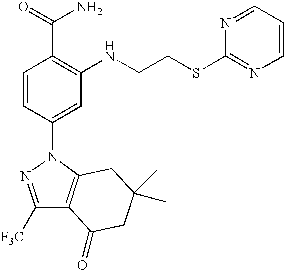 | 4-(6,6-dimethyl-4-oxo-3-(trifluoromethyl)-4,5,6,7-tetrahydro-1H-indazol-1-yl)-2-(2-(pyrimidin-2-ylthio)ethylamino)benzamide8.54-8.44 (m, 3H), 7.98 (b,1H), 7.80-7.73 (m, 1H), 7.34(b, 1H), 7.20-7.11 (m, 1H),7.09-7.04 (m, 1H), 6.77-6.70(m, 1H), 3.55-3.52 (m, 2H),2.93 (s, 2H), 2.52-2.44 (m,2H), 2.40 (s, 2H), 0.97 (s,3H) | 578(M + H) |
| 125 | 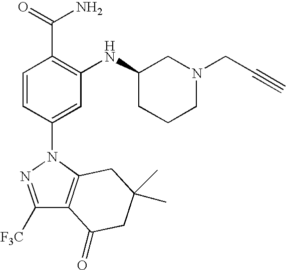 | (R)-4-(6,6-dimethyl-4-oxo-3-(trifluoromethyl)-4,5,6,7-tetrahydro-1H-indazol-1-yl)-2-(1-(prop-2-ynyl)piperidin-3-ylamino)benzamide8.50 (d, 1H), 7.95 (b, 1H),7.76 (d, 1H), 7.28 (b, 1H),6.85 (d, 1H), 6.70 (dd, 1H),3.65-3.58 (m, 1H), 3.29 (d,2H), 3.10 (s, 1H), 2.95 (s,1H), 2.81-2.71 (m, 1H), 2.42(s, 2H), 2.39-2.31 (m, 1H),2.28-2.21 (m, 1H), 1.78-1.61(m, 2H), 1.58-1.47 (m, 1H),1.38-1.28 (m, 1H), 1.27-1.19(m, 2H), 1.02 (s, 3H), 1.01(s, 3H) | 488(M + H) |
| 126 | 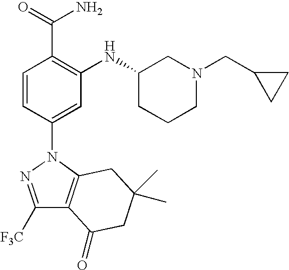 | (S)-2-(1-(cyclopropylmethyl)piperidin-3-ylamino)-4-(6,6-dimethyl-4-oxo-3-(trifluoromethyl)-4,5,6,7-tetrahydro-1H-indazol-1-yl)benzamide8.37 (d, 1H), 7.84 (b, 1H),7.62 (d, 1H), 7.18 (b, 1H),6.72 (d, 1H), 6.58 (dd, 1H),3.60-3.50 (m, 1H), 2.84 (s,2H), 2.37 (s, 2H), 2.29-2.21(m, 4H), 1.74-1.65 (m, 1H),1.64-1.55 (m, 1H), 1.52-1.43(m, 1H), 1.29-1.20 (m, 1H),1.08 (d, 2H), 0.87 (s, 3H),0.84 (s, 3H), 0.35-0.30 (m,2H), 0.02-0.03 (m, 2H) | 504(M + H) |
| 127 | 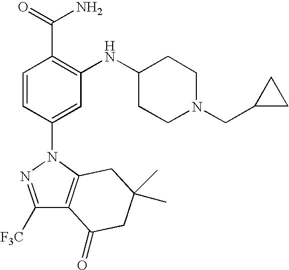 | 2-(1-(cyclopropylmethyl)piperidin-4-ylamino)-4-(6,6-dimethyl-4-oxo-3-(trifluoromethyl)-4,5,6,7-tetrahydro-1H-indazol-1-yl)benzamide8.46 (d, 1H), 7.95 (b, 1H),7.75 (d, 1H), 7.29 (b, 1H),6.83 (d, 1H), 6.68 (dd, 1H),3.43-3.34 (m, 1H), 2.92 (s,2H), 2.80-2.72 (m, 2H), 2.40(s, 2H), 2.22-2.11 (m, 2H),2.14 (d, 2H), 1.95-1.87 (m,2H), 1.45-1.34 (m, 2H), 0.99(s, 6H), 0.80-0.76 (m, 1H),0.43-0.39 (m, 2H), 0.04-0.00(m, 2H) | 504(M + H) |
| 128 | 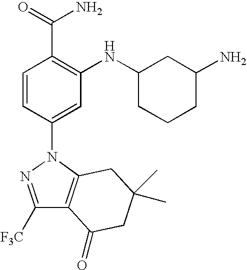 | 2-(3-aminocyclohexylamino)-4-(6,6-dimethyl-4-oxo-3-(trifluoromethyl)-4,5,6,7-tetrahydro-1H-indazol-1-yl)benzamide8.32 (d, 1H), 8.05 (br, 1H),7.78 (d, 1H), 7.32 (br, 1H),6.86 (s, 1H), 6.70 (d, 1H),3.12 (br, 1H), 3.05-2.84 (m,3H), 2.44-2.38 (d, 3H), 2.30-2.25 (br, 1H), 2.20-1.87 (br,2H), 1.75-1.70 (br, 1H), 1.30-1.20 (m, 1H), 1.05-0.95 (t,10H) | 464(M + H) |
| 129 | 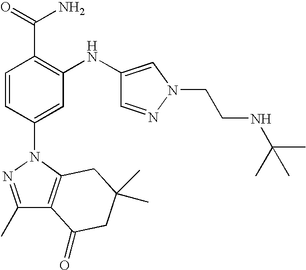 | 2-(1-(2-(tert-butylamino)ethyl)-1H-pyrazol-4-ylamino)-4-(3,6,6-trimethyl-4-oxo-4,5,6,7-tetrahydro-1H-indazol-1-yl)benzamide9.90 (s, 1H), 8.75 (b, 1H),8.11 (b, 1H), 7.96 (s, 1H),7.85 (d, 1H), 7.59 (s, 1H),7.74 (b, 1H), 6.96 (s, 1H),6.84 (d, 1H), 4.38 (m, 1H),3.59 (m, 1H), 3.37 (d, 1H),3.11 (m, 1H), 2.84 (s, 2H),2.35 (s, 3H), 2.29 (s, 2H),1.27 (s, 9H), 0.99 (s, 6H) | 478(M + H) |
| 130 | 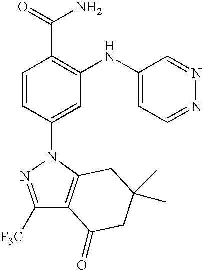 | 4-(6,6-dimethyl-4-oxo-3-(trifluoromethyl)-4,5,6,7-tetrahydro-1H-indazol-1-yl)-2-(pyridazin-4-ylamino)benzamide10.07 (s, 1H), 9.04 (d, 1H),8.76 (d, 1H), 8.23 (b, 1H),7.90 (d, 1H), 7.76 (b, 1H),7.67 (d, 1H), 7.40 (dd, 1H),7.28-7.26 (m, 1H), 3.01 (s,2H), 2.42 (s, 2H), 1.04 (s,6H) | 445(M + H) |
| 131 | 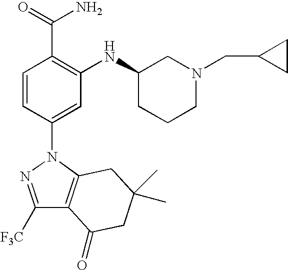 | (R)-2-(1-(cyclopropylmethyl)piperidin-3-ylamino)-4-(6,6-dimethyl-4-oxo-3-(trifluoromethyl)-4,5,6,7-tetrahydro-1H-indazol-1-yl)benzamide8.28-8.19 (m, 1H), 7.76 (b,1H), 7.52 (d, 1H), 7.12 (b,1H), 6.64 (d, 1H), 6.49 (dd,1H), 3.67 (b, 1H), 3.34-3.25(m, 2H), 2.86-2.78 (m, 2H),2.69-2.38 (m, 3H), 2.11 (d,2H), 1.77-1.49 (m, 3H), 1.18(b, 1H), 0.99-0.89 (m, 2H),0.71 (s, 3H), 0.70 (s, 3H),0.31-0.24 (m, 2H), 0.08-0.04(m, 2H) | 504(M + H) |
| 132 | 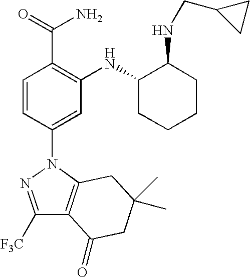 | 2-((1S,2S)-2-(cyclopropylmethylamino)cyclo-hexylamino)-4-(6,6-dimethyl-4-oxo-3-(trifluoromethyl)-4,5,6,7-tetrahydro-1H-indazol-1-yl)benzamide | 518(M + H) |
| 133 | 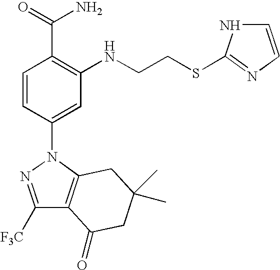 | 2-(2-(1H-imidazol-2-ylthio)ethylamino)-4-(6,6-dimethyl-4-oxo-3-(trifluoromethyl)-4,5,6,7-tetrahydro-1H-indazol-1-yl)benzamide9.50 (b, 1H), 8.31-8.29 (m,1H), 7.51 (d, 1H), 7.05-6.95(m, 3H), 6.68 (dd, 1H), 5.76(b, 2H), 3.52 (q, 2H), 3.25(t, 2H), 2.86 (s, 2H), 2.03(s, 2H), 1.11 (s, 6H) | 493(M + H) |
| 134 | 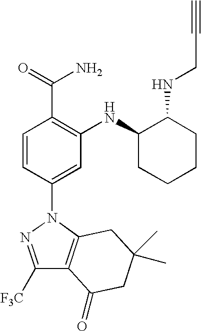 | 4-(6,6-dimethyl-4-oxo-3-(trifluoromethyl)-4,5,6,7-tetrahydro-1H-indazol-1-yl)-2-((1R,2R)-2-(prop-2-ynylamino)cyclohexylamino)benzamide | 502(M + H) |
| 135 | 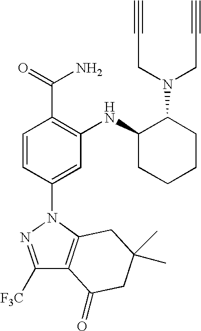 | 4-(6,6-dimethyl-4-oxo-3-(trifluoromethyl)-4,5,6,7-tetrahydro-1H-indazol-1-yl)-2-((1R,2R)-2-(diprop-2-ynylamino)cyclohexylamino)benzamide | 540(M + H) |
| 136 | 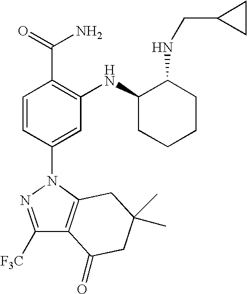 | 2-((1R,2R)-2-(cyclopropylmethylamino)cyclo-hexylamino)-4-(6,6-dimethyl-4-oxo-3-(trifluoromethyl)-4,5,6,7-tetrahydro-1H-indazol-1-yl)benzamide | 518(M + H) |
| 137 | 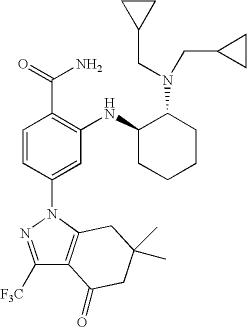 | 2-((1R,2R)-2-(bis(cyclopropylmethyl)amino)cyclohexylamino)-4-(6,6-dimethyl-4-oxo-3-(trifluoromethyl)-4,5,6,7-tetrahydro-1H-indazol-1-yl)benzamide | 572(M + H) |
| 138 | 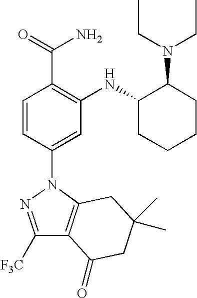 | 2-((1S,2S)-2-(diethylamino)cyclohexylamino)-4-(6,6-dimethyl-4-oxo-3-(trifluoromethyl)-4,5,6,7-tetrahydro-1H-indazol-1-yl)benzamide8.43 (br, 1H), 7.93 (br, 1H),7.65-7.55 (m, 1H), 7.30-7.20(br, 1H), 6.95 (br, 1H), 6.65(s, 1H), 3.09 (s, 3H), 2.61(s, 4H), 2.58 (s, 2H), 2.30-2.05 (br, 2H), 1.89 (s, 2H),1.75 (s, 2H), 1.51-1.20 (br,9H), 1.09 (s, 6H) | 520(M + H) |
| 139 | 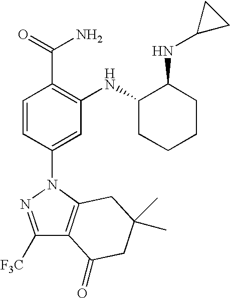 | 2-((1S,2S)-2-(cyclopropylamino)cyclohexyl-amino)-4-(6,6-dimethyl-4-oxo-3-(trifluoromethyl)-4,5,6,7-tetrahydro-1H-indazol-1-yl)benzamide | 504(M + H) |
| 140 | 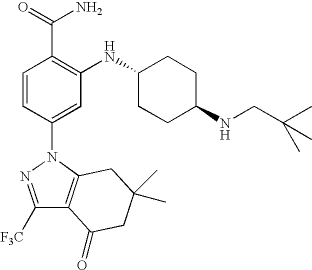 | 4-(6,6-dimethyl-4-oxo-3-(trifluoromethyl)-4,5,6,7-tetrahydro-1H-indazol-1-yl)-2-(trans-4-(neopentylamino)cyclohexylamino)benzamide8.37 (s, 1H), 8.35 (s, 1H),7.96 (b, 1H), 7.75 (d, 1H),7.29 (b, 1H), 6.33 (d, 1H),6.68 (dd, 1H), 2.97-2.95 (m,2H), 2.48-2.47 (m, 2H), 2.43(s, 2H), 2.07-1.94 (m, 2H),1.93-1.82 (m, 2H) 1.21-1.12(m, 4H), 1.02 (s, 6H), 0.82(s, 9H) | 534(M + H) |
| 141 | 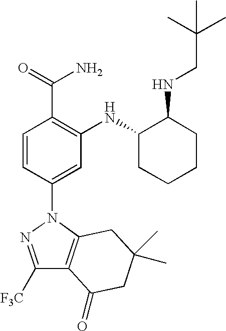 | 4-(6,6-dimethyl-4-oxo-3-(trifluoromethyl)-4,5,6,7-tetrahydro-1H-indazol-1-yl)-2-((1S,2S)-2-(neopentylamino)cyclohexylamino)benzamide8.42 (s, 1H), 8.39 (s, 1H),8.05 (b, 1H), 7.73 (d, 1H),7.30 (b, 1H), 6.95 (d, 1H),6.68 (dd, 1H), 3.02-2.87 (m,2H), 2.49-2.48 (m, 2H), 2.41-2.42 (m, 2H), 2.38-2.19 (m,2H), 2.04-1.86 (m, 2H), 1.68-1.54 (m, 2H), 1.39-1.11 (m,4H), 1.00 (d, 6H), 0.75 (s,9H) | 534(M + H) |
| 216 | 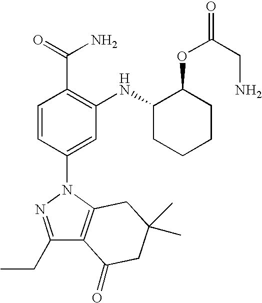 | (1S,2S)-2-(2-carbamoyl-5-(3-ethyl-6,6-dimethyl-4-oxo-4,5,6,7-tetrahydro-1H-indazol-1-yl)phenylamino)cyclohexyl 2-aminoacetate | 482(M + H) |
| 217 | 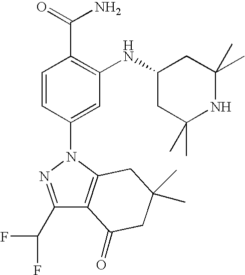 | 4-(3-(difluoromethyl)-6,6-dimethyl-4-oxo-4,5,6,7-tetrahydro-1H-indazol-1-yl)-2-(2,2,6,6-tetramethylpiperidin-4-ylamino)benzamide | 488(M + H) |
| 218 | 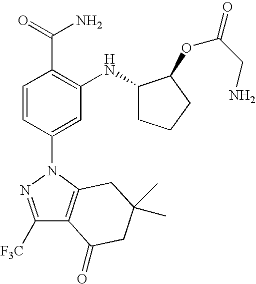 | (1S,2S)-2-(2-carbamoyl-5-(6,6-dimethyl-4-oxo-3-(trifluoromethyl)-4,5,6,7-tetrahydro-1H-indazol-1-yl)phenylamino)cyclopentyl 2-aminoacetate8.13 (s, 3H), 7.88 (s, H),7.80 (d, 1H), 6.99 (d, 1H),6.77 (q, 1H), 5.01 (m, 1H),3.91 (s, 1H), 3.78 (m, 2H),3.05 (s, 1H), 2.95 (m, 2H),2.44 (d, 2H), 2.28 (s, 4H),2.12 (m, 2H), 1.75 (m, 3H),1.52 (m, 1H), 1.08 (s, 3H),1.02 (s, 6H) | 508(M + H) |
| 219 | 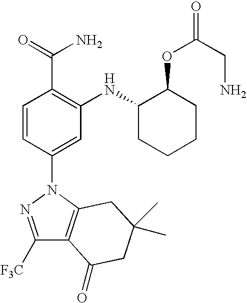 | (1S,2S)-2-(2-carbamoyl-5-(6,6-dimethyl-4-oxo-3-(trifluoromethyl)-4,5,6,7-tetrahydro-1H-indazol-1-yl)phenylamino)cyclohexyl 2-aminoacetate8.13 (b, 3H), 8.01 (b, 1H),7.78 (d, 1H), 7.33 (b, 1H),7.03 (d, 1H), 6.74 (dd, 1H),4.85-4.78 (m, 1H), 3.82-3.45(m, 3H), 3.06 (s, 1H), 3.04-2.86 (m, 2H), 2.46-2.42 (m,2H), 2.31 (s, 6H), 2.03-1.86(m, 2H), 1.72-1.34 (m, 6H),1.04 (s, 3H), 1.01 (s, 3H) | 522(M + H) |
| 251 | 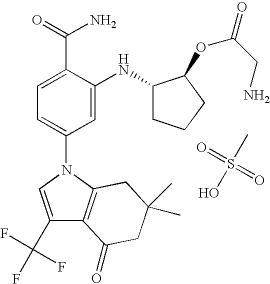 | (1S,2S)-2-(2-carbamoyl-5-(6,6-dimethyl-4-oxo-3-(trifluoromethyl)-4,5,6,7-tetrahydro-1H-indol-1-yl)phenylamino)cyclopentyl 2-aminoacetate methanesulfonate8.13 (s, 3H), 7.80 (d, 1H),7.65 (s, 1H), 6.88 (d, 1H),6.68 (q, 1H), 5.04 (m, 1H),3.92 (s, 1H), 3.78 (m, 2H),3.54 (s, 1H), 2.74 (q, 2H),2.34 (d, 2H), 2.28 (s, 8H),2.04-2.20 (m, 3H), 1.64-1.83(m, 4H), 1.52 (m, 1H), 1.08(s, 1H), 0.99 (d, 9H) | 507(M + H) |
| 252 | 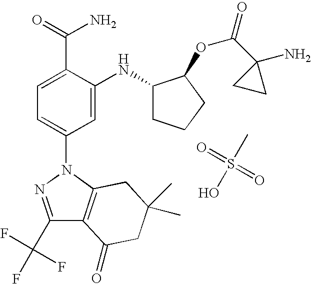 | (1S,2S)-2-(2-carbamoyl-5-(6,6-dimethyl-4-oxo-3-(trifluoromethyl)-4,5,6,7-tetrahydro-1H-indazol-1-yl)phenylamino)cyclopentyl 1-aminocyclopropanecarboxylatemethanesulfonate | 534(M + H) |
| 254 | 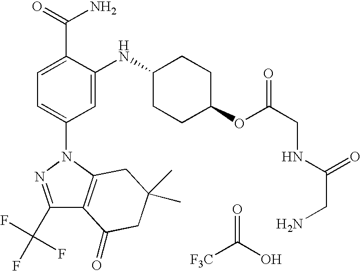 | trans-4-(2-carbamoyl-5-(6,6-dimethyl-4-oxo-3-(trifluoromethyl)-4,5,6,7-tetrahydro-1H-indazol-1-yl)phenylamino)cyclohexyl 2-(2-aminoacetamido)acetate2,2,2-trifluoroacetate | 579(M + H) |

Examples 142-183, 221-225, 229-230, 255-262, 264-272 shown below in Table 3, were prepared essentially according to the synthetic methodology described herein, Examples 1-47 and Schemes 1-5, and/or by using methodology well known in the art.

TABLE 3

| | | Nomeclature | |
|---|---|---|---|
| Ex. | Structure | NMR (400 MHz, DMSO) δ (ppm) | MS |
| 142 | 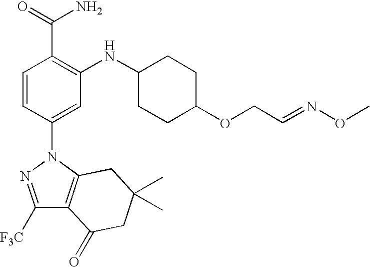 | 4-(6,6-dimethyl-4-oxo-3-(trifluoromethyl)-4,5,6,7-tetrahydro-1H-indazol-1-yl)-2-(4-(2-(methoxyimino)ethoxy)cyclohexyl-amino)benzamide8.40(d, 1H), 7.95(br, 1H),7.76(d, 1H), 7.35(m, 1H),6.85(s, 1H), 6.70(d, 1H),4.20(s, 1H), 4.01(d, 1H),3.74(d, 3H), 2.95(s, 2H),2.48(s, 2H), 2.41(s, 2H),1.95(br, 5H), 1.35(m, 2H),1.21(m, 2H), 1.02(s, 6H) | 536(M + H) |
| 143 | 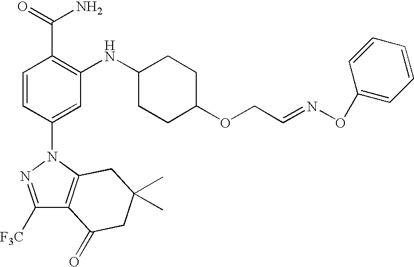 | 4-(6,6-dimethyl-4-oxo-3-(trifluoromethyl)-4,5,6,7-tetrahydro-1H-indazol-1-yl)-2-(4-(2-(phenoxyimino)ethoxy)cyclohexyl-amino)benzamide9.36(m, 1H), 8.48(m, 1H),8.05(br, 1H), 7.85(m, 1H),7.40(br, 1H), 7.20(m, 2H),6.95(nbr, 1H), 6.77(m, 5H),4.53(m, 2H), 3.02(br, 2H),2.60-2.50(m, 3H), 2.07(br,4H), 1.44(m, 2H), 1.32(m,2H), 1.02(br, 6H) | 598(M + H) |
| 144 | 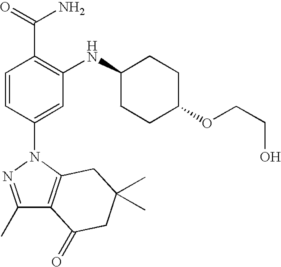 | 2-(trans-4-(2-hydroxyethoxy)cyclohexylamino)44-(3,6,6-trimethyl-4-oxo-4,5,6,7-tetrahydro-1H-indazol-1-yl)benzamide | 455(M + H) |
| 145 | 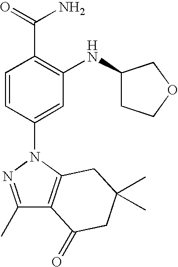 | (R)-2-(tetrahydrofuran-3-ylamino)-4-(3,6,6-trimethyl-4-oxo-4,5,6,7-tetrahydro-1H-indazol-1-yl)benzamide | 383(M + H) |
| 146 | 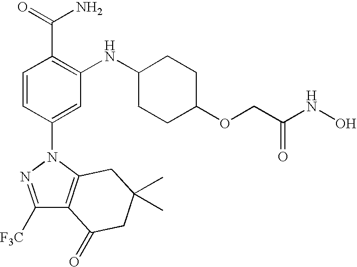 | 4-(6,6-dimethyl-4-oxo-3-(trifluoromethyl)-4,5,6,7-tetrahydro-1H-indazol-1-yl)-2-(4-(2-(hydroxyamino)-2-oxoethoxy)cyclohexylamino)benz-amide8.10(d, 1H), 7.93(d, 1H),7.72(d, 1H), 7.68(br, 1H),7.55(br, 1H), 7.45(s, 1H), 7.25(d, 1H), 3.95(m, 1H),2.95(s, 2H), 2.48(s, 2H),2.43(s, 2H), 2.30(m, 1H),1.83(m, 1H), 1.75(br, 1H),1.60(m, 1H), 1.35(s, 3H),1.30(t, 3H), 1.02(s, 6H) | 538(M + H) |
| 147 | 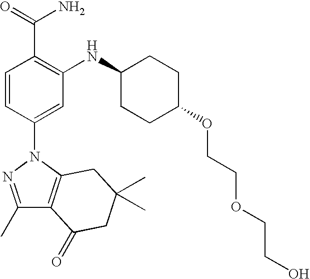 | 2-(trans-4-(2-(2-hydroxyethoxy)ethoxy)cyclohexyl-amino)-4-(3,6,6-trimethyl-4-oxo-4,5,6,7-tetrahydro-1H-indazol-1-yl)benzamide | 499(M + H) |
| 148 | 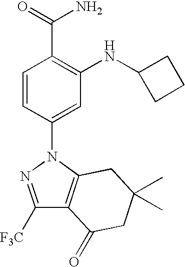 | 2-(cyclobutylamino)-4-(6,6-dimethyl-4-oxo-3-(trifluoromethyl)-4,5,6,7-tetrahydro-1H-indazol-1-yl)benzamide | 421(M + H) |
| 149 | 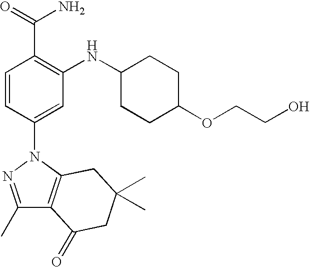 | 2-(4-(2-hydroxyethoxy)cyclohexylamino)-4-(3,6,6-trimethyl-4-oxo-4,5,6,7-tetrahydro-1H-indazol-1-yl)benzamide8.45(b, 1H), 7.81(d, 1H),7.73(d, 1H), 7.22(b, 1H),6.75(d, 1H), 6.65(dd, 1H),4.39(m, 1H), 3.61-3.39(m,2H), 2.91(s, 2H), 2.47(t,1H), 2.39(s, 2H), 2.38(s,3H), 2.30(s, 2H), 2.01-1.19(m, 8H), 1.00(s, 6H) | 455(M + H) |
| 150 | 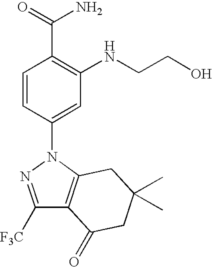 | 4-(6,6-dimethyl-4-oxo-3-(trifluoromethyl)-4,5,6,7-tetrahydro-1H-indazol-1-yl)-2-(2-hydroxyethylamino)benzamide8.41(t, 1H), 7.94(b, 1H), 7.75(d, 1H), 7.29(b, 1H)6.81(d, 1H), 6.70(dd, 1H),4.82(t, 1H), 3.58(q, 2H),3.19(q, 2H), 2.94(s, 2H),2.42(s, 2H), 1.01(s, 6H) | 411(M + H) |
| 151 | 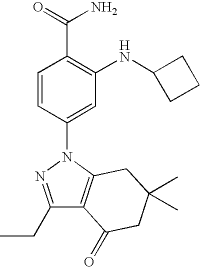 | 2-(cyclobutylamino)-4-(3-ethyl-6,6-dimethyl-4-oxo-4,5,6,7-tetrahydro-1H-indazol-1-yl)benzamide8.49(b, 1H), 7.91(b, 1H),7.73(d, 1H), 7.26(b, 1H),6.69(dd, 1H), 6.60(d, 1H),3.97-3.86(m, 1H), 2.90(s,2H), 2.79(q, 2H), 2.43-2.34(m, 2H), 2.31(s, 2H), 1.88-1.68(m, 4H), 1.18(t, 3H),0.99(s, 6H) | 381(M + H) |
| 152 | 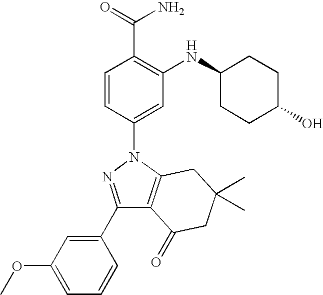 | 2-(trans-4-hydroxycyclohexylamino)-4-(3-(3-methoxyphenyl)-6,6-dimethyl-4-oxo-4,5,6,7-tetrahydro-1H-indazol-1-yl)benzamide8.33-8.28(m, 1H), 7.92(b,1H), 7.76-7.70(m, 2H), 7.65-7.60(m, 1H), 7.36-7.30(m,1H), 7.23(b, 1H), 6.97(dd,1H), 6.85(d, 1H), 6.74(dd,1H), 3.78(s, 3H), 3.39-3.29(m, 1H), 2.96(s, 2H), 2.43(s, 2H), 2.03-1.94(m, 2H),1.84-1.76(m, 2H), 1.38-1.13(m, 3H), 1.02(s, 6H) | 503(M + H) |
| 153 | 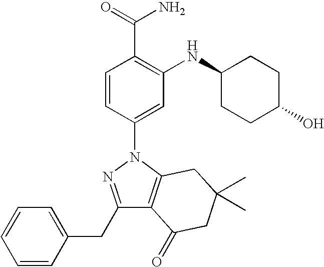 | 4-(3-benzyl-6,6-dimethyl-4-oxo-4,5,6,7-tetrahydro-1H-indazol-1-yl)-2-(trans-4-hydroxycyclohexylamino)benz-amide8.35(d, 1H), 7.88(b, 1H),7.71(d, 1H), 7.31-7.18(m,5H), 7.17-7.12(m, 1H), 6.76(d, 1H), 6.66(dd, 1H), 4.55(d, 1H), 4.17(s, 2H), 3.51-3.41(m, 1H), 2.93(s, 2H),2.32(s, 2H), 2.01-1.93(m,2H), 1.85-1.76(m, 2H), 1.37-1.13(m, 4H), 0.99(s, 6H) | 487(M + H) |
| 154 | 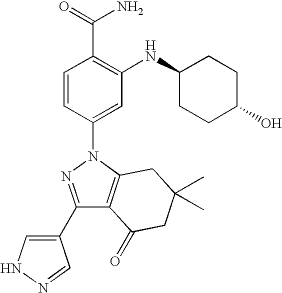 | 4-(6,6-dimethyl-4-oxo-3-(1H-pyrazol-4-yl)-4,5,6,7-tetrahydro-1H-indazol-1-yl)-2-(trans-4-hydroxycyclohexylamino)benz-amide13.0(b, 1H), 8.56(b, 1H),8.30(d, 1H), 8.10(b, 1H),7.91(b, 1H), 7.72(d, 1H),7.21(b, 1H), 6.82(d, 1H),6.72(dd, 1H), 4.65(b, 1H),3.41-3.27(m, 1H), 2.94(s,2H), 2.41(s, 2H), 2.04-1.94(m, 2H), 1.84-1.75(m, 2H),1.38-1.11(m, 3H), 1.01(s,6H) | 463(M + H) |
| 155 | 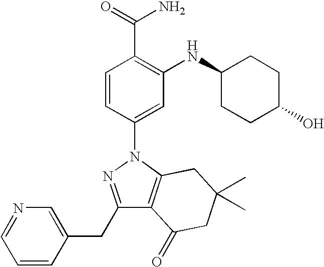 | 4-(6,6-dimethyl-4-oxo-3-(pyridin-3-ylmethyl)-4,5,6,7-tetrahydro-1H-indazol-1-yl)-2-(trans-4-hydroxycyclohexylamino)benz-amide8.50(d, 1H), 8.39-8.32(m,2H), 7.88(b, 1H), 7.71(d,1H), 7.68-7.63(m, 1H), 7.30-7.25(m, 1H), 7.22(b, 1H),6.76(d, 1H), 6.66(dd, 1H),4.56(d, 1H), 4.19(s, 2H),3.50-3.41(m, 1H), 2.93(s,2H), 2.33(s, 2H), 2.01-1.92(m, 2H), 1.85-1.77(m, 2H),1.36-1.13(m, 4H), 0.99(s,6H) | 488(M + H) |
| 156 | 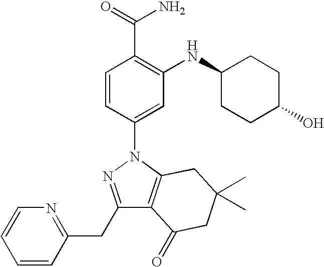 | 4-(6,6-dimethyl-4-oxo-3-(pyridin-2-ylmethyl)-4,5,6,7-tetrahydro-1H-indazol-1-yl)-2-(trans-4-hydroxycyclohexylamino)benz-amide8.41(dd, 1H), 8.35(d, 1H),7.88(b, 1H), 7.74-7.63(m,2H), 7.26(d, 1H), 7.20(b,1H), 7.19-7.15(m, 1H), 6.77(d, 1H), 6.66(dd, 1H), 4.56(d, 1H), 4.34(s, 2H), 3.51-3.41(m, 1H), 2.95(s, 2H),2.32(s, 2H), 2.01-1.93(m,2H), 1.85-1.77(m, 2H), 1.36-1.13(m, 4H), 1.00(s, 6H) | 488(M + H) |
| 157 | 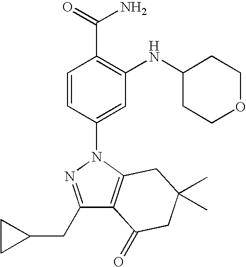 | 4-(3-(cyclopropylmethyl)-6,6-dimethyl-4-oxo-4,5,6,7-tetrahydro-1H-indazol-1-yl)-2-(tetrahydro-2H-pyran-4-ylamino)benzamide8.31(d, 1H), 7.76(b, 1H),7.57(d, 1H), 7.09(b, 1H),6.68(d, 1H), 6.52(dd, 1H),3.69-3.63(m, 2H), 3.51-3.42(m, 1H), 3.34-3.26(m, 2H),2.75(s, 2H), 2.54(d, 2H),2.15(s, 2H), 1.81-1.74(m,2H), 1.28-1.17(m, 2H), 1.00-0.90(m, 1H), 0.83(s, 6H),0.24-0.18(m, 2H), 0.03-0.02(m, 2H) | 437(M + H) |
| 158 | 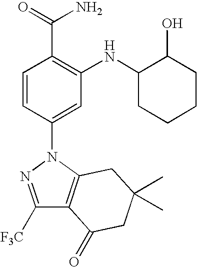 | 4-(6,6-dimethyl-4-oxo-3-(trifluoromethyl)-4,5,6,7-tetrahydro-1H-indazol-1-yl)-2-(2-hydroxycyclohexylamino)benz-amide | 465(M + H) |
| 159 | 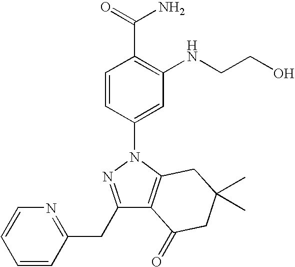 | 4-(6,6-dimethyl-4-oxo-3-(pyridin-2-ylmethyl)-4,5,6,7-tetrahydro-1H-indazol-1-yl)-2-(2-hydroxyethylamino)benzamide8.45-8.38(m, 2H), 7.88(b,1H), 7.71(d, 1H), 7.69-7.62(m, 1H), 7.27-7.20(m, 2H),7.19-7.14(m, 1H), 6.77(d,1H), 6.66(dd, 1H), 4.79(t,1H), 4.33(s, 2H), 3.62-3.55(m, 2H), 3.21-3.14(m, 2H),2.94(s, 2H), 2.31(s, 2H),1.00(s, 6H) | 434(M + H) |
| 160 | 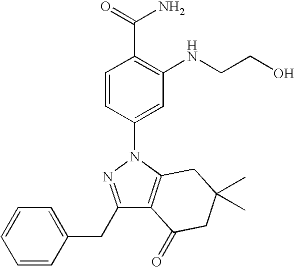 | 4-(3-benzyl-6,6-dimethyl-4-oxo-4,5,6,7-tetrahydro-1H-indazol-1-yl)-2-(2-hydroxyethylamino)benzamide8.42(b, 1H), 7.91(b, 1H),7.71(d, 1H), 7.30-7.20(m,5H), 7.17-7.11(m, 1H), 6.76(d, 1H), 6.66(dd, 1H), 4.17(s, 2H), 3.61-3.55(m, 2H),3.21-3.14(m, 2H), 2.91(s,2H), 2.31(s, 2H), 0.99(s,6H) | 433(M + H) |
| 161 | 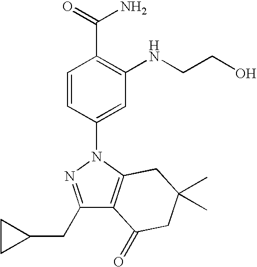 | 4-(3-cyclopropylmethyl)-6,6-dimethyl-4-oxo-4,5,6,7-tetrahydro-1H-indazol-1-yl)-2-(2-hydroxyethylamino)benzamide8.30-8.25(m, 1H), 7.73(b,1H), 7.56(d, 1H), 7.07(b,1H), 6.62(d, 1H), 6.52(s,1H), 4.64(t, 1H), 3.46-3.41(m, 2H), 3.05-3.01(m, 2H),2.76(s, 2H), 2.55(d, 2H),2.17(s, 2H), 1.00-0.92(m,1H), 0.84(s, 3H), 0.24-0.19(m, 2H), 0.04-0.01(m, 2H) | 397(M + H) |
| 162 | 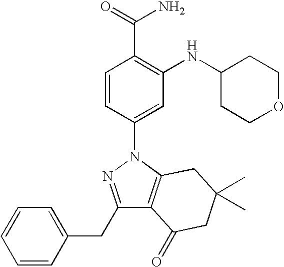 | 4-(3-benzyl-6,6-dimethyl-4-oxo-4,5,6,7-tetrahydr-1H-indazol-1-yl)-2-(tetrahydro-2H-pyran-4-ylamino)benzamide8.01(b, 1H), 7.40(b, 1H),7.33(s, 1H), 7.31-7.14(m,7H), 4.86(d, 1H), 4.19(s,2H), 3.75-3.68(m, 2H), 3.59-3.48(m, 1H), 3.40-3.30(m,2H), 2.59(s, 2H), 2.32(s,2H), 1.81-1.73(m, 2H), 1.24-1.11(m, 2H), 0.98(s, 6H) | 473(M + H) |
| 163 | 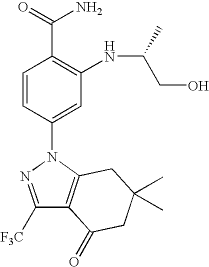 | (R)-4-(6,6-dimethyl-4-oxo-3-(trifluoromethyl)-4,5,6,7-tetrahydro-1H-indazol-1-yl)-2-(1-hydroxypropan-2-ylamino)benzamide8.04(b, 1H), 7.90(d, 1H),7.67(b, 1H), 7.36(s, 1H),7.27(d, 1H), 4.14-4.05(m,1H), 3.97-3.89(m, 1H), 2.96(s, 2H), 2.54-2.51(m, 1H),2.50-2.46(m, 3H), 2.43(s,2H), 1.02(s, 6H) | 425(M + H) |
| 164 | 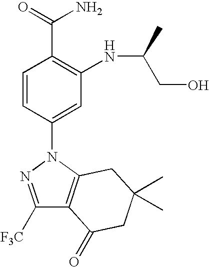 | (S)-4-(6,6-dimethyl-4-oxo-3-(trifluoromethyl)-4,5,6,7-tetrahydro-1H-indazol-1-yl)-2-(1-hydroxypropan-2-ylamino)benzamide8.04(b, 1H), 7.90(d, 1H),7.67(b, 1H), 7.36(s, 1H),7.27(d, 1H), 4.14-4.05(m,1H), 3.97-3.89(m, 1H), 2.96(s, 2H), 2.54-2.51(m, 1H),2.50-2.46(m, 3H), 2.43(s,2H), 1.02(s, 6H) | 425(M + H) |
| 165 | 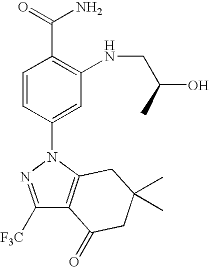 | (S)-4-(6,6-dimethyl-4-oxo-3-(trifluoromethyl)-4,5,6,7-tetrahydro-1H-indazol-1-yl)-2-(2-hydroxypropylamino)benzamide | 425(M + H) |
| 166 | 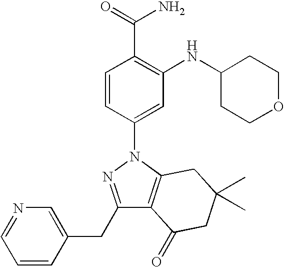 | 4-(6,6-dimethyl-4-oxo-3-(pyridin-3-ylmethyl)-4,5,6,7-tetrahydro-1H-indazol-1-yl)-2-(tetrahydro-2H-pyran-4-ylamino)benzamide8.50(d, 1H), 8.38(dd, 1H),7.99(b, 1H), 7.69-7.65(m,1H), 7.40(b, 1H), 7.32(s,1H_), 7.31-7.27(m, 1H), 7.19(s, 2H), 3.85(d, 1H), 4.21(s, 2H), 3.76-3.69(m, 2H),3.57-3.48(m, 1H), 3.39-3.29(m, 2H), 2.59(s, 2H), 2.32(s, 2H), 1.80-1.73(m, 2H),1.25-1.14(m, 1H), 0.98(s,6H) | 474(M + H) |
| 167 | 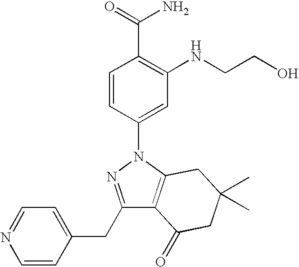 | 4-(6,6-dimethyl-4-oxo-3-(pyridin-4-ylmethyl)-4,5,6,7-tetrahydro-1H-indazol-1-yl)-2-(2-hydroxyethylamino)benzamide8.46-8.40(m, 3H), 7.88(b,1H), 7.71(d, 1H), 7.31-7.19(m, 3H), 6.77(d, 1H), 6.67(dd, 1H), 4.79(t, 1H0, 4.19(s, 2H), 3.62-3.56(m, 2H),3.21-3.15(m, 2H), 2.92(s,2H), 2.32(s, 2H), 0.99(s,6H) | 434(M + H) |
| 168 | 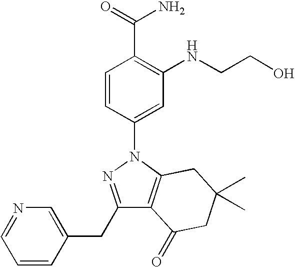 | 4-(6,6-dimethyl-4-oxo-3-(pyridin-3-ylmethyl)-4,5,6,7-tetrahydro-1H-indazol-1-yl)-2-(2-hydroxyethylamino)benzamide8.49(d, 1H), 8.44-8.39(m,1H), 8.36(dd, 1H), 7.88(b,1H), 7.71(d, 1H), 7.67-7.63(m, 1H), 7.30-7.25(m, 1H),7.23(b, 1H), 6.76(d, 1H),6.66(dd, 1H), 4.79(t, 1H),4.19(s, 2H), 3.61-3.55(m,2H), 3.21-3.14(m, 2H), 2.91(s, 2H), 2.32(s, 2H), 0.99(s, 6H) | 434(M + H) |
| 169 | 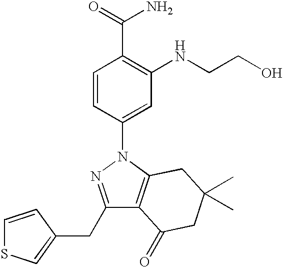 | 4-(6,6-dimethyl-4-oxo-3-(thiophen-3-ylmethyl)-4,5,6,7-tetrahydro-1H-indazol-1-yl)-2-(2-hydroxyethylamino)benzamide8.42(t, 1H), 7.88(b, 1H),7.71(d, 1H), 7.38(dd, 1H),7.22(b, 1H), 7.18(d, 1H),7.01(dd, 1H), 6.77(d, 1H),6.66(dd, 1H), 4.80(t, 1H),4.17(s, 2H), 3.59(q, 2H),3.18(q, 2H), 2.91(s, 2H),2.33(s, 2H), 1.00(s, 6H) | 439(M + H) |
| 170 | 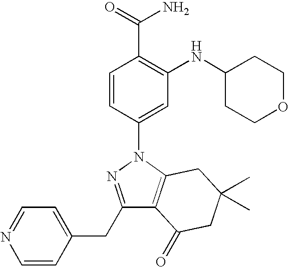 | 4-(6,6-dimethyl-4-oxo-3-(pyridin-4-ylmethyl)-4,5,6,7-tetrahydro-1H-indazol-1-yl)-2-(tetrahydro-2H-pyran-4-ylamino)benzamide8.48(d, 1H), 8.44-8.41(m,2H), 7.93(b, 1H), 7.74(d,1H), 7.30-7.23(m, 3H), 6.83(d, 1H), 6.68(dd, 1H), 4.19(s, 2H), 3.86-3.78(m, 2H),3.65-3.56(m, 1H), 3.49-3.40(m, 2H), 2.92(s, 2H), 2.32(s, 2H), 1.95-1.88(m, 2H),1.44-1.32(m, 2H), 0.98(s,6H) | 474(M + H) |
| 171 | 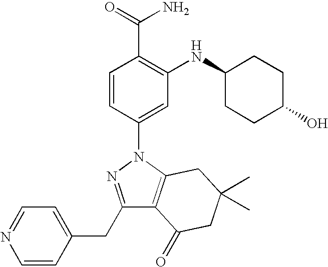 | 4-(6,6-dimethyl-4-oxo-3-(pyridin-4-ylmethyl)-4,5,6,7-tetrahydro-1H-indazol-1-yl)-2-(trans-4-hydroxycyclohexylamino)benz-amide | 488(M + H) |
| 172 | 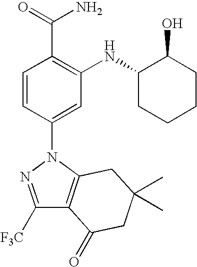 | 4-(6,6-dimethyl-4-oxo-3-(trifluoromethyl)-4,5,6,7-tetrahydro-1H-indazol-1-yl)-2-((1S,2S)-2-hydroxycyclohexylamino)benz-amide8.51(d, 1H), 7.93(b, 1H),7.73(d, 1H), 7.26(b, 1H),6.91(d, 1H), 6.66(dd, 1H),4.78(d, 1H), 3.37-3.27(m,1H), 3.23-3.14(m, 1H), 3.00-2.88(m, 2H), 2.46-2.36(m,2H), 2.02-1.94(m, 1H), 1.85-1.78(m, 1H), 1.66-1.51(m,2H), 1.38-1.21(m, 3H), 1.19-1.08(m, 1H), 1.03(s, 3H),1.00(s, 3H) | 465(M + H) |
| 173 | 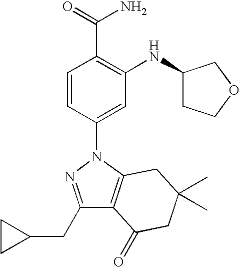 | (R)-4-(3-(cyclopropylmethyl)-6,6-dimethyl-4-oxo-4,5,6,7-tetrahydro-1H-indazol-1-yl)-2-(tetrahydrofuran-3-ylamino)benzamide7.86(b, 1H), 7.17(b, 1H),7.11(s, 1H), 7.09(s, 2H),5.20(d, 1H), 4.02-3.95(m,1H), 3.73-3.68(m, 1H), 3.61-3.51(m, 2H), 3.30(dd, 1H),2.60-2.56(m, 2H), 2.48(s,1H), 2.46(s, 1H), 2.16(s,2H), 2.08-2.00(m, 1H), 1.58-1.49(m, 1H), 1.01-0.92(m,1H), 0.84(s, 3H), 0.83(s,3H), 0.28-0.22(m, 2H), 0.04-0.01(m, 2H) | 423(M + H) |
| 174 | 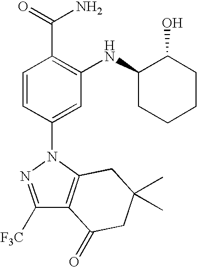 | 4-(6,6-dimethyl-4-oxo-3-(trifluoromethyl)-4,5,6,7-tetrahydro-1H-indazol-1-yl)-2-((1R,2R)-2-hydroxycyclohexylamino)benz-amide8.47(d, 1H), 7.92(b, 1H),7.72(d, 1H), 7.25(b, 1H),6.89(d, 1H), 6.65(dd, 1H),4.82(d, 1H), 3.37-3.27(m,1H), 3.22-3.13(m, 1H), 2.99-2.87(m, 2H), 2.47-2.37(m,2H), 2.01-1.92(m, 1H), 1.86-1.77(m, 1H), 1.65-1.50(m,2H), 1.37-1.07(m, 4H), 1.02(s, 2H), 0.99(s, 3H) | 465(M + H) |
| 175 | 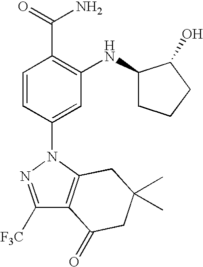 | 4-(6-6-dimethyl-4-oxo-3-(trifluoromethyl)-4,5,6,7-tetrahydro-1H-indazol-1-yl)-2-((1R,2R)-2-hydroxycyclohexylamino)benz-amide8.44(d, 1H), 7.97(b, 1H),7.77(d, 1H), 7.31(b, 1H),6.93(d, 1H), 6.72(dd, 1H),4.88(d, 1H), 3.88-3.82(m,1H), 3.55-3.48(m, 1H), 2.96(s, 2H), 2.47-2.37(m, 2H),2.20-2.08(m, 1H), 1.85-1.57(m, 3H), 1.56-1.48(m, 1H),1.42-1.31(m, 1H), 1.03(s,3H), 1.01(s, 3H) | 451(M + H) |
| 176 | 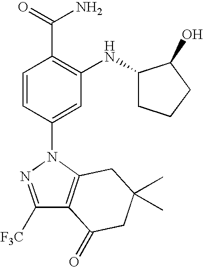 | 4-(6,6-dimethyl-4-oxo-3-(trifluoromethyl)-4,5,6,7-tetrahydro-1H-indazol-1-yl)-2-((1S,2S)-2-hydroxycyclohexylamino)benz-amide8.44(d, 1H), 7.97(b, 1H),7.77(d, 1H), 7.31(b, 1H),6.93(d, 1H), 6.72(dd, 1H),4.88(d, 1H), 3.88-3.82(m,1H), 3.55-3.48(m, 1H), 2.96(s, 2H), 2.46-2.37(m, 2H),2.21-2.08(m, 1H), 1.85-1.57(m, 3H), 1.56-1.47(m, 1H),1.42-1.32(m, 1H), 1.03(s,3H), 1.01(s, 3H) | 451(M + H) |
| 177 | 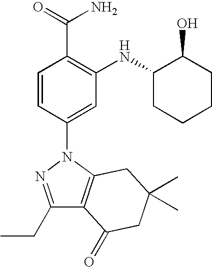 | 4-(3-ethyl-6,6-dimethyl-4-oxo-4,5,6,7-tetrahydro-1H-indazol-1-yl)-2-((1S,2S)-2-hydroxycyclohexylamino)benz-amide8.50(d, 1H), 7.86(b, 1H),7.69(d, 1H), 7.18(b, 1H),6.84(d, 1H), 6.63(dd, 1H),4.77(d, 1H), 3.38-3.29(m,1H), 3.23-3.12(m, 1H), 2.98-2.83(m, 2H), 2.83-2.76(m,2H), 2.38-2.25(m, 2H), 2.04-1.95(m, 1H), 1.86-1.78(m,1H), 1.66-1.52(m, 2H), 1.38-1.22(m, 3H), 1.22-1.11(m,4H), 1.01(s, 3H), 0.98(s,3H) | 425(M + H) |
| 178 | 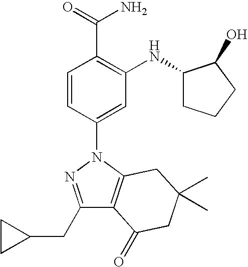 | 4-(3-(cyclopropylmethyl)-6,6-dimethyl-4-oxo-4,5,6,7-tetrahydro-1H-indazol-1-yl)-2-((1S,2S)-2-hydroxycyclopentylamino)benz-amide8.26(d, 1H), 7.75(b, 1H),7.56(d, 1H), 7.07(b, 1H),6.73(d, 1H), 6.53(dd, 1H0,4.71(d, 1H), 3.72-3.67(m,1H), 3.39-3.32(m, 1H), 2.78(d, 2H), 2.55(dd, 2H), 2.16(d, 2H), 2.05-1.94(m, 1H),1.68-1.44(m, 3H), 1.41-1.33(m, 1H), 1.27-1.17(m, 1H),0.86(s, 3H), 0.83(s, 3H),0.25-0.19(m, 2H), 0.04-0.02(m, 2H) | 437(M + H) |
| 179 | 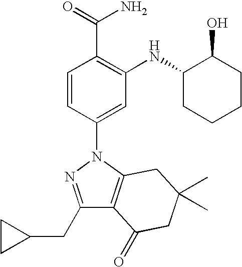 | 4-(3-(cyclopropylmethyl)-6,6-dimethyl-4-oxo-4,5,6,7-tetrahydro-1H-indazol-1-yl)-2-((1S,2S)-2-hydroxycyclohexylamino)benz-amide8.29(d, 1H), 7.71(b, 1H),7.53(d, 1H0, 7.00(b, 1H),6.68(d, 1H), 6.47(dd, 1H),4.67(d, 1H), 3.21-3.14(m,1H), 2.83-2.67(m, 2H), 2.57-2.52(m, 2H), 2.23-2.10(m,2H), 1.87-1.80(m, 1H), 1.70-1.62(m, 1H), 1.50-1.36(m,2H), 1.21-1.06(m, 3H), 1.00-0.91(m, 2H), 0.85(s, 3H)0.81(s, 3H), 0.24-0.18(m,2H), 0.03-0.02(m, 2H) | 451(M + H) |
| 180 | 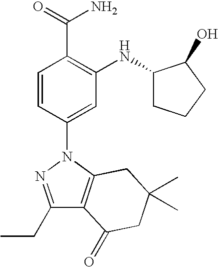 | 4-(3-ethyl-6,6-dimethyl-4-oxo-4,5,6,7-tetrahydro-1H-indazol-1-yl)-2-((1S,2S)-2-hydroxycyclopentylamino)benz-amide7.94(b, 1H), 7.39(b, 1H),7.37(b, 1H), 7.18(s, 1H),4.81(d, 1H), 3.81-3.77(m,1H), 3.62-3.54(m, 1H), 2.81(q, 2H), 2.64-2.54(m, 2H),2.29(s, 2H), 2.14-2.05(m,1H), 1.74-1.63(m, 2H), 1.56-1.42(m, 2H), 1.33-1.23(m,1H), 1.18(t, 3H), 0.98(s,6H) | 411(M + H) |
| 181 | 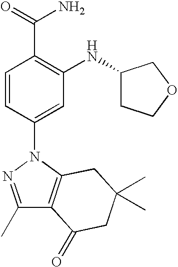 | (S)-2-(tetrahydrofuran-3-ylamino)-4-(3,6,6-trimethyl-4-oxo-4,5,6,7-tetrahydro-1H-indazol-1-yl)benzamide8.55(d, 1H), 7.94(b, 1H),7.75(d, 1H), 7.28(b, 1H),6.77-6.70(m, 2H), 4.17-4.09(m, 1H), 3.91-3.69(m, 3H),3.57-3.52(m, 1H), 2.92(s,2H), 2.38(s, 3H), 2.31(s,2H), 2.29-1.19(m, 1H), 1.79-1.70(m, 1H), 1.00(s, 6H) | 383(M + H) |
| 182 | 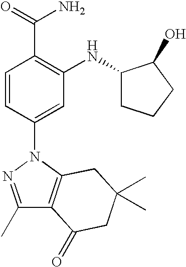 | 2-((1S,2S)-2-hydroxycyclopentylamino)-4-(3,6,6-trimethyl-4-oxo-4,5,6,7-tetrahydro-1H-indazol-1-yl)benzamide8.41(d, 1H), 7.90(b, 1H),7.71(d, 1H), 7.21(b, 1H),6.87(d, 1H), 6.67(dd, 1H),4.87(d, 1H), 3.87-3.81(m,1H), 3.54-3.47(m, 1H), 2.94-2.91(m, 2H), 2.38(s, 3H),2.34-2.29(m, 2H), 2.20-2.10(m, 1H), 1.84-1.58(m, 3H),1.56-1.48(m, 1H), 1.42-1.31(m, 1H), 1.01(s, 3H), 0.99(s, 3H) | 397(M + H) |
| 183 | 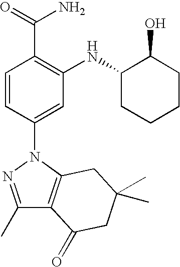 | 2-((1S,2S)-2-hydrocycyclohexylamino)-4-(3,6,6-trimethyl-4-oxo-4,5,6,7-tetrahydro-1H-indazol-1-yl)benzamide8.50(d, 1H), 7.86(b, 1H),7.69(d, 1H), 7.18(b, 1H),6.84(d, 1H), 6.61(dd, 1H),4.76(d, 1H), 3.37-3.26(m,1H), 3.23-3.13(m, 1H), 2.98-2.82(m, 2H), 2.38(s, 3H),2.37-2.25(m, 2H), 2.03-1.94(m, 1H), 1.85-1.78(m, 1H),1.66-1.52(m, 2H), 1.38-1.21(m, 3H), 1.20-1.09(m, 1H),1.01(s, 3H), 0.98(s, 3H) | 411 (M + H) |
| 221 | 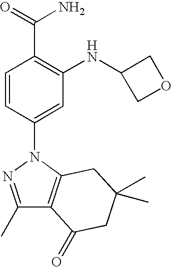 | 2-(oxetan-3-ylamino)-4-(3,6,6-trimethyl-4-oxo-4,5,6,7-tetrahydro-1H-indazol-1-yl)benzamide8.81(d, 1H), 7.99(br s, 1H),7.78(d, 1H), 7.36(br s, 1H),6.78(dd, 1H), 6.44(d, 1h),4.89(ps t, 2H), 4.65(m, 1H),4.40(ps t, 2H), 2.89(s, 2H),2.38(s, 3H), 2.31, (s, 2H),1.00(s, 6H) | 369(M + H) |
| 222 | 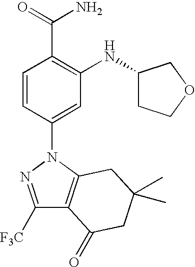 | (S)-4-(6,6-dimethyl-4-oxo-3-(trifluoromethyl)-4,5,6,7-tetrahydro-1H-indazol-1-yl)-2-(tetrahydrofuran-3-ylamino)benzamide8.58(d, 1H), 8.01(b, 1H),7.79(d, 1H), 7.36(b, 1H),6.83(d, 1H), 6.77(dd, 1H),4.17-4.10(m, 1H), 3.91-3.70(m, 3H), 3.57-3.52(m, 1H),2.97(s, 2H), 2.43(s, 2H),2.29-2.19(m, 1H), 1.79-1.70(m, 1H), 1.02(s, 6H) | 437(M + H) |
| 223 | 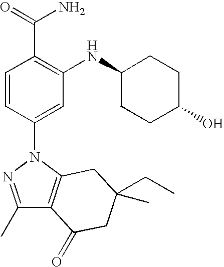 | 4-(6-ethyl-3,6-dimethyl-4-oxo-4,5,6,7-tetrahydro-1H-indazol-1-yl)-2-((trans-4-hydroxycyclohexylamino)benz-amide8.35(d, 1H), 7.87(br s, 1H),7.71(d, 1H), 7.20(br s, 1H),6.75(d, 1H), 6.66(dd, 1H),4.56(d, 1H), 3.47(m, 1H),3.32(m, 1H), 3.00(d, 1H),2.78(d, 1H), 2.40(d, 1H),2.38(s, 3H), 2.22(d, 1H),1.97(m, 2H), 1.80(m, 2H),1.15-1.40(m, 4H), 0.90(s,3H), 0.78(t, 3H) | 425(M + H) |
| 224 | 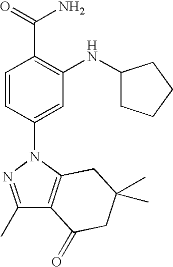 | 2-(cyclopentylamino)-4-(3,6,6-trimethyl-4-oxo-4,5,6,7-tetrahydro-1H-indazol-1-yl)benzamide8.43(d, 1H), 7.89(b, 1H),7.71(d, 1H), 7.21(b, 1H),6.74(d, 1H), 6.66(dd, 1H),3.85-3.75(m, 1H), 2.91(s,2H), 2.38(s, 3H), 2.31(s,2H), 2.02-1.92(m, 2H), 1.71-1.51(m, 4H), 1.47-1.38(m,2H), 1.00(s, 6H) | 381(M + H) |
| 225 | 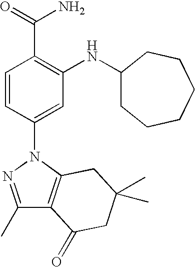 | 2-(cycloheptylamino)-4-(3,6,6-trimethyl-4-oxo-4,5,6,7-tetrahydro-1H-indazol-1-yl)benzamide8.47(d, 1H), 7.88(b, 1H),7.71(d, 1H), 7.20(b, 1H),6.68-6.64(m, 2H), 3.58-3.49(m, 1H), 2.92(s, 2H), 2.38(s, 3H), 2.32(s, 2H), 1.94-1.85(m, 2H), 1.65-1.46(m,10H), 1.00(s, 6H) | 409(M + H) |
| 229 | 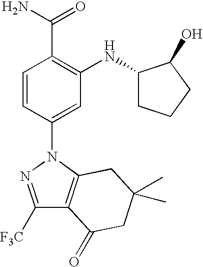 | 4-(6,6-dimethyl-4-oxo-3-(trifluoromethyl)-4,5,6,7-tetrahydro-1H-indol-1-yl)-2-((1S,2S)-2-hydroxycyclopentylamino)benz-amide8.42(d, 1H), 7.93(s, 1H),7.72(d, 1H), 7.65(s, 1H),7.25(s, 1H), 6.82(d, 1H),6.60(m, 1H), 4.90(d, 1H),3.84(m, 1H), 3.54(m, 1H),2.73(s, 2H), 2.33(s, 2H),2.14(m, 1H), 1.60-1.81(m,2H), 1.51(m, 1H), 1.36(m,1H), 0.99(s, 6H) | 450(M + H) |
| 230 | 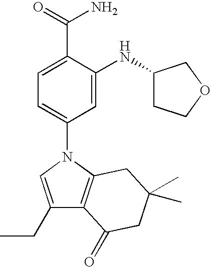 | (S)-4-(3-ethyl-6,6-dimethyl-4-oxo-4,5,6,7-tetrahydro-1H-indol-1-yl)-2-(tetrahydrofuran-3-ylamino)benzamide8.54(d, 1H), 7.72(d, 1H),6.83(s, 1H), 6.61(s, 1H),6.57(d, 1H), 3.88-3.55(m,2H), 2.80(s, 4H), 2.63(q,4H), 2.23(s, 4H), 1.80-1.70(m, 1H), 1.13(t, 3H), 0.97(s,6H) | 396(M + H) |
| 255 | 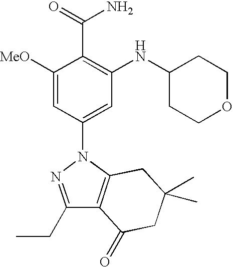 | 4-(3-ethyl-6,6-dimethyl-4-oxo-4,5,6,7-tetrahydro-1H-indazol-1-yl)-2-methoxy-6-(tetrahydro-2H-pyran-4-ylamino)benzamide | 441(M + H) |
| 256 | 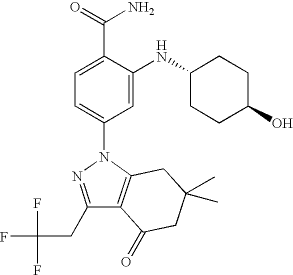 | 4-(6,6-dimethyl-4-oxo-3-(2,2,2-trifluoroethyl)-4,5,6,7-tetrahydro-1H-indazol-1-yl)-2-(trans-4-hydroxycyclohexylamino)benz-amide8.36(d, 1H), 7.90(br s, 1H),7.74(d, 1H), 7.25(br s, 1H),6.80(d, 1H), 6.67(dd, 1H),4.56(d, 1H), 3.93(q, 2H),3.46(m, 1H), 3.33(m underH₂O, 1H), 2.97(s, 2H), 2.37(s,2H), 1.98(m, 2H), 1.81(m,2H), 1.31(m, 2H), 1.2(m, 2H),1.00(s, 6H) | 479(M + H) |
| 257 | 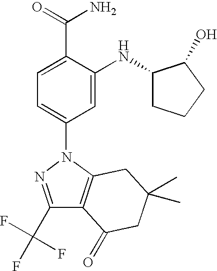 | 4-(6,6-dimethyl-4-oxo-3-(trifluoromethyl)-4,5,6,7-tetrahydro-1H-indazol-1-yl)-2-((1S,2R)-2-hydroxycyclopentylamino)benza-amide8.54(d, 1H), 7.71(s, 1H),6.79(d, 1H), 6.65(m, 1H), 4.67(d, 1H), 4.05(m, 1H), 3.60(m,1H), 2.93(q, 2H), 2.42(d,2H), 2.0(m, 1H), 1.7-1.8(m,2H), 1.35-1.62(m, 3H), 1.02(s, 3H), 1.00(s, 6H) | 451(M + H) |
| 258 | 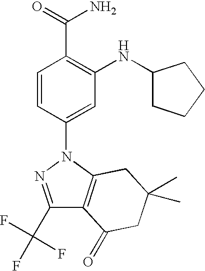 | 2-(cyclopentylamino)-4-(6,6-dimethyl-4-oxo-3-(trifluoromethyl)-4,5,6,7-tetrahydro-1H-indazol-1-yl)benzamide8.45(d, 1H), 7.97(br s, 1H),7.77(d, 1H), 7.30(br s, 1H),6.80(d, 1H), 6.71, (dd, 1H),3.81(m, 1H), 2.96(s, 2H), 2.43(s, 2H), 1.97(m, 2H),1.53-1.70(m, 4H), 1.42(m,2H), 1.02(s, 6H) | 435(M + H) |
| 259 | 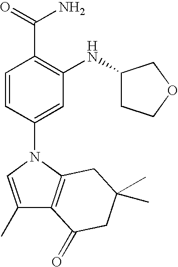 | (S)-2-(tetrahydrofuran-3-ylamino)-4-(3,6,6-trimethyl-4-oxo-4,5,6,7-tetrahydro-1H-indol-1-yl)benzamide8.54(d, 1H), 7.72(d, 1H),6.83(s, 1H), 6.59(s, 1H),6.56(d, 1H), 3.88-3.65(m,3H), 3.53(dd, 1H), 2.97(s,2H), 2.71(s, 2H), 2.23(s,3H), 2.20(s, 4H), 1.80-1.70(mn, 1H), 0.97(s, 6H) | 382(M + H) |
| 260 | 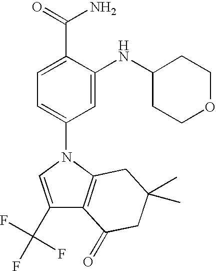 | 4-(6,6-dimethyl-4-oxo-3-(trifluoromethyl)-4,5,6,7-tetrahydro-1H-indol-1-yl)-2-(tetrahydro-2H-pyran-4-ylamino)benzamide8.48(d, 1H), 7.96(bs, 1H),7.74(d, 1H), 7.67(s, 1H),7.29(bs, 1H), 6.82(d, 1H),6.59(dd, 1H), 3.82(m, 2H),3.68(m, 1H), 3.45(t, 2H),2.71(s, 2H), 2.32(s, sH),1.91(d, 2H), 1.37(m, 2H),0.98(s, 6H) | 450(M + H) |
| 261 | 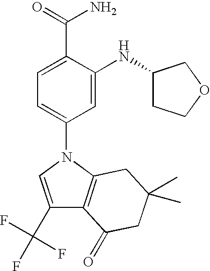 | (S)-4-(6,6-dimethyl-4-oxo-3-(trifluoromethyl)-4,5,6,7-tetrahydro-1H-indol-1-yl)-2-(tetrahydrofuran-3-ylamino)benzamide8.56(d, 1H), 7.97(bs, 1H),7.75(d, 1H), 7.69(s, 1H),7.31(bs, 1H), 6.73(s, 1H),6.65(dd, 1H), 4.16(m, 1H),3.85(m, 1H), 3.82(m, 1H),3.71(m, 1H), 3.53(dd, 1H),2.74(s, 2H), 2.33(s, 2H),1.73(m, 1H), 0.99(s, 6H) | 436(M + H) |
| 262 | 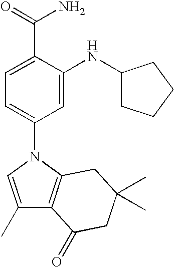 | 2-(cyclopentylamino)-4-(3,.6,6-trimethyl-4-oxo-4,5,6,7-tetrahydro-1H-indol-1-yl)benzamide8.42(d, 1H), 7.85(br, 1H),7.69(d, 1H), 7.18(br, 1H),6.81(s, 1H), 6.56(s, 1H),6.50(d, 1H), 3.82(m, 1H),2.70(s, 2H), 2.23(s, 2H),2.19(s, 3H), 1.99-1.90(m,2H), 1.70-1.50(m, 4H), 1.45-1.35(m, 2H), 0.99(s, 6H) | 380(M + H) |
| 264 | 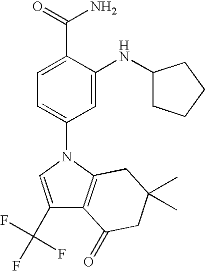 | 2-(cyclopentylamino)-4-(6,6-dimethyl-4-oxo-3-(trifluoromethyl)-4,5,6,7-tetrahydro-1H-indol-1-yl)benzamide8.44(d, 1H), 7.92(br, 1H),7.72(s, 1H), 7.67(s, 1H),7.25(br, 1H), 6.68(s, 1H),6.57(d, 1H), 3.86(m, 1H),2.73(s, 2H), 2.33(s, 2H),1.99-1.90(m, 2H), 1.70-1.50(m, 4H), 1.45-1.35(m, 2H),0.99(s, 6H) | 434(M + H) |
| 265 | 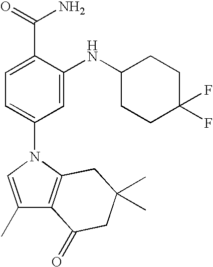 | 2-(4,4-difluorocyclohexylamino)-4-(3,6,6-trimethyl-4-oxo-4,5,6,7-tetrahydro-1H-indol-1-yl)benzamide | 430(M + H) |
| 266 | 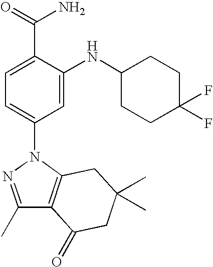 | 2-(4,4-difluorocyclohexylamino)-4-(3,6,6-trimethyl-4-oxo-4,5,6,7-tetrahydro-1H-indazol-1-yl)benzamide | 431(M + H) |
| 267 | 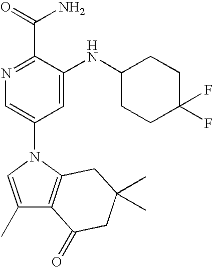 | 3-(4,4-difluorocyclohexylamino)-5-(3,6,6-trimethyl-4-oxo-4,5,6,7-tetrahydro-1H-indol-1-yl)picolinamide | 431(M + H) |
| 268 | 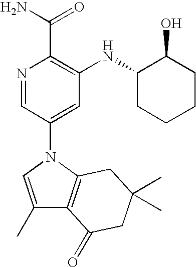 | 3-((1S,2S)-2-hydroxycyclohexylamino)-5-(3,6,6-trimethyl-4-oxo-4,5,6,7-tetrahydro-1H-indol-1-yl)picolinamide | 411(M + H) |
| 269 | 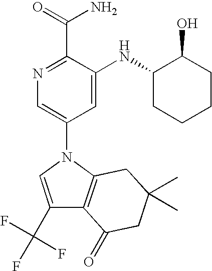 | 5-(6,6-dimethyl-4-oxo-3-(trifluoromethyl)-4,5,6,7-tetrahydro-1H-indol-1-yl)-3-((1S,2S)-2-hydroxycyclohexylamino)picolin-amide | 465(M + H) |
| 270 | 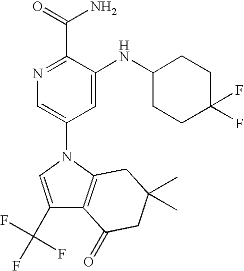 | 3-(4,4-difluorocyclohexylamino)-5-(6,6-dimethyl-4-oxo-3-(trifluoromethyl)-4,5,6,7-tetrahydro-1H-indol-1-yl)picolinamide | 485(M + H) |
| 271 | 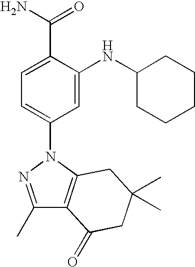 | 2-(cyclohexylamino)-4-(3,6,6-trimethyl-4-oxo-4,5,6,7-tetrahydro-1H-indazol-1-yl)benzamide | 395(M + H) |
| 272 | 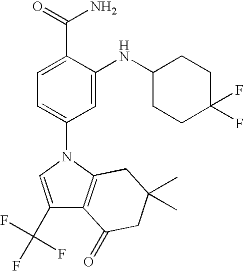 | 2-(4,4-difluorocyclohexylamino)-4-(6,6-dimethyl-4-oxo-3-(trifluoromethyl)-4,5,6,7-tetrahydro-1H-indol-1-yl)benzamide | 484(M + H) |

Examples 184-188, shown below in Table 4, were prepared essentially according to the synthetic methodology described herein, Examples 1-47 and Schemes 1-5, and/or by using methodology well known in the art.

TABLE 4

| | | Nomenclature | |
|---|---|---|---|
| Ex. | Structure | NMR (400 MHz, DMSO) δ (ppm) | |
| 184 | 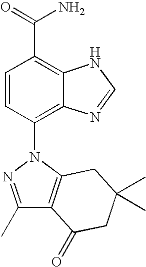 | 4-(3,6,6-trimethyl-4-oxo-4,5,6,7-tetrahydro-1H-indazol-1-yl)-1H-benzo[d]imidazole-7-carboxamide12.8(b, 1H), 8.38(b, 1H),7.94(d, 1H), 7.74(b, 1H),7.42(d, 1H), 2.80(b, 2H),2.44(s, 3H), 2.34(s, 2H),0.98(s, 6H) | 338(M + H) |
| 185 | 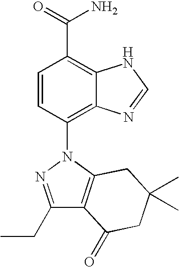 | 4-(3-ethyl-6,6-dimethyl-4-oxo-4,5,6,7-tetrahydro-1H-indazol-1-yl)-1H-benzo[d]imidazol-7-carboxamide12.8(s, 1H), 8.53-8.19(m,1H), 7.95(b, 1H), 7.89-7.57(m, 1H), 7.43(b, 1H), 2.98(s,2H), 2.95-2.76(m, 2H), 2.34(s, 2H), 1.23(t, 3H), 0.98(s,6H) | 352(M + H) |
| 186 | 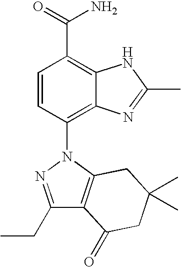 | 4-(3-ethyl-6,6-dimethyl-4-oxo-4,5,6,7-tetrahydro-1H-indazol-1-yl)-2-methyl-1H-benzo[d]imidazole-7-carboxamide12.5(s, 1H), 9.28-9.22(m,1H), 7.89(d, 1H), 7.82-7.77(m, 1H), 7.36-7.27(m, 1H),2.93-2.78(m, 3H), 2.69(s,1H), 2.59(s, 2H), 2.53(s,1H), 2.37-2.30(m, 2H), 1.27-1.17(m, 3H), 0.99(s, 3H),0.97(s, 3H) | 366(M + H) |
| 187 | 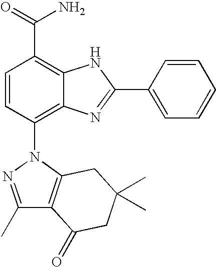 | 2-phenyl-4-(3,6,6-trimethyl-4-oxo-4,5,6,7-tetrahydro-1H-indazol-1-yl)-1H-benzo[d]imidazole-7-carboxamide13.1(b, 1H), 9.31(b, 1H),8.34-8.27(m, 2H), 8.01-7.81(m, 2H), 7.61-7.54(m, 2H),7.53-7.49(m, 1H), 7.40(d,1H), 2.98(s, 2H), 2.48(s,3H), 2.35(s, 2H), 1.00(s, 6H) | 414(M + H) |
| 188 | 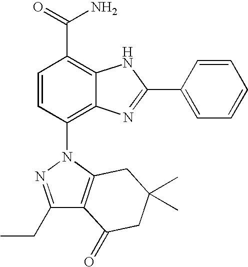 | 4-(3-ethyl-6,6-dimethyl-4-oxo-4,5,6,7-tetrahydro-1H-indazol-1-yl)-2-phenyl-1H-benzo[d]imidazole-7-carboxamide10.2(b, 1H), 9.32(b, 1H),8.34-8.28(m, 2H), 8.04-8.78(m, 2H), 7.63-7.49(m, 3H),7.40(d, 1H), 2.97(s, 2H),2.93-2.83(m, 2H), 2.36(s,2H), 1.25(t, 3H), 1.00(s, 6H) | 428(M + H) |

### Cell Proliferation Assays

A panel of cancer cell lines was obtained from the DCTP Tumor Repository, National Cancer Institute (Frederick, Md.) or ATCC (Rockville, Md.). Cell cultures were maintained in Hyclone RPMI 1640 medium (Logan, Utah) supplemented with 10% fetal bovine serum and 20 mM HEPES buffer, final pH 7.2, at 37° C. with a 5% CO₂ atmosphere. Cultures were maintained at sub-confluent densities. Human umbilical vein endothelial cells (HUVEC) were purchased from Clonetics, a division of Cambrex (Walkersville, Md.). Cultures were established from cryopreserved stocks using Clonetics EGM-2 medium supplemented with 20 mM HEPES, final pH 7.2, at 37° C. with a 5% CO₂ atmosphere.

For proliferation assays, cells were seeded with the appropriate medium into 96 well plates at 1,000-2,500 cells per well, depending on the cell line, and were incubated overnight. The following day, test compound, DMSO solution (negative control), or Actinomycin D (positive control) was added to the appropriate wells as 10× concentrated stocks prepared in phosphate buffered saline. The cell plates were then incubated for an additional 2-5 days, depending on the cell line, to allow proliferation to occur. To measure cell density, 50 μL of WST-1 solution (Roche Applied Science, Ind.) diluted 1:5 in phosphate buffered saline was added to each well, and the cells incubated for an additional 1-5 hrs., again depending on the cell line. Optical density was determined for each well at 450 nM using a Tecan GeniosPro plate reader (RTP, NC). The percentage of cell growth was determined by comparing the cell growth in the presence of test compounds to the cells treated with DMSO vehicle (control, 100% growth) and cells treated with Actinomycin D (10 μM, 0% growth).

Immediately after the WST-1 determination, the medium was removed from the PC-3, NCI-H460 and HUVEC cell lines, and the plates stored at −80° C. Using these assay plates, relative amounts of DNA in each well were determined using the Cyquant DNA assay kit from R&D Systems (Eugene, Oreg.) following the manufacturer's directions. Results for each compound treatment were compared to DMSO vehicle control (100%) and 10 μM Actinomycin D treated cells (0%).

Several exemplary compounds useful in the methods of the invention are listed below. The range of their inhibitory activity against PC-3 cell proliferation is demonstrated, where +++ stands for an IC₅₀ value that is less than 0.5 μM, ++ between 0.5 and 5 μM, + between 5 and 50 μM.

| | | | |
|---|---|---|---|
| Example 51 | +++ | Example 89 | +++ |
| Example 97 | +++ | Example 119 | +++ |
| Example 124 | ++ | Example 140 | +++ |
| Example 157 | +++ | Example 165 | ++ |
| Example 180 | +++ | Example 186 | + |

### Determination of Affinity for HSP-90

### (Heat Shock Protein 90)

Affinity of test compounds for HSP-90 was determined as follows: Protein mixtures obtained from a variety of organ tissues (for example: spleen, liver and lung) were reversibly bound to a purine affinity column to capture purine-binding proteins, especially HSP-90. The purine affinity column was washed several times, and then eluted with 20 μM, 100 μM, and 500 μM of test compound. Compounds of Formula I elute HP-90 in a dose-dependent manner vs. a control elution using dimethylsulfoxide. The elution profile of Formula I compounds was determined by 1-dimensional SDS polyacrylamide gel electrophoresis. Gels were stained with a fluorescent stain such as sypro ruby (a highly sensitive fluorescent protein stain that can readily detect less than 1 fmol of total protein, i.e., less than 0.04 ng for a 40 kDa protein) or silver nitrate. The gels were imaged using a standard flat bed gel imager and the amount of protein estimated by densitometry. The percent of HSP-90 protein eluted from the column at each concentration was determined and IC₅₀ values were calculated from these estimates.

Compounds of the invention are inhibitors of HSP-90 (heat shock protein 90). Several exemplary compounds useful in the methods of the invention are listed below. The range of their relative binding affinity to HSP-90 is demonstrated, where +++ stands for very high, ++ for high and + for moderate.

| | | | |
|---|---|---|---|
| Example 51 | +++ | Example 89 | +++ |
| Example 97 | +++ | Example 119 | +++ |
| Example 124 | +++ | Example 140 | +++ |
| Example 157 | +++ | Example 165 | +++ |
| Example 180 | +++ | Example 186 | ++ |

The invention and the manner and process of making and using it, are now described in such full, clear, concise and exact terms as to enable any person skilled in the art to which it pertains, to make and use the same. It is to be understood that the foregoing describes preferred embodiments of the invention and that modifications may be made therein without departing from the spirit or scope of the invention as set forth in the claims. To particularly point out and distinctly claim the subject matter regarded as invention, the following claims conclude this specification.

What is claimed is:

1. A compound of the formula 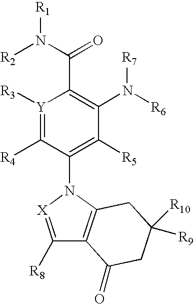
or a pharmaceutically acceptable salt thereof, wherein
R₁ and R₂ are independently —H or C₁-C₈ alkyl;
R₃ is absent or —H, —F, —OCH₃;
R₄ and R₅ are independently —H, —F, —OCH₃;
R₆ is (C₂-C₁₄)alkenyl, (C₁-C₁₄)alkyl, (C₂-C₁₄)alkynyl, aryl, aryl(C₁-C₈) alkyl, (C₃-C₈) cycloalkenyl, (C₃-C₈)cycloalkenyl(C₁-C₈)alkyl, (C₃-C₈)cycloalkyl, (C₃-C₈)cycloalkyl(C₁-C₈)alkyl, heteroaryl, heteroaryl(C₁-C₈)alkyl, heteroaryl(C₁-C₈)alkylthio(C₁-C₈)alkyl, heteroarylthio(C₁-C₈)alkyl, heterocyclyl, heterocycle(C₁-C₈)alkyl, or hydroxy(C₁-C₈)alkyl, wherein the R₆ group is optionally substituted with 1, 2, 3, 4, or 5 groups which are (C₂-C₈)alkenyl, (C₁-C₈)alkoxy, (C₁-C₈)alkoxy(C₁-C₈)alkyl, (C₁-C₈)alkoxycarbonyl, (C₁-C₈)alkyl, (C₁-C₈)alkylcarbonyl, (C₁-C₈)alkylcarbonyloxy, (C₁-C₈)alkylsulfinyl, (C₁-C₈)alkylsulfonyl, (C₁-C₈)alkylthio, (C₂-C₈)alkynyl, carboxy, carboxy(C₁-C₈)alkyl, cyano, cyano(C₁-C₈)alkyl, (C₃-C₈)cycloalkyl, (C₃-C₈)cycloalkyl(C₁-C₈)alkyl, formyl, halo(C₁-C₈)alkoxy, halo(C₁-C₈)alkyl, halogen, hydroxy, hydroxy(C₁-C₈)alkoxy, hydroxy(C₁-C₈)alkoxy(C₁-C₈)alkoxy, hydroxy(C₁-C₈)alkyl, mercapto, nitro, —NR₁₁R₁₂, (NR₁₁R₁₂) (C₁-C₈)alkyl, (NR₁₁R₁₂)carbonyl, oxo, HOCH₂CH(NH₂) C(O)O—, NH₂ (CH₂)ₘC(O)O—, CH₃NH(CH₂)ₘC(O)O—, (CH₃)₂N(CH₂)ₘC(O)O—, NH₂ (CH₂)ₜC(O)NH(CH₂)ₘC(O)O—, R₁₂CH(NH₂) C(O)O—, NH₂ (CH₂)ₘC(R₂)₂ (CH₂)ₘC(O)O—, NH₂CH₂CH₂C(O)O—, R₁₁ON═CH(CH₂)ₙO—, or HONHC(O) (CH₂)ₙO—; or
R₅ and R₆ taken together with the nitrogen atom to which they are attached form a heteroaryl, wherein the heteroaryl is imidazolyl, oxazolyl, pyrazolyl, pyridazinyl, pyridinyl, pyrazinyl, pyrrolyl, or thiazolyl, wherein the heteroaryl is optionally substituted with(C₁-C₈)alkoxy, (C₁-C₈)alkoxycarbonyl, (C₁-C₈alkyl, (C₁-C₈) alkylcarbonyl, (C₁-C₈) alkylcarbonyloxy, aryl, aryl(C₁-C₈)alkyl, cyano, cyano(C₁-C₈)alkyl, (C₃-C₈) cycloalkyl, (C₃-C₈) cycloalkyl (C₁-C₈) alkyl, halo (C₁-C₈) alkoxy, halo (C₁-C₈) alkyl, halogen, heteroaryl, heteroaryl (C₁-C₈) alkyl, heterocyclyl, heterocycle (C₁-C₈) alkyl, hydroxy, hydroxy(C₁-C₈) alkyl, mercapto, nitro, —NR₁₁R₁₂, (NR₁₁R₁₂) (C₁-C₈) alkyl, or (NR₁₁R₁₂) carbonyl;
R₇ is —H or (C₁-C₈) alkyl;
R₈ is (C₂-C₈) alkenyl, (C₁-C₈) alkyl, (C₂-C₈) alkynyl, (C₃-C₈) cycloalkenyl, (C₃-C₈) cycloalkenyl (C₁-C₈) alkyl, (C₃-C₈) cycloalkyl, (C₃-C₈) cycloalkyl (C₁-C₈) alkyl, aryl, aryl (C₁-C₈) alkyl, halo (C₁-C₈) alkyl, heteroaryl, heteroaryl (C₁-C₈)alkyl, heterocyclyl, heterocycle (C₁-C₈)alkyl, or hydroxy (C₁-C₈) alkyl;
R₉ and R₁₀ are independently —H or (C₁-C₈) alkyl; or
R₉ and R₁₀ taken together with the carbon atom to which they are attached form (C₃-C₈)cycloalkyl;
R₁₁ and R₁₂ are independently —H, (C₂-C₈) alkenyl, (C₁-C₈)alkyl, (C₁-C₈)alkylcarbonyl, (C₂-C₈)alkynyl, aryl, aryl(C₁-C₈)alkyl, (C₃-C₈)cycloalkyl, (C₃-C₈)cycloalkyl(C₁-C₈)alkyl, formyl, heteroaryl, heteroaryl(C₁-C₈)alkyl, heterocyclyl, or heterocycle(C₁-C₈)alkyl; or
two R₁₂ groups together with the carbon to which they are attached form a (C₃-C₈)cycloalkyl group;
n is 1, 2, 3, 4, 5, or 6;
each m is independently 1, 2, 3, or 4;
each t is independently 1, 2, 3, or 4;
X is N or CR₁₃;
Y is C or N; and
R₁₃ is —H or (C₁-C₈) alkyl.

2. A compound according to claim 1 wherein
R₁ and R₂ are —H;
R₃ is —H, —F. or —OCH₃;
R₄, and R₅ are independently —H or —F;
R₆ is (C₁-C₁₄)alkyl, (C₃-C₈)cycloalkenyl, (C₃-C₈)cycloalkyl, (C₃-C₈)cycloalkyl(C₁-C₈)alkyl, heteroaryl, heteroaryl(C₁-C₈)alkylthio(C₁-C₈)alkyl, heteroarylthio(C₁-C₈)alkyl, heterocyclyl, or hydroxy(C₁-C₈)alkyl, wherein the R₆ group is optionally substituted with 1, 2, 3, or 4 groups which are (C₂-C₈)alkenyl, (C₁-C₈)alkoxy, (C₁-C₈)alkoxy(C₁-C₈)alkyl, (C₁-C₈)alkoxycarbonyl, (C₁-C₈)alkyl, (C₁-C₈)alkylcarbonyl, (C₂-C₈)alkynyl, (C₃-C₈) cycloalkyl, (C₃-C₈) cycloalkyl(C₁-C₈) alkyl, hydroxy, hydroxy(C₁-C₈)alkoxy, hydroxy(C₁-C₈)alkoxy(C₁-C₈)alkoxy, hydroxy(C₁-C₈)alkyl, —NR₁₁R₁₂, (NR₁₁R₁₂) (C₁-C₈)alkyl, oxo, HOCH₂CH(NH₂)C(O)O—, NH₂(CH₂)ₘC(O)O—, CH₃NH(CH₂)ₘC(O)O—, (CH₃)₂N(CH₂)ₘC(O)O—, NH₂(CH₂)ₜC(O)NH(CH₂)ₘC(O)O—, R₁₂CH(NH₂)C(O)O—, NH₂ (CH₂)ₘC(R₂)₂ (CH₂)ₘC(O)O—, NH₂CH₂CH₂C(O)O—, R₁₁ON═CH(CH₂)ₙO—, or HONHC(O) (CH₂)ₙO—; or
R₅ and R₆ taken together with the nitrogen atom to which they are attached form a heteroaryl, wherein the heteroaryl is imidazolyl optionally substituted with (C₁-C₈)alkyl or aryl;
R₇ is —H;
R₈ is (C₁-C₈)alkyl, (C₃-C₈)cycloalkyl(C₁-C₈)alkyl, aryl, aryl(C₁-C₈)alkyl, halo(C₁-C₈)alkyl, heteroaryl(C₁-C₈)alkyl, or hydroxy (C₁-C₈) alkyl;
R₉ and R₁₀ are independently (C₁-C₈)alkyl;
R₁₁ and R₁₂ are independently —H, (C₁-C₈)alkyl, (C₂-C₈) alkynyl, (C₃-C₈)cycloalkyl, or (C₃-C₈)cycloalkyl(C₁-C₈)alkyl; or
two R₁₂ groups together with the carbon to which they are attached form a (C₃-C₈)cycloalkyl group;
n is 1;
each m is independently 1 or 2;
each t is independently 1 or 2;
X is N or CR₁₃;
Y is C; and
R₁₃ is —H or (C₁-C₈) alkyl.

3. A compound according to claim 1 wherein
R₁ and R₂ are —H;
R₃ is —F;
R₄ and R₅ are —H;
R₆ is (C₁-C₁₄)alkyl, (C₃-C₈)cycloalkenyl, (C₃-C₈)cycloalkyl, (C₃-C₈) cycloalkyl (C₁-C₈) alkyl, heteroaryl (C₁-C₈)alkylthio (C₁-C₈)alkyl, or heterocyclyl, wherein the R₆ group is optionally substituted with 1 or 2 groups that are (C₁-C₈) alkoxy, (C₁-C₈) alkoxycarbonyl, (C₁-C₈) alkylcarbonyl, hydroxy, oxo, R₁₂CH(NH₂) C(O)O—, NH₂CH₂C(O)O—, NH₂CH₂CH₂C(O)O—, or —NR₁₁R₁₂;
R₇ is —H;
R₈ is (C₁-C₈) alkyl, (C₃-C₈) cycloalkyl (C₁-C₈) alkyl, or halo (C₁-C₈) alkyl;
R₉ and R₁₀ are independently (C₁-C₈)alkyl;
R₁₁ and R₁₂ are independently —H or (C₁-C₈) alkyl;
X is N; and
Y is C.

4. A compound according to claim 1 wherein
R₁ and R₂ are —H;
R₄ is —F;
R₃ and R₅ are —H;
R₆ is (C₁-C₁₄)alkyl, (C₃-C₈)cycloalkenyl, (C₃-C₈)cycloalkyl, (C₃-C₈) cycloalkyl (C₁-C₈) alkyl, heteroaryl (C₁-C₈)alkylthio (C₁-C₈)alkyl, or heterocyclyl, wherein the R₆ group is optionally substituted with 1 or 2 groups that are (C₁-C₈) alkoxy, (C₁-C₈) alkoxycarbonyl, (C₁-C₈) alkylcarbonyl, hydroxy, oxo, R₁₂CH(NH₂) C(O)O—, NH₂CH₂CH₂C(O)O— or —NR₁₁R₁₂;
R₇ is —H;
R₈ is (C₁-C₈)alkyl, (C₃-C₈)cycloalkyl(C₁-C₈)alkyl, or halo (C₁-C₈) alkyl;
R₉ and R₁₀ are independently (C₁-C₈)alkyl;
R₁₁ and R₁₂ are independently —H or (C₁-C₈)alkyl;
X is N or CH; and
Y is C.

5. A compound according to claim 1 wherein
R₁ and R₂ are —H;
R₃ and R₄ are —H;
R₅ is —F;
R₆ is (C₁-C₁₄)alkyl, (C₃-C₈)cycloalkenyl, (C₃-C₈)cycloalkyl, (C₃-C₈)cycloalkyl(C₁-C₈)alkyl,
heteroaryl(C₁-C₈)alkylthio(C₁-C₈)alkyl, or heterocyclyl, wherein the R₆ group is optionally substituted with 1 or 2 groups that are (C₁-C₈)alkoxy, (C₁-C₈)alkoxycarbonyl, (C₁-C₈)alkylcarbonyl, hydroxy, oxo, R₁₂CH(NH₂)C(O)O—, NH₂CH₂CH₂C(O)O—, or —NR₁₁R₁₂;
R₇ is —H;
R₈ is (C₁-C₈)alkyl, (C₃-C₈)cycloalkyl(C₁-C₈)alkyl, or halo(C₁-C₈)alkyl;
R₉ and R₁₀ are independently (C₁-C₈)alkyl;
R₁₁ and R₁₂ are independently —H or (C₁-C₈)alkyl;
X is N or CH; and
Y is C.

6. A compound according to claim 1 wherein
R₁ and R₂ are —H;
R₃ is —H;
R₄ and R₅ are —F;
R₆ is (C₁-C₁₄)alkyl, (C₃-C₈)cycloalkenyl, (C₃-C₈)cycloalkyl, (C₃-C₈) cycloalkyl (C₁-C₈) alkyl, heteroaryl (C₁-C₈)alkylthio (C₁-C₈)alkyl, or heterocyclyl, wherein the R₆ group is optionally substituted with 1 or 2 groups which are (C₁-C₈) alkoxy, (C₁-C₈) alkoxycarbonyl, (C₁-C₈)alkylcarbonyl, hydroxy, oxo, R₁₂CH(NH₂) C(O)O—, NH₂CH₂CH₂C(O)O—, or —NR₁₁R₁₂;
R₇ is —H;
R₈ is (C₁-C₈) alkyl, (C₃-C₈) cycloalkyl (C₁-C₈)alkyl, or halo(C₁-C₈)alkyl;
R₉ and R₁₀ are independently (C₁-C₈)alkyl;
R₁₁ and R₁₂ are independently —H or (C₁-C₈) alkyl;
X is N or CH; and
Y is C.

7. A compound according to claim 1 wherein
R₁ and R₂ are —H;
R₃, R₄, and R₅ are —F;
R₆ is (C₁-C₁₄)alkyl, (C₃-C₈)cycloalkenyl, (C₃-C₈)cycloalkyl, (C₃-C₈) cycloalkyl (C₁-C₈) alkyl, heteroaryl (C₁-C₈)alkylthio (C₁-C₈)alkyl, or heterocyclyl, wherein the R₆ group is optionally substituted with 1 or 2 groups which are (C₁-C₈) alkoxy, (C₁-C₈) alkoxycarbonyl, (C₁-C₈)alkylcarbonyl, hydroxy, oxo, R₁₂CH(NH₂) C(O)O—, NH₂CH₂CH₂C(O)O—, or —NR₁₁R₁₂;
R₇ is —H;
R₈ is (C₁-C₈) alkyl, (C₃-C₈) cycloalkyl (C₁-C₈)alkyl, or halo (C₁-C₈) alkyl;
R₉ and R₁₀ are independently (C₁-C₈)alkyl;
R₁₁ and R₁₂ are independently —H or (C₁-C₈) alkyl;
X is N or CH; and
Y is C.

8. A compound according to claim 1 wherein
R₁ and R₂ are —H;
R₃ is —F;
R₄ and R₅ are —H;
R₆ is (C₁-C₁₄)alkyl, (C₃-C₈)cycloalkenyl, (C₃-C₈)cycloalkyl, (C₃-C₈) cycloalkyl (C₁-C₈) alkyl, heteroaryl (C₁-C₈)alkylthio (C₁-C₈)alkyl, or heterocyclyl, wherein the R₆ group is optionally substituted with 1 or 2 groups which are (C₁-C₈) alkoxy, (C₁-C₈) alkoxycarbonyl, (C₁-C₈)alkylcarbonyl, hydroxy, oxo, R₁₂CH(NH₂) C(O)O—, NH₂CH₂CH₂C(O)O—, or —NR₁₁R₁₂;
R₇ is —H;
R₈ is (C₁-C₈) alkyl, (C₃-C₈) cycloalkyl (C₁-C₈)alkyl, or halo (C₁-C₈) alkyl;
R₉ and R₁₀ are independently (C₁-C₈)alkyl;
R₁₁ and R₁₂ are independently —H or (C₁-C₈) alkyl;
X is CR₁₃;
Y is C; and
R₁₃ is —H or (C₁-C₈) alkyl.

9. A compound according to claim 1 wherein
R₁, R₂, R₃, R₄, and R₅ are —H;
R₆ is (C₁-C₁₄)alkyl, (C₃-C₈)cycloalkyl, heteroaryl, heteroarylthio(C₁-C₈)alkyl, or heterocyclyl, wherein the R₆ group is optionally substituted with 1 group which is (C₂-C₈)alkenyl, (C₁-C₈)alkoxy(C₁-C₈)alkyl, (C₁-C₈)alkyl, (C₂-C₈) alkynyl, (C₃-C₈) cycloalkyl, (C₃-C₈) cycloalkyl (C₁-C₈)alkyl, hydroxy(C₁-C₈)alkoxy, —NR₁₁R₁₂, (NR₁₁R₁₂) (C₁-C₈)alkyl, HOCH₂CH(NH₂) C(O)O—, R₁₂CH(NH₂) C(O)O—, NH₂CH₂C(O)O—, NH₂CH₂CH₂C(O)O—, or (CH₃)₂NCH₂C(O)O—;
R₇ is —H;
R₈ is (C₁-C₈) alkyl, (C₃-C₈) cycloalkyl(C₁-C₈) alkyl, halo (C₁-C₈) alkyl;
R₉ and R₁₀ are independently (C₁-C₈)alkyl;
R₁₁ and R₁₂ are independently —H, (C₁-C₈) alkyl, (C₂-C₈) alkynyl, (C₃-C₈) cycloalkyl, or (C₃-C₈)cycloalkyl(C₁-C₈)alkyl;
X is N or CH; and
Y is C.

10. A compound according to claim 1 wherein
R₁, R₂, R₃, R₄, and R₅ are —H;
R₆ is (C₃-C₈)cycloalkyl, heteroaryl, heterocyclyl, or hydroxy(C₁-C₈)alkyl, wherein the R₆ group is optionally substituted with 1 group which is hydroxy, hydroxy(C₁-C₈)alkoxy(C₁-C₈)alkoxy, hydroxy(C₁-C₈)alkyl, R₁₁ON═CH(CH₂)ₙO—, or HONHC(O) (CH₂)ₙO—;
R₇ is —H;
R₈ is (C₁-C₈) alkyl, (C₃-C₈) cycloalkyl (C₁-C₈)alkyl, aryl, aryl (C₁-C₈) alkyl, halo (C₁-C₈) alkyl, heteroaryl, heteroaryl (C₁-C₈) alkyl, or hydroxy (C₁-C₈) alkyl;
R₉ and R₁₀ are independently (C₁-C₈)alkyl;
R₁₁ is (C₁-C₈) alkyl or aryl;
n is 1, 2, 3, 4, 5, or 6;
X is N or CH; and
Y is C.

11. A compound according to claim 1 wherein
R₁ and R₂ are independently —H or C₁-C₈ alkyl;
R₃ and R₄ are independently —H or —F;
R₅ and R₆ taken together with the nitrogen atom to which they are attached form a heteroaryl, wherein the heteroaryl is imidazolyl, oxazolyl, pyrazolyl, pyridazinyl, pyridinyl, pyrazinyl, pyrrolyl, or thiazolyl, wherein the heteroaryl is optionally substituted with(C₁-C₈)alkoxy, (C₁-C₈)alkoxycarbonyl, (C₁-C₈) alkyl, (C₁-C₈) alkylcarbonyl, (C₁-C₈)alkylcarbonyloxy, aryl, aryl (C₁-C₈) alkyl, cyano, cyano (C₁-C₈) alkyl, (C₃-C₈) cycloalkyl, (C₃-C₈) cycloalkyl (C₁-C₈)alkyl, halo (C₁-C₈) alkoxy, halo (C₁-C₈) alkyl, halogen, heteroaryl, heteroaryl (C₁-C₈) alkyl, heterocyclyl, heterocycle (C₁-C₈)alkyl, hydroxy, hydroxy(C₁-C₈)alkyl, mercapto, nitro, —NR₁₁R₁₂, (NR₁₁R₁₂) (C₁-C₈) alkyl, or (NR₁₁R₁₂) carbonyl;
R₇ is —H or (C₁-C₈)alkyl;
R₈ is (C₂-C₈) alkenyl, (C₁-C₈) alkyl, (C₂-C₈) alkynyl, (C₃-C₈) cycloalkenyl, (C₃-C₈) cycloalkenyl (C₁-C₈) alkyl, (C₃-C₈) cycloalkyl, (C₃-C₈) cycloalkyl (C₁-C₈) alkyl, aryl, aryl (C₁-C₈) alkyl, halo (C₁-C₈) alkyl, heteroaryl, heteroaryl (C₁-C₈)alkyl, heterocyclyl, heterocycle (C₁-C₈)alkyl, or hydroxy (C₁-C₈) alkyl;
R₉ and R₁₀ are independently —H or (C₁-C₈) alkyl; or
R₉ and R₁₀ taken together with the carbon atom to which they are attached form (C₃-C₈)cycloalkyl; R₁₁ and R₁₂ are independently —H, (C₂-C₈)alkenyl, (C₁-C₈)alkyl, (C₁-C₈)alkylcarbonyl, (C₂-C₈)alkynyl, aryl, aryl(C₁-C₈)alkyl, (C₃-C₈)cycloalkyl, (C₃-C₈)cycloalkyl(C₁-C₈)alkyl, formyl, heteroaryl, heteroaryl(C₁-C₈)alkyl, heterocyclyl, or heterocycle (C₁-C₈) alkyl;
n is 1, 2, 3, 4, 5, or 6;
X is N or CR₁₃;
Y is C; and
R₁₃ is —H or (C₁-C₈) alkyl.

12. A compound according to claim 1 which is 2-fluoro-6-(tetrahydro-2H-pyran-4-ylamino)-4-(2,3,6,6-tetramethyl-4-oxo-4,5,6,7-tetrahydro-1H-indol-1-yl)benzamide;
4-(6,6-dimethyl-4-oxo-3-(trifluoromethyl)-4,5,6,7-tetrahydro-1H-indazol-1-yl)-2-fluoro-6-(4-hydroxycyclohexylamino)benzamide;
2-fluoro-6-(4-methoxycyclohexylamino)-4-(3,6,6-trimethyl-4-oxo-4,5,6,7-tetrahydro-1H-indazol-1-yl)benzamide;
4-(3-ethyl-6,6-dimethyl-4-oxo-4,5,6,7-tetrahydro-1H-indazol-1-yl)-3-fluoro-2-(2-hydroxycyclohexylamino)benzamide;
4-(3-ethyl-6,6-dimethyl-4-oxo-4,5,6,7-tetrahydro-1H-indazol-1-yl)-2,3,5-trifluoro-6-(4-hydroxycyclohexylamino)benzamide;
2-fluoro-6-(2-oxoazepan-3-ylamino)-4-(3,6,6-trimethyl-4-oxo-4,5,6,7-tetrahydro-1H-indazol-1-yl)benzamide;
2-(1,3-dihydroxypropan-2-ylamino)-6-fluoro-4-(3,6,6-trimethyl-4-oxo-4,5,6,7-tetrahydro-1H-indazol-1-yl)benzamide;
2-fluoro-6-(tetrahydrofuran-3-ylamino)-4-(3,6,6-trimethyl-4-oxo-4,5,6,7-tetrahydro-1H-indol-1-yl)benzamide;
4-(3-(cyclopropylmethyl)-6,6-dimethyl-4-oxo-4,5,6,7-tetrahydro-1H-indazol-1-yl)-5-fluoro-2-(tetrahydrofuran-3-ylamino)benzamide;
2-(2-aminocyclohexylamino)-6-fluoro-4-(3,6,6-trimethyl-4-oxo-4,5,6,7-tetrahydro-1H-indazol-1-yl)benzamide;
2-fluoro-6-(2-(neopentylamino)cyclohexylamino)-4-(3,6,6-trimethyl-4-oxo-4,5,6,7-tetrahydro-1H-indazol-1-yl)benzamide;
2-(2-((1H-imidazol-2-yl)methylthio)ethylamino)-4-(3-ethyl-6,6-dimethyl-4-oxo-4,5,6,7-tetrahydro-1H-indazol-1-yl)-6-fluorobenzamide;
2-(cyclopent-3-enylamino)-6-fluoro-4-(3,6,6-trimethyl-4-oxo-4,5,6,7-tetrahydro-1H-indazol-1-yl)benzamide;
2-fluoro-6-(2-hydroxycyclopentylamino)-4-(3,6,6-trimethyl-4-oxo-4,5,6,7-tetrahydro-1H-indol-1-yl)benzamide;
2-(cyclobutylamino)-4-(3-ethyl-6,6-dimethyl-4-oxo-4,5,6,7-tetrahydro-1H-indazol-1-yl)-6-fluorobenzamide;
4-(3-(cyclopropylmethyl)-6,6-dimethyl-4-oxo-4,5,6,7-tetrahydro-1H-indazol-1-yl)-2-fluoro-6-(tetrahydro-2H-pyran-4-ylamino)benzamide;
4-(3-ethyl-6,6-dimethyl-4-oxo-4,5,6,7-tetrahydro-1H-indazol-1-yl)-2-fluoro-6-(tetrahydro-2H-pyran-4-ylamino)benzamide;
2-fluoro-6-(tetrahydro-2H-pyran-4-ylamino)-4-(3,6,6-trimethyl-4-oxo-4,5,6,7-tetrahydro-1H-indazol-1-yl)benzamide;
4-(3-ethyl-6,6-dimethyl-4-oxo-4,5,6,7-tetrahydro-1H-indazol-1-yl)-3-fluoro-2-(4-hydroxycyclohexylamino)benzamide
2-fluoro-6-(tetrahydrofuran-3-ylamino)-4-(3,6,6-trimethyl-4-oxo-4,5,6,7-tetrahydro-1H-indazol-1-yl)benzamide;
2-fluoro-6-(tetrahydrofuran-3-ylamino)-4-(3,6,6-trimethyl-4-oxo-4,5,6,7-tetrahydro-1H-indazol-1-yl)benzamide;
2-(2-aminocyclohexylamino)-4-(3-ethyl-6,6-dimethyl-4-oxo-4,5,6,7-tetrahydro-1H-indazol-1-yl)-6-fluorobenzamide;
4-(3-(cyclopropylmethyl)-6,6-dimethyl-4-oxo-4,5,6,7-tetrahydro-1H-indazol-1-yl)-2-fluoro-6-(2-hydroxycyclopentylamino)benzamide;
4-(3-(cyclopropylmethyl)-6,6-dimethyl-4-oxo-4,5,6,7-tetrahydro-1H-indazol-1-yl)-2-fluoro-6-(2-hydroxycyclohexylamino)benzamide;
4-(3-(cyclopropylmethyl)-6,6-dimethyl-4-oxo-4,5,6,7-tetrahydro-1H-indazol-1-yl)-2-fluoro-6-(4-hydroxycyclohexylamino)benzamide;
4-(3-(cyclopropylmethyl)-6,6-dimethyl-4-oxo-4,5,6,7-tetrahydro-1H-indazol-1-yl)-5-fluoro-2-(4-hydroxycyclohexylamino)benzamide;
4-(3-ethyl-6,6-dimethyl-4-oxo-4,5,6,7-tetrahydro-1H-indazol-1-yl)-5-fluoro-2-(4-hydroxycyclohexylamino)benzamide;
2-fluoro-6-(2-hydroxycyclopentylamino)-4-(3,6,6-trimethyl-4-oxo-4,5,6,7-tetrahydro-1H-indazol-1-yl)benzamide;
2-(cyclopropylmethylamino)-4-(3-ethyl-6,6-dimethyl-4-oxo-4,5,6,7-tetrahydro-1H-indazol-1-yl)-6-fluorobenzamide;
2-fluoro-6-(2-hydroxycyclohexylamino)-4-(3,6,6-trimethyl-4-oxo-4,5,6,7-tetrahydro-1H-indazol-1-yl)benzamide;
4-(3-ethyl-6,6-dimethyl-4-oxo-4,5,6,7-tetrahydro-1H-indazol-1-yl)-2-fluoro-6-(tetrahydrofuran-3-ylamino)benzamide;
4-(3-ethyl-6,6-dimethyl-4-oxo-4,5,6,7-tetrahydro-1H-indazol-1-yl)-2-fluoro-6-(2-hydroxycyclohexylamino)benzamide;
4-(3-ethyl-6,6-dimethyl-4-oxo-4,5,6,7-tetrahydro-1H-indazol-1-yl)-2-fluoro-6-(tetrahydrofuran-3-ylamino)benzamide;
4-(3-(cyclopropylmethyl)-6,6-dimethyl-4-oxo-4,5,6,7-tetrahydro-1H-indazol-1-yl)-2-fluoro-6-(tetrahydrofuran-3-ylamino)benzamide;
4-(3-(cyclopropylmethyl)-6,6-dimethyl-4-oxo-4,5,6,7-tetrahydro-1H-indazol-1-yl)-2-fluoro-6-(tetrahydrofuran-3-ylamino)benzamide;
4-(3-ethyl-6,6-dimethyl-4-oxo-4,5,6,7-tetrahydro-1H-indazol-1-yl)-3-fluoro-2-(tetrahydro-2H-pyran-4-ylamino)benzamide;
4-(3-(cyclopropylmethyl)-6,6-dimethyl-4-oxo-4,5,6,7-tetrahydro-1H-indazol-1-yl)-3-fluoro-2-(tetrahydro-2H-pyran-4-ylamino)benzamide;
4-(3-ethyl-6,6-dimethyl-4-oxo-4,5,6,7-tetrahydro-1H-indazol-1-yl)-3-fluoro-2-(2-hydroxyethylamino)benzamide;
4-(3-(cyclopropylmethyl)-6,6-dimethyl-4-oxo-4,5,6,7-tetrahydro-1H-indazol-1-yl)-3-fluoro-2-(4-hydroxycyclohexylamino)benzamide;
4-(3-(cyclopropylmethyl)-6,6-dimethyl-4-oxo-4,5,6,7-tetrahydro-1H-indazol-1-yl)-3-fluoro-2-(2-hydroxycyclohexylamino)benzamide;
4-(3-ethyl-6,6-dimethyl-4-oxo-4,5,6,7-tetrahydro-1H-indazol-1-yl)-2-fluoro-6-(2-hydroxycyclopentylamino)benzamide;
4-(3-(cyclopropylmethyl)-6,6-dimethyl-4-oxo-4,5,6,7-tetrahydro-1H-indazol-1-yl)-5-fluoro-2-(tetrahydro-2H-pyran-4-ylamino)benzamide;
4-(3-ethyl-6,6-dimethyl-4-oxo-4,5,6,7-tetrahydro-1H-indazol-1-yl)-5-fluoro-2-(tetrahydro-2H-pyran-4-ylamino)benzamide;
4-(3-ethyl-6,6-dimethyl-4-oxo-4,5,6,7-tetrahydro-1H-indazol-1-yl)-5-fluoro-2-(tetrahydrofuran-3-ylamino)benzamide;
4-(3-(cyclopropylmethyl)-6,6-dimethyl-4-oxo-4,5,6,7-tetrahydro-1H-indazol-1-yl)-5-fluoro-2-(2-hydroxycyclohexylamino)benzamide;
4-(3-(cyclopropylmethyl)-6,6-dimethyl-4-oxo-4,5,6,7-tetrahydro-1H-indazol-1-yl)-5-fluoro-2-(2-hydroxycyclopentylamino)benzamide;
4-(3-ethyl-6,6-dimethyl-4-oxo-4,5,6,7-tetrahydro-1H-indazol-1-yl)-5-fluoro-2-(2-hydroxycyclopentylamino)benzamide;
4-(3-ethyl-6,6-dimethyl-4-oxo-4,5,6,7-tetrahydro-1H-indazol-1-yl)-3,5-difluoro-2-(tetrahydro-2H-pyran-4-ylamino)benzamide;
4-(3-ethyl-6,6-dimethyl-4-oxo-4,5,6,7-tetrahydro-1H-indazol-1-yl)-3,5-difluoro-2-(tetrahydrofuran-3-ylamino)benzamide;
4-(3-(cyclopropylmethyl)-6,6-dimethyl-4-oxo-4,5,6,7-tetrahydro-1H-indazol-1-yl)-3,5-difluoro-2-(tetrahydrofuran-3-ylamino)benzamide;
4-(3-(cyclopropylmethyl)-6,6-dimethyl-4-oxo-4,5,6,7-tetrahydro-1H-indazol-1-yl)-3,5-difluoro-2-(tetrahydro-2H-pyran-4-ylamino)benzamide;
4-(3-ethyl-6,6-dimethyl-4-oxo-4,5,6,7-tetrahydro-1H-indazol-1-yl)-2-fluoro-6-(2-(neopentylamino)cyclohexylamino)benzamide;
4-(3-ethyl-6,6-dimethyl-4-oxo-4,5,6,7-tetrahydro-1H-indazol-1-yl)-2-fluoro-6-(4-(neopentylamino)cyclohexylamino)benzamide;
4-(3-ethyl-6,6-dimethyl-4-oxo-4,5,6,7-tetrahydro-1H-indazol-1-yl)-2-fluoro-6-(2-oxotetrahydrofuran-3-ylamino)benzamide;
4-(3-(cyclopropylmethyl)-6,6-dimethyl-4-oxo-4,5,6,7-tetrahydro-1H-indazol-1-yl)-3,5-difluoro-2-(4-hydroxycyclohexylamino)benzamide;
methyl 2-(2-carbamoyl-5-(3-ethyl-6,6-dimethyl-4-oxo-4,5,6,7-tetrahydro-1H-indazol-1-yl)-3-fluorophenylamino)-4-hydroxybutanoate;
2-(cyclopent-3-enylamino)-4-(3-ethyl-6,6-dimethyl-4-oxo-4,5,6,7-tetrahydro-1H-indazol-1-yl)-6-fluorobenzamide;
2-(1-acetylpiperidin-4-ylamino)-4-(3-ethyl-6,6-dimethyl-4-oxo-4,5,6,7-tetrahydro-1H-indazol-1-yl)-6-fluorobenzamide;
4-(3-(cyclopropylmethyl)-6,6-dimethyl-4-oxo-4,5,6,7-tetrahydro-1H-indazol-1-yl)-2-fluoro-6-(2-oxotetrahydrofuran-3-ylamino)benzamide;
2-fluoro-6-(4-hydroxycyclohexylamino)-4-(3,6,6-trimethyl-4-oxo-4,5,6,7-tetrahydro-1H-indazol-1-yl)benzamide;
2-fluoro-6-(2-oxotetrahydrofuran-3-ylamino)-4-(3,6,6-trimethyl-4-oxo-4,5,6,7-tetrahydro-1H-indazol-1-yl)benzamide;
4-(3-ethyl-6,6-dimethyl-4-oxo-4,5,6,7-tetrahydro-1H-indazol-1-yl)-2-fluoro-6-(2-oxoazepan-3-ylamino)benzamide;
2-fluoro-6-(tetrahydrofuran-3-ylamino)-4-(2,3,6,6-tetramethyl-4-oxo-4,5,6,7-tetrahydro-1H-indol-1-yl)benzamide;
2-fluoro-6-(tetrahydro-2H-pyran-4-ylamino)-4-(3,6,6-trimethyl-4-oxo-4,5,6,7-tetrahydro-1H-indol-1-yl)benzamide;
2-(2,2-dimethoxyethylamino)-4-(3-ethyl-6,6-dimethyl-4-oxo-4,5,6,7-tetrahydro-1H-indazol-1-yl)-6-fluorobenzamide;
4-(3-ethyl-6,6-dimethyl-4-oxo-4,5,6,7-tetrahydro-1H-indazol-1-yl)-2-fluoro-6-(4-hydroxycyclohexylamino)benzamide;
2-fluoro-6-(4-hydroxycyclohexylamino)-4-(3,6,6-trimethyl-4-oxo-4,5,6,7-tetrahydro-1H-indol-1-yl)benzamide;
4-(3-ethyl-6,6-dimethyl-4-oxo-4,5,6,7-tetrahydro-1H-indazol-1-yl)-2-fluoro-6-(4-methoxycyclohexylamino)benzamide;
or a pharmaceutically acceptable salt thereof.

13. A compound according to claim 1 which is 2-(4-(cyclopropylmethylamino)cyclohexylamino)-4-(6,6-dimethyl-4-oxo-3-(trifluoromethyl)-4,5,6,7-tetrahydro-1H-indazol-1-yl)benzamide;
2-(1-(2-(isobutylamino)ethyl)-1H-pyrazol-4-ylamino)-4-(3,6,6-trimethyl-4-oxo-4,5,6,7-tetrahydro-1H-indazol-1-yl)benzamide;
4-(6,6-dimethyl-4-oxo-3-(trifluoromethyl)-4,5,6,7-tetrahydro-1H-indazol-1-yl)-2-(piperidin-3-ylamino)benzamide;
4-(6,6-dimethyl-4-oxo-3-(trifluoromethyl)-4,5,6,7-tetrahydro-1H-indazol-1-yl)-2-(1-neopentylpiperidin-3-ylamino)benzamide;
4-(6,6-dimethyl-4-oxo-3-(trifluoromethyl)-4,5,6,7-tetrahydro-1H-indazol-1-yl)-2-(1-(prop-2-ynyl)pyrrolidin-3-ylamino)benzamide;
2-(4-aminocyclohexylamino)-4-(6,6-dimethyl-4-oxo-3-(trifluoromethyl)-4,5,6,7-tetrahydro-1H-indazol-1-yl)benzamide;
(4-(2-carbamoyl-5-(6,6-dimethyl-4-oxo-3-(trifluoromethyl)-4,5,6,7-tetrahydro-1H-indazol-1-yl)phenylamino)cyclohexyl) 2-amino-3-hydroxypropanoate;
4-(2-carbamoyl-5-(6,6-dimethyl-4-oxo-3-(trifluoromethyl)-4,5,6,7-tetrahydro-1H-indazol-1-yl)phenylamino)cyclohexyl 2-(dimethylamino)acetate;
4-(6,6-dimethyl-4-oxo-3-(trifluoromethyl)-4,5,6,7-tetrahydro-1H-indazol-1-yl)-2-(pyrimidin-2-ylamino)benzamide;
4-(6,6-dimethyl-4-oxo-3-(trifluoromethyl)-4,5,6,7-tetrahydro-1H-indazol-1-yl)-2-(pyrimidin-4-ylamino)benzamide;
4-(6,6-dimethyl-4-oxo-3-(trifluoromethyl)-4,5,6,7-tetrahydro-1H-indazol-1-yl)-2-(pyrimidin-5-ylamino)benzamide;
4-(3-(difluoromethyl)-6,6-dimethyl-4-oxo-4,5,6,7-tetrahydro-1H-indazol-1-yl)-2-(1-(prop-2-ynyl)piperidin-4-ylamino)benzamide;
2-(1-cyclopropylpiperidin-4-ylamino)-4-(3-(difluoromethyl)-; 6,6-dimethyl-4-oxo-4,5,6,7-tetrahydro-1H-indazol-1-yl)benzamide;
4-(3-(difluoromethyl)-6,6-dimethyl-4-oxo-4,5,6,7-tetrahydro-1H-indazol-1-yl)-2-(1-(2-methoxyethyl)piperidin-4-ylamino)benzamide;
4-(6,6-dimethyl-4-oxo-3-(trifluoromethyl)-4,5,6,7-tetrahydro-1H-indazol-1-yl)-2-(2-(2-hydroxyethoxy)pyridin-4-ylamino)benzamide;
2-(1-(2-(methylamino)ethyl)-1H-pyrazol-4-ylamino)-4-(3,6,6-trimethyl-4-oxo-4,5,6,7-tetrahydro-1H-indazol-1-yl)benzamide;
4-(6,6-dimethyl-4-oxo-3-(trifluoromethyl)-4,5,6,7-tetrahydro-1H-indazol-1-yl)-2-(4-(prop-2-ynylamino)cyclohexylamino)benzamide;
4-(6,6-dimethyl-4-oxo-3-(trifluoromethyl)-4,5,6,7-tetrahydro-1H-indazol-1-yl)-2-(1-(prop-2-ynyl)piperidin-4-ylamino)benzamide;
2-(azetidin-3-ylamino)-4-(6,6-dimethyl-4-oxo-3-(trifluoromethyl)-4,5,6,7-tetrahydro-1H-indazol-1-yl)benzamide;
2-(2-aminocyclohexylamino)-4-(6,6-dimethyl-4-oxo-3-(trifluoromethyl)-4,5,6,7-tetrahydro-1H-indazol-1-yl)benzamide;
4-(6,6-dimethyl-4-oxo-3-(trifluoromethyl)-4,5,6,7-tetrahydro-1H-indazol-1-yl)-2-(2-(diprop-2-ynylamino)ethylamino)benzamide;
4-(6,6-dimethyl-4-oxo-3-(trifluoromethyl)-4,5,6,7-tetrahydro-1H-indazol-1-yl)-2-(2-(prop-2-ynylamino)ethylamino)benzamide;
4-(6,6-dimethyl-4-oxo-3-(trifluoromethyl)-4,5,6,7-tetrahydro-1H-indazol-1-yl)-2-(2-(prop-2-ynylamino)cyclohexylamino)benzamide;
2-(1-allylpiperidin-4-ylamino)-4-(6,6-dimethyl-4-oxo-3-(trifluoromethyl)-4,5,6,7-tetrahydro-1H-indazol-1-yl)benzamide;
2-(2-aminocyclohexylamino)-4-(6,6-dimethyl-4-oxo-3-(trifluoromethyl)-4,5,6,7-tetrahydro-1H-indazol-1-yl)benzamide;
2-(2-aminocyclohexylamino)-4-(6,6-dimethyl-4-oxo-3-(trifluoromethyl)-4,5,6,7-tetrahydro-1H-indazol-1-yl)benzamide;
4-(6,6-dimethyl-4-oxo-3-(trifluoromethyl)-4,5,6,7-tetrahydro-1H-indazol-1-yl)-2-(1-(prop-2-ynyl)piperidin-3-ylamino)benzamide;
2-(1-allylpiperidin-3-ylamino)-4-(6,6-dimethyl-4-oxo-3-(trifluoromethyl)-4,5,6,7-tetrahydro-1H-indazol-1-yl)benzamide;
4-(6,6-dimethyl-4-oxo-3-(trifluoromethyl)-4,5,6,7-tetrahydro-1H-indazol-1-yl)-2-(piperidin-3-ylamino)benzamide;
4-(6,6-dimethyl-4-oxo-3-(trifluoromethyl)-4,5,6,7-tetrahydro-1H-indazol-1-yl)-2-(2-(pyrimidin-2-ylthio)ethylamino)benzamide;
4-(6,6-dimethyl-4-oxo-3-(trifluoromethyl)-4,5,6,7-tetrahydro-1H-indazol-1-yl)-2-(1-(prop-2-ynyl)piperidin-3-ylamino)benzamide;
2-(1-(cyclopropylmethyl)piperidin-3-ylamino)-4-(6,6-dimethyl-4-oxo-3-(trifluoromethyl)-4,5,6,7-tetrahydro-1H-indazol-1-yl)benzamide;
2-(1-(cyclopropylmethyl)piperidin-4-ylamino)-4-(6,6-dimethyl-4-oxo-3-(trifluoromethyl)-4,5,6,7-tetrahydro-1H-indazol-1-yl)benzamide;
2-(3-aminocyclohexylamino)-4-(6,6-dimethyl-4-oxo-3-(trifluoromethyl)-4,5,6,7-tetrahydro-1H-indazol-1-yl)benzamide;
2-(1-(2-(tert-butylamino)ethyl)-1H-pyrazol-4-ylamino)-4-(3,6,6-trimethyl-4-oxo-4,5,6,7-tetrahydro-1H-indazol-1-yl)benzamide;
4-(6,6-dimethyl-4-oxo-3-(trifluoromethyl)-4,5,6,7-tetrahydro-1H-indazol-1-yl)-2-(pyridazin-4-ylamino)benzamide;
2-(1-(cyclopropylmethyl)piperidin-3-ylamino)-4-(6,6-dimethyl-4-oxo-3-(trifluoromethyl)-4,5,6,7-tetrahydro-1H-indazol-1-yl)benzamide;
2-(2-(cyclopropylmethylamino)cyclohexylamino)-4-(6,6-dimethyl-4-oxo-3-(trifluoromethyl)-4,5,6,7-tetrahydro-1H-indazol-1-yl)benzamide;
2-(2-(1H-imidazol-2-ylthio)ethylamino)-4-(6,6-dimethyl-4-oxo-3-(trifluoromethyl)-4,5,6,7-tetrahydro-1H-indazol-1-yl)benzamide;
4-(6,6-dimethyl-4-oxo-3-(trifluoromethyl)-4,5,6,7-tetrahydro-1H-indazol-1-yl)-2-(2-(prop-2-ynylamino)cyclohexylamino)benzamide;
4-(6,6-dimethyl-4-oxo-3-(trifluoromethyl)-4,5,6,7-tetrahydro-1H-indazol-1-yl)-2-(2-(diprop-2-ynylamino)cyclohexylamino)benzamide;
2-(2-(cyclopropylmethylamino)cyclohexylamino)-4-(6,6-dimethyl-4-oxo-3-(trifluoromethyl)-4,5,6,7-tetrahydro-1H-indazol-1-yl)benzamide;
2-(2-(bis(cyclopropylmethyl)amino)cyclohexylamino)-4-(6,6-dimethyl-4-oxo-3-(trifluoromethyl)-4,5,6,7-tetrahydro-1H-indazol-1-yl)benzamide;
2-(2-(diethylamino)cyclohexylamino)-4-(6,6-dimethyl-4-oxo-3-(trifluoromethyl)-4,5,6,7-tetrahydro-1H-indazol-1-yl)benzamide;
2-(2-(cyclopropylamino)cyclohexylamino)-4-(6,6-dimethyl-4-oxo-3-(trifluoromethyl)-4,5,6,7-tetrahydro-1H-indazol-1-yl)benzamide;
4-(6,6-dimethyl-4-oxo-3-(trifluoromethyl)-4,5,6,7-tetrahydro-1H-indazol-1-yl)-2-(4-(neopentylamino)cyclohexylamino)benzamide;
4-(6,6-dimethyl-4-oxo-3-(trifluoromethyl)-4,5,6,7-tetrahydro-1H-indazol-1-yl)-2-(2-(neopentylamino)cyclohexylamino)benzamide;
or a pharmaceutically acceptable salt thereof.

14. A compound according to claim 1 which is 2-(4-hydroxycyclohexylamino)-4-(3-(hydroxymethyl)-6,6-dimethyl-4-oxo-4,5,6,7-tetrahydro-1H-indazol-1-yl)benzamide;
2-(1-(2-hydroxyethyl)-1H-pyrazol-4-ylamino)-4-(3,6,6-trimethyl-4-oxo-4,5,6,7-tetrahydro-1H-indazol-1-yl)benzamide;
4-(6,6-dimethyl-4-oxo-3-(pyridin-2-ylmethyl)-4,5,6,7-tetrahydro-1H-indazol-1-yl)-2-(tetrahydro-2H-pyran-4-ylamino)benzamide;
4-(3-ethyl-6,6-dimethyl-4-oxo-4,5,6,7-tetrahydro-1H-indazol-1-yl)-2-(tetrahydrofuran-3-ylamino)benzamide;
4-(6,6-dimethyl-4-oxo-3-(trifluoromethyl)-4,5,6,7-tetrahydro-1H-indazol-1-yl)-2-(2-hydroxypropylamino)benzamide;
4-(6,6-dimethyl-4-oxo-3-(trifluoromethyl)-4,5,6,7-tetrahydro-1H-indazol-1-yl)-2-(4-(2-(methoxyimino)ethoxy)cyclohexylamino)benzamide;
4-(6,6-dimethyl-4-oxo-3-(trifluoromethyl)-4,5,6,7-tetrahydro-1H-indazol-1-yl)-2-(4-(2-(phenoxyimino)ethoxy)cyclohexylamino)benzamide;
2-(4-(2-hydroxyethoxy)cyclohexylamino)-4-(3,6,6-trimethyl-4-oxo-4,5,6,7-tetrahydro-1H-indazol-1-yl)benzamide;
2-(tetrahydrofuran-3-ylamino)-4-(3,6,6-trimethyl-4-oxo-4,5,6,7-tetrahydro-1H-indazol-1-yl)benzamide;
4-(6,6-dimethyl-4-oxo-3-(trifluoromethyl)-4,5,6,7-tetrahydro-1H-indazol-1-yl)-2-(4-(2-(hydroxyamino)-2-oxoethoxy)cyclohexylamino)benzamide;
2-(4-(2-(2-hydroxyethoxy)ethoxy)cyclohexylamino)-4-(3,6,6-trimethyl-4-oxo-4,5,6,7-tetrahydro-1H-indazol-1-yl)benzamide;
2-(cyclobutylamino)-4-(6,6-dimethyl-4-oxo-3-(trifluoromethyl)-4,5,6,7-tetrahydro-1H-indazol-1-yl)benzamide;
2-(4-(2-hydroxyethoxy)cyclohexylamino)-4-(3,6,6-trimethyl-4-oxo-4,5,6,7-tetrahydro-1H-indazol-1-yl)benzamide;
4-(6,6-dimethyl-4-oxo-3-(trifluoromethyl)-4,5,6,7-tetrahydro-1H-indazol-1-yl)-2-(2-hydroxyethylamino)benzamide;
2-(cyclobutylamino)-4-(3-ethyl-6,6-dimethyl-4-oxo-4,5,6,7-tetrahydro-1H-indazol-1-yl)benzamide;
2-(−4-hydroxycyclohexylamino)-4-(3-(3-methoxyphenyl)-6,6-dimethyl-4-oxo-4,5,6,7-tetrahydro-1H-indazol-1-yl)benzamide;
4-(3-benzyl-6,6-dimethyl-4-oxo-4,5,6,7-tetrahydro-1H-indazol-1-yl)-2-(4-hydroxycyclohexylamino)benzamide;
4-(6,6-dimethyl-4-oxo-3-(1H-pyrazol-4-yl)-4,5,6,7-tetrahydro-1H-indazol-1-yl)-2-(4-hydroxycyclohexylamino)benzamide;
4-(6,6-dimethyl-4-oxo-3-(pyridin-3-ylmethyl)-4,5,6,7-tetrahydro-1H-indazol-1-yl)-2-(4-hydroxycyclohexylamino)benzamide;
4-(6,6-dimethyl-4-oxo-3-(pyridin-2-ylmethyl)-4,5,6,7-tetrahydro-1H-indazol-1-yl)-2-(4-hydroxycyclohexylamino)benzamide;
4-(3-(cyclopropylmethyl)-6,6-dimethyl-4-oxo-4,5,6,7-tetrahydro-1H-indazol-1-yl)-2-(tetrahydro-2H-pyran-4-ylamino)benzamide;
4-(6,6-dimethyl-4-oxo-3-(trifluoromethyl)-4,5,6,7-tetrahydro-1H-indazol-1-yl)-2-(2-hydroxycyclohexylamino)benzamide;
4-(6,6-dimethyl-4-oxo-3-(pyridin-2-ylmethyl)-4,5,6,7-tetrahydro-1H-indazol-1-yl)-2-(2-hydroxyethylamino)benzamide;
4-(3-benzyl-6,6-dimethyl-4-oxo-4,5,6,7-tetrahydro-1H-indazol-1-yl)-2-(2-hydroxyethylamino)benzamide;
4-(3-(cyclopropylmethyl)-6,6-dimethyl-4-oxo-4,5,6,7-tetrahydro-1H-indazol-1-yl)-2-(2-hydroxyethylamino)benzamide;
4-(3-benzyl-6,6-dimethyl-4-oxo-4,5,6,7-tetrahydro-1H-indazol-1-yl)-2-(tetrahydro-2H-pyran-4-ylamino)benzamide;
4-(6,6-dimethyl-4-oxo-3-(trifluoromethyl)-4,5,6,7-tetrahydro-1H-indazol-1-yl)-2-(1-hydroxypropan-2-ylamino)benzamide;
4-(6,6-dimethyl-4-oxo-3-(trifluoromethyl)-4,5,6,7-tetrahydro-1H-indazol-1-yl)-2-(1-hydroxypropan-2-ylamino)benzamide;
4-(6,6-dimethyl-4-oxo-3-(trifluoromethyl)-4,5,6,7-tetrahydro-1H-indazol-1-yl)-2-(2-hydroxypropylamino)benzamide;
4-(6,6-dimethyl-4-oxo-3-(pyridin-3-ylmethyl)-4,5,6,7-tetrahydro-1H-indazol-1-yl)-2-(tetrahydro-2H-pyran-4-ylamino)benzamide
4-(6,6-dimethyl-4-oxo-3-(pyridin-4-ylmethyl)-4,5,6,7-tetrahydro-1H-indazol-1-yl)-2-(2-hydroxyethylamino)benzamide;
4-(6,6-dimethyl-4-oxo-3-(pyridin-3-ylmethyl)-4,5,6,7-tetrahydro-1H-indazol-1-yl)-2-(2-hydroxyethylamino)benzamide;
4-(6,6-dimethyl-4-oxo-3-(thiophen-3-ylmethyl)-4,5,6,7-tetrahydro-1H-indazol-1-yl)-2-(2-hydroxyethylamino)benzamide;
4-(6,6-dimethyl-4-oxo-3-(pyridin-4-ylmethyl)-4,5,6,7-tetrahydro-1H-indazol-1-yl)-2-(tetrahydro-2H-pyran-4-ylamino)benzamide;
4-(6,6-dimethyl-4-oxo-3-(pyridin-4-ylmethyl)-4,5,6,7-tetrahydro-1H-indazol-1-yl)-2-(4-hydroxycyclohexylamino)benzamide;
4-(6,6-dimethyl-4-oxo-3-(trifluoromethyl)-4,5,6,7-tetrahydro-1H-indazol-1-yl)-2-(2-hydroxycyclohexylamino)benzamide;
4-(3-(cyclopropylmethyl)-6,6-dimethyl-4-oxo-4,5,6,7-tetrahydro-1H-indazol-1-yl)-2-(tetrahydrofuran-3-ylamino)benzamide;
4-(6,6-dimethyl-4-oxo-3-(trifluoromethyl)-4,5,6,7-tetrahydro-1H-indazol-1-yl)-2-(2-hydroxycyclohexylamino)benzamide;
4-(6,6-dimethyl-4-oxo-3-(trifluoromethyl)-4,5,6,7-tetrahydro-1H-indazol-1-yl)-2-(2-hydroxycyclopentylamino)benzamide;
4-(6,6-dimethyl-4-oxo-3-(trifluoromethyl)-4,5,6,7-tetrahydro-1H-indazol-1-yl)-2-(2-hydroxycyclopentylamino)benzamide;
4-(3-ethyl-6,6-dimethyl-4-oxo-4,5,6,7-tetrahydro-1H-indazol-1-yl)-2-(2-hydroxycyclohexylamino)benzamide;
4-(3-(cyclopropylmethyl)-6,6-dimethyl-4-oxo-4,5,6,7-tetrahydro-1H-indazol-1-yl)-2-(2-hydroxycyclopentylamino)benzamide;
4-(3-(cyclopropylmethyl)-6,6-dimethyl-4-oxo-4,5,6,7-tetrahydro-1H-indazol-1-yl)-2-(2-hydroxycyclohexylamino)benzamide;
4-(3-ethyl-6,6-dimethyl-4-oxo-4,5,6,7-tetrahydro-1H-indazol-1-yl)-2-(2-hydroxycyclopentylamino)benzamide;
2-(tetrahydrofuran-3-ylamino)-4-(3,6,6-trimethyl-4-oxo-4,5,6,7-tetrahydro-1H-indazol-1-yl)benzamide;
2-(2-hydroxycyclopentylamino)-4-(3,6,6-trimethyl-4-oxo-4,5,6,7-tetrahydro-1H-indazol-1-yl)benzamide;
2-(2-hydroxycyclohexylamino)-4-(3,6,6-trimethyl-4-oxo-4,5,6,7-tetrahydro-1H-indazol-1-yl)benzamide;
or a pharmaceutically acceptable salt thereof.

15. A compound according to claim 1 which is 2-methyl-4-(3,6,6-trimethyl-4-oxo-4,5,6,7-tetrahydro-1H-indazol-1-yl)-1H-benzo[d]imidazole-7-carboxamide;
4-(3,6,6-trimethyl-4-oxo-4,5,6,7-tetrahydro-1H-indazol-1-yl)-1H-benzo[d]imidazole-7-carboxamide;
4-(3-ethyl-6,6-dimethyl-4-oxo-4,5,6,7-tetrahydro-1H-indazol-1-yl)-1H-benzo[d]imidazole-7-carboxamide;
4-(3-ethyl-6,6-dimethyl-4-oxo-4,5,6,7-tetrahydro-1H-indazol-1-yl)-2-methyl-1H-benzo[d]imidazole-7-carboxamide;
2-phenyl-4-(3,6,6-trimethyl-4-oxo-4,5,6,7-tetrahydro-1H-indazol-1-yl)-1H-benzo[d]imidazole-7-carboxamide;
4-(3-ethyl-6,6-dimethyl-4-oxo-4,5,6,7-tetrahydro-1H-indazol-1-yl)-2-phenyl-1H-benzo[d]imidazole-7-carboxamide;
or a pharmaceutically acceptable salt thereof.

16. A compound according to claim 1 which is 5-(6,6-dimethyl-4-oxo-3-(trifluoromethyl)-4,5,6,7-tetrahydro-1H-indazol-1-yl)-3-(-4-hydroxycyclohexylamino)picolinamide.

17. A compound according to claim 1 which is 2-fluoro-6-(2-hydroxycyclohexylamino)-4-(3,6,6-trimethyl-4-oxo-4,5,6,7-tetrahydro-1H-indol-1-yl)benzamide;
4-(3-ethyl-6,6-dimethyl-4-oxo-4,5,6,7-tetrahydro-1H-indazol-1-yl)-2-fluoro-6-(2-hydroxycyclohexylamino)benzamide;
4-(3-ethyl-6,6-dimethyl-4-oxo-4,5,6,7-tetrahydro-1H-indazol-1-yl)-2-fluoro-6-(2-hydroxycyclopentylamino)benzamide;
2-(cyclopentylamino)-4-(3-ethyl-6,6-dimethyl-4-oxo-4,5,6,7-tetrahydro-1H-indazol-1-yl)-6-fluorobenzamide;
2-(2-carbamoyl-3-fluoro-5-(3,6,6-trimethyl-4-oxo-4,5,6,7-tetrahydro-1H-indol-1-yl)phenylamino)cyclohexyl 2-aminoacetate;
2-(2-carbamoyl-3-fluoro-5-(3,6,6-trimethyl-4-oxo-4,5,6,7-tetrahydro-1H-indol-1-yl)phenylamino)cyclopentyl 2-aminoacetate;
2-(2-carbamoyl-5-(3-ethyl-6,6-dimethyl-4-oxo-4,5,6,7-tetrahydro-1H-indazol-1-yl)-3-fluorophenylamino)cyclohexyl 2-aminoacetate;
2-(2-carbamoyl-5-(3-ethyl-6,6-dimethyl-4-oxo-4,5,6,7-tetrahydro-1H-indazol-1-yl)-3-fluorophenylamino)cyclopentyl 2-aminoacetate;
2-(2-carbamoyl-3-fluoro-5-(3,6,6-trimethyl-4-oxo-4,5,6,7-tetrahydro-1H-indazol-1-yl)phenylamino)cyclopentyl 2-aminoacetate;
2-(2-carbamoyl-3-fluoro-5-(3,6,6-trimethyl-4-oxo-4,5,6,7-tetrahydro-1H-indazol-1-yl)phenylamino)cyclohexyl 2-aminoacetate;
4-(2-carbamoyl-3-fluoro-5-(3,6,6-trimethyl-4-oxo-4,5,6,7-tetrahydro-1H-indol-1-yl)phenylamino)cyclohexyl 2-aminoacetate;
4-(2-carbamoyl-5-(3-ethyl-6,6-dimethyl-4-oxo-4,5,6,7-tetrahydro-1H-indazol-1-yl)-3-fluorophenylamino)cyclohexyl 2-aminoacetate;
4-(2-carbamoyl-3-fluoro-5-(3,6,6-trimethyl-4-oxo-4,5,6,7-tetrahydro-1H-indazol-1-yl)phenylamino)cyclohexyl 2-aminoacetate;
2-fluoro-6-(6-oxotetrahydro-2H-pyran-3-ylamino)-4-(3,6,6-trimethyl-4-oxo-4,5,6,7-tetrahydro-1H-indazol-1-yl)benzamide;
2-fluoro-6-(6-oxotetrahydro-2H-pyran-3-ylamino)-4-(3,6,6-trimethyl-4-oxo-4,5,6,7-tetrahydro-1H-indazol-1-yl)benzamide;
2-fluoro-6-(4-hydroxycyclohexylamino)-4-(3,6,6-trimethyl-4-oxo-4,5,6,7-tetrahydro-1H-indazol-1-yl)benzamide;
4-(3-ethyl-6,6-dimethyl-4-oxo-4,5,6,7-tetrahydro-1H-indol-1-yl)-2-fluoro-6-(4-hydroxycyclohexylamino)benzamide;
2-fluoro-6-(oxetan-3-ylamino)-4-(3,6,6-trimethyl-4-oxo-4,5,6,7-tetrahydro-1H-indazol-1-yl)benzamide;
2-(cyclopentylamino)-6-fluoro-4-(3,6,6-trimethyl-4-oxo-4,5,6,7-tetrahydro-1H-indazol-1-yl)benzamide;
2-(cycloheptylamino)-6-fluoro-4-(3,6,6-trimethyl-4-oxo-4,5,6,7-tetrahydro-1H-indazol-1-yl)benzamide;
4-(2-carbamoyl-5-(6,6-dimethyl-4-oxo-3-(trifluoromethyl)-4,5,6,7-tetrahydro-1H-indazol-1-yl)-3-fluorophenylamino)cyclohexyl 2-aminoacetate;
4-(6,6-dimethyl-4-oxo-3-(trifluoromethyl)-4,5,6,7-tetrahydro-1H-indazol-1-yl)-2-fluoro-6-(2-hydroxycyclopentylamino)benzamide;
2-(2-carbamoyl-5-(6,6-dimethyl-4-oxo-3-(trifluoromethyl)-4,5,6,7-tetrahydro-1H-indazol-1-yl)-3-fluorophenylamino)cyclopentyl 2-aminoacetate;
4-(6,6-dimethyl-4-oxo-3-(trifluoromethyl)-4,5,6,7-tetrahydro-1H-indazol-1-yl)-2-fluoro-6-(tetrahydro-2H-pyran-4-ylamino)benzamide;
4-(6,6-dimethyl-4-oxo-3-(trifluoromethyl)-4,5,6,7-tetrahydro-1H-indazol-1-yl)-2-fluoro-6-(2-hydroxycyclohexylamino)benzamide;
2-(2-carbamoyl-5-(6,6-dimethyl-4-oxo-3-(trifluoromethyl)-4,5,6,7-tetrahydro-1H-indazol-1-yl)-3-fluorophenylamino)cyclohexyl 2-aminoacetate;
4-(6,6-dimethyl-4-oxo-3-(trifluoromethyl)-4,5,6,7-tetrahydro-1H-indazol-1-yl)-2-fluoro-6-(tetrahydrofuran-3-ylamino)benzamide;
4-(3-ethyl-6,6-dimethyl-4-oxo-4,5,6,7-tetrahydro-1H-indazol-1-yl)-2-fluoro-6-(2-hydroxycyclopentylamino)benzamide;
4-(3-ethyl-6,6-dimethyl-4-oxo-4,5,6,7-tetrahydro-1H-indol-1-yl)-2-fluoro-6-(tetrahydrofuran-3-ylamino)benzamide;
2-(4,4-Difluoro-cyclohexylamino)-6-fluoro-4-(3,6,6-trimethyl-4-oxo-4,5,6,7-tetrahydro-indazol-1-yl)-benzamide;
2-fluoro-6-(2-hydroxycyclopentylamino)-4-(3,6,6-trimethyl-4-oxo-4,5,6,7-tetrahydro-1H-indazol-1-yl)benzamide;
2-(4,4-difluorocyclohexylamino)-6-fluoro-4-(3,6,6-trimethyl-4-oxo-4,5,6,7-tetrahydro-1H-indazol-1-yl)benzamide;
4-(6,6-dimethyl-4-oxo-3-(trifluoromethyl)-4,5,6,7-tetrahydro-1H-indol-1-yl)-2-fluoro-6-(2-hydroxycyclopentylamino)benzamide;
2-(cyclopentylamino)-4-(6,6-dimethyl-4-oxo-3-(trifluoromethyl)-4,5,6,7-tetrahydro-1H-indazol-1-yl)-6-fluorobenzamide;
2-(cyclohexylamino)-6-fluoro-4-(3,6,6-trimethyl-4-oxo-4,5,6,7-tetrahydro-1H-indazol-1-yl)benzamide;
2-(2-carbamoyl-5-(6,6-dimethyl-4-oxo-3-(trifluoromethyl)-4,5,6,7-tetrahydro-1H-indol-1-yl)-3-fluorophenylamino)cyclopentyl 2-aminoacetate methanesulfonate;
4-(6,6-dimethyl-4-oxo-3-(trifluoromethyl)-4,5,6,7-tetrahydro-1H-indol-1-yl)-2-fluoro-6-(4-hydroxycyclohexylamino)benzamide;
4-(6,6-dimethyl-4-oxo-3-(trifluoromethyl)-4,5,6,7-tetrahydro-1H-indol-1-yl)-2-fluoro-6-(2-hydroxycyclohexylamino)benzamide;
2-(2-carbamoyl-5-(6,6-dimethyl-4-oxo-3-(trifluoromethyl)-4,5,6,7-tetrahydro-1H-indol-1-yl)-3-fluorophenylamino)cyclohexyl 2-aminoacetate methanesulfonate;
4-(6,6-dimethyl-4-oxo-3-(trifluoromethyl)-4,5,6,7-tetrahydro-1H-indol-1-yl)-2-fluoro-6-(tetrahydro-2H-pyran-4-ylamino)benzamide;
4-(6,6-dimethyl-4-oxo-3-(trifluoromethyl)-4,5,6,7-tetrahydro-1H-indol-1-yl)-2-fluoro-6-(tetrahydrofuran-3-ylamino)benzamide;
2-(4,4-difluorocyclohexylamino)-6-fluoro-4-(3,6,6-trimethyl-4-oxo-4,5,6,7-tetrahydro-1H-indol-1-yl)benzamide;
2-fluoro-6-(2-hydroxycyclopentylamino)-4-(3,6,6-trimethyl-4-oxo-4,5,6,7-tetrahydro-1H-indazol-1-yl)benzamide;
2-(cyclopentylamino)-6-fluoro-4-(3,6,6-trimethyl-4-oxo-4,5,6,7-tetrahydro-1H-indol-1-yl)benzamide;
2-(2-carbamoyl-3-fluoro-5-(3,6,6-trimethyl-4-oxo-4,5,6,7-tetrahydro-1H-indazol-1-yl)phenylamino)cyclopentyl 2-aminoacetate;
2-(cyclopentylamino)-4-(6,6-dimethyl-4-oxo-3-(trifluoromethyl)-4,5,6,7-tetrahydro-1H-indol-1-yl)-6-fluorobenzamide;
4-(2-carbamoyl-5-(6,6-dimethyl-4-oxo-3-(trifluoromethyl)-4,5,6,7-tetrahydro-1H-indol-1-yl)-3-fluorophenylamino)cyclohexyl 2-aminoacetate hydrochloride;
2-(4,4-difluorocyclohexylamino)-4-(6,6-dimethyl-4-oxo-3-(trifluoromethyl)-4,5,6,7-tetrahydro-1H-indol-1-yl)-6-fluorobenzamide;
2-(cyclohexylamino)-4-(6,6-dimethyl-4-oxo-3-(trifluoromethyl)-4,5,6,7-tetrahydro-1H-indol-1-yl)-6-fluorobenzamide;
or a pharmaceutically acceptable salt thereof.

18. A compound according to claim 1 which is
2-(2-carbamoyl-5-(3-ethyl-6,6-dimethyl-4-oxo-4,5,6,7-tetrahydro-1H-indazol-1-yl)phenylamino)cyclohexyl 2-aminoacetate;
4-(3-(difluoromethyl)-6,6-dimethyl-4-oxo-4,5,6,7-tetrahydro-1H-indazol-1-yl)-2-(2,2,6,6-tetramethylpiperidin-4-ylamino)benzamide;
2-(2-carbamoyl-5-(6,6-dimethyl-4-oxo-3-(trifluoromethyl)-4,5,6,7-tetrahydro-1H-indazol-1-yl)phenylamino)cyclopentyl 2-aminoacetate;
2-(2-carbamoyl-5-(6,6-dimethyl-4-oxo-3-(trifluoromethyl)-4,5,6,7-tetrahydro-1H-indazol-1-yl)phenylamino)cyclohexyl 2-aminoacetate;
2-(2-carbamoyl-5-(6,6-dimethyl-4-oxo-3-(trifluoromethyl)-4,5,6,7-tetrahydro-1H-indol-1-yl)phenylamino)cyclopentyl 2-aminoacetate methanesulfonate;
2-(2-carbamoyl-5-(6,6-dimethyl-4-oxo-3-(trifluoromethyl)-4,5,6,7-tetrahydro-1H-indazol-1-yl)phenylamino)cyclopentyl 1-aminocyclopropanecarboxylate methanesulfonate;
4-(2-carbamoyl-5-(6,6-dimethyl-4-oxo-3-(trifluoromethyl)-4,5,6,7-tetrahydro-1H-indol-1-yl)phenylamino)cyclohexyl 2-aminoacetate methanesulfonate;
4-(2-carbamoyl-5-(6,6-dimethyl-4-oxo-3-(trifluoromethyl)-4,5,6,7-tetrahydro-1H-indazol-1-yl)phenylamino)cyclohexyl 2-(2-aminoacetamido)acetate 2,2,2-trifluoroacetate;
or a pharmaceutically acceptable salt thereof.

19. A compound according to claim 1 which is 4-(3-ethyl-6,6-dimethyl-4-oxo-4,5,6,7-tetrahydro-1H-indazol-1-yl)-2-(2-hydroxycyclopentylamino)-6-methoxybenzamide;
2-(oxetan-3-ylamino)-4-(3,6,6-trimethyl-4-oxo-4,5,6,7-tetrahydro-1H-indazol-1-yl)benzamide;
4-(6,6-dimethyl-4-oxo-3-(trifluoromethyl)-4,5,6,7-tetrahydro-1H-indazol-1-yl)-2-(tetrahydrofuran-3-ylamino)benzamide;
4-(6-ethyl-3,6-dimethyl-4-oxo-4,5,6,7-tetrahydro-1H-indazol-1-yl)-2-(4-hydroxycyclohexylamino)benzamide;
4-(6,6-dimethyl-4-oxo-3-(trifluoromethyl)-4,5,6,7-tetrahydro-1H-indol-1-yl)-2-(2-hydroxycyclopentylamino)benzamide;
4-(3-ethyl-6,6-dimethyl-4-oxo-4,5,6,7-tetrahydro-1H-indol-1-yl)-2-(tetrahydrofuran-3-ylamino)benzamide;
4-(3-ethyl-6,6-dimethyl-4-oxo-4,5,6,7-tetrahydro-1H-indazol-1-yl)-2-methoxy-6-(tetrahydro-2H-pyran-4-ylamino)benzamide;
4-(6,6-dimethyl-4-oxo-3-(2,2,2-trifluoroethyl)-4,5,6,7-tetrahydro-1H-indazol-1-yl)-2-(4-hydroxycyclohexylamino)benzamide;
4-(6,6-dimethyl-4-oxo-3-(trifluoromethyl)-4,5,6,7-tetrahydro-1H-indazol-1-yl)-2-(2-hydroxycyclopentylamino)benzamide;
2-(cyclopentylamino)-4-(6,6-dimethyl-4-oxo-3-(trifluoromethyl)-4,5,6,7-tetrahydro-1H-indazol-1-yl)benzamide;
2-(tetrahydrofuran-3-ylamino)-4-(3,6,6-trimethyl-4-oxo-4,5,6,7-tetrahydro-1H-indol-1-yl)benzamide;
4-(6,6-dimethyl-4-oxo-3-(trifluoromethyl)-4,5,6,7-tetrahydro-1H-indol-1-yl)-2-(tetrahydro-2H-pyran-4-ylamino)benzamide;
4-(6,6-dimethyl-4-oxo-3-(trifluoromethyl)-4,5,6,7-tetrahydro-1H-indol-1-yl)-2-(tetrahydrofuran-3-ylamino)benzamide;
2-(cyclopentylamino)-4-(3,6,6-trimethyl-4-oxo-4,5,6,7-tetrahydro-1H-indol-1-yl)benzamide;
4-(6,6-dimethyl-4-oxo-3-(trifluoromethyl)-4,5,6,7-tetrahydro-1H-indol-1-yl)-2-(4-hydroxycyclohexylamino)benzamide;
2-(cyclopentylamino)-4-(6,6-dimethyl-4-oxo-3-(trifluoromethyl)-4,5,6,7-tetrahydro-1H-indol-1-yl)benzamide;
2-(4,4-difluorocyclohexylamino)-4-(3,6,6-trimethyl-4-oxo-4,5,6,7-tetrahydro-1H-indol-1-yl)benzamide;
2-(4,4-difluorocyclohexylamino)-4-(3,6,6-trimethyl-4-oxo-4,5,6,7-tetrahydro-1H-indazol-1-yl)benzamide;
3-(4,4-difluorocyclohexylamino)-5-(3,6,6-trimethyl-4-oxo-4,5,6,7-tetrahydro-1H-indol-1-yl)picolinamide;
3-(2-hydroxycyclohexylamino)-5-(3,6,6-trimethyl-4-oxo-4,5,6,7-tetrahydro-1H-indol-1-yl)picolinamide;
5-(6,6-dimethyl-4-oxo-3-(trifluoromethyl)-4,5,6,7-tetrahydro-1H-indol-1-yl)-3-(2-hydroxycyclohexylamino)picolinamide;
3-(4,4-difluorocyclohexylamino)-5-(6,6-dimethyl-4-oxo-3-(trifluoromethyl)-4,5,6,7-tetrahydro-1H-indol-1-yl)picolinamide;
2-(cyclohexylamino)-4-(3,6,6-trimethyl-4-oxo-4,5,6,7-tetrahydro-1H-indazol-1-yl)benzamide;
2-(4,4-difluorocyclohexylamino)-4-(6,6-dimethyl-4-oxo-3-(trifluoromethyl)-4,5,6,7-tetrahydro-1H-indol-1-yl)benzamide;
4-(2-carbamoyl-3-fluoro-5-(3,6,6-trimethyl-4-oxo-4,5,6,7-tetrahydro-1H-indazol-1-yl)phenylamino)cyclohexyl 2-(tert-butoxycarbonylamino)acetate;
2-(2-carbamoyl-5-(6,6-dimethyl-4-oxo-3-(trifluoromethyl)-4,5,6,7-tetrahydro-1H-indazol-1-yl)phenylamino)cyclopentyl 2-(tert-butoxycarbonylamino)acetate;
or a pharmaceutically acceptable salt thereof.

20. A compound according to claim 1 which is 2-(cyclopentylamino)-4-(3,6,6-trimethyl-4-oxo-4,5,6,7-tetrahydro-1H-indazol-1-yl)benzamide;
2-(cycloheptylamino)-4-(3,6,6-trimethyl-4-oxo-4,5,6,7-tetrahydro-1H-indazol-1-yl)benzamide;
or a pharmaceutically acceptable salt thereof.

21. A compound according to claim 1 which is 5-(6,6-dimethyl-4-oxo-3-(trifluoromethyl)-4,5,6,7-tetrahydro-1H-indazol-1-yl)-3-((trans)-4-hydroxycyclohexylamino)picolinamide or a pharmaceutically acceptable salt thereof.

22. A method of treating a disease or disorder related to cell proliferation comprising administering a therapeutically effective amount of a compound of claim 1, or a pharmaceutically acceptable salt thereof, to a patient in need of such treatment.

23. A method of treating a disease or disorder related to Heat-shock protein 90 comprising administering a therapeutically effective amount of a compound of claim 1, or a pharmaceutically acceptable salt thereof, to a patient in need of such treatment.

24. A method of treating cancer comprising administering a therapeutically effective amount of a compound of claim 1, or a pharmaceutically acceptable salt thereof, to a patient in need of such treatment.

25. A pharmaceutical composition comprising at least one compound or salt according to claim 1 and a pharmaceutically acceptable solvent, carrier, excipient, adjuvant or a combination thereof.

26. A method of treating cancer, inflammation, infection, angiogenesis, or arthritis comprising administering to a patient in need of such treatment a therapeutically effective amount of a compound or salt of claim 1.

27. A method for treating a subject suffering from a disease or disorder of proteins that are either client proteins for HSP-90 or indirectly affect its client proteins, wherein disorder is selected from the group of inflammatory diseases, infections, autoimmune disorders, stroke, ischemia, cardiac disorders, neurological disorders, fibrogenetic disorders, proliferative disorders, tumors, leukemias, neoplasms, cancers, carcinomas, metabolic diseases, malignant disease, scleroderma, polymyositis, systemic lupus, rheumatoid arthritis, liver cirrhosis, keloid formation, interstitial nephritis, pulmonary fibrosis, and sepsis, comprising administering to a subject in need of such treatment a therapeutically effective amount of a compound or salt of claim 1.

28. A method of reducing the level of infection in a subject where the infection is caused by an organism selected from Plasmodium species, the method comprising administering to an infected subject an effective amount of a compound or salt according to claim 1.

29. A method for treating a fungal infection in a patient in need of such treatment, comprising administering an effective amount of a compound or salt according to claim 1 and an optional anti-fungal agent or drug.

30. A method according to claim 26, for the treatment of cancer and further comprising administration of (a) at least one additional anti-cancer agent or composition or (b) radiation therapy.

31. A method of treating a patient suffering from a viral infection comprising administering to the patient a therapeutically effective amount of a compound of claim 1.

32. A method of preparing a compound of the formula 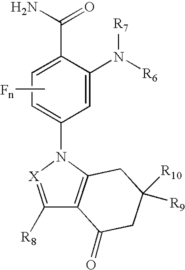
or a pharmaceutically acceptable salt thereof, wherein n is 0, 1, 2, or 3; and R₆, R₇, R₈, R₉, R₁₀, and X are as defined in claim 1, the method comprising:
(1) treating a compound of the formula 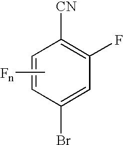
with a compound of formula HNR₆R₇ and a base to provide a compound of the formula 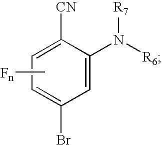
(2) treating the product or (1) with N,N′-dimethylethane-1,2-diamine, Cu⁺¹, a base, and a compound of formula 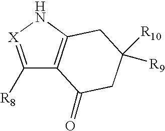
to provide a compound of formula 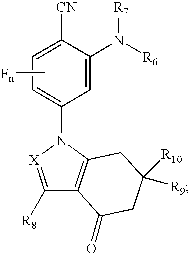
(3) treating the product of (2) with aqueous base and a peroxide to provide a compound of the formula

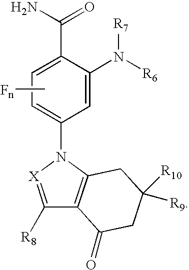

33. A method of preparing a compound of the formula 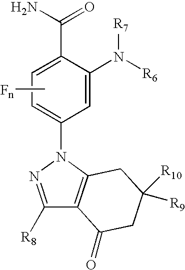
or a pharmaceutically acceptable salt thereof, wherein n is 0, 1, 2, or 3; and R₆, R₇, R₈, R₉, and R₁₀ are as defined in claim 1, the method comprising:
(1) treating a compound of the formula 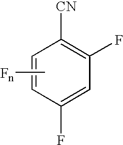
with a base and a compound of formula H₂NNH—R, wherein R is H or tert-butoxycarbonyl (Boc) to provide a compound of the formula 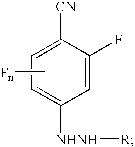
(2) treating the product of (1) with an acid, heat, and a compound of the formula 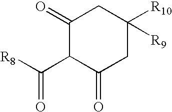
to provide a compound of the formula 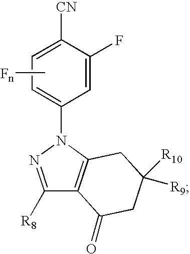
(3) treating the product of (2) with a base and a compound of formula HNR₆R₇ to provide a compound of formula 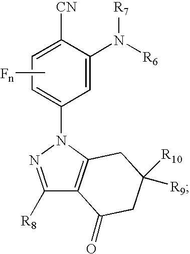
(4) treating the product of (3) with aqueous base and a peroxide to provide a compound of formula

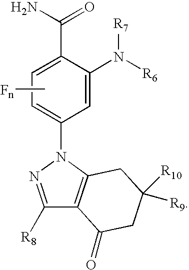

34. A method of preparing a compound of formula 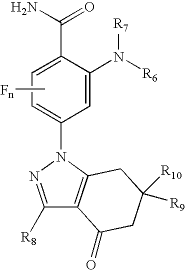
or a pharmaceutically acceptable salt thereof, wherein
n is 0, 1, 2, or 3; and R₆, R₇, R₈, R₉, and R₁₀ are as defined in claim 1, the method comprising:
(1) treating a compound of the formula 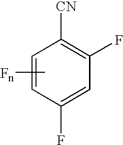
with a base and a compound of formula HNR₆R₇ to provide a compound of the formula 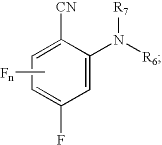
(2) treating the product of (1) with a base and a compound of formula H₂NNH—R, wherein R is H or tert-butoxycarbonyl, to provide a compound of the formula 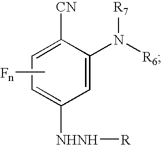
(3) treating the product of (2) with an acid, heat, and a compound of formula 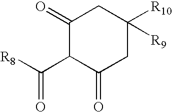
to provide a compound of the formula 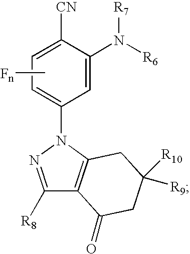
(4) treating the product of (3) with aqueous base and a peroxide to provide a compound of the formula

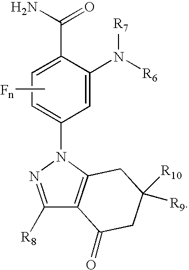

35. A method of preparing a compound of formula 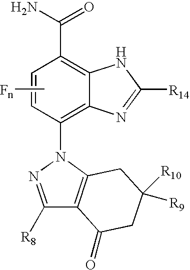
or a pharmaceutically acceptable salt thereof, wherein R₈, R₉, and R₁₀ are as defined in claim 1; and R₁₄ is H, (C₁-C₈)alkyl, (C₃-C₈) cycloalkyl, aryl, aryl (C₁-C₈) alkyl, heteroaryl, heteroaryl (C₁-C₈) alkyl, heterocyclyl, or heterocycle (C₁-C₈) alkyl, the method comprising:
(1) treating a compound of the formula 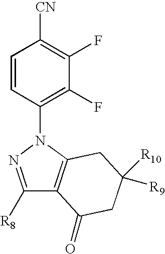
with sodium azide to provide a compound of the formula 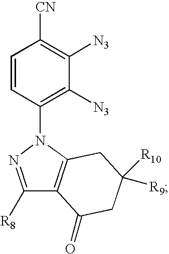
(2) treating the product of (1) with a metal catalyst under a hydrogen atmosphere, and optionally with pressure to provide a compound of the formula 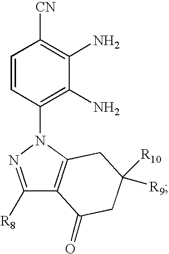
(3) treating the product of (2) with ytterbium triflate and a compound of formula (R₁₀)₃CR₁₄, wherein R′ is methyl or ethyl, to provide a compound of the formula 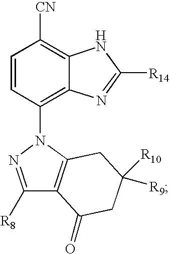
(4) treating the product of (3) with aqueous base and a peroxide to provide a compound of the formula [chemical structure]
